(19) United States
(12) Patent Application Publication
(10) Pub. No.: US 2021/0253562 A1
(43) Pub. Date: Aug. 19, 2021

(54) ACYLSUFONAMIDE COMPOUNDS USEFUL AS EP3 RECEPTOR ANTAGONISTS

(71) Applicant: Janssen Pharmaceutica NV, Beerse (BE)

(72) Inventors: Bin Zhu, Newtown, PA (US); Mark J. Macielag, Gwynedd Valley, PA (US)

(21) Appl. No.: 17/250,146

(22) PCT Filed: Jun. 28, 2019
(86) PCT No.: PCT/IB2019/055516

§ 371 Date: Dec. 7, 2020

Related U.S. Application Data
(60) Provisional application No. 62/693,466, filed on Jul. 3, 2018.

(51) Int. Cl. C07D 409/12; C07D 231/56; C07D 405/12; C07D 417/12; C07D 401/12; C07D 409/14; C07D 401/04; C07D 403/12; C07D 413/12; C07D 231/54; C07D 491/052; C07D 498/04; C07D 261/20; C07D 471/04; A61P 3/10

(52) U.S. Cl. CPC C07D 409/12; A61P 3/10; C07D 405/12; C07D 417/12; C07D 401/12; C07D 409/14; C07D 401/04; C07D 403/12; C07D 413/12; C07D 231/54; C07D 491/052; C07D 498/04; C07D 261/20; C07D 471/04; C07D 231/56

(57) ABSTRACT

The present invention is directed to acylsulfonamide derivatives, pharmaceutical compositions containing them and their use as antagonists of the EP3 receptor, for the treatment of for example, impaired oral glucose tolerance, elevated fasting glucose, Type II Diabetes Mellitus, Syndrome X (also known as Metabolic Syndrome) and related disorders and complications thereof.

### FIELD OF THE INVENTION

The present invention is directed to acylsulfonamide derivatives, pharmaceutical compositions containing them and their use as antagonists of the EP3 receptor, for the treatment of for example, impaired oral glucose tolerance, elevated fasting glucose, Type II Diabetes Mellitus, Syndrome X (also known as Metabolic Syndrome) and related disorders and complications thereof.

### BACKGROUND OF THE INVENTION

Type I diabetes represents about 5-10% of all diabetes cases and occurs as a result of destruction of the pancreatic beta cells, which produce the hormone insulin, by the body's own immune system. The patients are completely dependent on insulin treatment for survival. Type II diabetes is more common (90-95% of all cases). It starts as insulin resistance particularly in the cells of liver, muscle, and adipose tissue that become resistant to the effects of insulin in stimulating glucose and lipid metabolism. As the disease progresses the pancreas gradually loses its ability to produce insulin and if not properly controlled with medication it may lead to pancreatic β-cell failure requiring complete dependence on insulin. While there are five different categories of Type II diabetes medications, they may be ineffective and/or cause undesirable adverse effects such as hypoglycemia, gastrointestinal disturbances, lactic acidosis, weight gain, edema, and anemia.

There continues to be a need to introduce new effective treatments that may be used less frequently, preferably causing fewer side effects and can act either by increasing the endogenous insulin secretion or independently from the actions of insulin.

Prostanoid receptors consist of EP, FP, IP, TP and DP receptors. The EP receptor family is divided into four distinct subtypes EP1, EP2, EP3 and EP4. The EP3 receptor is a 7-transmembrane G-protein coupled receptor found in various human tissues including the kidney, uterus, bladder, stomach and brain. Prostaglandin E2 (PGE2), a primary product of arachidonic acid metabolism by the cyclooxygenase pathway, is the natural ligand of EP3 as well as other EP receptor subtypes. Clinical studies have provided strong evidence of the role of increased levels of PGE2 as a contributor to defective insulin secretion in diabetic patients. Recently, the functional link between PGE2 suppression of glucose-stimulated insulin secretion (GSIS) and the EP3 receptor was confirmed using β-cell lines and isolated islets. It is hypothesized that increased PGE2 signaling through the EP3 receptor might be coincident with the development of diabetes and contribute to β-cell dysfunction. Therefore, EP3 receptor antagonists, may be an effective treatment for Type I and Type II Diabetes Mellitus, by relieving the inhibitory action of PGE2 to partially restore defective GSIS in diabetic patients. EP3 receptor antagonists may also be useful for the treatment of bladder over-activity, cerebrovascular disease, coronary artery disease, hypertension, neurodegenerative disorders, pain, premature labor, restenosis, thrombosis and colon cancer (KAWAMORI, T., et al., “Prostanoid receptors and colon carcinogenesis”, Carcinogenesis and Modification of Carcinogenesis (2005), pp 243-251.).

SINGH, J., et al., in J. Med. Chem., 2010, pp 18-36, Vol. 53 describe selective prostanoid EP3 receptor antagonists, including (2E)-3-[I-[(2,4-Dichlorophenyl)methyl]-5-fluoro-3-methyl-IH-indol-7-yl]-N-[(4,5-dichloro-2-thienyl)sulfonyl]-2-propenamide (DG-041), as antiplatelet agent that do not promote prolonged bleeding. ZHOU, N., et al., in Bioorg & Med. Chem. Ltrs., 2009, pp 123-126, Vol 19 describe 3,4-disubstituted indole acylsulfonamides, selective human EP3 receptor antagonists. HATEGAN, G., et al., in Bioorg & Med. Chem. Ltrs., 2009, pp 6797-6800, Vol 19 describe heterocyclic 1,7-disubstituted indole sulfonamides, selective human EP3 receptor antagonists. GALLANT, M., et al., in Bioorg & Med. Chem. Ltrs., 2002, pp 2583-2586, Vol 12 describe the structure-activity relationship of biaryl acylsulfonamide analogues on the human EP3 prostanoid receptor.

SINGH, J., et al., in U.S. Pat. No. 7,589,397, issued Oct. 6, 2009 describe acyl sulfonamide, peri-substituted, fused bicyclic ring compounds useful for the treatment or prophylaxis of a prostagladin-mediated disease or condition.

### SUMMARY OF THE INVENTION

The present invention is directed to compounds of formula (I)

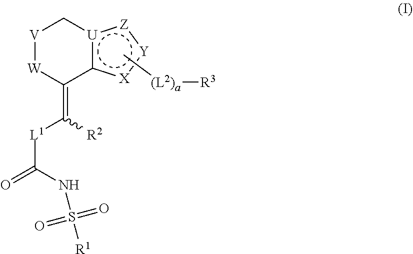

wherein

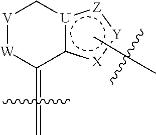

is an 8- to 10-membered, partially unsaturated ring structure selected from the group consisting of

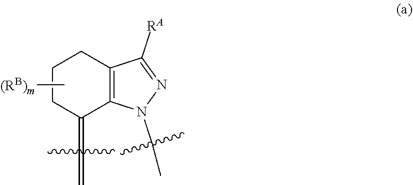

(wherein U is C, V is C(R^{B})₂, W is C(R^{B})₂, X is N, Y is N and Z is C(R^{A}) and wherein -(L²)ₐ-R³ is bound to X);

wherein R^{A} is selected from the group consisting of hydrogen, C₁₋₂alkyl and fluorinated C₁₋₂alkyl;

m is an integer from 0 to 2;

each R^{B} is selected from the group consisting of fluoro and C₁₋₂alkyl;

provided that when R^{A} is other than hydrogen, then m is 0;

provided further that each R^{B} is bound at the 5- or 6-position of the 4,5,6,7-tetrahydroindazole ring structure; and that when m is 2, then both R^{B} groups are bound to the same 5- or 6-position carbon atom;

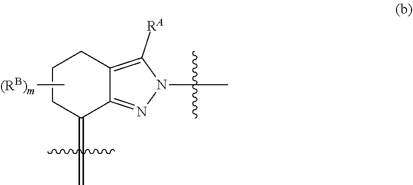

(wherein U is C, V is CH₂, W is CH₂, X is N, Y is N and Z is CH and wherein -(L²)ₐ-R³ is bound to Y);

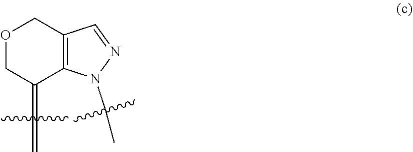 [SMILES: *n1ncc2c1C(=*)COC2]

(wherein U is C, V is O, W is CH₂, X is N, Y is N and Z is CH and wherein -(L²)ₐ-R³ is bound to X);

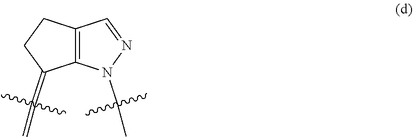 [SMILES: *n1ncc2c1C(=*)CC2]

(wherein U is C, V is absent, W is CH₂, X is N, Y is N and Z is CH and wherein -(L²)ₐ-R³ is bound to X);

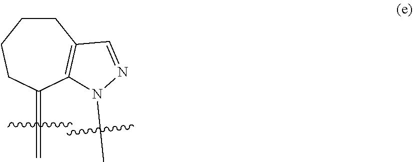 [SMILES: *n1ncc2c1C(=*)CCCC2]

(wherein U is C, V is CH₂CH₂, W is CH₂, X is N, Y is N and Z is CH and wherein -(L²)ₐ-R³ is bound to X);

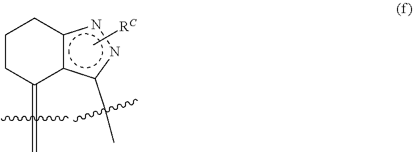

(wherein U is C, V is CH₂, W is CH₂, X is C, Y is N and Z is N and wherein -(L²)ₐ-R³ is bound to X);

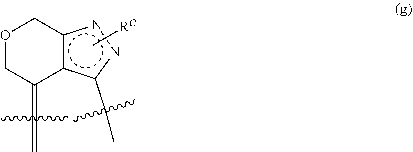

(wherein U is C, V is 0, W is CH₂, X is C, Y is N and Z is N and wherein -(L²)ₐ-R³ is bound to X);

wherein R^{c} is bound to either nitrogen atom and is selected from the group consisting of hydrogen, C₁₋₂ alkyl and —(C₁₋₂ alkyl)-O—(C₁₋₂alkyl),

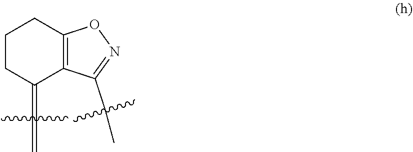 [SMILES: *c1noc2c1C(=*)CCC2]

(wherein U is C, V is CH₂, W is CH₂, X is C, Y is N and Z is O and wherein -(L²)ₐ-R³ is bound to X);

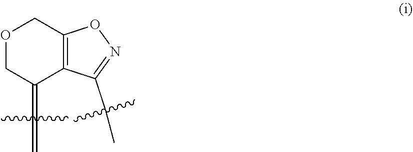 [SMILES: *c1noc2c1C(=*)COC2]

(wherein U is C, V is O, W is CH₂, X is C, Y is N and Z is O and wherein -(L²)ₐ-R³ is bound to X); and

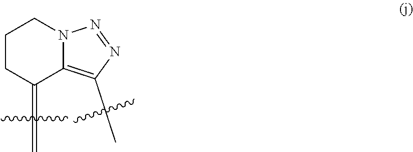 [SMILES: *c1nnn2c1C(=*)CCC2]

(wherein U is N, V is CH₂, W is CH₂, X is C, Y is N and Z is N and wherein -(L²)ₐ-R³ is bound to X);

R¹ is selected from the group consisting of C₁₋₄alkyl, phenyl, naphthyl and heterocyclyl;

wherein the phenyl, naphthyl or heterocyclyl is optionally substituted with one or more substituents independently selected from the group consisting of halogen, oxo, C₁₋₄alkyl, fluorinated C₁₋₂alkyl, C₁₋₄alkoxy, fluorinated C₁₋₂alkoxy, cyano, nitro, —NR^{D}R^{E}, —C(O)—NR^{D}R^{E}, —NH—C(O)—C₁₋₄alkyl, —S—C₁₋₂alkyl and C₃₋₅cycloalkyl;

wherein R^{D} and R^{E} are each independently selected from the group consisting of hydrogen, methyl and ethyl;

L¹ is selected from the group consisting of —CH₂—, —CH₂CH₂—, —CH═CH—, —O—CH₂—, —NH—CH₂—, —N(CH₃)—CH₂— and —N(CH₂CH₃)—CH₂— wherein the —CH═ or —CH₂— portion of the L¹ group is bound to the double bond;

R² is selected from the group consisting of hydrogen and fluoro;

a is an integer from 0 to 1;

L² is selected from the group consisting of —CH₂— and —CH₂CH₂—;

R³ is selected from the group consisting of C₁₋₄alkyl, phenyl, naphthyl and heterocyclyl;

wherein the phenyl, naphthyl or heterocyclyl is optionally substituted with one or more substituents independently selected from the group consisting of halogen, oxo, C₁₋₄alkyl, fluorinated C₁₋₄alkyl, C₁₋₄alkoxy, fluorinated C₁₋₄alkoxy, cyano, —NR^{F}R^{G}, —C(O)—NR^{F}R^{G}, —NH—C(O)—C₁₋₄alkyl, —S—C₁₋₂alkyl, —SO—C₁₋₂alkyl, —SO₂—C₁₋₂alkyl, phenyl, benzyl, phenylethyl, and 5- to 6-membered heteroaryl;

wherein R^{F} and R^{G} are each independently selected from the group consisting of hydrogen and C₁₋₄alkyl;

and wherein the phenyl, benzyl, phenylethyl or 5- to 6-membered heteroaryl is further optionally substituted with one to two substituents independently selected from the group consisting of halogen and C₁₋₄alkyl;

and stereoisomers and pharmaceutically acceptable salts thereof.

The present invention is further directed to processes for the preparation of the compounds of formula (I). The present invention is further directed to a product prepared according to the process described herein.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

Exemplifying the invention are methods of treating a disorder mediated by the EP3 receptor (selected from the group consisting Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders (such as Alzheimer's disease, intracerebral hemorrhage, and the like), non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain (for example, arthritic pain, neuropathic pain, and the like) and cancer (for example, prostate cancer, pancreatic cancer, colon cancer, liver cancer, thyroid cancer, breast cancer, and the like)) comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder mediated by the EP3 receptor (selected from the group consisting of Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders (such as Alzheimer's disease, intracerebral hemorrhage, and the like), non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain (for example, arthritic pain, neuropathic pain, and the like) and cancer (for example, prostate cancer, pancreatic cancer, colon cancer, liver cancer, thyroid cancer, breast cancer, and the like)).

In another embodiment, the present invention is directed to a composition comprising a compound of formula (I) for the treatment of a disorder mediated by the EP3 receptor (selected from the group consisting of Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders (such as Alzheimer's disease, intracerebral hemorrhage, and the like), non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain (for example, arthritic pain, neuropathic pain, and the like) and cancer (for example, prostate cancer, pancreatic cancer, colon cancer, liver cancer, thyroid cancer, breast cancer, and the like)).

Another example of the invention is the use of any of the compounds described herein in the preparation of a medicament for treating: (a) Type I diabetes mellitus, (b) impaired glucose tolerance (IGT), (c) impaired fasting glucose (IFG), (d) gestational diabetes, (e) Type II diabetes mellitus, (f) Syndrome X (also known as Metabolic Syndrome), (g) obesity, (h) nephropathy, (i) neuropathy, (j) retinopathy, (k) restenosis, (l) thrombosis, (m) coronary artery disease, (n) hypertension, (o) angina, (p) atherosclerosis, (q) heart disease, (r) heart attack, (s) ischemia, (t) stroke, (u) nerve damage or poor blood flow in the feet, (v) neurodegenerative disorders (such as Alzheimer's disease, intracerebral hemorrhage, and the like), (w) non-alcoholic steatohepatitis (NASH), (x) non-alcoholic fatty liver disease (NAFLD), (y) liver fibrosis, (z) cataracts, (aa) polycystic ovarian syndrome, (ab) premature labor, (ac) irritable bowel syndrome, (ad) bladder over-activity, (ae) inflammation, (af) pain (for example, arthritic pain, neuropathic pain, and the like) and (ag) cancer (for example, prostate cancer, pancreatic cancer, colon cancer, liver cancer, thyroid cancer, breast cancer, and the like), in a subject in need thereof.

In another example, the present invention is directed to a compound as described herein for use in a methods for treating a disorder selected from the group consisting of Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders (such as Alzheimer's disease, intracerebral hemorrhage, and the like), non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain (for example, arthritic pain, neuropathic pain, and the like) and cancer (for example, prostate cancer, pancreatic cancer, colon cancer, liver cancer, thyroid cancer, breast cancer, and the like), in a subject in need thereof.

### DETAILED DESCRIPTION OF THE INVENTION

The present invention is directed to compounds of formula (I)

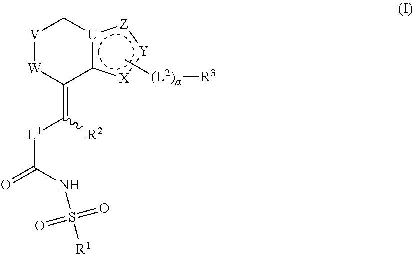

wherein

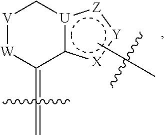

R¹, L¹, R², a, L² and R³ are as herein defined; and stereoisomers and pharmaceutically acceptable salts thereof. The compounds of formula (I) of the present invention are antagonists of the EP3 receptor, useful in the treatment of disorders and conditions that respond to antagonism of the EP3 receptor, including, but not limited to: Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders (such as Alzheimer's disease, intracerebral hemorrhage, and the like), non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain (for example, arthritic pain, neuropathic pain, and the like) and cancer (for example, prostate cancer, pancreatic cancer, colon cancer, liver cancer, thyroid cancer, breast cancer, and the like).

In an embodiment, the present invention is directed to compounds of formula (I-A)

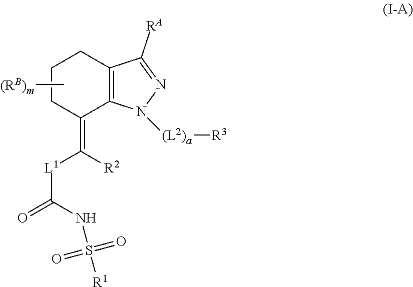

and stereoisomers and pharmaceutically acceptable salts thereof. In another embodiment, the present invention is directed to compounds of formula (I-B)

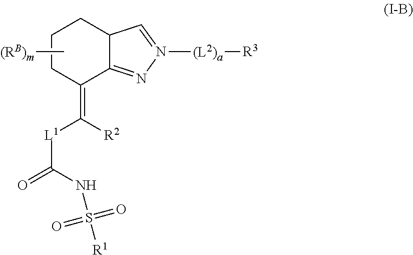

and stereoisomers and pharmaceutically acceptable salts thereof. In another embodiment, the present invention is directed to compounds of formula (I-C)

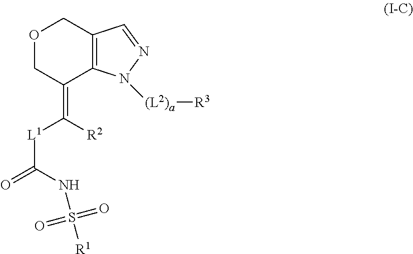

and stereoisomers and pharmaceutically acceptable salts thereof. In another embodiment, the present invention is directed to compounds of formula (I-D)

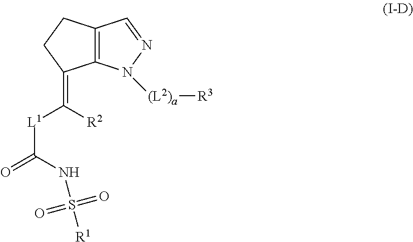

and stereoisomers and pharmaceutically acceptable salts thereof. In another embodiment, the present invention is directed to compounds of formula (I-E)

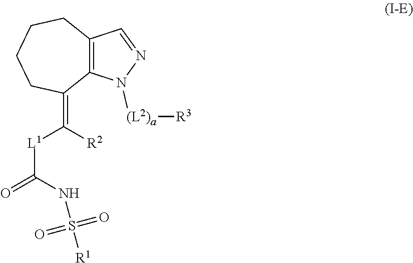

and stereoisomers and pharmaceutically acceptable salts thereof. In another embodiment, the present invention is directed to compounds of formula (I-F)

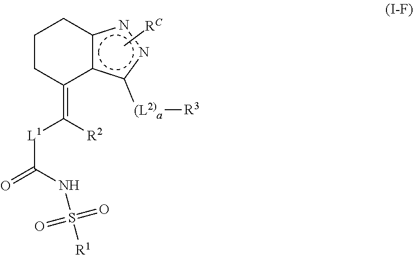

and stereoisomers and pharmaceutically acceptable salts thereof. In another embodiment, the present invention is directed to compounds of formula (I-G)

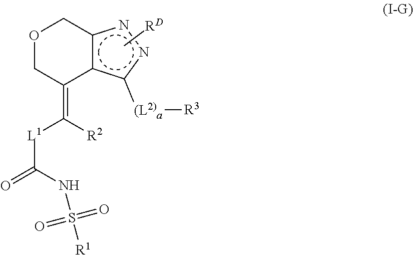

and stereoisomers and pharmaceutically acceptable salts thereof. In another embodiment, the present invention is directed to compounds of formula (I-H)

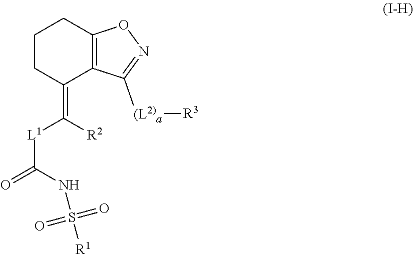

and stereoisomers and pharmaceutically acceptable salts thereof. In another embodiment, the present invention is directed to compounds of formula (I-I)

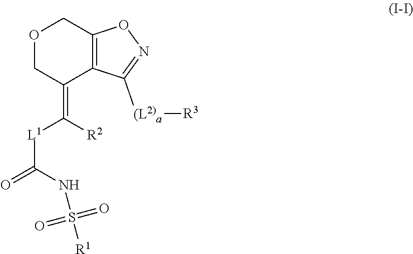

and stereoisomers and pharmaceutically acceptable salts thereof. In another embodiment, the present invention is directed to compounds of formula (I-J)

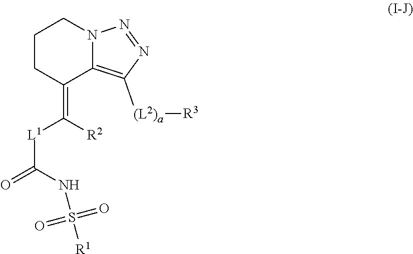

and stereoisomers and pharmaceutically acceptable salts thereof.

In certain embodiments, the present invention is directed to compounds of formula (I) wherein

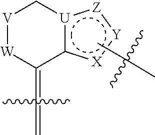

is any one or more 8- to 10-membered, partially unsaturated ring structures selected from the group consisting of

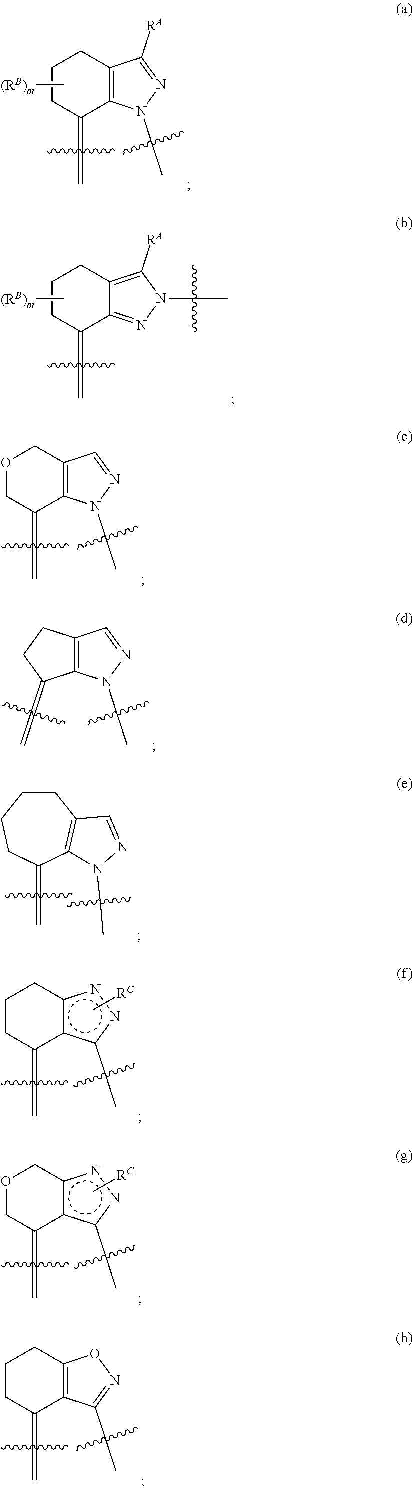

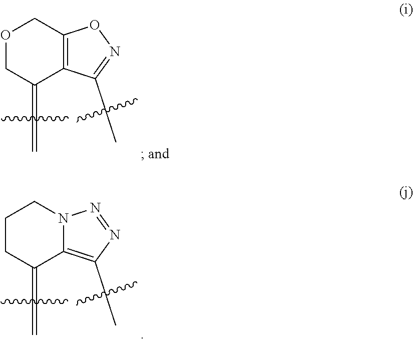 [SMILES: *c1noc2c1C(=*)COC2 ; "; and" ; *c1nnn2c1C(=*)CCC2 ; "."]

In certain embodiments, the present invention is directed to compounds of formula (I) wherein

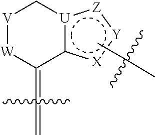

is an 8- to 10-membered, partially unsaturated ring structure selected from the group consisting of

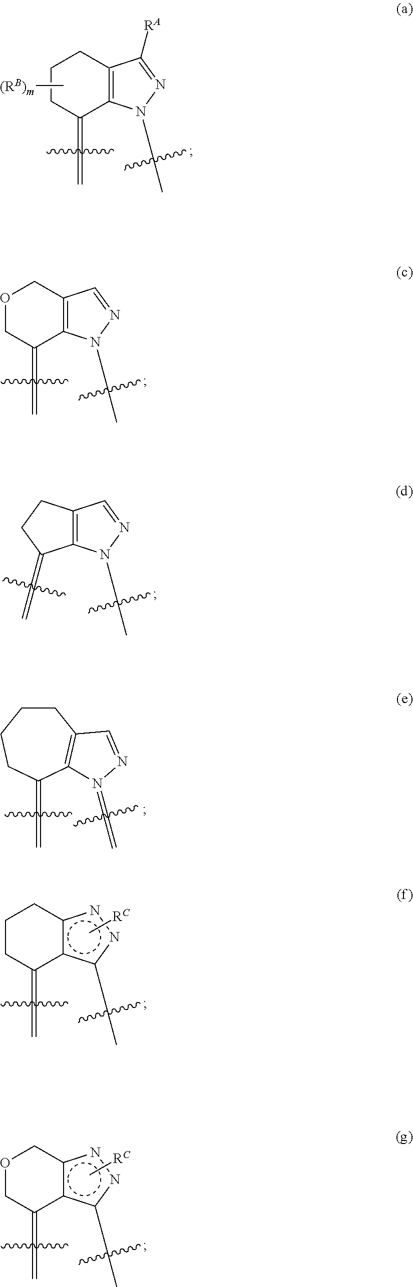

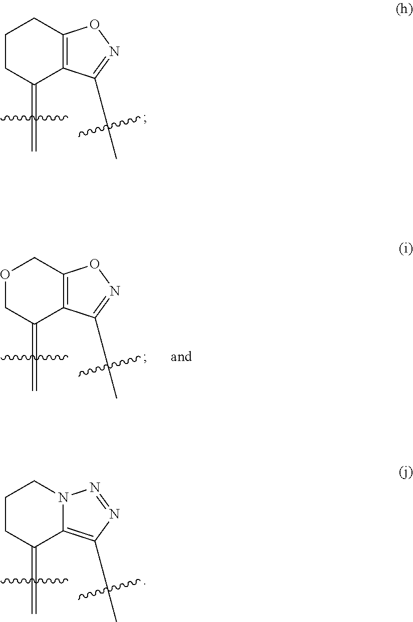 [SMILES: *c1noc2c1C(=*)CCC2 ; *c1noc2c1C(=*)COC2 ; *c1nnn2c1C(=*)CCC2]

In certain embodiments, the present invention is directed to compounds of formula (I) wherein

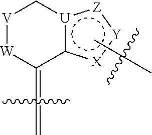

is an 8- to 10-membered, partially unsaturated ring structure selected from the group consisting of

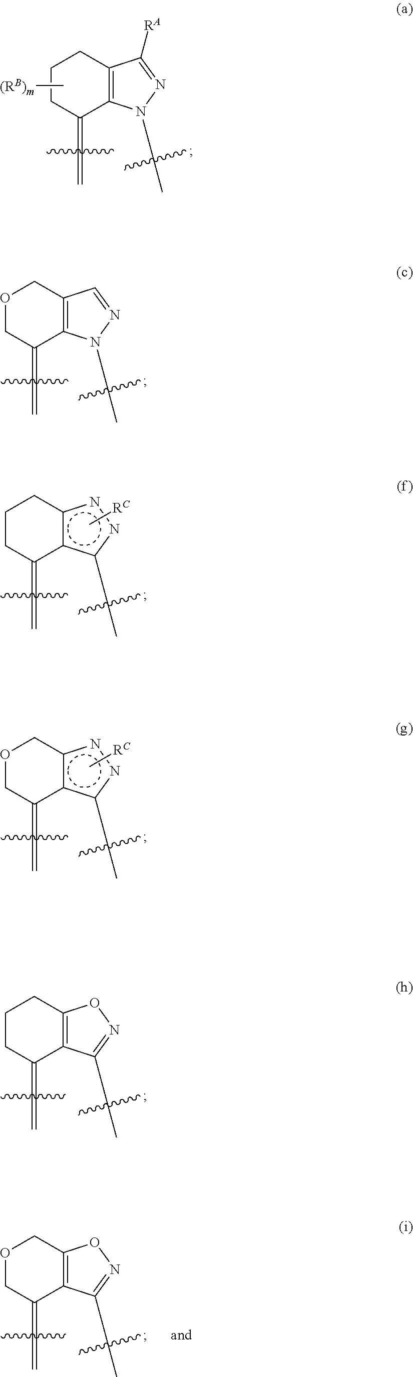

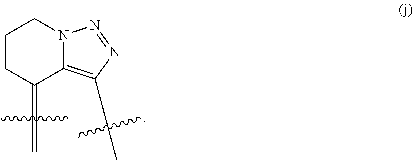 [SMILES: *c1nnn2c1C(=*)CCC2]

In certain embodiments, the present invention is directed to compounds of formula (I) wherein

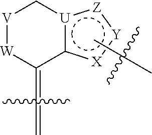

is an 8- to 10-membered, partially unsaturated ring structure selected from the group consisting of

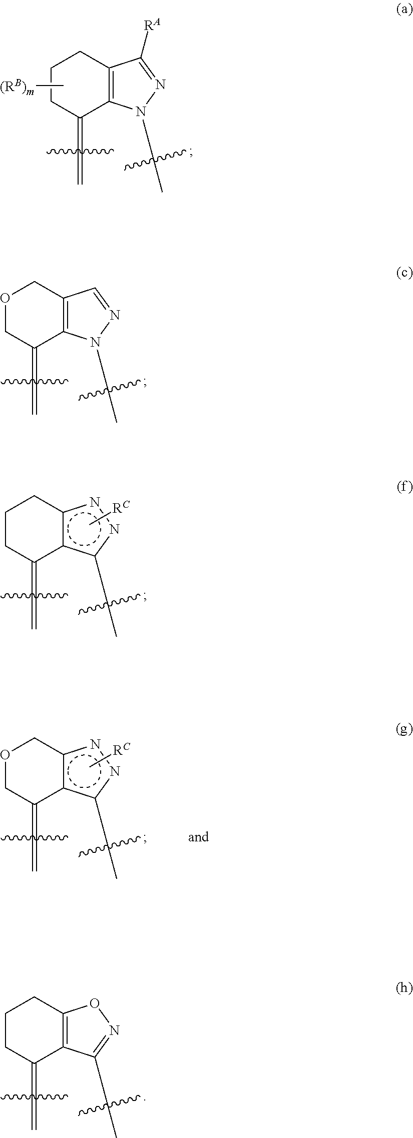

In certain embodiments, the present invention is directed to compounds of formula (I) wherein

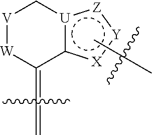

is an 8- to 10-membered, partially unsaturated ring structure selected from the group consisting of

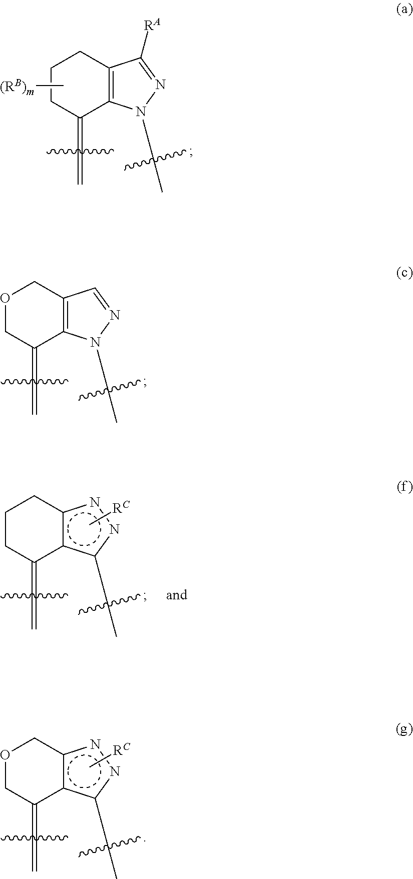

In certain embodiments, the present invention is directed to compounds of formula (I) wherein

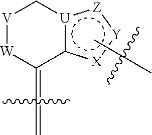

is an 8- to 10-membered, partially unsaturated ring structure selected from the group consisting of

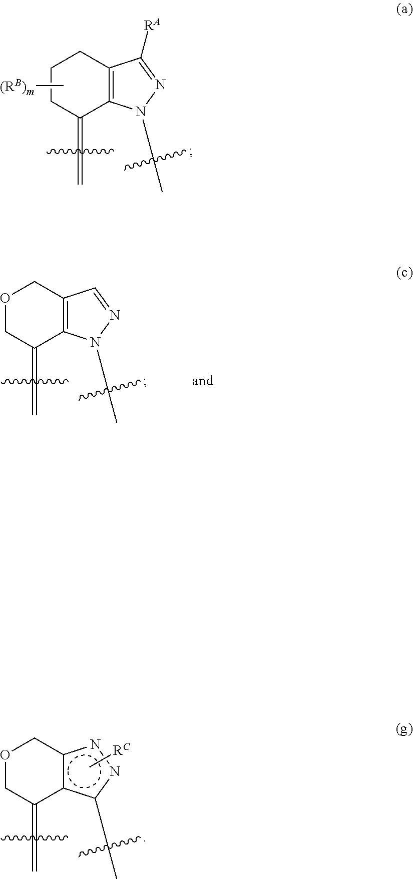

In certain embodiments, the present invention is directed to compounds of formula (I) wherein

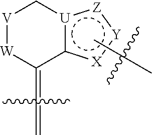

is an 8- to 10-membered, partially unsaturated ring structure selected from the group consisting of

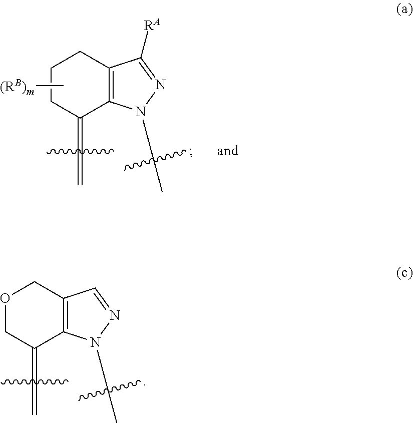

In certain embodiments, the present invention is directed to compounds of formula (I) wherein R^{A} is selected from the group consisting of hydrogen, C₁₋₂alkyl and fluorinated C₁₋₂alkyl. In certain embodiments, the present invention is directed to compounds of formula (I) wherein R^{A} is selected from the group consisting of hydrogen, methyl and difluoromethyl. In certain embodiments, the present invention is directed to compounds of formula (I) wherein R^{A} is selected from the group consisting of hydrogen and methyl. In certain embodiments, the present invention is directed to compounds of formula (I) wherein R^{A} is hydrogen.

In certain embodiments, the present invention is directed to compounds of formula (I) wherein m is an integer from 0 to 2. In certain embodiments, the present invention is directed to compounds of formula (I) wherein m is 0. In certain embodiments, the present invention is directed to compounds of formula (I) wherein m is an integer from 1 to 2. In certain embodiments, the present invention is directed to compounds of formula (I) wherein m is 1. In certain embodiments, the present invention is directed to compounds of formula (I) wherein m is 2.

In certain embodiments, the present invention is directed to compound of formula (I) wherein each R^{B} is fluoro. In certain embodiments, the present invention is directed to compound of formula (I) wherein each R^{B} is selected from the group consisting of 5-fluoro and 6-fluoro. In certain embodiments, the present invention is directed to compound of formula (I) wherein when m is 1, R^{B} is 6-fluoro; and when m is 2, both R^{B} groups are the same and are 5-fluoro.

In certain embodiments, the present invention is directed to compounds of formula (I) wherein R^{C} is selected from the group consisting of hydrogen, C₁₋₂alkyl and —(C₁₋₂alkyl)-O—(C₁₋₂alkyl). In certain embodiments, the present invention is directed to compounds of formula (I) wherein R^{C} is selected from the group consisting of hydrogen, methyl and —CH₂—OCH₃. In certain embodiments, the present invention is directed to compounds of formula (I) wherein R^{C} is bound to either nitrogen atom and is selected from the group consisting of hydrogen and methyl. In certain embodiments, the present invention is directed to compounds of formula (I) wherein R^{C} is hydrogen.

In certain embodiments, the present invention is directed to compounds of formula (I) wherein R¹ is selected from the group consisting of C₁₋₄alkyl, phenyl, naphthyl and heterocyclyl; wherein the phenyl, naphthyl or heterocyclyl is optionally substituted with one to two substituents independently selected from the group consisting of halogen, oxo, C₁₋₄alkyl, fluorinated C₁₋₂alkyl, C₁₋₄alkoxy, fluorinated C₁₋₂alkoxy, —NR^{D}R^{E}, —C(O)—NR^{D}R^{E} and —NH—C(O)—C₁₋₄alkyl, and wherein R^{D} and R^{E} are each independently selected from the group consisting of hydrogen and methyl.

In certain embodiments, the present invention is directed to compounds of formula (I) wherein R¹ is selected from the group consisting of C₁₋₄alkyl, phenyl, thienyl, thiazolyl, pyrazolyl, pyridiyl, indazolyl, benzofuryl, benzothienyl, benzothiazolyl, benzoxazolyl, quiunolinyl and 2,3-dihydrobenzo[b][1.4]dioxin-6-yl, wherein the phenyl, thienyl, thiazolyl, pyrazolyl, pyridiyl, indazolyl, benzofuryl, benzothienyl, benzothiazolyl, benzoxazolyl or quinolinyl is optionally substituted with one to two substituents independently selected from the group consisting of halogen, C₁₋₃alkyl, C₁₋₂alkoxy, oxo and —NH—C(O)—(C₁₋₂alkyl).

In certain embodiments, the present invention is directed to compounds of formula (I) wherein R¹ is selected from the group consisting of isopropyl, 3-chloro-phenyl, 4-chloro-phenyl, 3,4-difluoro-phenyl, 3,5-difluoro-phenyl, 2-methoxy-phenyl, 3-methoxy-phenyl, 4-methoxy-phenyl, 3,4-dimethoxy-phenyl, 2-methoxy-4-chloro-phenyl, 2-methoxy-5-bromo-phenyl, 2-methoxy-5-chloro-phenyl, 2-methoxy-5-fluoro-phenyl, 2-chloro-5-methoxy-phenyl, 2-fluoro-5-methoxy-phenyl, 3-chloro-4-methoxy-phenyl, 3-fluoro-4-methoxy-phenyl, 3-methoxy-4-chloro-phenyl, thien-2-yl, 5-chloro-thien-2-yl, 4,5-dichloro-thien-2-yl, 4,5-dimethyl-thienyl, 4-methyl-5-chloro-thien-2-yl, 2-methyl-thiazol-5-yl, 2,4-dimethyl-thiazol-5-yl, 1-isopropyl-thiazol-4-yl, 2-(methyl-carbonyl-amino)-4-methyl-thiazol-5-yl, 1-methyl-pyrazol-3-yl, 1-methyl-pyrazol-4-yl, 1,3-dimethyl-pyrazol-4-yl, 1-isopropyl-pyrazol-4-yl, 1,5-dimethyl-pyrazol-4-yl, 1-methyl-pyrazol-5-yl, 1,3-dimethyl-pyrazol-5-yl, 5-chloro-6-methyl-pyrid-3-yl, 6-methoxy-pyrid-3-yl, 1-methyl-indazol-5-yl, 1-methyl-indazol-6-yl, benzofur-2-yl, benzothien-2-yl, benzothiazol-2-yl, 2-methyl-benzoxazol-5-yl, 2-methoxy-benzoxazol-6-yl, benzoxazol-6-yl-2-one, quinolin-3-yl and 2,3-dihydrobenzo[b][1.4]dioxin-6-yl.

In certain embodiments, the present invention is directed to compounds of formula (I) wherein R¹ is selected from the group consisting of 3-chloro-phenyl, 4-chloro-phenyl, 3,4-difluoro-phenyl, 3,5-difluoro-phenyl, 3-methoxy-phenyl, 4-methoxy-phenyl, 2-methoxy-4-chloro-phenyl, 2-methoxy-5-bromo-phenyl, 2-methoxy-5-chloro-phenyl, 2-methoxy-5-fluoro-phenyl, 2-chloro-5-methoxy-phenyl, 2-fluoro-5-methoxy-phenyl, 3-chloro-4-methoxy-phenyl, 3-methoxy-4-chloro-phenyl, thien-2-yl, 5-chloro-thien-2-yl, 4,5-dichloro-thien-2-yl, 4,5-dimethyl-thienyl, 4-methyl-5-chloro-thien-2-yl, 2-methyl-thiazol-5-yl, 2,4-dimethyl-thiazol-5-yl, 1-methyl-pyrazol-3-yl, 1-methyl-pyrazol-4-yl, 1,3-dimethyl-pyrazol-4-yl, 1,5-dimethyl-pyrazol-4-yl, 1-methyl-pyrazol-5-yl, 1,3-dimethyl-pyrazol-5-yl, 5-chloro-6-methyl-pyrid-3-yl, 6-methoxy-pyrid-3-yl, 1-methyl-indazol-5-yl, 1-methyl-indazol-6-yl, benzofur-2-yl, benzothien-2-yl, benzothiazol-2-yl, 2-methoxy-benzoxazol-6-yl, benzoxazol-6-yl-2-one, quinolin-3-yl and 2,3-dihydrobenzo[b][1.4]dioxin-6-yl.

In certain embodiments, the present invention is directed to compounds of formula (I) wherein R¹ is selected from the group consisting of 4-chloro-phenyl, 3,4-difluoro-phenyl, 2-methoxy-4-chloro-phenyl, 2-methoxy-5-bromo-phenyl, 2-methoxy-5-chloro-phenyl, 3-chloro-4-methoxy-phenyl, 5-chloro-thien-2-yl, 4,5-dichloro-thien-2-yl, 4-methyl-5-chloro-thien-2-yl, 2,4-dimethyl-thiazol-5-yl, benzothien-2-yl, benzothiazol-2-yl, 2-methoxy-benzoxazol-6-yl and benzoxazol-6-yl-2-one.

In certain embodiments, the present invention is directed to compounds of formula (I) wherein R¹ is selected from the group consisting of 3-chloro-4-methoxy-phenyl, 5-chloro-thien-2-yl, 4,5-dichloro-thien-2-yl, 4-methyl-5-chloro-thien-2-yl, benzothiazol-2-yl, 2-methoxy-benzoxazol-6-yl and benzoxazol-6-yl-2-one.

In certain embodiments, the present invention is directed to compounds of formula (I) wherein R¹ is selected from the group consisting of 4-chloro-phenyl, 3-chloro-4-methoxy-phenyl, 4,5-dichloro-thien-2-yl 4-methyl-5-chloro-thien-2-yl and 1-methyl-pyrazol-4-yl.

In certain embodiments, the present invention is directed to compounds of formula (I) wherein L¹ is selected from the group consisting of —CH₂—, —CH₂CH₂—, —CH═CH—, —O—CH₂—, —NH—CH₂— and —N(CH₃)—CH₂—, wherein the —CH═ or —CH₂— portion of the L¹ group is bound to the double bond. In certain embodiments, the present invention is directed to compounds of formula (I) wherein L¹ is selected from the group consisting of —CH₂—, —CH₂CH₂—, —OCH₂—, —NH—CH₂— and —N(CH₃)—CH₂—, wherein the —CH₂— portion of the L¹ group is bound to the double bond. In certain embodiments, the present invention is directed to compounds of formula (I) wherein L¹ is selected from the group consisting of —CH₂CH₂—, —OCH₂— and —NH—CH₂—, wherein the —CH₂— portion of the L¹ group is bound to the double bond. In certain embodiments, the present invention is directed to compounds of formula (I) wherein L¹ is —NH—CH₂—, wherein the —CH₂— portion of the L¹ group is bound to the double bond.

In certain embodiments, the present invention is directed to compounds of formula (I) wherein R² is selected from the group consisting of hydrogen and fluoro. In certain embodiments, the present invention is directed to compounds of formula (I) wherein R² is hydrogen. In certain embodiments, the present invention is directed to compounds of formula (I) wherein R² is fluoro.

In certain embodiments, the present invention is directed to compounds of formula (I) wherein a is an integer from 0 to 1. n certain embodiments, the present invention is directed to compounds of formula (I) wherein a is 0. n certain embodiments, the present invention is directed to compounds of formula (I) wherein a is 1.

In certain embodiments, the present invention is directed to compounds of formula (I) wherein L² is selected from the group consisting of —CH₂— and —CH₂CH₂—. In certain embodiments, the present invention is directed to compounds of formula (I) wherein L² is —CH₂—. In certain embodiments, the present invention is directed to compounds of formula (I) wherein L² is —CH₂CH₂—.

In certain embodiments, the present invention is directed to compounds of formula (I) wherein R³ is selected from the group consisting of C₁₋₄alkyl, phenyl, naphthyl and heterocyclyl; wherein the phenyl, naphthyl or heterocyclyl is optionally substituted with one to two substituents independently selected from the group consisting of halogen, C₁₋₄alkyl, fluorinated C₁₋₄alkyl, C₁₋₄alkoxy, fluorinated C₁₋₄alkoxy, —NR^{F}R^{G}, —C(O)—NR^{F}R^{G}, —NH—C(O)—C₁₋₄alkyl, —S—C₁₋₂alkyl, —SO—C₁₋₂alkyl, —SO₂—C₁₋₂alkyl, phenyl, benzyl and 5- to 6-membered heteroaryl; wherein R^{F} and R^{G} are each independently selected from the group consisting of hydrogen and methyl; and wherein the phenyl, benzyl or 5- to 6-membered heteroaryl is further optionally substituted with one to two substituents independently selected from the group consisting of halogen and C₁₋₂alkyl.

In certain embodiments, the present invention is directed to compounds of formula (I) wherein R³ is selected from the group consisting of C₁₋₄alkyl, phenyl, naphthyl, pyrimidinyl, pyridyl, pyrazolyl and piperidinyl; wherein the phenyl, naphthyl, pyrimidinyl, pyrazolyl or piperidinyl is optionally substituted with one to two substituents independently selected from the group consisting of halogen, C₁₋₂alkyl, fluorinated C₁₋₂alkyl, C₁₋₂alkoxy, —SO₂—(C₁₋₂alkyl), phenyl, benzyl and pyridyl; and wherein the phenyl, benzyl or pyridyl substituent is further optionally substituted with one to two substituents independently selected from the group consisting of halogen.

In certain embodiments, the present invention is directed to compounds of formula (I) wherein R³ is selected from the group consisting of isopropyl, phenyl, 3-bromo-phenyl, 4-bromo-phenyl, 4-chloro-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, pyridimidin-2-yl, 5-bromo-pyrimidin-2-yl, 2-phenyl-pyrimidin-5-yl, 5-phenyl-pyrimidin-2-yl, 6-methoxy-pyrid-3-yl, 1-phenyl-pyrazol-4-yl, 1-methyl-piperidin-4-yl, 1-(4-fluoro-phenyl)-piperidin-4-yl, 1-(benzyl)-piperidin-4-yl and 1-(pyrid-2-yl)-piperidin-4-yl.

In certain embodiments, the present invention is directed to compounds of formula (I) wherein R³ is selected from the group consisting of phenyl, 3-bromo-phenyl, 4-bromo-phenyl, 4-chloro-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl, 1-phenyl-pyrazol-4-yl, 1-methyl-piperidin-4-yl, 1-(4-fluoro-phenyl)-piperidin-4-yl, 1-(benzyl)-piperidin-4-yl and 1-(pyrid-2-yl)-piperidin-4-yl.

In certain embodiments, the present invention is directed to compounds of formula (I) wherein R³ is selected from the group consisting of phenyl, 3-bromo-phenyl, 4-bromo-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl, 1-phenyl-pyrazol-4-yl, 1-(4-fluoro-phenyl)-piperidin-4-yl, 1-(benzyl)-piperidin-4-yl and 1-(pyrid-2-yl)-piperidin-4-yl.

In certain embodiments, the present invention is directed to compounds of formula (I) wherein R³ is selected from the group consisting of 4-bromo-phenyl, 2,4-dichloro-phenyl, 4-trifluoromethyl-phenyl, 4-methylsulfonyl-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl and 1-(4-fluoro-phenyl)-piperidin-4-yl.

In certain embodiments, the present invention is directed to compounds of formula (I) wherein R³ is selected from the group consisting of 4-bromo-phenyl, 2,4-dichloro-phenyl, 4-trifluoromethyl-phenyl, 4-methylsulfonyl-phenyl, 3-phenyl-phenyl, 4-phenyl-phenyl, naphth-1-yl, naphth-2-yl and 1-(4-fluoro-phenyl)-piperidin-4-yl.

In certain embodiments, the present invention is directed to compounds of formula (I) wherein R³ is selected from the group consisting of 2,4-dichloro-phenyl, 4-trifluoromethyl-phenyl and naphth-2-yl.

In certain embodiments, the present invention is directed to any one or more compounds of formula (I) selected from the group consisting of
(E)-4-chloro-N-((2-(1-(2,4-dichlorobenzyl)-1,4,5,6-tetrahydro-7H-indazol-7-ylidene)ethyl)carbamoyl)benzenesulfonamide,
(Z)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide,
(E)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide,
(Z)-4,5-dichloro-N-((2-(1-(2,4-dichlorobenzyl)-1,4,5,6-tetrahydro-7H-indazol-7-ylidene)-2-fluoroethyl)(methyl)carbamoyl)thiophene-2-sulfonamide,
E)-4,5-dichloro-N-((2-(1-(4-(trifluoromethyl)phenyl)-1,4,5,6-tetrahydro-7H-indazol-7-ylidene)ethyl)carbamoyl)thiophene-2-sulfonamide,
Z)-3-chloro-N-((2-(1-(2,4-dichlorobenzyl)-1,4,5,6-tetrahydro-7H-indazol-7-ylidene)-2-fluoroethyl)carbamoyl)-4-methoxybenzenesulfonamide,
(Z)-5-chloro-N-((2-(1-(2,4-dichlorobenzyl)-1,4,5,6-tetrahydro-7H-indazol-7-ylidene)-2-fluoroethyl)carbamoyl)-4-methylthiophene-2-sulfonamide,
(E)-N-((2-(1-(2,4-dichlorobenzyl)-1,4,5,6-tetrahydro-7H-indazol-7-ylidene)ethyl)carbamoyl)-1-methyl-1H-pyrazole-4-sulfonamide,
E)-5-chloro-N-((2-(1-(2,4-dichlorobenzyl)-1,4-dihydropyrano[4,3-c]pyrazol-7(6H)-ylidene)-2-fluoroethyl)carbamoyl)-4-methylthiophene-2-sulfonamide;
(Z)-5-chloro-N-((2-(3-(2,4-dichlorobenzyl)-1,5,6,7-tetrahydro-4H-indazol-4-ylidene)-2-fluoroethyl)carbamoyl)-4-methylthiophene-2-sulfonamide,
E)-5-chloro-N-((2-(3-(2,4-dichlorobenzyl)-1,7-dihydropyrano[3,4-c]pyrazol-4(5H)-ylidene)-2-fluoroethyl)carbamoyl)-4-methylthiophene-2-sulfonamide;

and stereoisomers and pharmaceutically acceptable salts thereof.

Additional embodiments of the present invention, include those wherein the substituents selected for one or more of the variables defined herein (i.e.

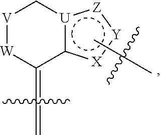

R¹, L¹, R², a, L² and R³, etc.) are independently selected to be any individual substituent or any subset of substituents independently selected from the complete list as defined herein.

Additional embodiments of the present invention, include those wherein the substituents selected for one or more of the variables defined herein (i.e.

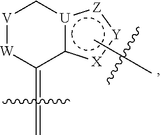

R¹, L¹, R², a, L² and R³, etc.) are independently selected to be any individual substituent or any subset of substituents independently selected from the substituents listed in Tables 1-7, below. In certain embodiments, the present invention is directed to any single compound or subset of compounds selected from the representative compounds listed in Tables 1-7, below.

Representative compounds of the present invention are as listed in Tables 1-7, below. In the Tables below, the column headed “stereo” lists the stereo-configuration of the double bond. Where one or more additional stereogenic center(s) are present in the listed compound, the compound was prepared as a racemic mixture.

TABLE 1
Representative Compounds of Formula (I)

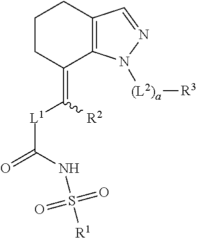

| ID No | Stereo | R¹ | L¹ | R² | (L²)ₐ | R³ |
|---|---|---|---|---|---|---|
| 2 | E | 4-chloro- | —OCH₂— | H | —CH₂— | 2,4-dichloro- |
| | | phenyl | | | | phenyl |
| 3 | E | 4,5-dichloro- | —CH₂— | H | —CH₂— | 2,4-dichloro- |
| | | thien-2-yl | | | | phenyl |
| 4 | E | 4-chloro- | —NH—CH₂— | H | —CH₂— | 2,4-dichloro- |
| | | phenyl | | | | phenyl |
| 5 | E | 4,5-dichloro- | —OCH₂— | H | —CH₂— | 2,4-dichloro- |
| | | thien-2-yl | | | | phenyl |
| 6 | E | 4,5-dichloro- | —CH═CH— | H | —CH₂— | 2,4-dichloro- |
| | | thien-2-yl | | | | phenyl |
| 7 | E | 4,5-dichloro- | —NH—CH₂— | H | —CH₂— | 2,4-dichloro- |
| | | thien-2-yl | | | | phenyl |
| 8 | E | 4,5-dichloro- | —CH₂—CH₂— | H | —CH₂— | 2,4-dichloro- |
| | | thien-2-yl | | | | phenyl |
| 9 | E | 3,5-difluoro- | —NH—CH₂— | H | —CH₂— | 2,4-dichloro- |
| | | phenyl | | | | phenyl |
| 10 | E | thien-2-yl | —NH—CH₂— | H | —CH₂— | 2,4-dichloro- |
| | | | | | | phenyl |
| 11 | E | 5-chloro- | —NH—CH₂— | H | —CH₂— | 2,4-dichloro- |
| | | thien-2-yl | | | | phenyl |
| 12 | E | quinolin-3-yl | —NH—CH₂— | H | —CH₂— | 2,4-dichloro- |
| | | | | | | phenyl |
| 13 | E | 4-methoxy- | —NH—CH₂— | H | —CH₂— | 2,4-dichloro- |
| | | phenyl | | | | phenyl |
| 14 | E | isopropyl | —CH₂— | H | —CH₂— | 2,4-dichloro- |
| | | | | | | phenyl |
| 15 | E | benzothiazol- | —NH—CH₂— | H | —CH₂— | 2,4-dichloro- |
| | | 2-yl | | | | phenyl |
| 16 | E | 2,3- | —NH—CH₂— | H | —CH₂— | 2,4-dichloro- |
| | | dihydrobenzo | | | | phenyl |
| | | [b][1,4]dioxin- | | | | |
| | | 6-yl | | | | |
| 17 | E | 5-chloro- | —CH₂— | H | —CH₂— | 2,4-dichloro- |
| | | thien-2-yl | | | | phenyl |
| 18 | E | 2,3- | —CH₂— | H | —CH₂— | 2,4-dichloro- |
| | | dihydrobenzo | | | | phenyl |
| | | [b][1,4]dioxin- | | | | |
| | | 6-yl | | | | |
| 19 | E | 3,5-difluoro- | —CH₂— | H | —CH₂— | 2,4-dichloro- |
| | | phenyl | | | | phenyl |
| 20 | E | thien-2-yl | —CH₂— | H | —CH₂— | 2,4-dichloro- |
| | | | | | | phenyl |
| 21 | E | benzothiazol- | —CH₂— | H | —CH₂— | 2,4-dichloro- |
| | | 2-yl | | | | phenyl |
| 22 | E | quinolin-3-yl | —CH₂— | H | —CH₂— | 2,4-dichloro- |
| | | | | | | phenyl |
| 23 | E | 4-methoxy- | —CH₂— | H | —CH₂— | 2,4-dichloro- |
| | | phenyl | | | | phenyl |
| 24 | E | 4-chloro- | —CH₂—CH₂— | H | —CH₂— | 2,4-dichloro- |
| | | phenyl | | | | phenyl |
| 25 | E | benzothien- | —CH₂— | H | —CH₂— | 2,4-dichloro- |
| | | 2-yl | | | | phenyl |
| 26 | E | benzofur-2-yl | —CH₂— | H | —CH₂— | 2,4-dichloro- |
| | | | | | | phenyl |
| 27 | E | benzothien- | —NH—CH₂— | H | —CH₂— | 2,4-dichloro- |
| | | 2-yl | | | | phenyl |
| 28 | E | benzofur-2-yl | —NH—CH₂— | H | —CH₂— | 2,4-dichloro- |
| | | | | | | phenyl |
| 29 | E | 5-chloro- | —CH₂—CH₂— | H | —CH₂— | 2,4-dichloro- |
| | | thien-2-yl | | | | phenyl |
| 30 | E | 4-methoxy- | —CH₂—CH₂— | H | —CH₂— | 2,4-dichloro- |
| | | phenyl | | | | phenyl |
| 31 | E | benzothien- | —CH₂—CH₂— | H | —CH₂— | 2,4-dichloro- |
| | | 2-yl | | | | phenyl |
| 33 | E | 4-chloro- | —N(CH₃)—CH₂— | H | —CH₂— | 2,4-dichloro- |
| | | phenyl | | | | phenyl |
| 34 | Z | 4,5-dichloro- | —OCH₂— | F | —CH₂— | 2,4-dichloro- |
| | | thien-2-yl | | | | phenyl |
| 35 | E | 4,5-dichloro- | —OCH₂— | F | —CH₂— | 2,4-dichloro- |
| | | thien-2-yl | | | | phenyl |
| 36 | E | 4-chloro- | —OCH₂— | H | —CH₂— | 2,4-dichloro- |
| | | phenyl | | | | phenyl |
| 38 | E | 2-methoxy- | —CH₂—CH₂— | H | —CH₂— | 2,4-dichloro- |
| | | 4-chloro- | | | | phenyl |
| | | phenyl | | | | |
| 39 | E | 2-methoxy- | —NH—CH₂— | H | —CH₂— | 2,4-dichloro- |
| | | phenyl | | | | phenyl |
| 40 | Z | 4,5-dichloro- | —NH—CH₂— | F | —CH₂— | 2,4-dichloro- |
| | | thien-2-yl | | | | phenyl |
| 41 | E | 2-methoxy- | —CH₂—CH₂— | H | —CH₂— | 2,4-dichloro- |
| | | 5-bromo- | | | | phenyl |
| | | phenyl | | | | |
| 42 | E | 2-methoxy- | —NH—CH₂— | H | —CH₂— | 2,4-dichloro- |
| | | 5-bromo- | | | | phenyl |
| | | phenyl | | | | |
| 43 | E | 2-methoxy- | —NH—CH₂— | H | —CH₂— | 2,4-dichloro- |
| | | 5-chloro- | | | | phenyl |
| | | phenyl | | | | |
| 44 | E | 2-methoxy- | —CH₂—CH₂— | H | —CH₂— | 2,4-dichloro- |
| | | phenyl | | | | phenyl |
| 45 | E | 4,5-dichloro- | —NH—CH₂— | F | —CH₂— | 2,4-dichloro- |
| | | thien-2-yl | | | | phenyl |
| 50 | Z | 4-chloro- | —OCH₂— | H | —CH₂— | 2,4-dichloro- |
| | | phenyl | | | | phenyl |
| 52 | E | 4,5-dichloro- | —CH₂—CH₂— | H | a = 0 | 3-phenyl- |
| | | thien-2-yl | | | | phenyl |
| 53 | E | 4,5-dichloro- | —CH₂—CH₂— | H | a = 0 | 3-(2,4- |
| | | thien-2-yl | | | | dichloro- |
| | | | | | | phenyl)- |
| | | | | | | phenyl |
| 56 | Z | 4,5-dichloro- | —CH₂—CH₂— | F | —CH₂— | 2,4-dichloro- |
| | | thien-2-yl | | | | phenyl |
| 59 | Z | 4,5-dichloro- | —N(CH₃)—CH₂— | F | —CH₂— | 2,4-dichloro- |
| | | thien-2-yl | | | | phenyl |
| 69 | E | isopropyl | —CH₂—CH₂— | H | —CH₂— | 2,4-dichloro- |
| | | | | | | phenyl |
| 75 | E | 4,5-dichloro- | —NH—CH₂— | H | a = 0 | 4-bromo- |
| | | thien-2-yl | | | | phenyl |
| 76 | E | 4,5-dichloro- | —NH—CH₂— | H | a = 0 | 3-(4-fluoro- |
| | | thien-2-yl | | | | phenyl)- |
| | | | | | | phenyl |
| 77 | E | 4,5-dichloro- | —NH—CH₂— | H | a = 0 | 3-bromo- |
| | | thien-2-yl | | | | phenyl |
| 78 | E | 4,5-dichloro- | —NH—CH₂— | H | a = 0 | naphth-1-yl |
| | | thien-2-yl | | | | |
| 79 | E | 4,5-dichloro- | —NH—CH₂— | H | a = 0 | naphth-2-yl |
| | | thien-2-yl | | | | |
| 80 | E | 4-chloro- | —NH—CH₂— | H | —CH₂— | isopropyl |
| | | phenyl | | | | |
| 84 | E | 4,5-dichloro- | —NH—CH₂— | H | a = 0 | 2,4-dichloro- |
| | | thien-2-yl | | | | phenyl |
| 85 | E | 4,5-dichloro- | —NH—CH₂— | H | a = 0 | 4-trifluoro- |
| | | thien-2-yl | | | | methyl-phenyl |
| 86 | Z | 4-chloro- | —NH—CH₂— | F | —CH₂— | 2,4-dichloro- |
| | | phenyl | | | | phenyl |
| 88 | E | 4,5-dichloro- | —NH—CH₂— | H | —CH₂— | naphth-2-yl |
| | | thien-2-yl | | | | |
| 89 | Z | 2-methoxy- | —NH—CH₂— | F | —CH₂— | 2,4-dichloro- |
| | | 5-chloro- | | | | phenyl |
| | | phenyl | | | | |
| 90 | Z | 2-methoxy- | —NH—CH₂— | F | —CH₂— | 2,4-dichloro- |
| | | 5-fluoro- | | | | phenyl |
| | | phenyl | | | | |
| 91 | Z | 3-chloro- | —NH—CH₂— | F | —CH₂— | 2,4-dichloro- |
| | | phenyl | | | | phenyl |
| 92 | E | 4,5-dichloro- | —NH—CH₂— | H | a = 0 | 3-trifluoro- |
| | | thien-2-yl | | | | methyl-phenyl |
| 93 | Z | 4,5-dichloro- | —CH₂— | F | —CH₂— | 2,4-dichloro- |
| | | thien-2-yl | | | | phenyl |
| 94 | E | 4-chloro- | —NH—CH₂— | H | a = 0 | 5-bromo- |
| | | phenyl | | | | pyrimidin-2-yl |
| 95 | E | 4-chloro- | —OCH₂— | H | a = 0 | pyrimidin-2-yl |
| | | phenyl | | | | |
| 99 | E | 4,5-dichloro- | —NH—CH₂— | H | a = 0 | 4-phenyl- |
| | | thien-2-yl | | | | phenyl |
| 105 | E | 4-chloro- | —NH—CH₂— | H | a = 0 | 6-methoxy- |
| | | phenyl | | | | pyrid-3-yl |
| 106 | E | 2-methoxy- | —NH—CH₂— | F | a = 0 | naphth-2-yl |
| | | 5-chloro- | | | | |
| | | phenyl | | | | |
| 107 | E | 4,5-dichloro- | —NH—CH₂— | H | a = 0 | 6-methoxy- |
| | | thien-2-yl | | | | pyrid-3-yl |
| 110 | E | 2,3- | —NH—CH₂— | F | a = 0 | naphth-2-yl |
| | | dihydrobenzo | | | | |
| | | [b][1,4]dioxin- | | | | |
| | | 6-yl | | | | |
| 111 | Z | benzofur-2-yl | —NH—CH₂— | F | —CH₂— | 2,4-dichloro- |
| | | | | | | phenyl |
| 112 | E | 2-methoxy- | —NH—CH₂— | F | a = 0 | naphth-2-yl |
| | | 5-fluoro- | | | | |
| | | phenyl | | | | |
| 113 | Z | 2-methoxy- | —NH—CH₂— | F | a = 0 | naphth-2-yl |
| | | 5-fluoro- | | | | |
| | | phenyl | | | | |
| 114 | Z | 2,3- | —NH—CH₂— | F | a = 0 | naphth-2-yl |
| | | dihydrobenzo | | | | |
| | | [b][1,4]dioxin- | | | | |
| | | 6-yl | | | | |
| 115 | Z | 2-methoxy- | —NH—CH₂— | F | a = 0 | naphth-2-yl |
| | | 5-chloro- | | | | |
| | | phenyl | | | | |
| 116 | Z | 2,3- | —NH—CH₂— | F | a = 0 | 4-trifluoro- |
| | | dihydrobenzo | | | | methyl-phenyl |
| | | [b][1,4]dioxin- | | | | |
| | | 6-yl | | | | |
| 117 | E | 2,3- | —NH—CH₂— | F | a = 0 | 4-trifluoro- |
| | | dihydrobenzo | | | | methyl-phenyl |
| | | [b][1,4]dioxin- | | | | |
| | | 6-yl | | | | |
| 121 | Z | benzothien- | —NH—CH₂— | F | —CH₂— | 2,4-dichloro- |
| | | 2-yl | | | | phenyl |
| 122 | E | 4-chloro- | —NH—CH₂— | H | a = 0 | 1-(4-fluoro- |
| | | phenyl | | | | phenyl)- |
| | | | | | | piperidin-4-yl |
| 123 | E | 4,5-dichloro- | —NH—CH₂— | H | a = 0 | 1-(4-fluoro- |
| | | thien-2-yl | | | | phenyl)- |
| | | | | | | piperidin-4-yl |
| 124 | Z | 5-chloro- | —NH—CH₂— | F | a = 0 | 4-trifluoro- |
| | | thien-2-yl | | | | methyl-phenyl |
| 125 | Z | 4,5-dichloro- | —NH—CH₂— | F | a = 0 | 4-trifluoro- |
| | | thien-2-yl | | | | methyl-phenyl |
| 126 | E | 4,5-dichloro- | —NH—CH₂— | H | a = 0 | 4-methyl- |
| | | thien-2-yl | | | | sulfonyl- |
| | | | | | | phenyl |
| 130 | Z | 4,5-dichloro- | —NH—CH₂— | F | a = 0 | 4-trifluoro- |
| | | thien-2-yl | | | | methyl-phenyl |
| 131 | Z | 2-methoxy- | —NH—CH₂— | F | —CH₂— | 4-trifluoro- |
| | | 5-chloro- | | | | methyl-phenyl |
| | | phenyl | | | | |
| 132 | Z | benzofur-2-yl | —NH—CH₂— | F | —CH₂— | 4-trifluoro- |
| | | | | | | methyl-phenyl |
| 133 | Z | benzothien- | —NH—CH₂— | F | —CH₂— | 4-trifluoro- |
| | | 2-yl | | | | methyl-phenyl |
| 134 | Z | 4,5-dichloro- | —NH—CH₂— | F | —CH₂— | 4-trifluoro- |
| | | thien-2-yl | | | | methyl-phenyl |
| 136 | Z | 4,5-dichloro- | —NH—CH₂— | F | —CH₂— | 2,4-dichloro- |
| | | thien-2-yl | | | | phenyl |
| 137 | E | 4,5-dichloro- | —NH—CH₂— | F | a = 0 | 4-trifluoro- |
| | | thien-2-yl | | | | methyl-phenyl |
| 138 | E | 4,5-dichloro- | —NH—CH₂— | H | —CH₂— | 2-phenyl- |
| | | thien-2-yl | | | | pyrimidin-5-yl |
| 139 | E | 2-methyl- | —NH—CH₂— | H | —CH₂— | 2,4-dichloro- |
| | | thiazol-5-yl | | | | phenyl |
| 140 | E | 4,5-dichloro- | —NH—CH₂— | H | a = 0 | 1-(pyrid-2-yl)- |
| | | thien-2-yl | | | | piperidin-4-yl |
| 141 | Z | 4,5-dichloro- | —NH—CH₂— | F | —CH₂— | 6-methoxy- |
| | | thien-2-yl | | | | pyrid-3-yl |
| 142 | Z | 4,5-dimethyl- | —NH—CH₂— | F | —CH₂— | 2,4-dichloro- |
| | | thien-2-yl | | | | phenyl |
| 143 | Z | 3-chloro-4- | —NH—CH₂— | F | —CH₂— | 2,4-dichloro- |
| | | methoxy- | | | | phenyl |
| | | phenyl | | | | |
| 148 | E | 4,5-dichloro- | —NH—CH₂— | H | a = 0 | 1-(benzyl)- |
| | | thien-2-yl | | | | piperidin-4-yl |
| 149 | Z | 4-methyl-5- | —NH—CH₂— | F | —CH₂— | 2,4-dichloro- |
| | | chloro-thien- | | | | phenyl |
| | | 2-yl | | | | |
| 150 | E | 2,4-dimethyl- | —NH—CH₂— | H | —CH₂— | 2,4-dichloro- |
| | | thiazol-5-yl | | | | phenyl |
| 151 | E | 4,5-dichloro- | —NH—CH₂— | H | a = 0 | 1-methyl- |
| | | thien-2-yl | | | | piperidin-4-yl |
| 152 | E | 1-methyl- | —NH—CH₂— | H | —CH₂— | 2,4-dichloro- |
| | | pyrazol-4-yl | | | | phenyl |
| 153 | E | 1-methyl- | —NH—CH₂— | H | —CH₂— | 2,4-dichloro- |
| | | pyrazol-3-yl | | | | phenyl |
| 154 | Z | 4,5-dichloro- | —NH—CH₂— | F | a = 0 | 1-(4-fluoro- |
| | | thien-2-yl | | | | phenyl)- |
| | | | | | | piperidin-4-yl |
| 156 | Z | 3,4-difluoro- | —NH—CH₂— | F | —CH₂— | 2,4-dichloro- |
| | | phenyl | | | | phenyl |
| 157 | Z | 4,5-dichloro- | —NH—CH₂— | F | —CH₂—CH₂— | 4-trifluoro- |
| | | thien-2-yl | | | | methyl-benzyl |
| 158 | Z | 2,4-dimethyl- | —NH—CH₂— | F | —CH₂— | 2,4-dichloro- |
| | | thiazol-5-yl | | | | phenyl |
| 159 | Z | 3-fluoro-4- | —NH—CH₂— | F | —CH₂— | 2,4-dichloro- |
| | | methoxy- | | | | phenyl |
| | | phenyl | | | | |
| 163 | E | 4,5-dichloro- | —NH—CH₂— | H | —CH₂— | 5-phenyl- |
| | | thien-2-yl | | | | pyrimidin-2-yl |
| 168 | Z | 1-methyl- | —NH—CH₂— | F | —CH₂— | 2,4-dichloro- |
| | | pyrazol-5-yl | | | | phenyl |
| 169 | Z | 1,5-dimethyl- | —NH—CH₂— | F | —CH₂— | 2,4-dichloro- |
| | | pyrazol-4-yl | | | | phenyl |
| 170 | Z | 1-isopropyl- | —NH—CH₂— | F | —CH₂— | 2,4-dichloro- |
| | | pyrazol-4-yl | | | | phenyl |
| 171 | E | 1-methyl- | —NH—CH₂— | H | —CH₂— | 2,4-dichloro- |
| | | pyrazol-5-yl | | | | phenyl |
| 172 | E | 1,5-dimethyl- | —NH—CH₂— | H | —CH₂— | 2,4-dichloro- |
| | | pyrazol-4-yl | | | | phenyl |
| 173 | E | 1-isopropyl- | —NH—CH₂— | H | —CH₂— | 2,4-dichloro- |
| | | pyrazol-4-yl | | | | phenyl |
| 174 | Z | 3,4- | —NH—CH₂— | F | —CH₂— | 2,4-dichloro- |
| | | dimethoxy- | | | | phenyl |
| | | phenyl | | | | |
| 175 | Z | 3-methoxy- | —NH—CH₂— | F | —CH₂— | 2,4-dichloro- |
| | | 4-chloro- | | | | phenyl |
| | | phenyl | | | | |
| 176 | E | benzoxazol- | —NH—CH₂— | H | —CH₂— | 2,4-dichloro- |
| | | 6-yl-2-one | | | | phenyl |
| 177 | E | 2-methoxy- | —NH—CH₂— | H | —CH₂— | 2,4-dichloro- |
| | | benzoxazol- | | | | phenyl |
| | | 6-yl | | | | |
| 178 | Z | 2-methoxy- | —NH—CH₂— | F | —CH₂— | 2,4-dichloro- |
| | | benzoxazol- | | | | phenyl |
| | | 6-yl | | | | |
| 180 | Z | 1,3-dimethyl- | —NH—CH₂— | F | —CH₂— | 2,4-dichloro- |
| | | pyrazol-4-yl | | | | phenyl |
| 182 | Z | 2-(methyl- | —NH—CH₂— | F | —CH₂— | 2,4-dichloro- |
| | | carbonyl- | | | | phenyl |
| | | amino)-4- | | | | |
| | | methyl- | | | | |
| | | thiazol-5-yl | | | | |
| 183 | Z | 2-methyl- | —NH—CH₂— | F | —CH₂— | 2,4-dichloro- |
| | | benzoxazol- | | | | phenyl |
| | | 5-yl | | | | |
| 184 | E | 1,3-dimethyl- | —NH—CH₂— | H | —CH₂— | 2,4-dichloro- |
| | | pyrazol-4-yl | | | | phenyl |
| 185 | E | 4-methyl-5- | —NH—CH₂— | H | —CH₂— | 2,4-dichloro- |
| | | chloro-thien- | | | | phenyl |
| | | 2-yl | | | | |
| 191 | Z | 4,5-dichloro- | —NH—CH₂— | F | —CH₂— | 1-phenyl- |
| | | thien-2-yl | | | | pyrazol-4-yl |
| 192 | E | 1,3-dimethyl- | —NH—CH₂— | H | —CH₂— | 2,4-dichloro- |
| | | pyrazol-5-yl | | | | phenyl |
| 193 | Z | 1,3-dimethyl- | —NH—CH₂— | F | —CH₂— | 2,4-dichloro- |
| | | pyrazol-5-yl | | | | phenyl |
| 194 | Z | 1-methyl- | —NH—CH₂— | F | —CH₂— | 2,4-dichloro- |
| | | indazol-6-yl | | | | phenyl |
| 195 | Z | 1-methyl- | —NH—CH₂— | F | —CH₂— | 2,4-dichloro- |
| | | indazol-5-yl | | | | phenyl |
| 196 | E | 4-methyl-5- | —NH—CH₂— | H | a = 0 | 2,4-dichloro- |
| | | chloro-thien- | | | | phenyl |
| | | 2-yl | | | | |
| 197 | Z | 5-chloro-6- | —NH—CH₂— | F | —CH₂— | 2,4-dichloro- |
| | | methyl-pyrid- | | | | phenyl |
| | | 3-yl | | | | |
| 198 | Z | 4,5-dichloro- | —NH—CH₂— | F | —CH₂— | 2-phenyl- |
| | | thien-2-yl | | | | pyrimidin-5-yl |
| 199 | Z | 3-chloro-4- | —NH—CH₂— | F | —CH₂— | 2-phenyl- |
| | | methoxy- | | | | pyrimidin-5-yl |
| | | phenyl | | | | |

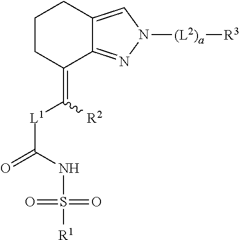

| ID No | Stereo | R¹ | L¹ | R² | (L²)ₐ | R³ |
|---|---|---|---|---|---|---|
| 87 | E | 4,5-dichloro- | —NH—CH₂— | H | —CH₂— | naphth-2-yl |
| | | thien-2-yl | | | | |

TABLE 2
Representative Compounds of Formula (I)

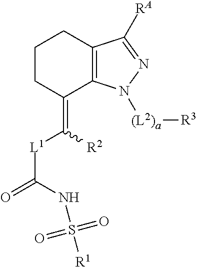

| ID No | R^{A} | Stereo | R¹ | L¹ | R² | (L²)ₐ | R³ |
|---|---|---|---|---|---|---|---|
| 32 | —CHF₂ | E | 4-chloro- | —OCH₂— | H | —CH₂— | 2,4-dichloro- |
| | | | phenyl | | | | phenyl |
| 47 | —CHF₂ | E | 4-chloro- | —NH—CH₂— | H | —CH₂— | 2,4-dichloro- |
| | | | phenyl | | | | phenyl |
| 109 | —CH₃ | Z | 4,5- | —NH—CH₂— | F | a = 0 | 2,4-dichloro- |
| | | | dichloro- | | | | phenyl |
| | | | thien-2-yl | | | | |

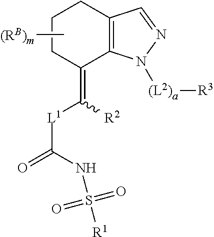

| ID No | (R^{B})ₘ | Stereo | R¹ | L¹ | R² | L²)ₐ | R³ |
|---|---|---|---|---|---|---|---|
| 37 | 5,5-di- | E | 4-chloro- | —OCH₂— | H | —CH₂— | 2,4-dichloro- |
| | fluoro | | phenyl | | | | phenyl |
| 48 | 5,5-di- | E | 4-chloro- | —NH—CH₂— | H | —CH₂— | 2,4-dichloro- |
| | fluoro | | phenyl | | | | phenyl |
| 51 | 6-fluoro | E | 4-chloro- | —OCH₂— | H | —CH₂— | 2,4-dichloro- |
| | | | phenyl | | | | phenyl |
| 70 | 5,5-di- | E | 4-chloro- | —NH—CH₂— | H | —CH₂— | 3-trifluoro- |
| | fluoro | | phenyl | | | | methyl- |
| | | | | | | | phenyl |
| 73 | 6-fluoro | Z | 4-chloro- | —NH—CH₂— | H | —CH₂— | 2,4-dichloro- |
| | | | phenyl | | | | phenyl |
| 81 | 6,6-di- | E | 4-chloro- | —OCH₂— | H | —CH₂— | 2,4-dichloro- |
| | fluoro | | phenyl | | | | phenyl |
| 82 | 6,6- | Z | 4-chloro- | —OCH₂— | H | —CH₂— | 2,4-dichloro- |
| | difluoro | | phenyl | | | | phenyl |
| 100 | 6-fluoro | E | 4,5- | —NH—CH₂— | F | —CH₂— | 2,4-dichloro- |
| | | | dichloro- | | | | phenyl |
| | | | thien-2-yl | | | | |
| 101 | 6-fluoro | E | 4-chloro- | —NH—CH₂— | F | —CH₂— | 2,4-dichloro- |
| | | | phenyl | | | | phenyl |
| 103 | 6-fluoro | E | 2-methoxy- | —NH—CH₂— | F | —CH₂— | 2,4-dichloro- |
| | | | 5-chloro- | | | | phenyl |
| | | | phenyl | | | | |

TABLE 3
Representative Compounds of Formula (I)

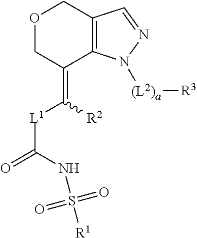

| ID No | Stereo | R¹ | L¹ | R² | (L²)ₐ | R³ |
|---|---|---|---|---|---|---|
| 46 | Z | 4,5-dichloro- | —OCH₂— | H | —CH₂— | 2,4-dichloro- |
| | | thien-2-yl | | | | phenyl |
| 49 | Z | 4,5-dichloro- | —NH—CH₂— | H | —CH₂— | 2,4-dichloro- |
| | | thien-2-yl | | | | phenyl |
| 54 | E | 4,5-dichloro- | —NH—CH₂— | F | —CH₂— | 2,4-dichloro- |
| | | thien-2-yl | | | | phenyl |
| 55 | Z | 4,5-dichloro- | —N(CH₃)—CH₂— | H | —CH₂— | 2,4-dichloro- |
| | | thien-2-yl | | | | phenyl |
| 57 | Z | 4,5-dichloro- | —CH₂—CH₂— | H | —CH₂— | 2,4-dichloro- |
| | | thien-2-yl | | | | phenyl |
| 58 | Z | 2-methoxy- | —CH₂— | H | —CH₂— | 2,4-dichloro- |
| | | phenyl | | | | phenyl |
| 60 | Z | 4,5-dichloro- | —NH—CH₂— | F | —CH₂— | 2,4-dichloro- |
| | | thien-2-yl | | | | phenyl |
| 61 | E | 4,5-dichloro- | —CH₂—CH₂— | F | —CH₂— | 2,4-dichloro- |
| | | thien-2-yl | | | | phenyl |
| 62 | Z | 4-methoxy- | —NH—CH₂— | H | —CH₂— | 2,4-dichloro- |
| | | phenyl | | | | phenyl |
| 63 | Z | 3-methoxy- | —NH—CH₂— | H | —CH₂— | 2,4-dichloro- |
| | | phenyl | | | | phenyl |
| 64 | Z | 2-methoxy-5- | —NH—CH₂— | H | —CH₂— | 2,4-dichloro- |
| | | fluoro-phenyl | | | | phenyl |
| 65 | Z | 2-chloro-5- | —NH—CH₂— | H | —CH₂— | 2,4-dichloro- |
| | | methoxy- | | | | phenyl |
| | | phenyl | | | | |
| 66 | Z | 2-methoxy-5- | —NH—CH₂— | H | —CH₂— | 2,4-dichloro- |
| | | chloro-phenyl | | | | phenyl |
| 67 | Z | 6-methoxy- | —NH—CH₂— | H | —CH₂— | 2,4-dichloro- |
| | | pyrid-3-yl | | | | phenyl |
| 68 | Z | 2-fluoro-5- | —NH—CH₂— | H | —CH₂— | 2,4-dichloro- |
| | | methoxy- | | | | phenyl |
| | | phenyl | | | | |
| 74 | E | 4,5-dichloro- | —N(CH₃)—CH₂— | F | —CH₂— | 2,4-dichloro- |
| | | thien-2-yl | | | | phenyl |
| 83 | E | 4-chloro- | —NH—CH₂— | F | —CH₂— | 2,4-dichloro- |
| | | phenyl | | | | phenyl |
| 104 | E | 4-chloro- | —NH—CH₂— | F | a = 0 | 3-trifluoro- |
| | | phenyl | | | | methyl-phenyl |
| 108 | Z | 4-chloro- | —OCH₂— | F | a = 0 | 3-trifluoro- |
| | | phenyl | | | | methyl-phenyl |
| 118 | E | 4-chloro- | —NH—CH₂— | F | a = 0 | 4-trifluoro- |
| | | phenyl | | | | methyl-phenyl |
| 119 | E | 4,5-dichloro- | —NH—CH₂— | F | a = 0 | 4-trifluoro- |
| | | thien-2-yl | | | | methyl-phenyl |
| 144 | E | 4,5-dichloro- | —NH—CH₂— | F | a = 0 | 1-(4-fluoro- |
| | | thien-2-yl | | | | phenyl)- |
| | | | | | | piperidin-4-yl |
| 160 | E | 2,4-dimethyl- | —NH—CH₂— | F | —CH₂— | 2,4-dichloro- |
| | | thiazol-5-yl | | | | phenyl |
| 161 | E | 3-fluoro-4- | —NH—CH₂— | F | —CH₂— | 2,4-dichloro- |
| | | methoxy- | | | | phenyl |
| | | phenyl | | | | |
| 165 | E | 3-chloro-4- | —NH—CH₂— | F | —CH₂— | 2,4-dichloro- |
| | | methoxy- | | | | phenyl |
| | | phenyl | | | | |
| 166 | E | 4-methyl-5- | —NH—CH₂— | F | —CH₂— | 2,4-dichloro- |
| | | chloro-thien- | | | | phenyl |
| | | 2-yl | | | | |

TABLE 4
Representative Compounds of Formula (I)

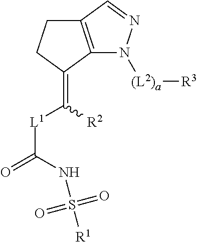

| ID No | Stereo | R¹ | L¹ | R² | (L²)ₐ | R³ |
|---|---|---|---|---|---|---|
| 36 | E | 4-chloro- | —O—CH₂— | H | —CH₂— | 2,4- |
| | | phenyl | | | | dichloro- |
| | | | | | | phenyl |
| 130 | Z | 4,5-dichloro- | —NH—CH₂— | F | a = 0 | 4-trifluoro- |
| | | thien-2-yl | | | | methyl- |
| | | | | | | phenyl |
| 137 | E | 4,5-dichloro- | —NH—CH₂— | F | a = 0 | 4-trifluoro- |
| | | thien-2-yl | | | | methyl- |
| | | | | | | phenyl |

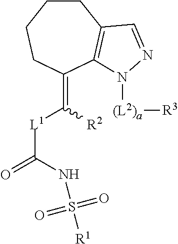

| ID No | Stereo | R¹ | L¹ | R² | (L²)ₐ | R³ |
|---|---|---|---|---|---|---|
| 98 | Z | 4-chloro- | —NH—CH₂— | F | —CH₂— | 2,4- |
| | | phenyl | | | | dichloro- |
| | | | | | | phenyl |
| 102 | E | 4-chloro- | —NH—CH₂— | F | —CH₂— | 2,4- |
| | | phenyl | | | | dichloro- |
| | | | | | | phenyl |
| 120 | Z | 4,5- | —NH—CH₂— | F | —CH₂— | 2,4- |
| | | dichloro- | | | | dichloro- |
| | | thien-2-yl | | | | phenyl |

TABLE 5
Representative Compounds of Formula (I)

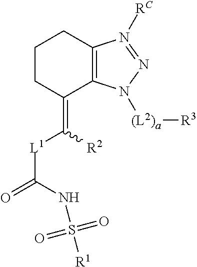

| ID No | R^{C} | Stereo | R¹ | L¹ | R² | (L²)ₐ | R³ |
|---|---|---|---|---|---|---|---|
| 187 | H | Z | 4-methyl- | —NH—CH₂— | F | —CH₂— | 2,4-dichloro- |
| | | | 5-chloro- | | | | phenyl |
| | | | thien-2-yl | | | | |

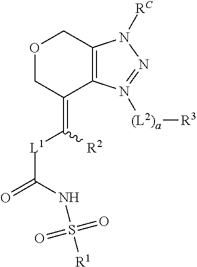

| ID No | R^{C} | Stereo | R¹ | L¹ | R² | (L²)ₐ | R³ |
|---|---|---|---|---|---|---|---|
| 146 | —CH₂—OCH₃ | E | 4,5- | —NH—CH₂— | F | —CH₂— | 2,4-dichloro- |
| | | | dichloro- | | | | phenyl |
| | | | thien-2-yl | | | | |
| 147 | H | E | 4,5- | —NH—CH₂— | F | —CH₂— | 2,4-dichloro- |
| | | | dichloro- | | | | phenyl |
| | | | thien-2-yl | | | | |
| 179 | H | E | 3-chloro-4- | —NH—CH₂— | F | —CH₂— | 2,4-dichloro- |
| | | | methoxy- | | | | phenyl |
| | | | phenyl | | | | |
| 186 | CH₃ | Z | 4,5- | —NH—CH₂— | F | —CH₂— | 2,4-dichloro- |
| | | | dichloro- | | | | phenyl |
| | | | thien-2-yl | | | | |
| 190 | H | E | 4-methyl- | —NH—CH₂— | F | —CH₂— | 2,4-dichloro- |
| | | | 5-chloro- | | | | phenyl |
| | | | thien-2-yl | | | | |

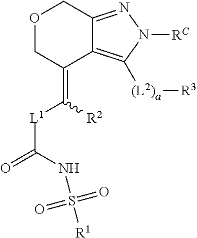

| ID No | R^{C} | Stereo | R¹ | L¹ | R² | (L²)ₐ | R³ |
|---|---|---|---|---|---|---|---|
| 189 | CH₃ | E | 4,5- | —NH—CH₂— | F | —CH₂— | 2,4-dichloro- |
| | | | dichloro- | | | | phenyl |
| | | | thien-2-yl | | | | |

TABLE 6
Representative Compounds of Formula (I)

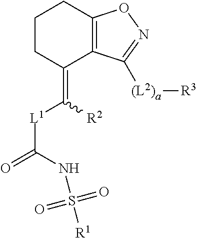

| ID No | Stereo | R¹ | L¹ | R² | (L²)ₐ | R³ |
|---|---|---|---|---|---|---|
| 96 | E | 4-chloro- | —NH—CH₂— | F | a = 0 | 4-chloro- |
| | | phenyl | | | | phenyl |
| 97 | Z | 4-chloro- | —NH—CH₂— | F | a = 0 | 4,5- |
| | | phenyl | | | | dichloro- |
| | | | | | | phenyl |
| 127 | Z | 4,5-dichloro- | —NH—CH₂— | F | —CH₂— | 2,4- |
| | | thien-2-yl | | | | dichloro- |
| | | | | | | phenyl |
| 128 | E | 4,5-dichloro- | —NH—CH₂— | F | —CH₂— | 2,4- |
| | | thien-2-yl | | | | dichloro- |
| | | | | | | phenyl |
| 181 | Z | 4-methyl-5- | —NH—CH₂— | F | —CH₂— | 2,4- |
| | | chloro-thien- | | | | dichloro- |
| | | 2-yl | | | | phenyl |

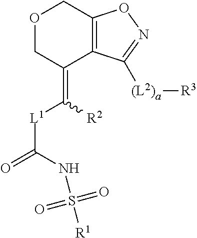

| ID No | Stereo | R¹ | L¹ | R² | (L²)ₐ | R³ |
|---|---|---|---|---|---|---|
| 145 | E | 4,5- | —NH—CH₂— | F | —CH₂— | 2,4- |
| | | dichloro- | | | | dichloro- |
| | | thien-2-yl | | | | phenyl |

TABLE 7
Representative Compounds of Formula (I)

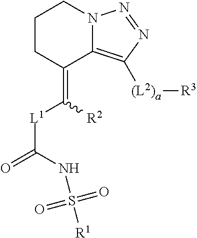

| ID No | Stereo | R¹ | L¹ | R² | (L²)ₐ | R³ |
|---|---|---|---|---|---|---|
| 129 | Z | 4,5-dichloro- | —NH—CH₂— | F | —CH₂— | phenyl |
| | | thien-2-yl | | | | |
| 135 | E | 4,5-dichloro- | —NH—CH₂— | F | —CH₂— | phenyl |
| | | thien-2-yl | | | | |
| 155 | E | 4,5-dichloro- | —NH—CH₂— | H | a = 0 | 4-trifluoro- |
| | | thien-2-yl | | | | methyl-phenyl |
| 164 | Z | 4,5-dichloro- | —NH—CH₂— | F | a = 0 | 4-trifluoro- |
| | | thien-2-yl | | | | methyl-phenyl |
| 167 | Z | 4,5-dichloro- | —NH—CH₂— | F | —CH₂— | 2-methyl-4- |
| | | thien-2-yl | | | | chloro-phenyl |
| 188 | Z | 4-methyl-5- | —NH—CH₂— | F | —CH₂— | 2-methyl-4- |
| | | chloro-thien-2-yl | | | | chloro-phenyl |

In certain embodiments, the present invention is directed to a compound of formula (I); wherein the compound has a measured K (nM) according to the EP3 competition binding assay procedure taught in Biological Example 1, which follows herein, less than about 500 nM, preferably less than about 250 nM, more preferably less than about 100 nM, more preferably less than about 50 nM, more preferably less than about 25 nM, more preferably less than about 10 nM, more preferably less than about 5 nM, more preferably less than about 1 nM, more preferably less than about 0.75 nM, more preferably less than about 0.5 nM.

In certain embodiments, the present invention is directed to a compound of formula (I); wherein the compound has a IC₅₀ (nM) according to the EP3 cAMP antagonist dose response (sulprostone reference) assay procedure taught in Biological Example 2, which follows herein, less than about 1000 nM, preferably less than about 500 nM, preferably less than about 250 nM, more preferably less than about 100 nM, more preferably less than about 50 nM, more preferably less than about 25 nM, more preferably less than about 10 nM, more preferably less than about 5 nM.

### Definitions

As used herein, unless otherwise noted, “halogen” shall mean chlorine, bromine, fluorine and iodine. Preferably, the halogen is fluorine, chlorine or bromine, more preferably fluorine.

As used herein when referring to a substituent group, unless otherwise noted, the term “oxo” shall mean a double bonded oxygen group, i.e. a substituent group of the formula ═O.

As used herein, unless otherwise noted, the term “C_{X-Y}alkyl” wherein X and Y are integers, whether used alone or as part of a substituent group, shall include straight and branched chains of between X and Y carbon atoms. For example, C₁₋₄alkyl shall include straight and branched chains of between one and four carbon atoms including methyl, ethyl, propyl, isopropyl, butyl, isobutyl, sec-butyl and t-butyl.

One skilled in the art will recognize that the terms “—(C_{X-Y}alkyl)- and —C_{X-Y}alkyl-” wherein X and Y are integers, shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the term “fluorinated C_{X-Y}alkyl” wherein X and Y are integers, shall mean any C_{X-Y}alkyl group as defined above substituted with at least one fluoro atom, preferably one to three fluoro atoms. Suitable examples include but are not limited to —CF₃, —CH₂—CF₃, —CF₂—CF₂—CF₂—CF₃, and the like.

As used herein, unless otherwise noted, “C_{X-Y}alkoxy” wherein X and Y are integers, whether used alone or as part of a substituent group, shall denote an oxygen ether radical of the above described C_{X-Y}alkyl straight or branched chain alkyl group. For example, C₁₋₄alkoxy shall include oxygen ether radicals of straight and branched alkyl chains of between one and four carbon atoms including methoxy, ethoxy, n-propoxy, iso-propoxy, n-butoxy, sec-butoxy and t-butoxy.

As used herein, unless otherwise noted, the term “fluorinated C_{X-Y}alkoxy” wherein X and Y are integers, shall denote an oxygen ether radical as defined described, substituted with at least one fluoro atom, preferably one to three fluoro atoms. Suitable examples include but are not limited to —OCF₃, —OCH₂—CF₃, —OCF₂—CF₂—CF₂—CF₃, and the like.

As used herein, unless otherwise noted, the term “C_{X-Y}cycloalkyl” wherein X and Y are integers, shall mean any stable monocyclic, bicyclic, polycyclic or bridged, saturated ring system consisting of between X and Y carbon atom. For example, the term C₃₋₆cycloalkyl shall include cyclopropyl, cyclobutyl, cyclopentyl and cyclohexyl.

As used herein, unless otherwise noted, the term “5- to 6-membered heteroaryl” shall denote any five or six membered monocyclic, aromatic ring structure, wherein the monocyclic ring structure contains at least one heteroatom selected from the group consisting of O, N and S, optionally containing one to three additional heteroatoms independently selected from the group consisting of O, N and S. The 5- to 6-membered heteroaryl group may be attached at any heteroatom or carbon atom of the ring such that the result is a stable structure. The 5- to 6-membered heteroaryl group may be further, optionally substituted, as herein defined. Suitably examples include, but are not limited to, pyridinyl, pyridazinyl, pyrimidyl, pyrazyl, triazinyl, pyrrolyl, pyrazolyl, imidazolyl, (1,2,3,)-triazolyl, (1,2,4)-triazolyl, pyrazinyl, pyrimidinyl, tetrazolyl, furyl, thienyl, isoxazolyl, thiazolyl, oxazolyl, and the like.

In certain embodiments of the present invention the 5- to 6-membered heteroaryl groups is selected from the group consisting of furyl, thienyl, pyridyl, pyrimidinyl, pyrazolyl, thiazolyl, oxazolyl, isoxazolyl, pyrrolyl and imidazolyl. In certain embodiments of the present invention the 5- to 6-membered heteroaryl groups is selected from the group consisting of pyrimidinyl, pyridyl, pyrazolyl and piperidinyl.

As used herein, unless otherwise noted, the term “heterocyclyl” shall denote any four or six membered monocyclic ring structure, wherein the ring structure may be saturated, partially unsaturated or aromatic, and wherein the monocyclic ring structure contains at least one heteroatom selected from the group consisting of O, N and S, optionally containing one to three additional heteroatoms independently selected from the group consisting of O, N and S, or any nine or ten membered bicyclic ring structure, wherein the ring structure may be saturated, partially unsaturated, partially aromatic, benzo-fused or aromatic, and wherein the bicyclic ring structure contains at least one heteroatom selected from the group consisting of O, N and S, optionally containing one to four additional heteroatoms independently selected from the group consisting of O, N and S. The heterocyclyl group may be attached at any heteroatom or carbon atom of the ring such that the result is a stable structure. The heterocyclyl group may be further, optionally substituted, as herein defined.

Suitable examples of four to six membered monocyclic heterocyclyl groups include, but are not limited to, azetidinyl, pyrrolidinyl, pyrrolinyl, furanyl, thienyl, pyrrolyl, isopuyrrolyl, pyrazlyl, imidazolyl, isoimidazolyl, triazolyl, tetrazolyl, oxazolyl, isoxazolyl, thiazolyl, isothiazolyl, oxadiazolyl, dioxazolyl, pyranyl, pyridinyl, pyridazinyl, pyrimidinyl, dioxalanyl, imidazolinyl, imidazolidinyl, pyrazolinyl, pyrazolidinyl, pyrazinyl, piperidinyl, piperazinyl, morpholinyl, thiomorpholinyl, tetrahydropyranyl, tetrahydrothiopyranyl, thiazinanyl, triazinyl, oxazinyl, isoxazinyl, and the like.

Suitable examples of nine to ten membered bicyclic heterocyclyl groups include, but are not limited to, indolizinyl, indolyl, isoindolinyl, indazolyl, benzofuryl, benzoxazolyl, anthracil, benzopyranyl, quinolinyl, isoquinolinyl, cinnolinyl, quinazolinyyl, benzothienyl, benzimidazolyl, benzothiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, benzofuryl, isobenzofuryl, indolinyl, chromanyl, chromenyl, 3,4-methylenedioxyphenyl, 2,3-dihydrobenzofuryl, oxetanyl, pyrido[3,4-b]pyridine, purinyl, quinozilinyl, quinoxalinyl, quinazolinyl, benzo[b][1,4]oxazinyl, 3,4-dihydro-benzo[b][1,4]oxazinyl, benzo[b][1,4]dioxinyl, 2,3-dihydro-benzo[b][1,4]dioxinyl, and the like.

In certain embodiments of the present invention the heterocyclyl is selected from the group consisting of thienyl, thiazolyl, pyrazolyl, pyridiyl, pyrimidinyl, piperidinyl, indazolyl, benzofuryl, benzothienyl, benzothiazolyl, benzoxazolyl, quiunolinyl and 2,3-dihydrobenzo[b][1.4]dioxin-6-yl. In certain embodiments of the present invention the heterocyclyl is selected from the group consisting of thiazolyl, pyrazolyl, pyridiyl, indazolyl, benzofuryl, benzothienyl, benzothiazolyl, benzoxazolyl, quiunolinyl and 2,3-dihydrobenzo[b][1.4]dioxin-6-yl.

When a particular group is “substituted” (e.g., alkyl, cycloalkyl, heterocyclyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Furthermore, it is intended that within the scope of the present invention, any element, in particular when mentioned in relation to a compound of formula (I), shall comprise all isotopes and isotopic mixtures of said element, either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radio-labelled compounds of formula (I) may comprise a radioactive isotope selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

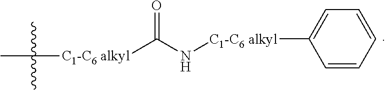

One skilled in the art will recognize that in the compounds of formula (I) of the present invention, the double bond bound to the

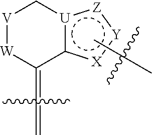

portion of the compound of formula (I) may exist in either the (E)- of (Z)-configuration or as a mixture thereof. It is intended that the present invention include all stereoisomers, mixture of stereoisomers and racemates thereof.

Abbreviations used in the specification, particularly the Schemes and Examples, are as listed in Table A, below.

TABLE A
Abbreviations

| | |
|---|---|
| AIR | Acute Insulin Response |
| ANOVA = | Analysis of Variance |
| aq. = | Aqueous |
| Boc = | tert-Butoxy-carbonyl- |
| BSA = | Bovine Serum Albumin |
| cAMP = | Cyclic Adenosine MonoPhosphate |
| CDI = | 1,1′carbonylsiimidazole |
| DBU = | 1,8-Diazabicyclo[5.4.0]undec-7-ene |
| DCC = | N,N'Dicyclohexylcarbodiimide |
| DCM = | Dichloromethane |
| DEAD = | Diethyl azodicarboxylate |
| Dess Martin Periodinone or | 1,1,1-Triacetoxy-1,1-dihydro-1,2- |
| DMP = | benziodoxol-3(1H)-one |
| DIAD = | Diisopropylazodicarboxylate |
| DIBAL of DIBAL—H = | Diisobutylammonium hydride |
| DIPEA or iPr₂EtN = | Diisopropylethylamine |
| DMAP = | 4-N,N-Dimethylaminopyridine |
| DMF = | N,N-Dimethylformamide |
| DMSO = | Dimethylsulfoxide |
| dppf = | 1,1′-Bis(diphenylphosphino)ferrocene |
| EDCl or EDC = | 1-Ethyl-3-(3-dimethylaminopropyl) |
| | carbodiimide |
| EDTA = | Ethylene Diamine Tetraacetic Acid |
| eq. or equiv. = | Equivalents (molar) |
| ESI = | Electrospray ionization |
| Et₂O = | Diethyl ether |
| EtOAc or EA = | Ethyl acetate |
| EtOH = | Ethanol |
| Et₃N or TEA = | Triethylamine |
| FFA = | Fatty Free Acid |
| FSK = | Forskolin |
| GSIS = | Glucose Stimulated Insulin Secretion |
| HATU = | O-(7-Azabenzotriazol-1-yl)-N,N,N″,N″- |
| | Tetramethyl Uronium Hexafluorophosphate |
| HBSS = | Hank's Buffered Salt Solution |
| HEPES = | 4-(2-Hydroxyethyl)-1-Piperizine Ethane |
| | Sulfonic Acid |
| HMDS = | Hexamethyldisilazane |
| HPBCD = | Hydroxypropyl-Beta-CycloDextrin |
| HPLC = | High Pressure Liquid Chromatography |
| IFG = | Impaired Fasting Glucose |
| IGT = | Impaired Glucose Tolerance |
| i-PrOH = | Isopropanol |
| LAH = | Lithium Aluminum Hydride |
| LCMS = | Liquid Chromatography/Mass |
| | Spectroscopy |
| LDA = | Lithium Diisopropyl Amide |
| LiHMDS = | Lithium bis(trimethylsilyl)amide |
| MeCN = | Acetonitrile |
| MeI = | Methyl Iodide |
| MeOH = | Methanol |
| Mesyl or Ms = | Methylsulfonyl |
| MS = | Mass Spectroscopy |
| MsCl = | Mesyl Chloride (i.e. CH₃—SO₂—Cl) |
| Mesylate = | Methane sulfonate (i.e. —O—SO₂—CH₃) |
| MOM = | Methoxymethyl acetal (protecting group) |
| MTBE = | Methyl tert-Butyl Ether |
| NAFLD = | non-alcoholic fatty liver disease |
| NaOEt = | Sodium Ethoxide |
| NaOt-Bu or tBuONa = | Sodium tert-Butoxide |
| NASH = | non-alcoholic steatohepatitis (NASH), |
| NBS = | N-Bromosuccinimide |
| n-BuLi = | n-Butyl Lithium |
| NMR = | Nuclear Magnetic Resonance |
| NFSI = | N-fluoro-N-(phenylsulfonyl)benzene |
| | sulfonamide |
| —OMs = | —O-mesyl |
| —OTs = | —O-tosyl |
| Pd(dppf)Cl₂ = | [1,1′-Bis(diphenylphosphino) |
| | ferrocene]dichloropalladium(II) |
| Pd(PPh₃)₄ = | Tetrakistriphenylphosphine palladium (0) |
| Pd(PPh₃)₂Cl₂ = | Bis(triphenylphosphine)palladium II |
| | dichloride |
| PE = | Petroleum Ether |
| Ph = | Phenyl |
| Phl(OAc)₂ = | (Diacetoxyiodo)benzene |
| PBu₃ = | Tri(n-butyl) Phosphine |
| PPh₃ = | Triphenyl Phosphine |
| SELECTFLUOR ® = | 1-Chloromethyl-4-fluoro-1,4- |
| | diazoniabicyclo[2.2.2]octane |
| | bis(tetrafluoroborate) |
| TBDPS = | tert-Butyldiphenyisilyl |
| TBS = | tert-Butyldimethylsilyl |
| t-BuOH = | tert-Butanol |
| TEA = | Triethylamine |
| TFA = | Trifluoroacetic Acid |
| THF = | Tetrahydrofuran |
| THP = | Tetrahydropyran |
| TLC = | Thin Layer Chromatography |
| TMS = | Trimethylsilyl |
| Tosyl or Ts = | para-Toluenesulfonyl |
| Tosylate = | para-Toluene sulfonate (i.e. —O—SO₂-(p- |
| | toluene) |
| TsCl = | para-Toluenesulfonic Chloride (i.e. (p- |
| | toluene)-SO₂—Cl) |

As used herein, unless otherwise noted, the term “isolated form” shall mean that the compound of formula (I) is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound of formula (I) is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to describe the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

One skilled in the art will further recognize that the reaction or process step(s) as herein described (or claimed) are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed description which follows herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, 1,1-dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention yield rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) wherein L¹ is —O—CH₂— may be prepared as described in Scheme 1, below.

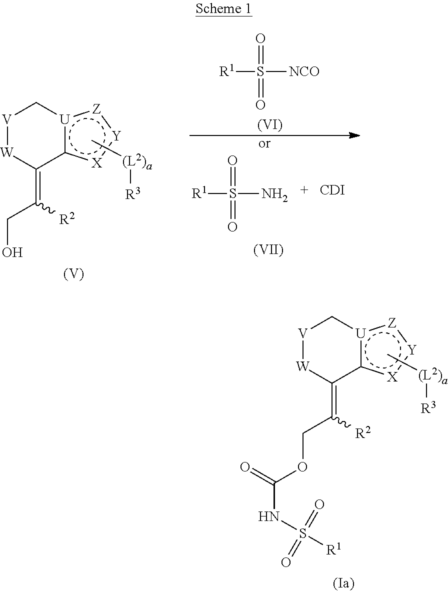

Accordingly, a suitably substituted compound of formula (V), a known compound, compound prepared by known methods or compound prepared as described herein, is reacted with a suitably substituted compound of formula (VI), a known compound or compound prepared by known methods; in a suitably selected organic solvent such as CH₂Cl₂, CHCl₃, toluene, and the like; at a temperature in the range of from about 0° C. to about room temperature; to yield the corresponding compound of formula (Ia).

Alternatively, a suitably substituted compound of formula (VII), a known compound or compound prepared by known methods, is reacted with di(1H-imidazol-1-yl)methanone (CDI); in a suitably selected organic solvent such as THF, and the like; at about room temperature; optionally in the presence of a suitably selected organic base, such as Et₃N, pyridine, DIPEA, and the like; and then reacted with a suitably substituted compound of formula (V), at about 100° C.; to yield the corresponding compound of formula (Ia).

Compounds of formula (I) wherein L¹ is —NH—CH₂— may be prepared as described in Scheme 2 below.

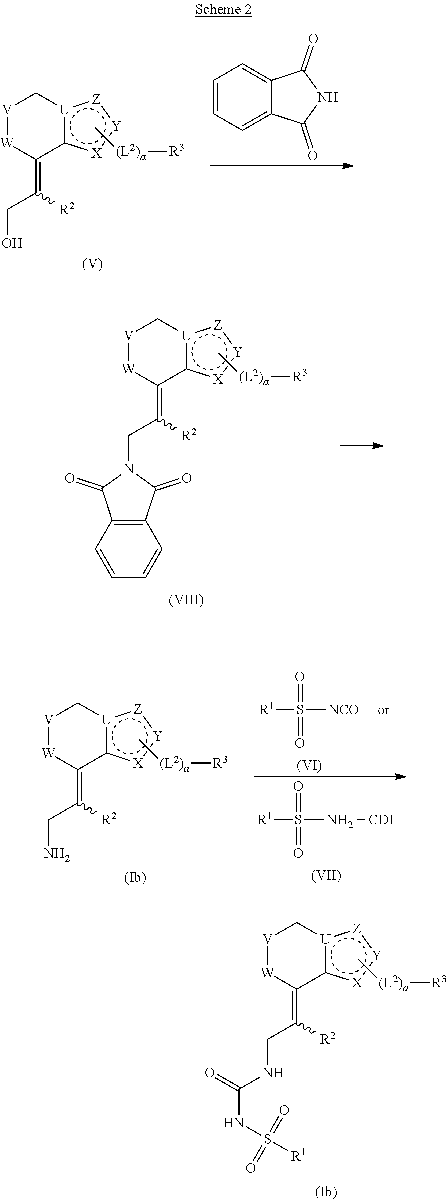

Accordingly, a suitably substituted compound of formula (V), a known compound, compound prepared by known methods or compound prepared as described herein, is reacted with isoindoline-1,3-dione, a known compound; in the presence of a suitably selected phosphine reagent such as PPh₃, PBu₃, and the like; in the presence of a suitably selected azodicarboxylate reagent such as DEAD, DIAD, and the like; in a suitably selected organic solvent such as THF, diethyl ether, CHCl₃, and the like; at about room temperature; to yield the corresponding compound of formula (VIII).

The compound of formula (VIII) is reacted with hydrazine; in a suitably selected organic solvent such as MeOH, EtOH, i-PrOH, and the like; at about room temperature; to yield the corresponding compound of formula (IX).

The compound of formula (IX) is reacted with a suitably substituted compound of formula (VI), a known compound or compound prepared by known methods; in a suitably selected organic solvent such as CH₂Cl₂, CHCl₃, toluene, and the like; at a temperature in the range of from about 0° C. to about room temperature; to yield the corresponding compound of formula (Ib).

Alternatively, a suitably substituted compound of formula (VII), a known compound or compound prepared by known methods, is reacted with di(1H-imidazol-1-yl)methanone (CDI); in a suitably selected organic solvent such as THF, and the like; at about room temperature; optionally in the presence of a suitably selected organic base, such as Et₃N, pyridine, DIPEA, and the like; and then reacted with the compound of formula (IX), at about 100° C.; to yield the corresponding compound of formula (Ib).

Compounds of formula (I) wherein L¹ is —NH—CH₂— may alternatively be prepared as described in Scheme 3 below.

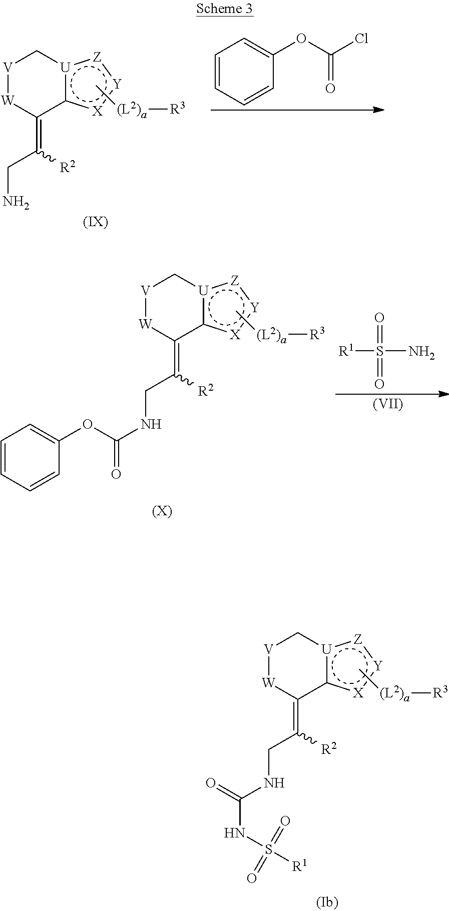

Accordingly, a suitably substituted compound of formula (IX), a known compound, a compound prepared for example as described in Scheme 2 above, or a compound prepared as described herein, is reacted with phenyl carbonochloridate, a known compound; in the presence of a suitably selected base, such as Et₃N, DIPEA, and the like; in a suitably selected organic solvent such as CH₂Cl₂, CHCl₃, toluene, and the like, at a temperature in the range of from about 0° C. to about room temperature; to yield the corresponding compound of formula (X).

The compound of formula (X) is reacted with a suitably substituted compound of formula (VII), a known compound or compound prepared by known methods, in the presence of a suitably selected base, such as DBU, DIPEA, and the like; in a suitably selected organic solvent such as CH₃CN, DMSO, DMF, and the like; at a temperature about room temperature; to yield the corresponding compound of formula (Ib).

Compounds of formula (I) wherein L¹ is selected from the group consisting of —N(CH₃)—CH₂— and —N(CH₂CH₃)—CH₂— (i.e. L¹ is —N(R^{X})—CH₂— wherein R^{X} is methyl or ethyl) may be prepared as described in Scheme 4, below.

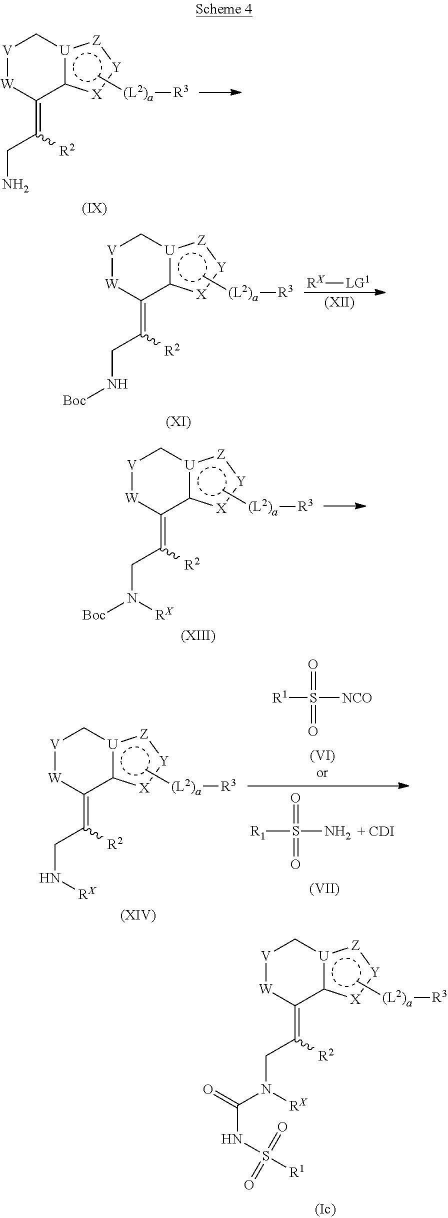

Accordingly, a suitably substituted compound of formula (IX), a compound prepared for example as described in Scheme 2 above or a compound prepared as described herein, is reacted with di-tert-butyl decarbonate, a known compound; in a suitably selected organic solvent such as THF, CH₂Cl₂, diethyl ether, and the like; at a temperature about room temperature; to yield the corresponding compound of formula (XI). (One skilled in the art will recognize that alternate suitably nitrogen protecting groups such as —C(O)CF₃, and the like, may be used instead of the Boc protecting group, and may be incorporated into the compound of formula (XI), according to known methods.)

The compound of formula (XI) is reacted with a suitably selected base such as LDA, LiHMDS, NaH, and the like; in a suitably selected organic solvent such as THF, DMF, and the like; at a temperature in the range of from about-78° C. to about room temperature; and is then reacted with a suitably substituted compound of formula (XII), wherein LG¹ is a suitably selected leaving group such as —Br, —I, —OMs, and the like; to yield the corresponding compound of formula (XIII).

The compound of formula (XIII) is deprotected, according to known methods, to yield the corresponding compound of formula (XIV). For example, the Boc-protected compound of formula (XIII) may be reacted with a suitably selected acid such as TFA, HCl, and the like; in a suitably selected organic solvent such as CH₂Cl₂, MeOH, 1,4-dioxane, and the like; at about room temperature; to yield the corresponding compound of formula (XIV).

The compound of formula (XIV) is reacted with a suitably substituted compound of formula (VI), a known compound or compound prepared by known methods; in a suitably selected organic solvent such as CH₂Cl₂, CHCl₃, toluene, and the like; at a temperature in the range of from about 0° C. to about room temperature; to yield the corresponding compound of formula (Ic).

Alternatively, a suitably substituted compound of formula (VII), a known compound or compound prepared by known methods, is reacted with di(1H-imidazol-1-yl)methanone (CDI); in a suitably selected organic solvent such as THF, and the like; at about room temperature; optionally in the presence of a suitably selected organic base, such as Et₃N, pyridine, DIPEA, and the like; and then reacted with the compound of formula (XIV), at about 100° C.; to yield the corresponding compound of formula (Ic).

Compounds of formula (I) wherein L¹ is —CH₂— may be prepared as described in Scheme 5 below.

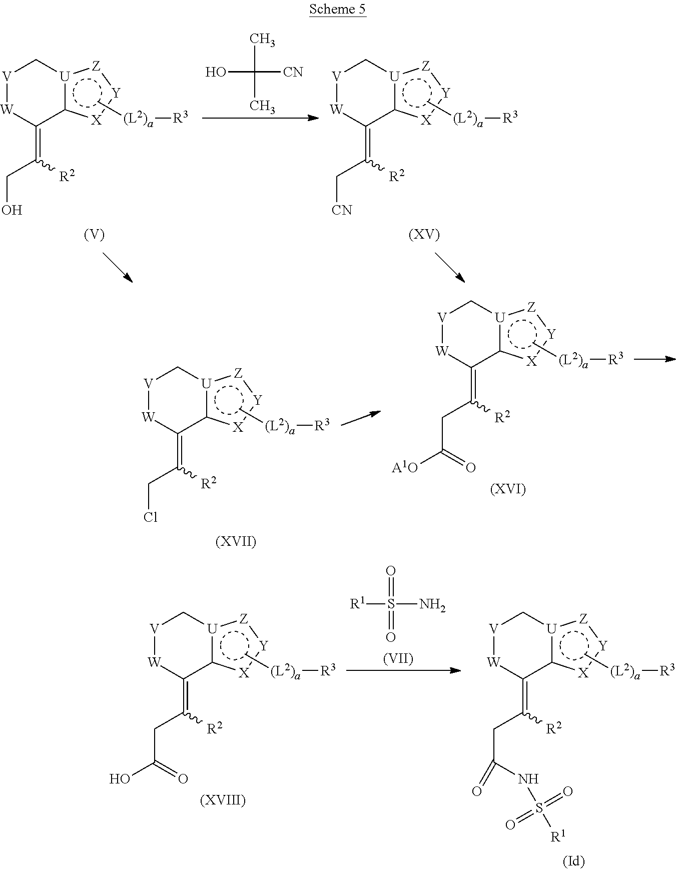

Accordingly, a suitably substituted compound of formula (V), a known compound, a compound prepared by known methods, or a compound prepared as described herein, is reacted with 2-hydroxy-2-methylpropanenitrile, a known compound; in the presence of a suitably selected phosphine reagent such as PPh₃, PBu₃, and the like; in the presence of a suitably selected azodicarboxylate reagent such as DEAD, DIAD, and the like; in a suitably selected organic solvent such as THF, diethyl ether, CHCl₃, and the like; at about room temperature; to yield the corresponding compound of formula (XV).

The compound of formula (XV) is reacted with SOCl₂ in the presence of a suitably selected alcohol such as MeOH, EtOH, and the like; at about room temperature; to yield the corresponding compound of formula (XVI), wherein A¹ is the corresponding alkyl (for example, A¹ is methyl when MeOH is used, A¹ is ethyl when EtOH is used, etc.).

Alternatively, a suitably substituted compound of formula (V) is reacted with SOCl₂ in a suitably selected organic solvent such as CH₂Cl₂, at a temperature in a range of from about 0° C. to about room temperature; to yield the corresponding compound of formula (XVII).

The compound of formula (XVII) is reacted with carbon monoxide (CO) under pressure (for example, at about 20 atm); in the presence of a suitably selected catalyst such as Pd(PPh₃)₂Cl₂, Pd(PPh₃)₄ and the like; in the presence of a suitably selected base such as Cs₂CO₃, K₂CO₃, and the like; in the presence of a suitably selected alcohol such as MeOH, EtOH, and the like; in a suitably selected organic solvent such as THF, 1,4-dioxane, and the like; at about 60° C.; to yield the corresponding compound of formula (XVI), wherein A¹ is the corresponding alkyl (for example, A¹ is methyl when MeOH is used, A¹ is ethyl when EtOH is used, etc.).

The compound of formula (XVI) is reacted with a suitably selected base such as LiOH, NaOH, KOH, and the like; in a suitably selected mixture of an organic solvent and water such as 1,4-dioxane and water, THF and water, MeOH and water, and the like; at about room temperature; to yield the corresponding compound of formula (XVIII).

The compound of formula (XVIII) is reacted with a suitably substituted compound of formula (VII), a known compound or compound prepared by known methods; in the presence of a suitably selected coupling reagent such as EDCI, HATU, and the like, in the presence of a suitably selected base such as DMAP, pyridine, Et₃N, and the like; in a suitably selected organic solvent such as CH₂Cl₂, DMF, and the like; at about room temperature; to yield the corresponding compound of formula (Id).

Compounds of formula (I) wherein L¹ is —CH₂CH₂— may be prepared as described in Scheme 6, below.

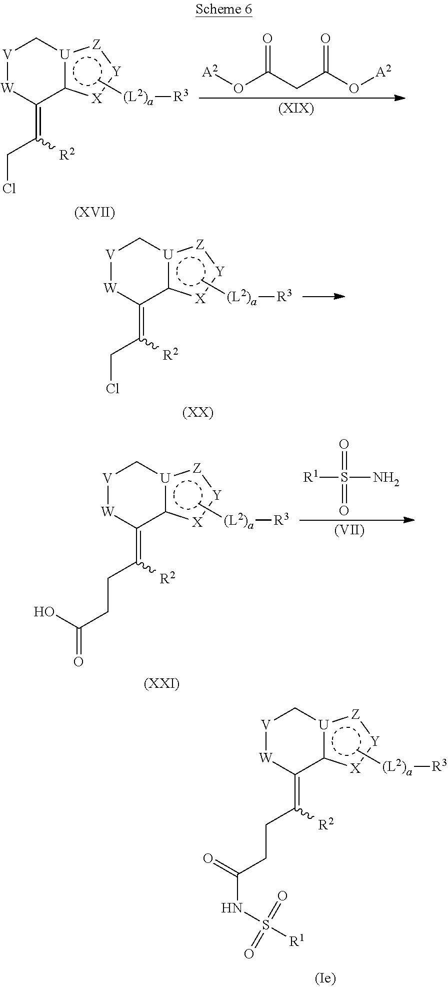

Accordingly, a suitably substituted compound of formula (XVII), a compound prepared as described in Scheme 5 above or a compound prepared as described herein, is reacted with a suitably substituted compound of formula (XIX), wherein both A² groups are the same and are selected from the group consisting of methyl, ethyl, and n-propyl, a known compound or compound prepared by known methods; in the presence of a suitably selected base such as Cs₂CO₃, K₂CO₃, NaH, and the like; in a suitably selected organic solvent such as THF, DMF, and the like; at a temperature in a range of from about 0° C. to about 60° C., to yield the corresponding compound of formula (XX).

The compound of formula (XX) is heated, in the presence of LiCl, in a suitably selected mixture of organic solvent and water, such as DMSO and water, 1,4-dioxane and water, and the like; at a temperature in a range of from about 150° C. to about 200° C., and then reacted with a suitably selected base such as NaOH, KOH, LiOH, and the like; in a suitably selected mixture of organic solvent and water such as MeOH and water, THF and water, and the like, at about room temperature; to yield the corresponding compound of formula (XXI).

The compound of formula (XXI) is reacted with a suitably substituted compound of formula (XVII), a known compound or compound prepared by known methods; in the presence of a suitably selected coupling reagent such as EDCI, HATU, and the like, in the presence of a suitably selected base such as DMAP, pyridine, Et₃N, and the like; in a suitably selected organic solvent such as CH₂Cl₂, DMF, and the like; at about room temperature; to yield the corresponding compound of formula (Ie).

Compounds of formula (I) wherein L¹ is —CH═CH— may be prepared as described in Scheme 7, below.

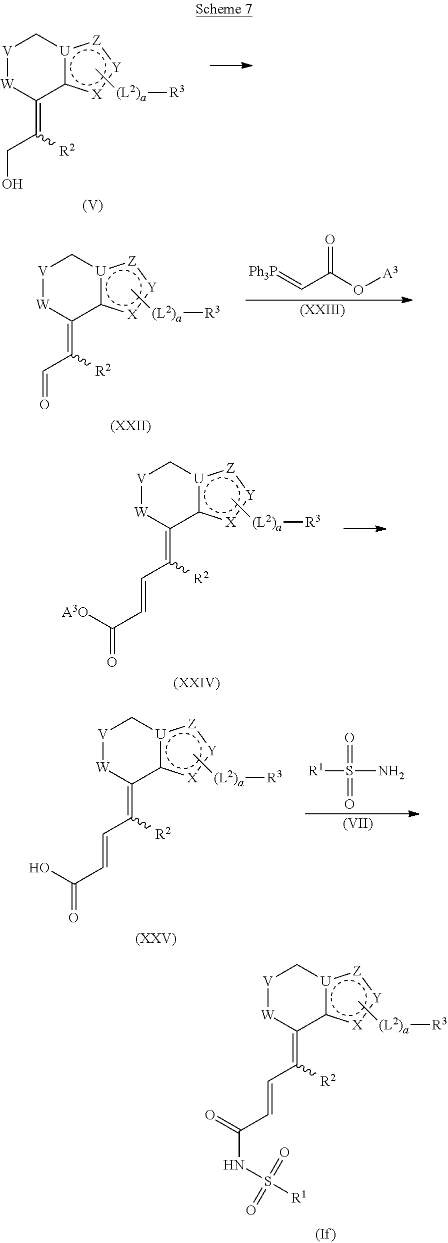

Accordingly, a suitably substituted compound of formula (V), a compound prepared by known methods, or a compound prepared as described herein, is reacted with a suitably selected oxidizing agent such as MnO₂, oxyl chloride/DMSO (Swern reagent), Dess-Martin reagent, and the like; in a suitably selected organic solvent such as CH₂Cl₂, THF, and the like; at a temperature in the range of from about 0° C. to about room temperature; to yield the corresponding compound of formula (XXII).

The compound of formula (XXII) is reacted with a suitable substituted compound of formula (XXIII), wherein A³ is for example, methyl, ethyl, and the like, a known compound or compound prepared by known methods; in a suitably selected organic solvent such as toluene, THF, EtOH, and the like; at a temperature in the range of from about room temperature to about 110° C.; to yield the corresponding compound of formula (XXIV).

The compound of formula (XXIV) is reacted with a suitably selected base such as NaOH, LiOH, KOH, and the like; in a suitably selected mixture of organic solvent and water such as THF and water, 1,4-dioxane and water, MeOH and water, and the like; at about room temperature; to yield the corresponding compound of formula (XXV).

The compound of formula (XXV) is reacted with a suitably substituted compound of formula (VII), a known compound or compound prepared by known methods; in the presence of a suitably selected coupling reagent such as EDCI, HATU, and the like, in the presence of a suitably selected base such as DMAP, pyridine, Et₃N, and the like; in a suitably selected organic solvent such as CH₂Cl₂, DMF, and the like; at a temperature about room temperature; to yield the corresponding compound of formula (If).

Representative compounds of formula (V) may be prepared as described in Schemes 8 through 11, which follow hereinafter.

Compounds of formula (V) wherein

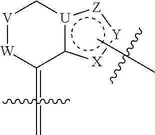

is for example,

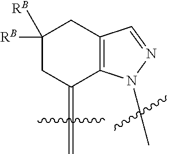

may be prepared as described in Scheme 8, below.

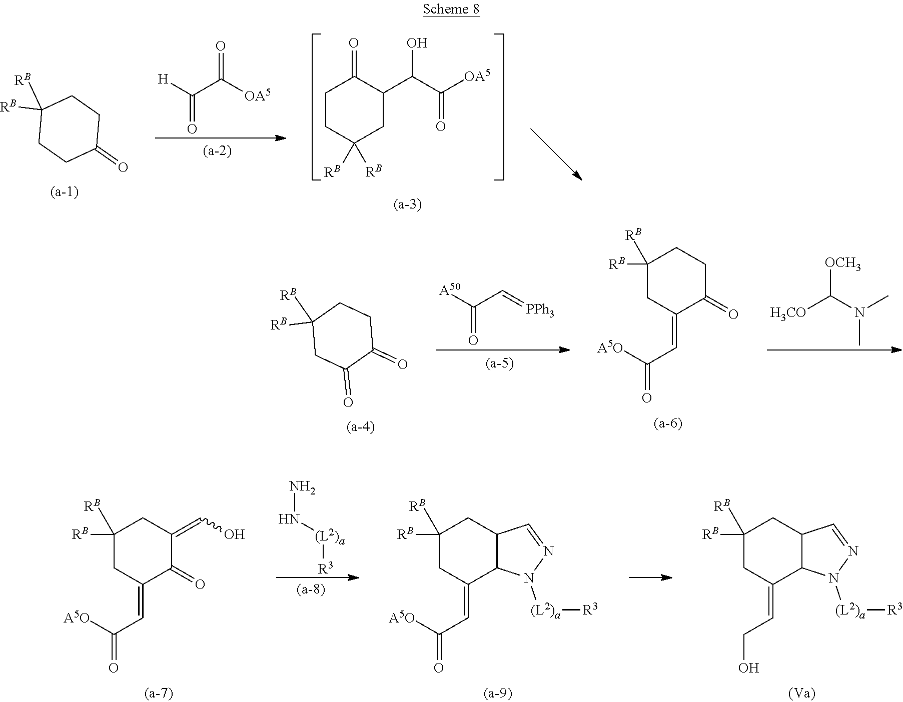

Accordingly, a suitably substituted compound of formula (a-1), a known compound or compound prepared by known methods, is reacted with a suitably selected base such as LiHMDS, LDA, and the like; in a suitably selected organic solvent such as THF, diethyl ether, and the like; at a temperature about −78° C.; and then reacted with a suitably substituted compound of formula (a-2), wherein A⁵ is a suitably selected alkyl group such as methyl, ethyl, and the like, a known compound or compound prepared by known methods, at a temperature in the range of from about −78° C. to 0° C.; to yield the corresponding compound of formula (a-3), which compound is not isolated.

The compound of formula (a-3) is reacted with SOCl₂ in the presence of a suitably selected base such as pyridine, Et₃N, and the like; in a suitably selected organic solvent such as CH₂Cl₂, and the like; at a temperature in the range of from about 0° C. to about room temperature; to yield the corresponding compound of formula (a-7).

Alternatively, a suitably substituted compound of formula (a-4), wherein preferably each R^{B} is hydrogen, is reacted with a suitably substituted compound of formula (a-5), wherein A⁵ is a suitably selected alkyl group such as methyl, ethyl, and the like, a known compound or compound prepared by known methods, in a suitably selected organic solvent such as EtOH, toluene, and the like; at a temperature in the range of from about 70° C. to about 110° C.; to yield the corresponding compound of formula (a-6).

The compound of formula (a-6) is reacted with 1,1-dimethoxy-N,N-dimethylmethanamine, a known compound, at about room temperature; and then reacted with a suitably selected acid such as HCl, H₂SO₄, and the like; in a suitably selected mixture of organic solvent and water such as CH₂Cl₂ and water, and the like; at a temperature about room temperature; to yield the corresponding compound of formula (a-7).

The compound of formula (a-7) is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to 80° C.; to yield the corresponding compound of formula (a-9).

The compound of formula (a-9) is reacted with a suitably selected reducing reagent such as DIBAL-H, LAH, LiBH₄, and the like; in a suitably selected organic solvent such as THF, CH₂Cl₂, toluene, and the like, at a temperature in the range of from about −78° C. to about room temperature; to yield the corresponding compound of formula (Va).

One skilled in the art will recognize that compounds of formula (V) wherein

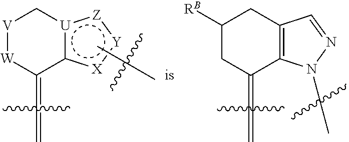

may be similarly prepared by selecting and substituting a suitably substituted compound of formula (a-20)

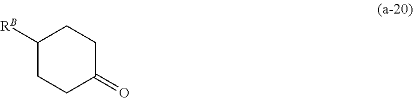

for the compound of formula (a-1), in Scheme 8 above, and reacting as described therein.

Compounds of formula (V) wherein

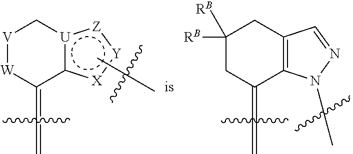

may alternatively be prepared as described in Scheme 9, below.

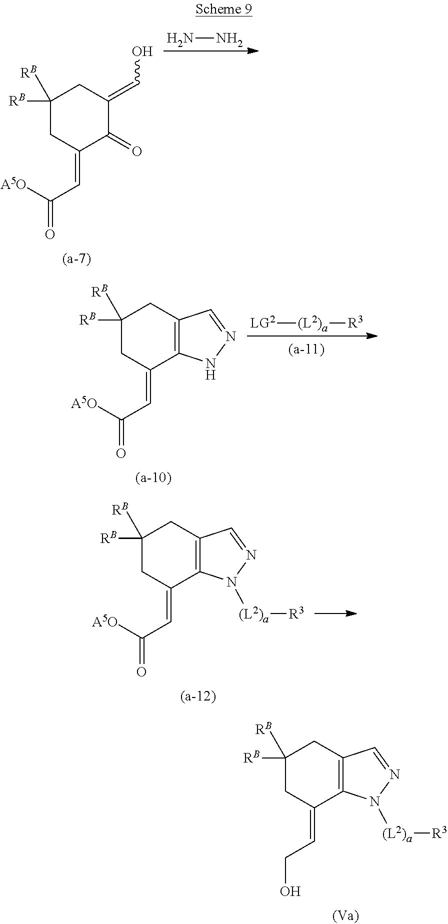

Accordingly, a suitably substituted compound of formula (a-7) is reacted with hydrazine, a known compound; in the presence of a suitably selected acid such as TFA, H₂SO₄, and the like; in a suitably selected organic solvent such as 1,4-dioxane, EtOH, and the like; at a temperature in the range of from about room temperature to 80° C.; to yield the corresponding compound of formula (a-10).

The compound of formula (a-10) is reacted with a suitably substituted compound of formula (a-11), wherein LG² is a suitably selected leaving group such as Br, Cl, I, OMs, and the like; in the presence of a suitably selected base such as Cs₂CO₃, K₂CO₃, and the like, in a suitably selected organic solvent such as DMF, THF, and the like; at a temperature in the range of from about room temperature to 100° C.; to yield the corresponding compound of formula (a-12).

The compound of formula (a-12) is reacted with a suitably selected reducing reagent such as DIBAL-H, LAH, LiBH₄, and the like; in a suitably selected organic solvent such as THF, CH₂Cl₂, toluene, and the like, at a temperature in the range of from about −78° C. to about room temperature; to yield the corresponding compound of formula (Va).

One skilled in the art will recognize that compounds of formula (V) wherein

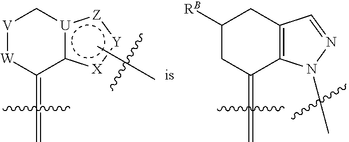

may be similarly prepared by selecting and substituting a suitably substituted compound of formula (a-21)

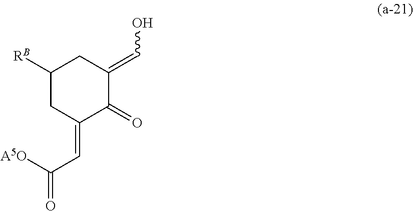

for the compound of formula (a-7), in Scheme 9 above, and reacting as described therein.

Compounds of formula (V) wherein

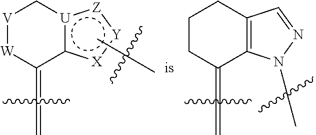

and wherein R² is fluoro may be prepared as described in Scheme 10, below.

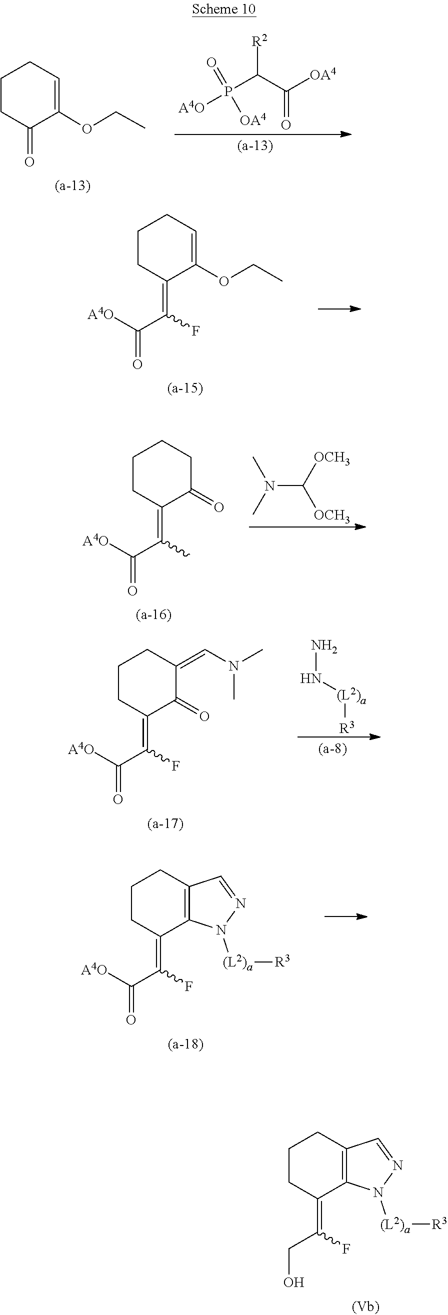

Accordingly, a suitably substituted compound of formula (a-14), wherein each A⁴ is the same and is selected from the group consisting of methyl, ethyl, a known compound or compound prepared by known methods, is reacted with a suitably selected base such as BuLi, LDA, NaH, and the like; in a suitably selected organic solvent such as THF, diethyl ether, and the like; at a temperature in the range of from about −78° C. to about 0° C.; and then reacted with a compound of formula (a-13), a known compound or compound prepared by known methods; at a temperature in the range of from about −78° C. to about room temperature; to yield the corresponding compound of formula (a-15).

The compound of formula (a-15) is reacted with a suitably selected acid such as HCl, H₂SO₄, and the like; in a suitably selected mixture of organic solvent and water such as CH₂Cl₂ and water, and the like; to yield the corresponding compound of formula (a-16).

The compound of formula (a-16) is reacted with 1,1-dimethoxy-N,N-dimethylmethanamine, a known compound; optionally in the presence of a suitably selected organic solvent such as DMF, and the like; at a temperature in the range of from about room temperature to about 100° C., to yield the corresponding compound of formula (a-17).

The compound of formula (a-17) is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to 80° C.; to yield the corresponding compound of formula (a-18).

The compound of formula (a-18) is reacted with a suitably selected reducing reagent such as DIBAL-H, LAH, LiBH₄, and the like; in a suitably selected organic solvent such as THF, CH₂Cl₂, toluene, and the like, at a temperature in the range of from about −78° C. to about room temperature; to yield the corresponding compound of formula (Vb).

Compounds of formula (V) may alternatively be prepared as described in Scheme 11, below.

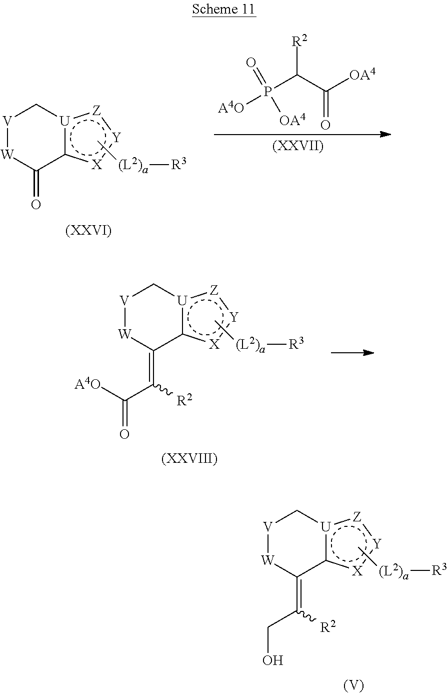

Accordingly, a suitably substituted compound of formula (XXVI), a known compound, a compound prepared by known methods or a compound prepared as described herein, is reacted with a suitably substituted compound of formula (XXVII), wherein each A⁴ is the same and is selected from the group consisting of methyl, ethyl, a known compound or compound prepared by known methods; in the presence of a suitably selected base such as BuLi, LDA, LiHMDS, NaH, and the like; in a suitably selected organic solvent such as THF, diethyl ether, DMF, and the like; at a temperature in the range of from about −78° C. to about room temperature; to yield the corresponding compound of formula (XXVIII).

The compound of formula (XXVIII) is reacted with a suitably selected reducing reagent such as DIBAL-H, LAH, LiBH₄, and the like; in a suitably selected organic solvent such as THF, CH₂Cl₂, toluene, and the like, at a temperature in the range of from about −78° C. to about room temperature; to yield the corresponding compound of formula (V).

Representative compounds of formula (XXVI) may be prepared as described in Schemes 12 through 19, which follow hereinafter.

Compounds of formula (XXVI) wherein

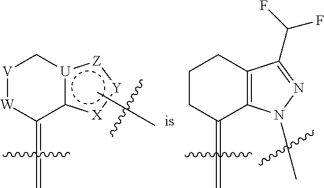

may be prepared as described in Scheme 12, below.

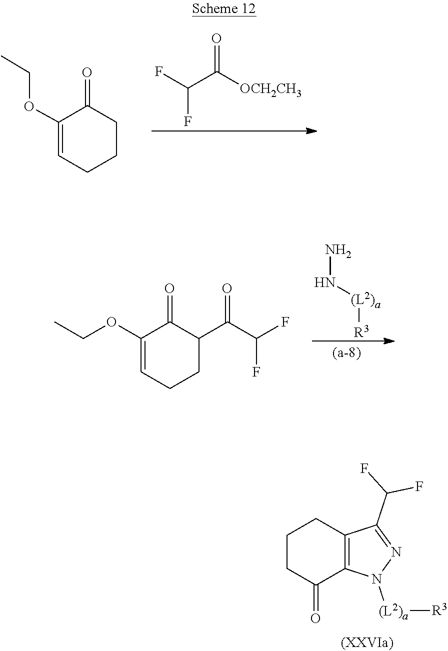

Accordingly, 2-ethoxycyclohex-2-en-1-one, a known compound, is reacted with ethyl 2,2-difluoroacetate, a known compound; in the presence of a suitably selected base such as LiHMDS, LDA, and the like; in a suitably selected organic solvent such as THF, diethyl ether, and the like; at a temperature in the range of from about −78° C. to about room temperature; to yield 6-(2,2-difluoroacetyl)-2-ethoxycyclohex-2-en-1-one.

The 6-(2,2-difluoroacetyl)-2-ethoxycyclohex-2-en-1-one is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to about 80° C., to yield the corresponding compound of formula (XXVIa).

Compounds of formula (XXVI) wherein

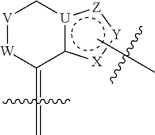

is selected from the group consisting of

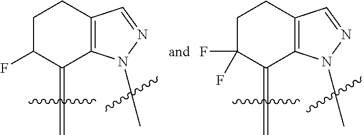 [SMILES: *n1ncc2c1C(=*)C(F)CC2 ; *n1ncc2c1C(=*)C(F)(F)CC2]

may be prepared as described in Scheme 13, below.

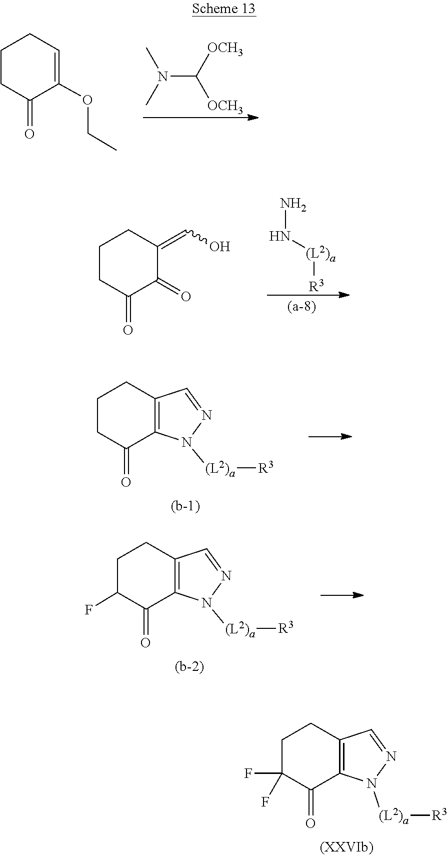

Accordingly, 2-ethoxycyclohex-2-en-1-one, a known compound, is reacted with 1,1-dimethoxy-N,N-dimethylmethanamine, a known compound, at about room temperature; and then reacted with a suitably selected acid such as HCl, H₂SO₄, and the like; in a suitably selected mixture of organic solvent and water such as CH₂Cl₂ and water, and the like; at a temperature about room temperature; to yield 3-(hydroxymethylene)cyclohexane-1,2-dione.

The 3-(hydroxymethylene)cyclohexane-1,2-dione is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to about 80° C., to yield the corresponding compound of formula (b-1).

The compound of formula (b-1) is reacted with a suitably selected fluorination reagent such as N-fluoro-N-(phenylsulfonyl)benzenesulfonamide (NFSI), SELECTFLUOR®, and the like; in the presence of a suitably selected base such as LiHMDS, LDA, and the like; in a suitably selected organic solvent such as THF, diethyl ether, and the like; at a temperature in the range of from about −78° C. to about room temperature; to yield the corresponding compound of formula (b-2). One skilled in the art will recognize that the compound of formula (b-2) corresponds to a compound of formula (XXVI), wherein m is 1 and R^{B} is bound at the 6-position.

The compound of formula (b-2) is reacted with a suitably selected fluorination reagent such as N-fluoro-N-(phenylsulfonyl)benzenesulfonamide (NFSI), SELECTFLUOR®, and the like; in the presence of a suitably selected base such as LiHMDS, LDA, and the like; in a suitably selected organic solvent such as THF, diethyl ether, and the like; at a temperature in the range of from about −78° C. to about room temperature; to yield the corresponding compound of formula (XXVIb).

One skilled in the art will recognize that compound of formula (XXVIb) may alternatively be prepared directly from the corresponding compound of formula (b-1) by reacting the compound of formula (b-1) with at least two molar equivalents, preferably more than two molar equivalents, of both the suitably selected base and suitably selected fluorinating.

One skilled in the art will further recognize that compounds of (XXVI) wherein

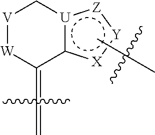

is selected from the group consisting of

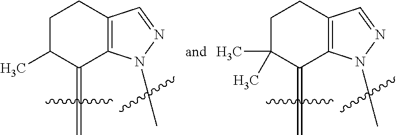 [SMILES: *n1ncc2c1C(=*)C(C)CC2 ; *n1ncc2c1C(=*)C(C)(C)CC2]

may be similarly prepared according to the procedure described in Scheme 13 above, by reacting the compound of formula (b-1) with a suitably selected methylating agent such as Mel, and the like (rather than with the fluorinating agent); in the presence of a suitably selected base such as LiHMDS, LDA, and the like; in a suitably selected organic solvent such as THF, diethyl ether, and the like; at a temperature in the range of from about −78° C. to about room temperature; to yield the corresponding mono- or di-methylated compound of formula (XXVI). One skilled in the art will further recognize that di-methylation may be achieved by introducing the two methyl groups sequentially or simultaneously, in a manner similar to that described for the di-fluorination of the compound of formula (b-1) in Scheme 13 above.

Compounds of formula (XXVI) wherein

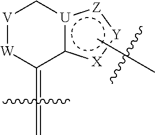

is selected from the group consisting of

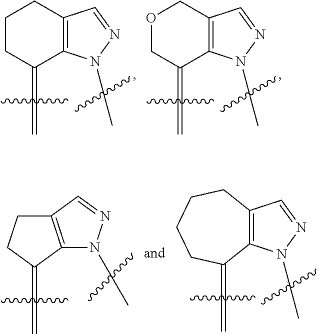

may be prepared as described in Scheme 14, below.

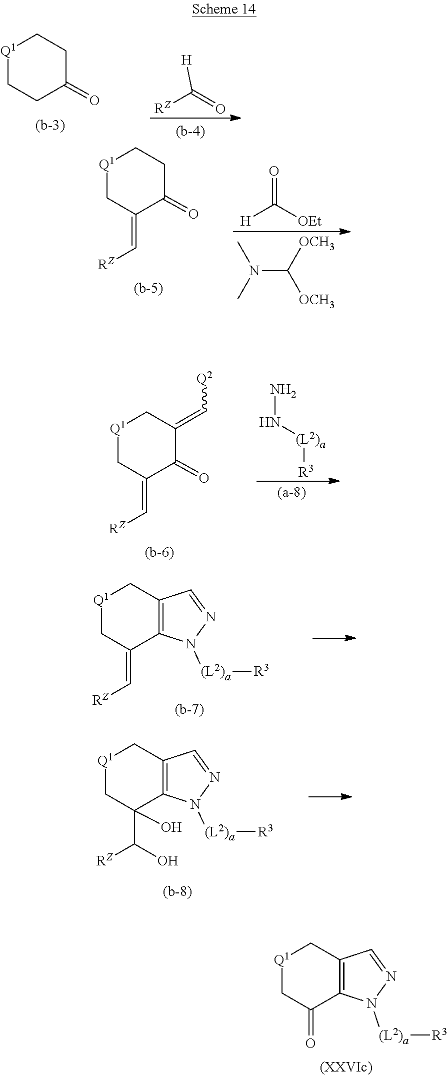

Accordingly, a suitably substituted compound of formula (b-3), wherein Q¹ is absent or is selected from the group consisting of —CH₂—, —O— and —CH₂CH₂—, a known compound or compound prepared by known methods, is reacted with a suitably substituted compound of formula (b-4), wherein R^{Z} is a suitably selected substituents such as phenyl, 4-chlorophenyl, 4-bromophenyl, and the like, a known compound or compound prepared by known methods; in the presence of a suitably selected base such as NaOH, KOH, and the like; in water or a suitably selected mixture of organic solvent and water such as EtOH and water, toluene and water, and the like; at a temperature in the range of from about room temperature to about 80° C., to yield the corresponding compound of formula (b-5).

The compound of formula (b-5) is reacted with ethyl formate, a known compound, in the presence of a suitably selected base such as NaOEt, NaH, and the like; in a suitably selected organic solvent such as toluene, EtOH, and the like; at a temperature in the range of from about 0° C. to about room temperature; to yield the corresponding compound of formula (b-6), wherein Q² is —OH.

Alternatively, the compound of formula (b-5) is reacted with 1,1-dimethoxy-N,N-dimethylmethanamine, a known compound, in a suitably selected organic solvent such as toluene, DMF, and the like; at a temperature in the range of from about 100° C. to 160° C., to yield the corresponding compound of formula (b-6), wherein Q² is —N(CH₃)₂. The compound of formula (b-6), wherein Q² is —N(CH₃)₂, may be further optionally reacted with a suitably selected acid such as HCl, H₂SO₄, and the like; in a suitably selected mixture of organic solvent and water such as CH₂Cl₂, CHCl₃, THF, and the like; at about room temperature; to yield the corresponding compound of formula (b-6), wherein Q² is —OH.

The compound of formula (b-6) is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to 80° C.; to yield the corresponding compound of formula (b-7).

The compound of formula (b-7) is reacted with a suitably selected oxidizing reagent such as OsO₄, K₂OsO₄, and the like; in the presence of 4-methylmorpholine 4-oxide, optionally in the presence of a base such as 2,6-lutidine, 2,4,6-collidine, and the like; in a suitably selected mixture of organic solvent and water such as acetone and water, and the like; at about room temperature; to yield the corresponding compound of formula (b-8).

The compound of formula (b-8) is reacted with a second, suitably selected oxidizing reagent such as NaIO₄, PhI(OAc)₂, and the like; at a temperature of about room temperature; to yield the corresponding compound of formula (XXVIc).

Compounds of formula (XXVI) wherein

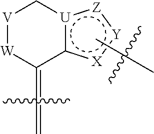

is selected from the group consisting of

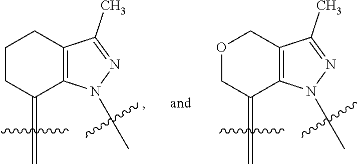 [SMILES: *n1nc(C)c2c1C(=*)CCC2 ; *n1nc(C)c2c1C(=*)COC2]

may be prepared as described in Scheme 15, below.

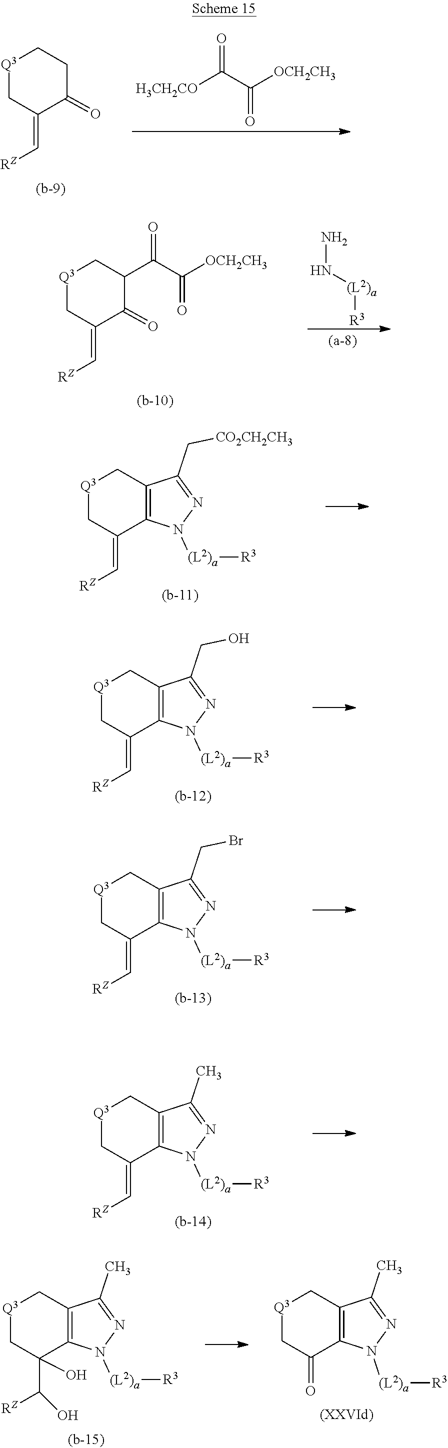

Accordingly, a suitably substituted compound of formula (b-9), wherein Q³ is selected from the group consisting of —CH₂— and —O— and wherein R^{Z} is a suitably selected substituent such as phenyl, 4-chlorophenyl, 4-bromophenyl, and the like, a known compound or compound prepared by known methods, is reacted with diethyl oxalate, a known compound, in the presence of a known base such as LiHMDS, LDA, and the like; in a suitably selected organic solvent such as THF, diethyl ether, and the like; at a temperature in the range of from about −78° C. to about room temperature; to yield the corresponding compound of formula (b-10).

The compound of formula (b-10) is reacted with a suitably substituted compound of formula (a-8), a known compound or compound prepared by known methods, in the presence of a suitably selected acid such as TFA, H₂SO₄, and the like; in a suitably selected organic solvent such as 1,4-dioxane, EtOH, and the like; at a temperature in the range of from about room temperature to about 80° C.; to yield the corresponding compound of formula (b-11).

The compound of formula (b-11) is reacted with a suitably selected reducing reagent such as DIBAL-H, LAH, LiBH₄, and the like; in a suitably selected organic solvent such as THF, CH₂Cl₂, toluene, and the like, at a temperature in the range of from about −78° C. to about room temperature; to yield the corresponding compound of formula (b-12).

The compound of formula (b-12) is reacted with a suitably selected brominating reagent such as NBS, CBr₄, Br₂, and the like; in the presence of a suitably selected phosphine such as PPh₃, and the like; in a suitably selected organic solvent such as CH₂Cl₂, CHCl₃, and the like; at a temperature in the range of from about 0° C. to about room temperature; to yield the corresponding compound of formula (b-13).

The compound of formula (b-13) is reacted with a suitably selected reducing reagent such as NaBH₄, LiAlH₄, and the like; in a suitably selected organic solvent such as THF, DMSO, and the like; at a temperature in the range of from about 0° C. to about room temperature; to yield the corresponding compound of formula (b-14).

The compound of formula (b-14) is reacted with a suitably selected oxidizing reagent such as OsO₄, K₂OsO₄, and the like; in the presence of 4-methylmorpholine 4-oxide, and optionally in the presence of a suitably selected base such as 2,6-lutidine, 2,4,6-collidine, and the like; in a suitably selected mixture of organic solvent and water such as acetone and water, t-butanol and water, and the like; at a temperature about room temperature; to yield the corresponding compound of formula (b-15).

The compound of formula (b-15) is reacted with a second, suitably selected oxidizing reagent such as NaIO₄, PhI(OAc)₂, and the like; at a temperature about room temperature; to yield the corresponding compound of formula (XXVId).

Compounds of formula (XXVI) wherein

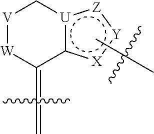

is selected from the group consisting of

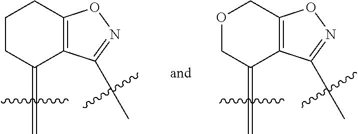 [SMILES: *c1noc2c1C(=*)CCC2 ; *c1noc2c1C(=*)COC2]

may be prepared as described in Scheme 16, below.

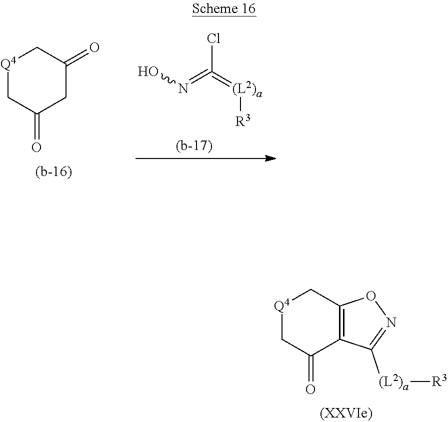

Accordingly, a suitably substituted compound of formula (b-19), wherein Q⁴ is selected from the group consisting of —CH₂— and —O—, a known compound or compound prepared by known methods, is reacted with a suitably substituted compound of formula (b-17), a known compound or compound prepared by known methods; in the presence of a suitably selected base such as NaH, NaOEt, Et₃N, and the like; in a suitably selected organic solvent such as iPrOH, EtOH, THF, and the like; at a temperature in the range of from about 0° C. to about room temperature; to yield the corresponding compound of formula (XXVId).

Compounds of formula (XXVI) wherein

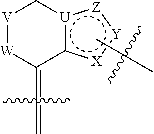

is selected from the group consisting of

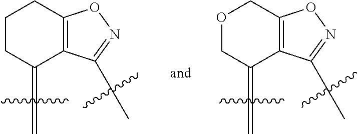 [SMILES: *c1noc2c1C(=*)CCC2 ; *c1noc2c1C(=*)COC2]

may alternatively be prepared as described in Scheme 17, below.

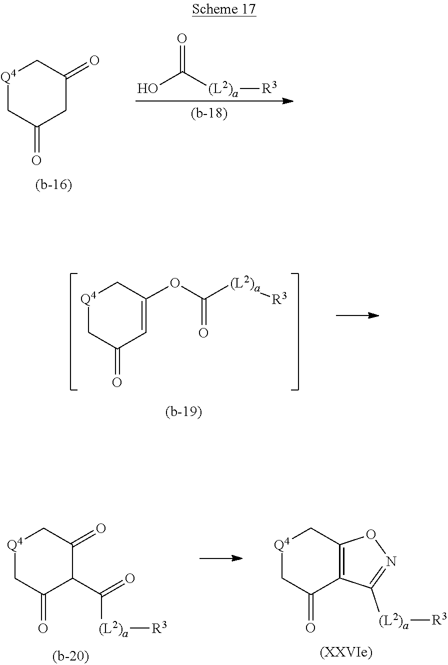

Accordingly, a suitably substituted compound of formula (b-16), wherein Q⁴ is selected from the group consisting of —CH₂— and —O—, a known compound or compound prepared by known methods, is reacted with a suitably substituted compound of formula (b-18), a known compound or compound prepared by known methods; in the presence of a suitably selected coupling reagent such as DCC, EDC, and the like; in a suitably selected organic solvent such as EtOAc, CH₂Cl₂, and the like; at a temperature about room temperature; to yield the corresponding compound of formula (b-19).

The compound of formula (b-19) is reacted with 2-hydroxy-2-methylpropanenitrile, a known compound, in the presence of a suitably selected base such as Et₃N, iPr₂EtN, and the like; in a suitably selected organic solvent such as CH₃CN, and the like; at a temperature about room temperature; to yield the corresponding compound of formula (b-20).

The compound of formula (b-20) is reacted with hydroxylamine, a known compound, optionally in the presence of a suitably selected base such as NaOH, KOH, and the like; in a suitably selected organic solvent such as EtOH, MeOH, and the like; at a temperature in the range of from about 40° C. to about 80° C., to yield the corresponding compound of formula (XXVId).

Compounds of formula (XXVI) wherein

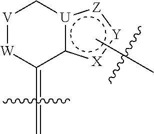

is selected from the group consisting of

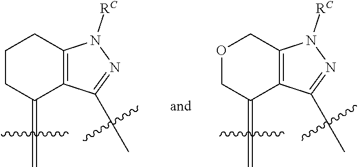

may alternatively be prepared as described in Scheme 18, below.

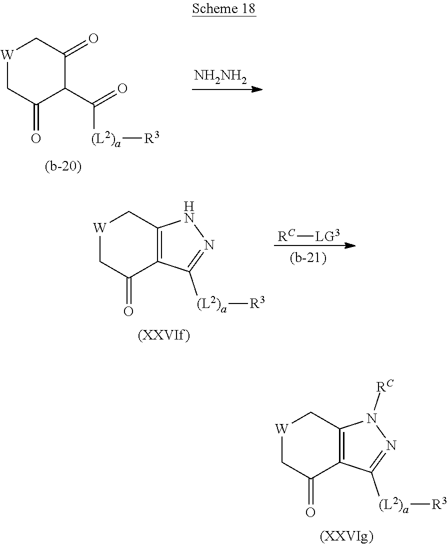

Accordingly, a suitably substituted compound of formula (b-20) wherein Q⁴ is selected from the group consisting of —CH₂— and —O—, a known compound or compound prepared by known methods (for example as described in Scheme 17 above), is reacted with hydrazine, a known compound, in a suitably selected organic solvent such as 1,4-dioxane, EtOH, MeOH, and the like; at about room temperature; to yield the corresponding compound of formula (XXVIf) (wherein R^{D} is hydrogen).

The compound of formula (XXVIf) is further, optionally reacted with a suitably substituted compound of formula (b-21), wherein LG³ is a suitably selected leaving group such as I, Br, Cl, OMs, OTs, and the like, a known compound or compound prepared by known methods; in the presence of a suitably selected base such as K₂CO₃, Cs₂CO₃, and the like; in a suitably selected organic solvent such as CH₃CN, acetone, 1,4-dioxane, and the like; at a temperature in the range of from 60° C. to about 100° C., to yield the corresponding compound of formula (XXVIg) (wherein R^{D} is other than hydrogen).

Compounds of formula (I) wherein

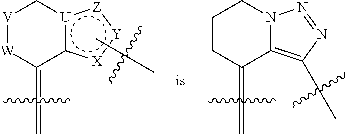

may be prepared as described in Scheme 19, below.

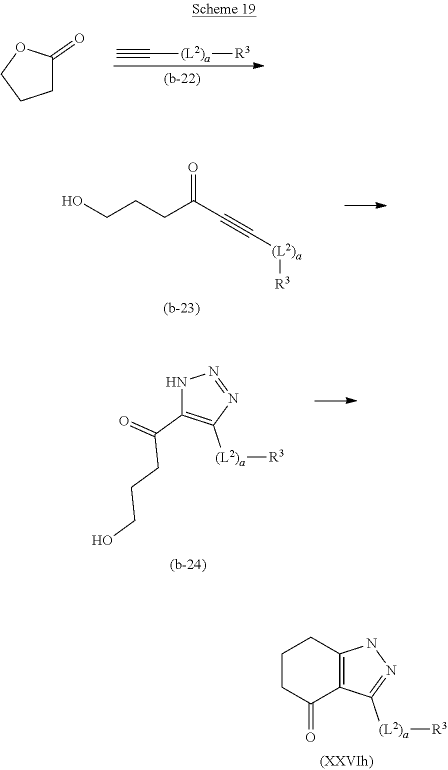

Accordingly, dihydrofuran-2(3H)-one, a known compound, is reacted with a suitably substituted compound of formula (b-22), a known compound or compound prepared by known methods; in the presence of a suitably selected base such as n-BuLi, LDA, LiHMDS, and the like; in a suitably selected organic solvent such as THF, diethyl ether, and the like; at a temperature in the range of from about −78° C. to about room temperature; to yield the corresponding compound of formula (b-23).

The compound of formula (b-23) is reacted with a suitably selected azide reagent such as NaN₃, (CH₃)₃SiN₃, and the like; optionally in the presence of a suitably selected catalyst such as CuI, CuCl, and the like; in a suitably selected organic solvent such as DMF, 1,4 dioxane, and the like; at a temperature in the range of from about room temperature to 80° C.; to yield the corresponding compound of formula (b-24).

The compound of formula (b-24) is reacted with a suitably selected azodicarboxylate reagent such as DEAD, DIAD, di-tert-butyl diazene-1,2-dicarboxylate and the like; in the presence of a suitably selected phosphine reagent such as PPh₃, PBu₃, and the like; in a suitably selected organic solvent such as THF, diethyl ether, CHCl₃, and the like; at about room temperature; to yield the corresponding compound of formula (XXVIh).

Compounds of formula (I) wherein

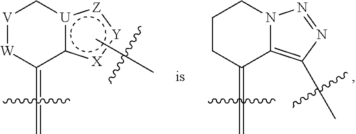

wherein (L²)ₐ is —CH₂— and wherein R³ is hydroxyl, substituted with a suitably selected protecting group may be prepared as described in Scheme 20, below.

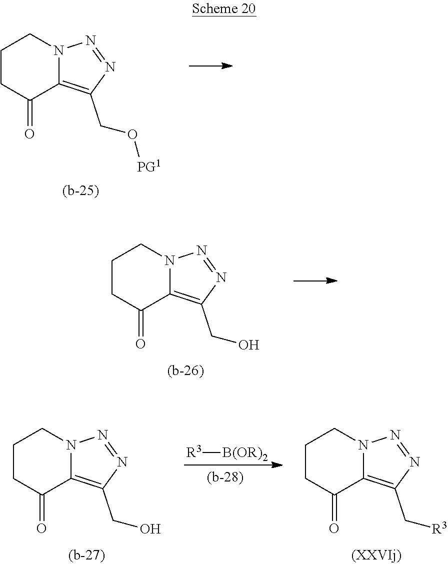

Accordingly, a suitably substituted compound of formula (b-25), wherein PG¹ is a suitably selected oxygen protecting group such as TBS, TBDPS, benzyl, prepared for example, as described in Scheme 19 above, is de-protecting according to known method; to yield the corresponding compound of formula (b-26). For example, wherein PG¹ is TBS, the compound of formula (b-25) is de-protected by reacting with conc. HCl.

The compound of formula (b-26) is reacted to yield the corresponding compound of formula (b-27), according to known methods, by for example reacting the compound of formula (b-26) such that the terminal hydroxy group is replaced with the a suitably selected LG⁴ leaving group such as Br, Cl, OTs, and the like. For example, wherein LG⁴ is Cl, the compound of formula (b-26) is react with SOCl₂. In another example, wherein LG⁴ is OTs, the compound of formula (b-26) is reacted with TsCl.

The compound of formula (b-27) is reacted with a suitably substituted boronic acid or boronic ester, a compound of formula (b-28), wherein the two R groups are each H, are each the same C₁₋₂alkyl or are taken together as —C(CH₃)₂—C(CH₃)₂— to form a ring (i.e. to form the

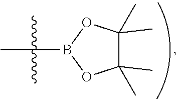 [SMILES: *B1OC(C)(C)C(C)(C)O1]

a known compound or compound prepared by known methods, under Suzuki coupling conditions, more particularly, in the presence of a suitably selected catalyst such as Pd(PPh₃)₄, Pd(dppf)Cl₂, and the like; in the presence of a suitably selected base such as K₂CO₃, Na₂CO₃, and the like; in a suitably selected solvent or mixture of organic solvent and water, such as 1,4-dioxane, 1,4-dioxane and water, and the like; at a temperature in the range of from about 80° C. to about 140° C.; to yield the corresponding compound of formula (XXVID.

One skilled in the art will recognize that compounds of formula (V) and/or compounds of formula (XXVI) wherein

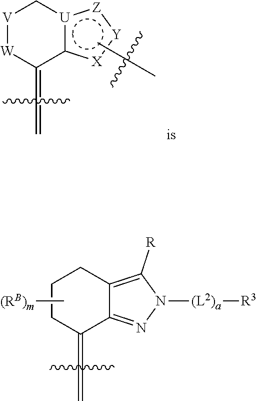

may be prepared as described in for example Schemes 8-10 and 12-15, respectively, as the regio-isomer product of the reaction of the suitably substituted compound of formula (a-8) or compound of formula (a-11) and the corresponding compound as defined in Schemes 8-10 and 12-15.

### Pharmaceutical Compositions

The present invention further comprises pharmaceutical compositions containing one or more compounds of formula (I), compounds of formula (II) and/or compounds of formula (III) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.01 mg/kg/day to about 300 mg/kg/day, or any amount or range therein, preferably from about 0.1 mg/kg/day to about 50 mg/kg/day, or any amount or range therein, preferably from about 0.05 mg/kg/day to about 15 mg/kg/day, or any amount or range therein, preferably from about 0.05 mg/kg/day to about 7.5 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective dosage forms such as tablets, pills and capsules. This solid pre-formulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably from about 1.0 mg to about 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I), compound of formula (II) or compound of formula (III), as the active ingredient(s) is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of the present invention may be administered in any of the foregoing compositions and according to dosage regimens established in the art whenever treatment of disorders mediated by the EP3 receptor is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 500 mg/kg of body weight per day, or any amount or range therein. Preferably, the range is from about 0.05 to about 50.0 mg/kg of body weight per day, or any amount or range therein. More preferably, from about 0.05 to about 15.0 mg/kg of body weight per day, or any amount or range therein. More preferably, from about 0.05 to about 7.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### SYNTHESIS EXAMPLES

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1: Compound #5

### (E)-2-(1-(2,4-dichlorobenzyl)-1,4,5,6-tetrahydro-7H-indazol-7-ylidene)ethyl ((4,5-dichlorothiophen-2-yl)sulfonyl)carbamate

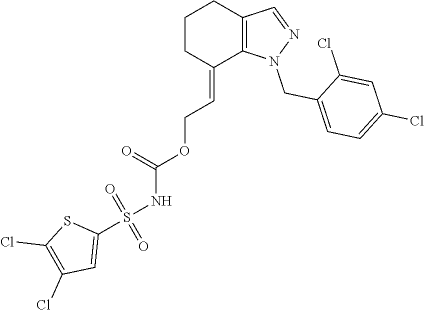 [SMILES: O=C(NS(=O)(=O)c1cc(Cl)c(Cl)s1)OC/C=C1\CCCc2cnn(Cc3ccc(Cl)cc3Cl)c21]

### Step 1. Synthesis of ethyl (E)-2-(2-oxocyclohexylidene)acetate

To a 250-mL round bottle was added cyclohexane-1,2-dione (5 g, 0.046 mol), EtOH (50 mL) and ethyl (triphenylphosphoranylidene)acetate (15.5 g, 0.044 mol). The resulting solution was stirred for 16 h at 90° C. The resulting solution was diluted with H₂O and extracted with EtOAc. The organic layers were combined and concentrated under vacuum. The resulting residue was purified by silica gel column with PE: EA=90/10 to yield ethyl (E)-2-(2-oxocyclohexylidene)acetate as yellow oil. Mass spectrum (ESI, m/z): Calculated for C₁₀H₁₅O₃, 183.1 [M+H], found 183.0.

### Step 2. Synthesis of ethyl (E)-2-((E)-3-(hydroxymethylene)-2-oxocyclohexylidene)acetate

A solution of ethyl (E)-2-(2-oxocyclohexylidene) acetate (20 g, 0.11 mol) in N,N-dimethylformamide dimethyl acetal (52.4 g, 0.44 mol) was stirred for 2 days at room temperature. The resulting solution was concentrated under vacuum and the resulting residue was stirred vigorously in DCM/HCl (2M) at room temperature for 8 h. The resulting mixture was extracted with DCM. The organic layers were combined, dried over Na₂SO₄, and concentrated under vacuum to yield ethyl (E)-2-((E)-3-(hydroxymethylene)-2-oxocyclohexylidene)acetate as yellow oil. Mass spectrum (ESI, m/z): Calculated for C₁₁H₁₅O₄, 211.1 [M+H], found 211.0.

### Step 3. Synthesis of ethyl (E)-2-(1-(2,4-dichlorobenzyl)-1,4,5,6-tetrahydro-7H-indazol-7-ylidene)acetate

A solution of (E)-ethyl 2-((E)-3-(hydroxymethylene)-2-oxocyclohexylidene)acetate (18 g, 85.6 mmol), (2,4-dichlorobenzyl)hydrazine hydrocholride (20 g, 87.9 mmol), and TFA (5 mL) in 1,4-dioxane (50 mL) was heated at 60° C. for 2 hours. The reaction mixture was concentrated under vacuum to remove most organic solvent. The resulting residue was neutralized with aq NaHCO₃ and extracted with EtOAc. The organic layer was washed with sat. aq. NaCl and dried over Na₂SO₄, and concentrated. Purification of the resulting residue by silica gel column with 2% EtOAc/heptane yielded ethyl (E)-2-(1-(2,4-dichlorobenzyl)-1,4,5,6-tetrahydro-7H-indazol-7-ylidene)acetate as a light yellow solid.

¹H NMR (300 MHz, CD₃OD, ppm) δ: 7.58 (s, 1H), 7.50 (s, 1H), 7.28-7.30 (m, 1H), 6.55 (d, J=8.4 Hz, 1H), 5.69 (s, 1H), 5.61 (s, 2H), 4.09-4.15 (m, 2H), 3.21 (t, J=4.8 Hz, 2H), 2.72 (t, J=6.0 Hz, 2H), 1.86-1.93 (m, 2H), 1.24 (t, J=6.8 Hz, 3H). Mass spectrum (ESI, m/z): Calculated for C₁₈H₁₉Cl₂N₂O₂, 365.1 [M+H], found 365.1.

### Step 4. Synthesis of (E)-2-(1-(2,4-dichlorobenzyl)-1,4,5,6-tetrahydro-7H-indazol-7-ylidene)ethan-1-ol

To a solution of ethyl (E)-2-(1-(2,4-dichlorobenzyl)-1,4,5,6-tetrahydro-7H-indazol-7-ylidene)acetate (1 g, 2.64 mmol) in THF (50 mL) at −78° C. was added DIBAL-H (13.7 mL, 13.7 mmol) in portions. The resulting solution was stirred for 30 min at −78° C. and stirred for 3 h at 0° C. The reaction progress was monitored by LCMS. The reaction was then quenched by the addition of MeOH (0.5 mL) and diluted with H₂O (50 mL). The resulting mixture was extracted with DCM and the organic layers were combined and concentrated under vacuum. The resulting residue was purified by silica gel column with PE:EA=50/50 to yield (E)-2-(1-(2,4-dichlorobenzyl)-1,4,5,6-tetrahydro-7H-indazol-7-ylidene)ethan-1-ol as white solid. Mass spectrum (ESI, m/z): Calculated for C₁₆H₁₇Cl₂N₂O, 323.1 [M+H], found 322.9.

### Step 5. Synthesis of (E)-2-(1-(2,4-dichlorobenzyl)-1,4,5,6-tetrahydro-7H-indazol-7-ylidene)ethyl ((4,5-dichlorothiophen-2-yl)sulfonyl)carbamate

To a solution of 4,5-dichlorothiophene-2-sulfonamide (1.0 g, 4.31 mmol) in THF (30 mL) was added di(1H-imidazol-1-yl)methanone (677 mg, 4.18 mmol). The reaction mixture was stirred for 16 hours at room temperature. (E)-2-(1-(2,4-dichlorobenzyl)-1,4,5,6-tetrahydro-7H-indazol-7-ylidene)ethan-1-ol (500 mg, 1.55 mmol) was then added. The resulting mixture was heated by microwave at 100° C. for 1 hour. The reaction mixture was concentrated under vacuum. The resulting residue was purified by Prep-HPLC with the following conditions (1#waters2767-5): Column, SunFire Prep C18, 19*150 mm Sum; mobile phase, water with 0.05% NH₄HCO₃ and CH₃CN (20% CH₃CN up to 60% in 10 min, up to 100% CH₃CN in 0.1 min, hold 100% in 1.9 min, down to 20% CH₃CN in 0.1 min, hold 20% in 1.9 min); Detector, UV 220 & 254 nm to yield (E)-2-(1-(2,4-dichlorobenzyl)-1,4,5,6-tetrahydro-7H-indazol-7-ylidene)ethyl ((4,5-dichlorothiophen-2-yl)sulfonyl)carbamate as a white solid.

¹H NMR (300 MHz, DMSO) δ: 7.88 (s, 1H), 7.61 (s, 1H), 7.41 (s, 1H), 7.34-7.40 (m, 1H), 6.49-6.59 (m, 1H), 5.37-5.47 (m, 3H), 4.72 (d, J=6.9 Hz, 2H), 2.56-2.60 (m, 2H), 2.36-2.43 (m, 2H), 1.71-1.75 (m, 2H). Mass spectrum (ESI, m/z): Calculated for C₂₁H₁₈Cl₄N₃O₄S₂, 582.3 (M+H), found 582.0.

### Example 2: Compound #2

### (E)-2-(1-(2,4-dichlorobenzyl)-1,4,5,6-tetrahydro-7H-indazol-7-ylidene)ethyl ((4-chlorophenyl)sulfonyl)carbamate

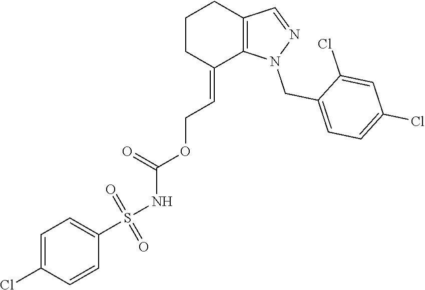 [SMILES: O=C(NS(=O)(=O)c1ccc(Cl)cc1)OC/C=C1\CCCc2cnn(Cc3ccc(Cl)cc3Cl)c21]

### Step 1. Synthesis of (E)-2-(1-(2,4-dichlorobenzyl)-1,4,5,6-tetrahydro-7H-indazol-7-ylidene)ethyl ((4-chlorophenyl)sulfonyl)carbamate

To a solution of (E)-2-(1-(2,4-dichlorobenzyl)-1,4,5,6-tetrahydro-7H-indazol-7-ylidene)ethan-1-ol (100 mg, 0.309 mmol) in CH₂Cl₂ (6 mL) at 0° C. was added 4-chlorobenzenesulfonyl isocyanate (0.07 mL, 0.47 mmol). The reaction was kept at 0° C. for 5 min before the reaction mixture was warmed up to room temperature and stirred at room temperature for 30 min. To the reaction was then added MeOH (0.5 mL) and the reaction was stirred for another 10 min. The reaction solution was concentrated and the residue was dissolved in CH₂Cl₂ and washed with aq. NaHCO₃. The organic layer was dried over Na₂SO₄ and concentrated. The resulting residue was purified by silica gel column with 40% EtOAc/heptane to yield (E)-2-(1-(2,4-dichlorobenzyl)-1,4,5,6-tetrahydro-7H-indazol-7-ylidene)ethyl ((4-chlorophenyl)sulfonyl)carbamate.

¹H NMR (400 MHz, CDCl₃) δ: 7.95 (d, J=7.9 Hz, 2H), 7.47-7.56 (m, 2H), 7.39-7.43 (m, 2H), 7.13 (dd, J=8.1, 2.0 Hz, 1H), 6.50 (d, J=8.1 Hz, 1H), 5.46 (s, 2H), 5.25-5.31 (m, 1H), 4.66 (d, J=7.1 Hz, 2H), 2.65 (t, J=6.1 Hz, 2H), 2.36-2.44 (m, 2H), 1.63-1.86 (m, 2H). Mass spectrum (ESI, m/z): Calculated for C₂₃H₂₁Cl₃N₃O₄S, 540.0 (M+H), found 540.0.

### Example 3: Compound #3

### (E)-3-(1-(2,4-dichlorobenzyl)-1,4,5,6-tetrahydro-7H-indazol-7-ylidene)-N-((4,5-dichlorothiophen-2-yl)sulfonyl)propanamide

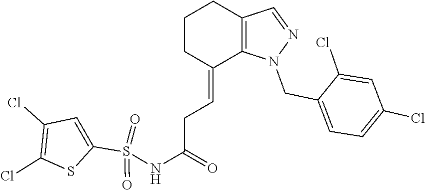 [SMILES: O=C(C/C=C1\CCCc2cnn(Cc3ccc(Cl)cc3Cl)c21)NS(=O)(=O)c1cc(Cl)c(Cl)s1]

### Step 1. Synthesis of (E)-3-(1-(2,4-dichlorobenzyl)-1,4,5,6-tetrahydro-7H-indazol-7-ylidene)propanenitrile\

To a solution of (E)-2-(1-(2,4-dichlorobenzyl)-1,4,5,6-tetrahydro-7H-indazol-7-ylidene)ethan-1-ol (716 mg, 2.22 mmol), 2-hydroxy-2-methylpropanenitrile (754 mg, 8.86 mmol), and PPh₃ (1161 mg, 4.43 mmol) in THF (20 mL) at room temperature was added DIAD (896 mg, 4.43 mmol). The reaction was stirred at room temperature overnight. The reaction was then quenched with H₂O. The resulting mixture was extracted with DCM, and the organic layers were combined and concentrated under vacuum. The resulting residue was purified by silica gel column with PE:EA=70:30 to yield (E)-3-(1-(2,4-dichlorobenzyl)-1,4,5,6-tetrahydro-7H-indazol-7-ylidene)propanenitrile as yellow solid. Mass spectrum (ESI, m/z): Calculated for C₁₇H₁₆Cl₂N₃, 332.1 [M+H], found 332.0.

### Step 2. Synthesis of ethyl (E)-3-(1-(2,4-dichlorobenzyl)-1,4,5,6-tetrahydro-7H-indazol-7-ylidene)propanoate

A solution of (E)-3-(1-(2,4-dichlorobenzyl)-1,4,5,6-tetrahydro-7H-indazol-7-ylidene)propanenitrile (100 mg, 0.301 mmol), SOCl₂ (0.5 mL) in EtOH (10 mL) was stirred at 80° C. overnight. The reaction was quenched by aq. NaHCO₃. The resulting mixture was diluted with EtOAc and washed with aq NaCl. The organic layer was concentrated under vacuum to yield ethyl (E)-3-(1-(2,4-dichlorobenzyl)-1,4,5,6-tetrahydro-7H-indazol-7-ylidene)propanoate as a yellow oil. Mass spectrum (ESI, m/z): Calculated for C₁₉H₂₁Cl₂N₂O₂, 379.1[M+H], found 379.1.

### Step 3. Synthesis of (E)-3-(1-(2,4-dichlorobenzyl)-1,4,5,6-tetrahydro-7H-indazol-7-ylidene)propanoic acid

A mixture of (E)-ethyl 3-(1-(2,4-dichlorobenzyl)-5,6-dihydro-1H-indazol-7(4H)-ylidene)propanoate (1 g, 2.64 mmol) and LiOH (0.5 g, 20.9 mmol) in H₂O (10 mL) and 1,4-dioxane (20 mL) was stirred at room temperature for 3 h. The reaction was diluted with H₂O and the resulting solution was extracted with Et₂O. The aqueous layer was collected and pH was adjusted to ˜5-6. The resulting mixture was extracted with EtOAc. The organic layer was washed with aq NaCl, and concentrated under vacuum to yield (E)-3-(1-(2,4-dichlorobenzyl)-1,4,5,6-tetrahydro-7H-indazol-7-ylidene)propanoic acid as a yellow oil. Mass spectrum (ESI, m/z): Calculated for C₁₇H₁₇Cl₂N₂O₂, 351.1[M+H], found 351.1.

### Step 4. Synthesis of (E)-3-(1-(2,4-dichlorobenzyl)-1,4,5,6-tetrahydro-7H-indazol-7-ylidene)-N-((4,5-dichlorothiophen-2-yl)sulfonyl)propenamide

A solution of (E)-3-(1-(2,4-dichlorobenzyl)-1,4,5,6-tetrahydro-7H-indazol-7-ylidene)propanoic acid (320 mg, 0.911 mmol), 4,5-dichlorothiophene-2-sulfonamide (275 mg, 1.19 mmol), DMAP (223 mg, 1.83 mmol), and EDCI (350 mg, 1.83 mmol) in DCM (10 mL) was stirred for 16 hours at room temperature. The reaction solution was then diluted with EtOAc and washed with aq NaCl. The organic layer was concentrated under vacuum. The resulting resulting residue was purified by Prep-HPLC with the following conditions (1#-Waters 2767-5): Column, SunFire Prep C18 5 um, 19*100 mm; mobile phase, Water with 0.05% TFA and CH₃CN (20% CH₃CN up to 53% in 10 min, up to 100% in 0.1 min, hold 100% in 0.9 min, down to 20% in 0.1 min, hold 20% in 1.4 min); Detector, UV 220 & 254 nm to yield (E)-3-(1-(2,4-dichlorobenzyl)-1,4,5,6-tetrahydro-7H-indazol-7-ylidene)-N-((4,5-dichlorothiophen-2-yl)sulfonyl)propenamide as a white solid.

¹H NMR (400 MHz, Methanol-d4) δ: 7.69 (s, 1H), 7.51 (s, 1H), 7.41 (s, 1H), 7.24 (d, J=8.4 Hz, 1H), 6.48 (d, J=8.4 Hz, 1H), 5.53 (s, 2H), 5.46 (t, J=2.6 Hz, 1H), 3.16 (d, J=6.8 Hz, 2H), 2.66 (t, J=6.0 Hz, 2H), 2.41 (t, J=5.6 Hz, 2H), 1.82-1.85 (m, 2H). Mass spectrum (ESI, m/z): Calculated for C₂₁H₁₈Cl₄N₃O₃S₂, 566.3 [M+H], found 566.0.

The following representative compounds of formula (I) were similarly prepared according to the procedure described in Example 3 above, selecting and substituting suitable starting materials and reagents, as would be readily recognized by those skilled in the art.

### (E)-3-(1-(2,4-dichlorobenzyl)-1,4,5,6-tetrahydro-7H-indazol-7-ylidene)-N-(isopropylsulfonyl)propenamide, Compound #14

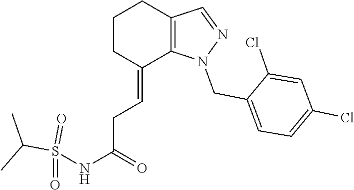 [SMILES: CC(C)S(=O)(=O)NC(=O)C/C=C1\CCCc2cnn(Cc3ccc(Cl)cc3Cl)c21]

¹H NMR (400 MHz, Methanol-d4) δ: 7.55 (s, 1H), 7.42 (s, 1H), 7.25 (d, J=8.4 Hz, 1H), 6.48 (d, J=8.4 Hz, 1H), 5.52-5.64 (m, 3H), 3.60-3.67 (m, 1H), 3.22 (d, J=7.2 Hz, 2H), 2.69 (t, J=6.4 Hz, 2H), 2.48 (t, J=6.4 Hz, 2H), 1.85-1.91 (m, 2H), 1.35 (d, J=6.4 Hz, 6H). Mass spectrum (ESI, m/z): Calculated for C₂₀H₂₄Cl₂N₃O₃S, 456.1 [M+H], found 456.1.

### (E)-N-((5-chlorothiophen-2-yl)sulfonyl)-3-(1-(2,4-dichlorobenzyl)-1,4,5,6-tetrahydro-7H-indazol-7-ylidene)propenamide, Compound #17

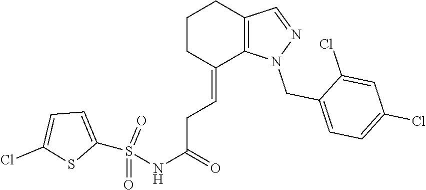 [SMILES: O=C(C/C=C1\CCCc2cnn(Cc3ccc(Cl)cc3Cl)c21)NS(=O)(=O)c1ccc(Cl)s1]

¹H NMR (300 MHz, Methanol-d4) δ: 7.59 (d, J=4.2 Hz, 1H), 7.47 (s, 1H), 7.36 (s, 1H), 7.19 (d, J=8.1 Hz, 1H), 7.07 (d, J=4.2 Hz, 1H), 6.42 (d, J=8.4 Hz, 1H), 5.47 (s, 2H), 5.40 (t, J=6.9 Hz, 1H), 3.10 (d, J=6.9 Hz, 2H), 2.61 (t, J=6.3 Hz, 2H), 2.36 (t, J=5.4 Hz, 2H), 1.74-1.82 (m, 2H). Mass spectrum (ESI, m/z): Calculated for C₂₁H₁₉Cl₃N₃O₃S₂, 531.9 (M+H), found 532.0.

### (E)-3-(1-(2,4-dichlorobenzyl)-1,4,5,6-tetrahydro-7H-indazol-7-ylidene)-N-((2,3-dihydrobenzo[b][1,4]dioxin-6-yl)sulfonyl)propenamide, Compound #18

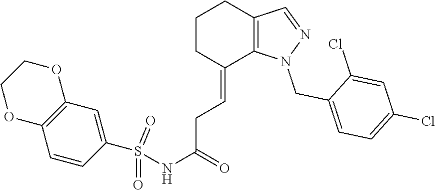 [SMILES: O=C(C/C=C1\CCCc2cnn(Cc3ccc(Cl)cc3Cl)c21)NS(=O)(=O)c1ccc2c(c1)OCCO2]

¹H NMR (300 MHz, Methanol-d4) δ: 7.34-7.45 (m, 4H), 7.18 (d, J=8.4 Hz, 1H), 6.95 (d, J=6.6 Hz, 1H), 6.42 (d, J=8.7 Hz, 1H), 5.44 (s, 2H), 5.36 (t, J=7.2 Hz, 1H), 4.25-4.32 (m, 4H), 3.05 (d, J=6.9 Hz, 2H), 2.59 (t, J=6.3 Hz, 2H), 2.32 (t, J=5.1 Hz, 2H), 1.71-1.79 (m, 2H). Mass spectrum (ESI, m/z): Calculated for C₂₅H₂₄Cl₂N₃O₅S, 548.1 (M+H), found 548.1.

### (E)-3-(1-(2,4-dichlorobenzyl)-1,4,5,6-tetrahydro-7H-indazol-7-ylidene)-N-((3,5-difluorophenyl)sulfonyl)propenamide, Compound #19

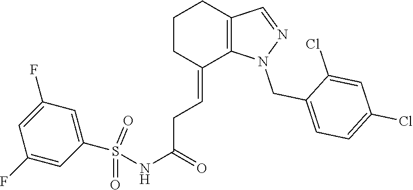 [SMILES: O=C(C/C=C1\CCCc2cnn(Cc3ccc(Cl)cc3Cl)c21)NS(=O)(=O)c1cc(F)cc(F)c1]

¹H NMR (300 MHz, Methanol-d4) δ: 7.52-7.57 (m, 2H), 7.43 (s, 1H), 7.30-7.37 (m, 2H), 7.17 (d, J=8.4 Hz, 1H), 6.40 (d, J=8.4 Hz, 1H), 5.45 (s, 2H), 5.37 (t, J=6.9 Hz, 1H), 3.10 (d, J=6.9 Hz, 2H), 2.60 (t, J=6.3 Hz, 2H), 2.34 (t, J=5.4 Hz, 2H), 1.73-1.83 (m, 2H). Mass spectrum (ESI, m/z): Calculated for C₂₃H₂₀Cl₂F₂N₃O₃S, 526.0 (M+H), found 526.1.

### (E)-3-(1-(2,4-dichlorobenzyl)-1,4,5,6-tetrahydro-7H-indazol-7-ylidene)-N-(thiophen-2-ylsulfonyl)propenamide, Compound #20

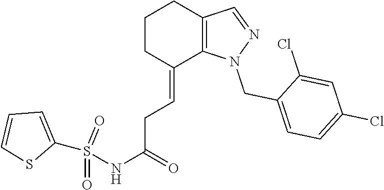 [SMILES: O=C(C/C=C1\CCCc2cnn(Cc3ccc(Cl)cc3Cl)c21)NS(=O)(=O)c1cccs1]

¹H NMR (300 MHz, Methanol-d4) δ: 7.85 (d, J=5.1 Hz, 1H), 7.76 (d, J=3.9 Hz, 1H), 7.47 (s, 1H), 7.35 (s, 1H), 7.11-7.19 (m, 2H), 6.41 (d, J=8.4 Hz, 1H), 5.46 (s, 2H), 5.37 (t, J=6.9 Hz, 1H), 3.09 (d, J=6.9 Hz, 2H), 2.60 (t, J=6.3 Hz, 2H), 2.34 (t, J=5.4 Hz, 2H), 1.75-1.81 (m, 2H). Mass spectrum (ESI, m/z): Calculated for C₂₁H₂₀Cl₂N₃O₃S₂, 497.4 (M+H), found 497.8.

### (E)-N-(benzo[d]thiazol-2-ylsulfonyl)-3-(1-(2,4-dichlorobenzyl)-1,4,5,6-tetrahydro-7H-indazol-7-ylidene)propenamide, Compound #21

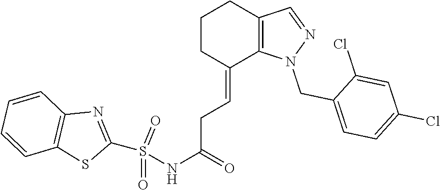 [SMILES: O=C(C/C=C1\CCCc2cnn(Cc3ccc(Cl)cc3Cl)c21)NS(=O)(=O)c1nc2ccccc2s1]

¹H NMR (300 MHz, Methanol-d4) δ: 8.05-8.13 (m, 2H), 7.59-7.67 (m, 2H), 7.33 (s, 1H), 7.25 (s, 1H), 7.09 (d, J=8.4 Hz, 1H), 6.38 (d, J=8.4 Hz, 1H), 5.31-5.40 (m, 3H), 3.19 (d, J=7.2 Hz, 2H), 2.56 (t, J=6.3 Hz, 2H), 2.35 (t, J=4.8 Hz, 2H), 1.69-1.77 (m, 2H). Mass spectrum (ESI, m/z): Calculated for C₂₄H₂₁Cl₂N₄O₃S₂, 547.0 (M+H), found 547.0.

### (E)-3-(1-(2,4-dichlorobenzyl)-1,4,5,6-tetrahydro-7H-indazol-7-ylidene)-N-(quinolin-3-ylsulfonyl)propenamide, Compound #22

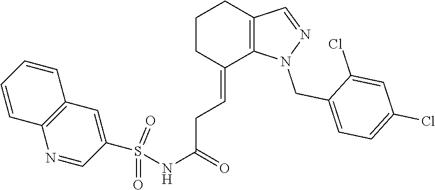 [SMILES: O=C(C/C=C1\CCCc2cnn(Cc3ccc(Cl)cc3Cl)c21)NS(=O)(=O)c1cnc2ccccc2c1]

¹H NMR (300 MHz, Methanol-d4) δ: 9.22 (s, 1H), 8.98 (s, 1H), 8.15 (d, J=8.4 Hz, 2H), 7.98 (t, J=8.4 Hz, 1H), 7.77 (t, J=8.4 Hz, 1H), 7.31 (s, 1H), 7.22 (s, 1H), 7.10 (d, J=8.4 Hz, 1H), 6.35 (d, J=8.4 Hz, 1H), 5.36 (s, 2H), 5.30 (t, J=6.9 Hz, 1H), 3.09 (d, J=6.9 Hz, 2H), 2.53 (t, J=6.3 Hz, 2H), 2.28 (t, J=5.4 Hz, 2H), 1.65-1.73 (m, 2H). Mass spectrum (ESI, m/z): Calculated for C₂₆H₂₃Cl₂N₄O₃S, 541.1 (M+H), found 541.1.

### (E)-3-(1-(2,4-dichlorobenzyl)-1,4,5,6-tetrahydro-7H-indazol-7-ylidene)-N-((4-methoxyphenyl)sulfonyl)propenamide, Compound #23

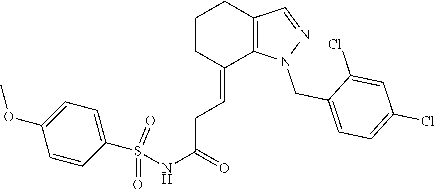 [SMILES: COc1ccc(S(=O)(=O)NC(=O)C/C=C2\CCCc3cnn(Cc4ccc(Cl)cc4Cl)c32)cc1]

¹H NMR (400 MHz, Methanol-d4) δ: 7.92 (d, J=8.8 Hz, 2H), 7.50 (s, 1H), 7.40 (s, 1H), 7.21 (d, J=8.4 Hz, 1H), 7.08 (d, J=8.8 Hz, 2H), 6.45 (d, J=8.4 Hz, 1H), 5.50 (s, 2H), 5.41 (t, J=6.4 Hz, 1H), 3.91 (s, 3H), 3.09 (d, J=6.8 Hz, 2H), 2.64 (t, J=6.0 Hz, 2H), 2.36 (t, J=5.6 Hz, 2H), 1.77-1.83 (m, 2H). Mass spectrum (ESI, m/z): Calculated for C₂₄H₂₄Cl₂N₃O₄S, 520.1 (M+H), found 520.1.

### (E)-N-(benzo[b]thiophen-2-ylsulfonyl)-3-(1-(2,4-dichlorobenzyl)-1,4,5,6-tetrahydro-7H-indazol-7-ylidene)propenamide, Compound #25

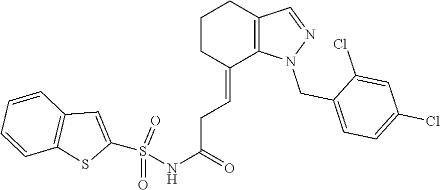 [SMILES: O=C(C/C=C1\CCCc2cnn(Cc3ccc(Cl)cc3Cl)c21)NS(=O)(=O)c1cc2ccccc2s1]

¹H NMR (300 MHz, Methanol-d4) δ: 8.10 (s, 1H), 7.92-7.99 (m, 2H), 7.46-7.56 (m, 2H), 7.33 (s, 1H), 7.28 (s, 1H), 7.12 (d, J=8.4 Hz, 1H), 6.38 (d, J=8.7 Hz, 1H), 5.39-5.40 (m, 3H), 3.11 (d, J=6.9 Hz, 2H), 2.54 (t, J=6.0 Hz, 2H), 2.31 (t, J=5.1 Hz, 2H), 1.69-1.73 (m, 2H). Mass spectrum (ESI, m/z): Calculated for C₂₅H₂₂Cl₂N₃O₃S₂, 546.0 (M+H), found 546.1.

### (E)-N-(benzofuran-2-ylsulfonyl)-3-(1-(2,4-dichlorobenzyl)-1,4,5,6-tetrahydro-7H-indazol-7-ylidene)propenamide, Compound #26

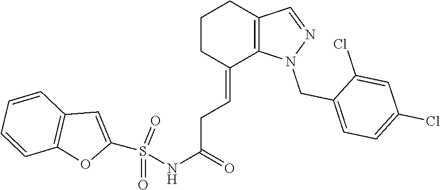 [SMILES: O=C(C/C=C1\CCCc2cnn(Cc3ccc(Cl)cc3Cl)c21)NS(=O)(=O)c1cc2ccccc2o1]

¹H NMR (300 MHz, Methanol-d4) δ: 7.77 (d, J=6.9 Hz, 1H), 7.63 (s, 1H), 7.48-7.54 (m, 2H), 7.35-7.40 (m, 1H), 7.33 (s, 1H), 7.28 (s, 1H), 7.12 (d, J=8.4 Hz, 1H), 6.38 (d, J=8.4 Hz, 1H), 5.34-5.39 (m, 3H), 3.15 (d, J=6.9 Hz, 2H), 2.55 (t, J=6.0 Hz, 2H), 2.32 (t, J=6.0 Hz, 2H), 1.67-1.75 (m, 2H). Mass spectrum (ESI, m/z): Calculated for C₂₅H₂₂Cl₂N₃O₄S, 530.1 (M+H), found 530.1.

### Example 4: Compound #8

### (E)-4-(1-(2,4-dichlorobenzyl)-1,4,5,6-tetrahydro-7H-indazol-7-ylidene)-N-((4,5-dichlorothiophen-2-yl)sulfonyl)butanamide

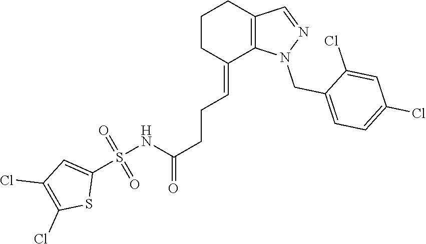 [SMILES: O=C(CC/C=C1\CCCc2cnn(Cc3ccc(Cl)cc3Cl)c21)NS(=O)(=O)c1cc(Cl)c(Cl)s1]

### Step 1. Synthesis of (E)-7-(2-chloroethylidene)-1-(2,4-dichlorobenzyl)-4,5,6,7-tetrahydro-1H-indazole

To a solution of (E)-2-(1-(2,4-dichlorobenzyl)-1,4,5,6-tetrahydro-7H-indazol-7-ylidene)ethan-1-olin (300 mg, 0.93 mmol) in CH₂Cl₂ (10 mL) at 0° C. was added thionyl chloride (0.2 mL, 2.78 mmol). The reaction was stirred at 0° C. for 30 min before the reaction mixture was diluted with EtOAc and washed with H₂O and sat. aq. NaCl. The organic layer was dried over Na₂SO₄ and concentrated to yield (E)-7-(2-chloroethylidene)-1-(2,4-dichlorobenzyl)-4,5,6,7-tetrahydro-1H-indazole.

### Step 2. Synthesis of diethyl (E)-2-(2-(1-(2,4-dichlorobenzyl)-1,4,5,6-tetrahydro-7H-indazol-7-ylidene)ethyl)malonate

To a solution of diethyl malonate (1.98 g, 12.4 mmol) in THF (100 mL) at 0° C. was added Cs₂CO₃ (5 g, 15.3 mmol). The resulting solution was stirred at 0° C. for 30 min before (E)-7-(2-chloroethylidene)-1-(2,4-dichlorobenzyl)-4,5,6,7-tetrahydro-1H-indazole (2.1 g, 6.15 mmol) was added. The resulting solution was then stirred overnight at 50° C. The reaction was quenched by the addition of H₂O. The mixture was diluted with EtOAc and washed with aq NaCl. The organic layer was concentrated under vacuum. The resulting residue was purified by silica gel column with PE:EA=80/20 to yield diethyl (E)-2-(2-(1-(2,4-dichlorobenzyl)-1,4,5,6-tetrahydro-7H-indazol-7-ylidene)ethyl)malonate as colorless oil.

### Step 3. Synthesis of (E)-4-(1-(2,4-dichlorobenzyl)-1,4,5,6-tetrahydro-7H-indazol-7-ylidene)butanoic acid

A mixture of diethyl (E)-2-(2-(1-(2,4-dichlorobenzyl)-1,4,5,6-tetrahydro-7H-indazol-7-ylidene)ethyl)malonate (1 g, 2.15 mmol) and LiCl (0.18 g, 4.25 mmol) in DMSO (10 mL) and H₂O (2 mL) was heated at 170° C. by microwave for 1.5 hour. The reaction was quenched with H₂O, diluted with EA and washed with aq NaCl. The organic solution was concentrated under vacuum to yield a resulting residue. This resulting residue was dissolved in MeOH (20 mL). To the resulting solution was added a solution of NaOH (0.43 g, 10.75 mmol) in H₂O (4 mL). The reaction was stirred overnight at room temperature. The reaction solution was concentrated under vacuum to remove most of the organic solvent. The remaining mixture was diluted with H₂O and the pH was adjusted to ˜4-5. The resulting mixture then was diluted with EtOAc and washed with aq NaCl. The organic layer was concentrated under vacuum to yield a residue. To the residue was added H₂O (10 mL) and the resulting mixture was filtered. The solid was washed with EtOAc and dried under vacuum to yield (E)-4-(1-(2,4-dichlorobenzyl)-1,4,5,6-tetrahydro-7H-indazol-7-ylidene)butanoic acid as white solid.

### Step 4. Synthesis of (E)-4-(1-(2,4-dichlorobenzyl)-1,4,5,6-tetrahydro-7H-indazol-7-ylidene)-N-((4,5-dichlorothiophen-2-yl)sulfonyl)butanamide

A solution of (E)-4-(1-(2,4-dichlorobenzyl)-1,4,5,6-tetrahydro-7H-indazol-7-ylidene)butanoic acid (150 mg, 0.411 mmol), 4,5-dichlorothiophene-2-sulfonamide (124 mg, 0.534 mmol), DMAP (101 mg, 0.828 mmol), and EDCI (158 mg, 0.827 mmol) in DCM (10 mL) was stirred for 16 hour at room temperature. The reaction was diluted with EtOAc and washed with aq NaCl. The organic solution was concentrated under vacuum to yield a residue. The residue was purified by Prep-HPLC with the following conditions (1#-Waters 2767-5): Column, SunFire Prep C18, 5 um, 19*100 mm; mobile phase, Water with 0.05% NH₄HCO₃ and CH₃CN (50% CH₃CN up to 60% in 17 min, up to 100% in 0.1 min, hold 100% in 0.9 min, down to 50% in 0.1 min, hold 50% in 1.4 min); Detector, UV 220 & 254 nm to yield (E)-4-(1-(2,4-dichlorobenzyl)-1,4,5,6-tetrahydro-7H-indazol-7-ylidene)-N-((4,5-dichlorothiophen-2-yl)sulfonyl)butanamide as a white solid.

¹H NMR (400 MHz, Methanol-d4) δ: 7.53 (s, 1H), 7.42 (s, 1H), 7.37 (s, 1H), 7.24 (d, J=8.4 Hz, 1H), 6.50 (d, J=8.4 Hz, 1H), 5.49 (s, 2H), 5.35 (t, J=7.2 Hz, 1H), 2.63 (t, J=6.4 Hz, 2H), 2.37-2.45 (m, 4H), 2.18 (t, J=7.2 Hz, 2H), 1.68-1.83 (m, 2H). Mass spectrum (ESI, m/z): Calculated for C₂₂H₂₀Cl₄N₃O₃S₂, 580.3 (M+H), found 580.0.

The following representative compounds of formula (I) were similarly prepared according to the procedure described in Example 4 above, selecting and substituting suitable starting materials and reagents, as would be readily recognized by those skilled in the art.

### (E)-N-((4-chlorophenyl)sulfonyl)-4-(1-(2,4-dichlorobenzyl)-1,4,5,6-tetrahydro-7H-indazol-7-ylidene)butanamide, Compound #24

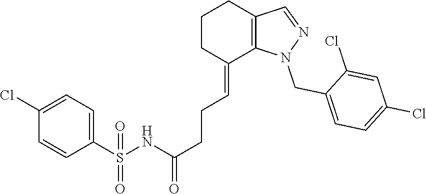 [SMILES: O=C(CC/C=C1\CCCc2cnn(Cc3ccc(Cl)cc3Cl)c21)NS(=O)(=O)c1ccc(Cl)cc1]

¹H NMR (300 MHz, Methanol-d4) δ: 7.94 (d, J=6.6 Hz, 2H), 7.52 (d, J=8.1 Hz, 3H), 7.38 (s, 1H), 7.24 (d, J=8.4 Hz, 1H), 6.45 (d, J=8.4 Hz, 1H), 5.42 (s, 2H), 5.21 (t, J=6.9 Hz, 1H), 2.61 (t, J=6.0 Hz, 2H), 2.31-2.35 (m, 4H), 2.22-2.26 (m, 2H), 1.71-1.75 (m, 2H). Mass spectrum (ESI, m/z): Calculated for C₂₄H₂₃Cl₃N₃O₃S, 539.9 (M+H), found 540.0.

### (E)-N-((5-chlorothiophen-2-yl)sulfonyl)-4-(1-(2,4-dichlorobenzyl)-1,4,5,6-tetrahydro-7H-indazol-7-ylidene)butanamide, Compound #29

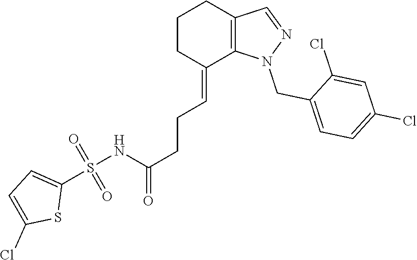 [SMILES: O=C(CC/C=C1\CCCc2cnn(Cc3ccc(Cl)cc3Cl)c21)NS(=O)(=O)c1ccc(Cl)s1]

¹H NMR (300 MHz, Methanol-d4) δ: 7.49 (d, J=1.8 Hz, 1H), 7.43 (d, J=7.8 Hz, 1H), 7.33 (s, 1H), 7.18-7.21 (m, 1H), 6.88 (d, J=3.9 Hz, 1H), 6.44 (d, J=8.4 Hz, 1H), 5.43 (s, 2H), 5.28 (t, J=7.2 Hz, 1H), 2.58 (t, J=6.3 Hz, 2H), 2.30-2.40 (m, 4H), 2.15 (t, J=7.2 Hz, 2H), 1.71-1.79 (m, 2H). Mass spectrum (ESI, m/z): Calculated for C₂₂H₂₁Cl₃N₃O₃S₂, 545.9 (M+H), found 545.9.

### (E)-4-(1-(2,4-dichlorobenzyl)-1,4,5,6-tetrahydro-7H-indazol-7-ylidene)-N-((4-methoxyphenyl)sulfonyl)butanamide, Compound #30

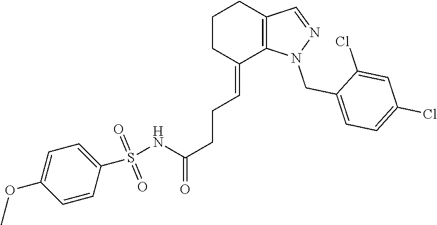 [SMILES: COc1ccc(S(=O)(=O)NC(=O)CC/C=C2\CCCc3cnn(Cc4ccc(Cl)cc4Cl)c32)cc1]

¹H NMR (300 MHz, Methanol-d4) δ: 7.84 (d, J=9.9 Hz, 2H), 7.49 (s, 1H), 7.33 (s, 1H), 7.19 (d, J=8.4 Hz, 1H), 6.94 (d, J=9.9 Hz, 2H), 6.41 (d, J=8.4 Hz, 1H), 5.35 (s, 2H), 5.17 (t, J=7.2 Hz, 1H), 3.81 (s, 3H), 2.56 (t, J=6.3 Hz, 2H), 2.26-2.31 (m, 4H), 2.16 (t, J=6.3 Hz, 2H), 1.67-1.75 (m, 2H). Mass spectrum (ESI, m/z): Calculated for C₂₅H₂₆Cl₂N₃O₄S, 534.1 (M+H), found 534.2.

### (E)-N-(benzo[b]thiophen-2-ylsulfonyl)-4-(1-(2,4-dichlorobenzyl)-1,4,5,6-tetrahydro-7H-indazol-7-ylidene)butanamide, Compound #31

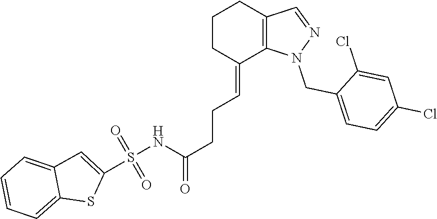 [SMILES: O=C(CC/C=C1\CCCc2cnn(Cc3ccc(Cl)cc3Cl)c21)NS(=O)(=O)c1cc2ccccc2s1]

¹H NMR (300 MHz, Methanol-d4) δ: 8.06 (s, 1H), 7.90 (d, J=6.3 Hz, 1H), 7.81 (d, J=7.2 Hz, 1H), 7.42-7.51 (m, 3H), 7.25 (s, 1H), 7.15 (d, J=8.4 Hz, 1H), 6.36 (d, J=8.4 Hz, 1H), 5.27 (s, 2H), 5.13 (t, J=6.9 Hz, 1H), 2.32-2.40 (m, 4H), 2.23-2.30 (m, 4H), 1.56-1.64 (m, 2H). Mass spectrum (ESI, m/z): Calculated for C₂₆H₂₄Cl₂N₃O₃S₂, 560.1 (M+H), found 560.1.

### (E)-N-((5-chloro-2-methoxyphenyl)sulfonyl)-4-(1-(2,4-dichlorobenzyl)-1,4,5,6-tetrahydro-7H-indazol-7-ylidene)butanamide, Compound #38

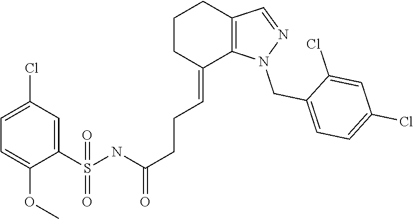 [SMILES: COc1ccc(Cl)cc1S(=O)(=O)NC(=O)CC/C=C1\CCCc2cnn(Cc3ccc(Cl)cc3Cl)c21]

¹H NMR (300 MHz, CD₃OD) δ: 7.91 (s, 1H), 7.61 (d, J=9.0 Hz, 1H), 7.54 (s, 1H), 7.41 (s, 1H), 7.25 (d, J=8.4 Hz, 1H), 7.08 (d, J=9.0 Hz, 1H), 6.47 (d, J=8.4 Hz, 1H), 5.42 (s, 2H), 5.30 (t, J=60 Hz, 1H), 3.87 (s, 3H), 2.62 (t, J=6.3 Hz, 2H), 2.27-2.40 (m, 6H), 1.67-1.77 (m, 2H). Mass spectrum (ESI, m/z): Calculated for C₂₅H₂₅Cl₃N₃O₄S, 570.1 [M+H], found 570.0.

### (E)-N-((5-bromo-2-methoxyphenyl)sulfonyl)-4-(1-(2,4-dichlorobenzyl)-1,4,5,6-tetrahydro-7H-indazol-7-ylidene)butanamide, Compound #41

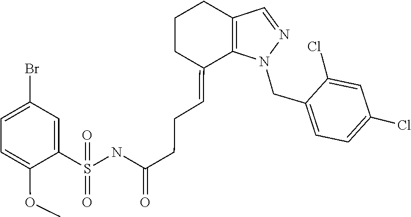

¹H NMR (300 MHz, CD₃OD) δ: 7.98 (s, 1H), 7.69 (d, J=8.7 Hz, 1H), 7.48 (s, 1H), 7.35 (s, 1H), 7.19 (d, J=8.4 Hz, 1H), 6.97 (d, J=8.7 Hz, 1H), 6.42 (d, J=8.4 Hz, 1H), 5.36 (s, 2H), 5.23 (t, J=6.3 Hz, 1H), 3.81 (s, 3H), 2.57 (t, J=6.0 Hz, 2H), 2.18-2.34 (m, 6H), 1.63-1.71 (m, 2H). Mass spectrum (ESI, m/z): Calculated for C₂₅H₂₅BrCl₂N₃O₄S, 614.0 [M+H], found 614.0.

### (E)-4-(1-(2,4-dichlorobenzyl)-1,4,5,6-tetrahydro-7H-indazol-7-ylidene)-N-((2-methoxyphenyl)sulfonyl)butanamide, Compound #44

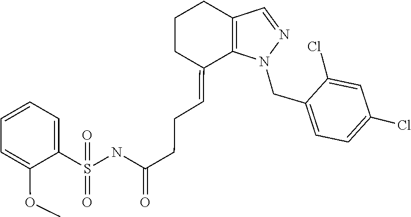

¹H NMR (300 MHz, CD₃OD) δ: 7.89 (d, J=8.4 Hz, 1H), 7.59 (t, J=8.4 Hz, 1H), 7.48 (s, 1H), 7.35 (s, 1H), 7.19 (d, J=8.4 Hz, 1H), 7.02-7.09 (m, 2H), 6.43 (d, J=8.4 Hz, 1H), 5.37 (s, 2H), 5.24 (t, J=6.6 Hz, 1H), 3.82 (s, 3H), 2.56 (t, J=6.3 Hz, 2H), 2.23-2.33 (m, 6H), 1.64-1.70 (m, 2H). Mass spectrum (ESI, m/z): Calculated for C₂₅H₂₆Cl₂N₃O₄S, 534.1 (M+H), found 534.1.

### (E)-4-(1-(2,4-dichlorobenzyl)-1,4,5,6-tetrahydro-7H-indazol-7-ylidene)-N-(isopropylsulfonyl)butanamide, Compound #69

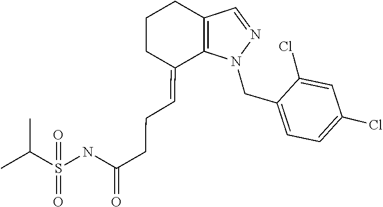

¹H NMR (400 MHz, CDCl₃) δ: 8.29 (br s, 1H), 7.42 (s, 1H), 7.38 (s, 1H), 7.14 (dd, J=8.3, 2.3 Hz, 1H), 6.54 (d, J=8.6 Hz, 1H), 5.47 (s, 2H), 5.18 (t, J=7.1 Hz, 1H), 3.69-3.79 (m, 1H), 2.63 (t, J=6.1 Hz, 2H), 2.38-2.49 (m, 4H), 2.24-2.36 (m, 2H), 2.05 (s, 1H), 1.75-1.88 (m, 2H), 1.38 (d, J=7.1 Hz, 6H). Mass spectrum (ESI, m/z): Calculated for C₂₁H₂₆Cl₂N₃O₃S, 470.1 (M+H), found 470.1.

### Example 5: Compound #7

### (E)-4,5-dichloro-N-((2-(1-(2,4-dichlorobenzyl)-1,4,5,6-tetrahydro-7H-indazol-7-ylidene)ethyl)carbamoyl)thiophene-2-sulfonamide

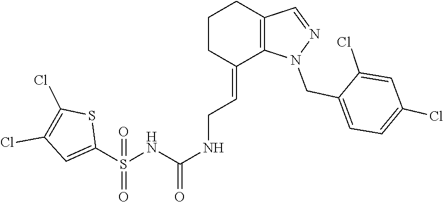 [SMILES: O=C(NC/C=C1\CCCc2cnn(Cc3ccc(Cl)cc3Cl)c21)NS(=O)(=O)c1cc(Cl)c(Cl)s1]

### Step 1. Synthesis of (E)-2-(2-(1-(2,4-dichlorobenzyl)-1,4,5,6-tetrahydro-7H-indazol-7-ylidene)ethyl)isoindoline-1,3-dione

To a solution of (E)-2-(1-(2,4-dichlorobenzyl)-1,4,5,6-tetrahydro-7H-indazol-7-ylidene)ethan-1-ol (2 g, 6.19 mmol), isoindoline-1,3-dione (2.73 g, 18.6 mmol) and PPh₃ (228 mg, 0.870 mmol) in THF (10 mL) was added DIAD (176 mg, 0.870 mmol). The reaction was stirred at room temperature overnight. The reaction was then quenched with H₂O and the resulting mixture was extracted with EtOAc. The organic layers were combined and concentrated under vacuum. The resulting residue was purified by silica gel column with PE:EA=70:30 to yield (E)-2-(2-(1-(2,4-dichlorobenzyl)-1,4,5,6-tetrahydro-7H-indazol-7-ylidene)ethyl)isoindoline-1,3-dione as yellow solid. Mass spectrum (ESI, m/z): Calculated for C₂₄H₂₀Cl₂N₃O₂, 452.1 [M+H], found 452.1.

### Step 2. Synthesis of (E)-2-(1-(2,4-dichlorobenzyl)-1,4,5,6-tetrahydro-7H-indazol-7-ylidene)ethan-1-amine

A solution of (E)-2-(2-(1-(2,4-dichlorobenzyl)-1,4,5,6-tetrahydro-7H-indazol-7-ylidene)ethyl)isoindoline-1,3-dione (2.1 g, 4.64 mmol) and NH₂NH₂ (5 mL) in MeOH (50 mL) was stirred at room temperature for 1 h. The reaction solution was concentrated under vacuum. The resulting residue was purified by silica gel column with PE:EA=30:70 to yield (E)-2-(1-(2,4-dichlorobenzyl)-1,4,5,6-tetrahydro-7H-indazol-7-ylidene)ethan-1-amine as yellow solid. Mass spectrum (ESI, m/z): Calculated for C₁₆H₁₈Cl₂N₃, 322.1 [M+H], found 322.0.

### Step 3. (E)-4,5-dichloro-N-((2-(1-(2,4-dichlorobenzyl)-1,4,5,6-tetrahydro-7H-indazol-7-ylidene)ethyl)carbamoyl)thiophene-2-sulfonamide

A solution of 4,5-dichlorothiophene-2-sulfonamide (500 mg, 2.15 mmol), CDI (350 mg, 2.16 mmol), and triethylamine (240 mg, 2.37 mmol) in THF (20 mL) was stirred for 16 hours at room temperature. To the resulting solution was added (E)-2-(1-(2,4-dichlorobenzyl)-1,4,5,6-tetrahydro-7H-indazol-7-ylidene)ethan-1-amine (250 mg, 0.776 mmol). The reaction was heated by microwave at 100° C. for 1 hour. The reaction solution was diluted with EtOAc and washed with aq NaCl. The organic solution was concentrated under vacuum. The resulting residue was purified by Prep-HPLC with the following conditions (1#-Waters 2767-5): Column, SunFire Prep C18, 5 um, 19*100 mm; mobile phase, Water with 0.05% TFA and CH₃CN (40% CH₃CN up to 100% in 10 min, up to 100% in 0.1 min, hold 100% in 0.9 min, down to 40% in 0.1 min, hold 40% in 1.4 min); Detector, UV 220 & 254 nm. The product was exchanged with 2N HCl three times to yield (E)-4,5-dichloro-N-((2-(1-(2,4-dichlorobenzyl)-1,4,5,6-tetrahydro-7H-indazol-7-ylidene)ethyl)carbamoyl)thiophene-2-sulfonamide as a white solid.

¹H NMR (400 MHz, Methanol-d4) δ: 7.65-7.69 (m, 2H), 7.50 (s, 1H), 7.26 (d, J=8.4 Hz, 1H), 6.61 (d, J=8.4 Hz, 1H), 5.57 (s, 2H), 5.43 (t, J=6.8 Hz, 1H), 3.86 (d, J=6.4 Hz, 2H), 2.68 (t, J=6.2 Hz, 2H), 2.52 (t, J=5.4 Hz, 2H), 1.83-1.89 (m, 2H). Mass spectrum (ESI, m/z): Calculated for C₂₂H₂₁Cl₄N₄O₃S, 581.3 [M+H], found 581.0.

The following representative compounds of formula (I) were similarly prepared according to the procedure described in Example 5 above, selecting and substituting suitable starting materials and reagents, as would be readily recognized by those skilled in the art.

### (E)-N-((2-(1-(2,4-dichlorobenzyl)-1,4,5,6-tetrahydro-7H-indazol-7-ylidene)ethyl)carbamoyl)-3,5-difluorobenzenesulfonamide, Compound #9

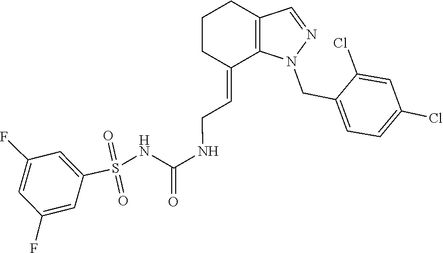 [SMILES: O=C(NC/C=C1\CCCc2cnn(Cc3ccc(Cl)cc3Cl)c21)NS(=O)(=O)c1cc(F)cc(F)c1]

¹H NMR (300 MHz, Methanol-d4) δ: 7.56 (d, J=8.4 Hz, 2H), 7.42 (s, 1H), 7.19-7.36 (m, 2H), 7.16 (d, J=8.4 Hz, 1H), 6.43 (d, J=8.4 Hz, 1H), 5.43 (s, 2H), 5.23 (t, J=6.6 Hz, 1H), 3.77 (d, J=6.6 Hz, 2H), 2.60 (t, J=6.3 Hz, 2H), 2.42 (t, J=4.8 Hz, 2H), 1.76-1.82 (m, 2H). ¹⁹F NMR (300 MHz, Methanol-d4) δ: −108.15. Mass spectrum (ESI, m/z): Calculated for C₂₃H₂₁Cl₂F₂N₄O₃S, 541.1 (M+H), found 541.1.

### (E)-N-((2-(1-(2,4-dichlorobenzyl)-1,4,5,6-tetrahydro-7H-indazol-7-ylidene)ethyl)carbamoyl)thiophene-2-sulfonamide, Compound #10

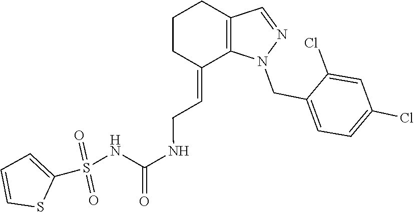 [SMILES: O=C(NC/C=C1\CCCc2cnn(Cc3ccc(Cl)cc3Cl)c21)NS(=O)(=O)c1cccs1]

¹H NMR (400 MHz, Methanol-d4) δ: 7.74 (d, J=4.8 Hz, 1H), 7.64 (s, 1H), 7.38 (s, 1H), 7.29 (s, 1H), 7.11 (d, J=8.8 Hz, 1H), 7.04 (d, J=4.0 Hz, 1H), 6.37 (d, J=8.4 Hz, 1H), 5.37 (s, 2H), 5.19 (t, J=6.4 Hz, 1H), 3.74 (d, J=6.4 Hz, 2H), 2.54 (t, J=6.0 Hz, 2H), 2.36 (t, J=4.2 Hz, 2H), 1.71-1.74 (m, 2H). Mass spectrum (ESI, m/z): Calculated for C₂₁H₂₁Cl₂N₄O₃S₂, 511.0 (M+H), found 510.9.

### (E)-5-chloro-N-((2-(1-(2,4-dichlorobenzyl)-1,4,5,6-tetrahydro-7H-indazol-7-ylidene)ethyl)carbamoyl)thiophene-2-sulfonamide, Compound #11

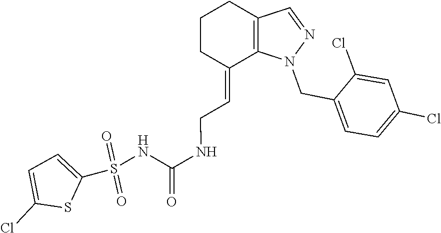 [SMILES: O=C(NC/C=C1\CCCc2cnn(Cc3ccc(Cl)cc3Cl)c21)NS(=O)(=O)c1ccc(Cl)s1]

¹H NMR (300 MHz, Methanol-d4) δ: 7.55 (d, J=4.2 Hz, 1H), 7.44 (s, 1H), 7.36 (s, 1H), 7.18 (d, J=8.4 Hz, 1H), 7.05 (d, J=3.9 Hz, 1H), 6.44 (d, J=8.4 Hz, 1H), 5.48 (s, 2H), 5.26 (t, J=6.6 Hz, 1H), 3.80 (d, J=6.3 Hz, 2H), 2.61 (t, J=6.0 Hz, 2H), 2.44 (t, J=6.0 Hz, 2H), 1.78-1.84 (m, 2H). Mass spectrum (ESI, m/z): Calculated for C₂₁H₂₀Cl₃N₄O₃S₂, 546.9 (M+H), found 546.9.

### (E)-N-((2-(1-(2,4-dichlorobenzyl)-1,4,5,6-tetrahydro-7H-indazol-7-ylidene)ethyl)carbamoyl)quinoline-3-sulfonamide, Compound #12

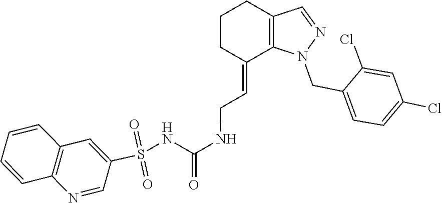 [SMILES: O=C(NC/C=C1\CCCc2cnn(Cc3ccc(Cl)cc3Cl)c21)NS(=O)(=O)c1cnc2ccccc2c1]

¹H NMR (300 MHz, Methanol-d4) δ: 9.29 (s, 1H), 9.00 (s, 1H), 8.11-8.18 (m, 2H), 8.00 (t, J=8.4 Hz, 1H), 7.78 (t, J=7.0 Hz, 1H), 7.34 (s, 1H), 7.26 (s, 1H), 7.16 (d, J=8.4 Hz, 1H), 6.40 (d, J=8.1 Hz, 1H), 5.57 (s, 2H), 5.18 (t, J=6.3 Hz, 1H), 3.77 (d, J=6.6 Hz, 2H), 2.57 (t, J=6.3 Hz, 2H), 2.38 (t, J=5.4 Hz, 2H), 1.71-1.77 (m, 2H). Mass spectrum (ESI, m/z): Calculated for C₂₆H₂₄Cl₂N₅O₃S, 556.1 (M+H), found 556.3.

### (E)-N-((2-(1-(2,4-dichlorobenzyl)-1,4,5,6-tetrahydro-7H-indazol-7-ylidene)ethyl)carbamoyl)-4-methoxybenzenesulfonamide, Compound #13

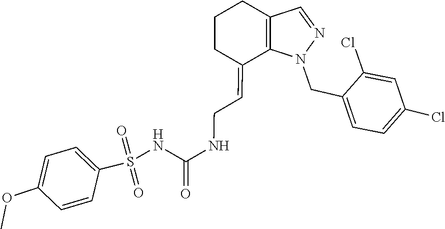 [SMILES: COc1ccc(S(=O)(=O)NC(=O)NC/C=C2\CCCc3cnn(Cc4ccc(Cl)cc4Cl)c32)cc1]

¹H NMR (300 MHz, Methanol-d4) δ: 7.88 (d, J=6.9 Hz, 2H), 7.46 (s, 1H), 7.45 (s, 1H), 7.22 (d, J=8.4 Hz, 1H), 7.06 (d, J=8.1 Hz, 2H), 6.47 (d, J=8.4 Hz, 1H), 5.44 (s, 2H), 5.27 (t, J=6.3 Hz, 1H), 3.87 (s, 3H), 3.80 (d, J=6.6 Hz, 2H), 2.63 (t, J=6.2 Hz, 2H), 2.43 (t, J=5.2 Hz, 2H), 1.77-1.85 (m, 2H). Mass spectrum (ESI, m/z): Calculated for C₂₄H₂₅Cl₂N₄O₄S, 535.1 (M+H), found 535.2.

### (E)-N-((2-(1-(2,4-dichlorobenzyl)-1,4,5,6-tetrahydro-7H-indazol-7-ylidene)ethyl)carbamoyl)benzo[d]thiazole-2-sulfonamide, Compound #15

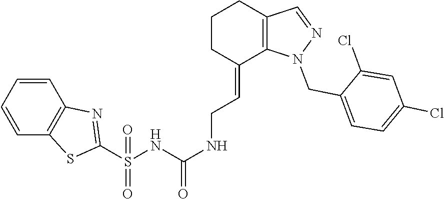 [SMILES: O=C(NC/C=C1\CCCc2cnn(Cc3ccc(Cl)cc3Cl)c21)NS(=O)(=O)c1nc2ccccc2s1]

¹H NMR (300 MHz, Methanol-d4) δ: 8.08-8.15 (m, 2H), 7.61-7.68 (m, 2H), 7.36-7.38 (m, 2H), 7.16 (d, J=8.4 Hz, 1H), 6.44 (d, J=8.4 Hz, 1H), 5.38 (s, 2H), 5.29 (t, J=6.3 Hz, 1H), 3.83 (d, J=6.6 Hz, 2H), 2.59 (t, J=6.0 Hz, 2H), 2.43 (t, J=5.4 Hz, 2H), 1.73-1.78 (m, 2H). Mass spectrum (ESI, m/z): Calculated for C₂₄H₂₂Cl₂N₅O₃S₂, 562.0 (M+H), found 563.8.

### (E)-N-((2-(1-(2,4-dichlorobenzyl)-1,4,5,6-tetrahydro-7H-indazol-7-ylidene)ethyl)carbamoyl)-2,3-dihydrobenzo[b][1,4]dioxine-6-sulfonamide, Compound #16

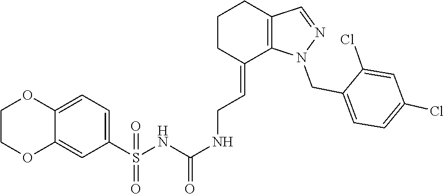 [SMILES: O=C(NC/C=C1\CCCc2cnn(Cc3ccc(Cl)cc3Cl)c21)NS(=O)(=O)c1ccc2c(c1)OCCO2]

¹H NMR (300 MHz, Methanol-d4) δ: 7.42-7.48 (m, 4H), 7.21 (d, J=8.4 Hz, 1H), 6.98 (d, J=7.5 Hz, 1H), 6.47 (d, J=8.4 Hz, 1H), 5.45 (s, 2H), 5.29 (t, J=6.3 Hz, 1H), 4.25-4.32 (m, 4H), 3.80 (d, J=6.6 Hz, 2H), 2.64 (t, J=6.3 Hz, 2H), 2.44 (t, J=5.4 Hz, 2H), 1.79-1.85 (m, 2H). Mass spectrum (ESI, m/z): Calculated for C₂₅H₂₅Cl₂N₄O₅S, 563.1 (M+H), found 563.1.

### (E)-N-((2-(1-(2,4-dichlorobenzyl)-1,4,5,6-tetrahydro-7H-indazol-7-ylidene)ethyl)carbamoyl)benzo[b]thiophene-2-sulfonamide, Compound #27

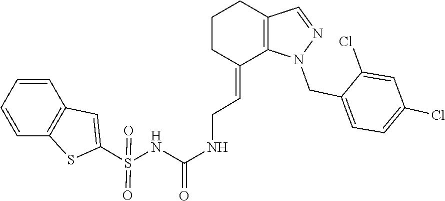 [SMILES: O=C(NC/C=C1\CCCc2cnn(Cc3ccc(Cl)cc3Cl)c21)NS(=O)(=O)c1cc2ccccc2s1]

¹H NMR (300 MHz, Methanol-d4) δ: 8.02 (s, 1H), 7.89-7.93 (m, 2H), 7.43-7.49 (m, 2H), 7.34 (s, 1H), 7.27 (s, 1H), 7.10 (d, J=8.4 Hz, 1H), 6.38 (d, J=8.7 Hz, 1H), 5.30 (s, 2H), 5.23 (t, J=6.6 Hz, 1H), 3.80 (d, J=6.6 Hz, 2H), 2.56 (t, J=6.3 Hz, 2H), 2.40 (t, J=6.0 Hz, 2H), 1.72-1.78 (m, 2H). Mass spectrum (ESI, m/z): Calculated for C₂₅H₂₃Cl₂N₄O₃S₂, 561.1 (M+H), found 561.1.

### (E)-N-((2-(1-(2,4-dichlorobenzyl)-1,4,5,6-tetrahydro-7H-indazol-7-ylidene)ethyl)carbamoyl)benzofuran-2-sulfonamide, Compound #28

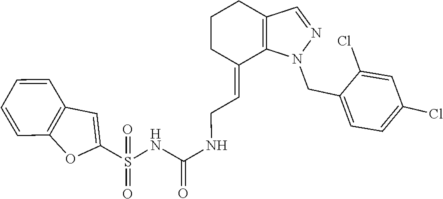 [SMILES: O=C(NC/C=C1\CCCc2cnn(Cc3ccc(Cl)cc3Cl)c21)NS(=O)(=O)c1cc2ccccc2o1]

¹H NMR (300 MHz, Methanol-d4) δ: 7.71 (d, J=8.4 Hz, 1H), 7.46-7.55 (m, 3H), 7.30-7.38 (m, 3H), 7.13 (d, J=8.4 Hz, 1H), 6.40 (d, J=8.4 Hz, 1H), 5.34 (s, 2H), 5.24 (t, J=6.6 Hz, 1H), 3.79 (d, J=6.6 Hz, 2H), 2.56 (t, J=6.3 Hz, 2H), 2.39 (t, J=5.4 Hz, 2H), 1.69-1.77 (m, 2H). Mass spectrum (ESI, m/z): Calculated for C₂₅H₂₃Cl₂N₄O₄S, 545.1 (M+H), found 545.2.

### (E)-N-((2-(1-(2,4-dichlorobenzyl)-1,4,5,6-tetrahydro-7H-indazol-7-ylidene)ethyl)carbamoyl)-2-methoxybenzenesulfonamide, Compound #39

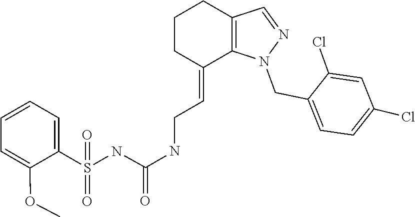

¹H NMR (300 MHz, Methanol-d4) δ: 7.91 (d, J=7.8 Hz, 1H), 7.64 (t, J=8.1 Hz, 1H), 7.51 (s, 1H), 7.41 (s, 1H), 7.20-7.27 (m, 2H), 7.08 (t, J=8.1 Hz, 1H), 6.51 (d, J=8.1 Hz, 1H), 5.48 (s, 2H), 5.32 (t, J=6.9 Hz, 1H), 3.94 (s, 3H), 3.83 (d, J=6.6 Hz, 2H), 2.66 (t, J=6.3 Hz, 2H), 2.56 (t, J=4.8 Hz, 2H), 1.78-1.86 (m, 2H). Mass spectrum (ESI, m/z): Calculated for C₂₄H₂₅Cl₂N₄O₄S, 535.1 (M+H), found 535.1.

### (E)-5-bromo-N-((2-(1-(2,4-dichlorobenzyl)-1,4,5,6-tetrahydro-7H-indazol-7-ylidene)ethyl)carbamoyl)-2-methoxybenzenesulfonamide, Compound #42

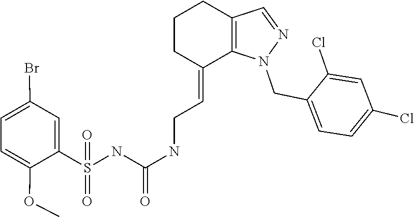

¹H NMR (300 MHz, Methanol-d₄, ppm) δ: 7.97 (s, 1H), 7.70 (d, J=8.7 Hz, 1H), 7.44 (s, 1H), 7.36 (s, 1H), 7.19 (d, J=8.7 Hz, 1H), 7.12 (d, J=8.7 Hz, 1H), 6.45 (d, J=8.1 Hz, 1H), 5.42 (s, 2H), 5.26 (t, J=7.5 Hz, 1H), 3.88 (s, 3H), 3.77 (d, J=6.6 Hz, 2H), 2.60 (t, J=6.0 Hz, 2H), 2.39 (t, J=6.0 Hz, 2H), 1.72-1.80 (m, 2H). Mass spectrum (ESI, m/z): Calculated for C₂₄H₂₄BrCl₂N₄O₄S, 615.0 (M+H), found 615.0.

### (E)-5-chloro-N-((2-(1-(2,4-dichlorobenzyl)-1,4,5,6-tetrahydro-7H-indazol-7-ylidene)ethyl)carbamoyl)-2-methoxybenzenesulfonamide, Compound #43

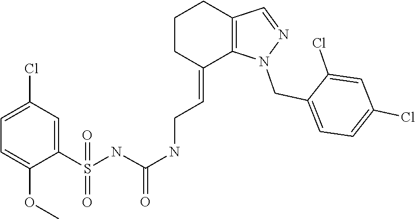

¹H NMR (300 MHz, Methanol-d₄, ppm) δ: 7.84 (s, 1H), 7.57 (d, J=9.0 Hz, 1H), 7.45 (s, 1H), 7.36 (s, 1H), 7.15-7.21 (m, 2H), 6.45 (d, J=8.7 Hz, 1H), 5.42 (s, 2H), 5.26 (t, J=6.0 Hz, 1H), 3.88 (s, 3H), 3.77 (d, J=6.6 Hz, 2H), 2.60 (t, J=6.0 Hz, 2H), 2.39 (t, J=6.0 Hz, 2H), 1.74-1.78 (m, 2H). Mass spectrum (ESI, m/z): Calculated for C₂₄H₂₄Cl₃N₄O₄S, 571.1 [M+H], found 571.0.

### (E)-N-((2-(1-(2,4-dichlorobenzyl)-1,4,5,6-tetrahydro-7H-indazol-7-ylidene)ethyl)carbamoyl)-2-methylthiazole-5-sulfonamide, Compound #139

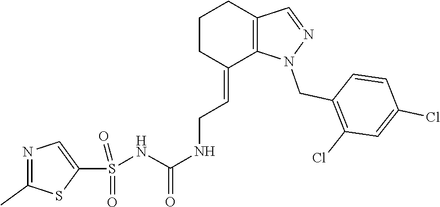 [SMILES: Cc1ncc(S(=O)(=O)NC(=O)NC/C=C2\CCCc3cnn(Cc4ccc(Cl)cc4Cl)c32)s1]

¹H NMR (300 MHz, CD₃OD) δ: 8.18 (s, 1H), 7.77 (s, 1H), 7.54 (s, 1H), 7.29 (d, J=8.4 Hz, 1H), 6.67 (d, J=8.4 Hz, 1H), 5.61 (s, 2H), 5.50 (t, J=6.0 Hz, 1H), 3.87 (t, J=6.6 Hz, 2H), 2.68-2.76 (m, 5H), 2.53 (t, J=5.7 Hz, 2H), 1.86-1.92 (m, 2H). Mass spectrum (ESI, m/z): Calculated for C₂₁H₂₃Cl₃N₅O₃S2, 526.0 [M+H], found 526.1.

### (E)-N-((2-(1-(4-bromophenyl)-1,4,5,6-tetrahydro-7H-indazol-7-ylidene)ethyl)carbamoyl)-4,5-dichlorothiophene-2-sulfonamide, Compound #75

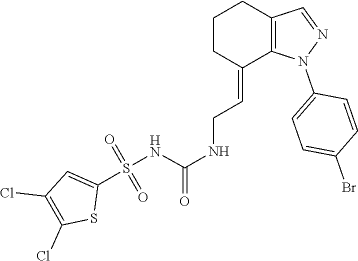 [SMILES: O=C(NC/C=C1\CCCc2cnn(-c3ccc(Br)cc3)c21)NS(=O)(=O)c1cc(Cl)c(Cl)s1]

¹H NMR (400 MHz, Methanol-d4) δ: 7.62-7.68 (m, 3H), 7.49 (s, 1H), 7.30 (d, J=9.6 Hz, 2H), 5.04 (t, J=6.8 Hz, 1H), 3.77 (d, J=6.8 Hz, 2H), 2.69 (t, J=6.0 Hz, 2H), 2.57 (t, J=5.2 Hz, 2H), 1.18-1.96 (m, 2H). Mass spectrum (ESI, m/z): Calculated for C₂₀H₁₈BrCl₂N₄O₃S₂: 574.9 (M+H), found: 576.9.

### (E)-N-((2-(1-(3-bromophenyl)-1,4,5,6-tetrahydro-7H-indazol-7-ylidene)ethyl)carbamoyl)-4,5-dichlorothiophene-2-sulfonamide, Compound #77

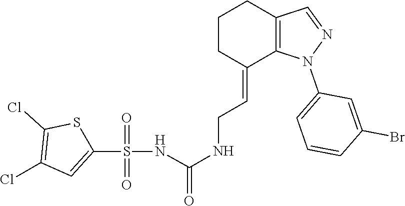 [SMILES: O=C(NC/C=C1\CCCc2cnn(-c3cccc(Br)c3)c21)NS(=O)(=O)c1cc(Cl)c(Cl)s1]

¹H NMR (300 MHz, CD₃OD) δ: 7.53-7.59 (m, 2H), 7.43-7.46 (m, 2H), 7.30-7.37 (m, 2H), 5.01-5.09 (m, 1H), 3.70-3.74 (m, 2H), 2.61-2.65 (m, 2H), 2.51-2.54 (m, 2H), 1.74-1.87 (m, 2H). Mass spectrum (ESI, m/z): Calculated for C₂₀H₁₈BrCl₂N₄O₃S₂: 574.9 (M+H), found: 576.8.

### (E)-4,5-dichloro-N-((2-(1-(naphthalen-1-yl)-1,4,5,6-tetrahydro-7H-indazol-7-ylidene)ethyl)carbamoyl)thiophene-2-sulfonamide, Compound #78

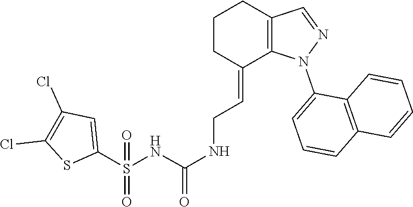 [SMILES: O=C(NC/C=C1\CCCc2cnn(-c3cccc4ccccc34)c21)NS(=O)(=O)c1cc(Cl)c(Cl)s1]

¹H NMR (400 MHz, Methanol-d4) δ: 8.06 (d, J=8.4 Hz, 1H), 8.00 (d, J=8.4 Hz, 1H), 7.70 (brs, 1H), 7.58-7.62 (m, 2H), 7.47-7.56 (m, 3H), 7.15 (d, J=8.8 Hz, 1H), 4.65 (t, J=6.4 Hz, 1H), 3.59-3.65 (m, 1H), 3.48-3.53 (m, 1H), 2.80 (t, J=5.6 Hz, 2H), 2.56 (t, J=3.2 Hz, 2H), 1.90-1.97 (m, 2H). Mass spectrum (ESI, m/z): Calculated for C₂₄H₂₁Cl₂N₄O₃S₂: 547.0 (M+H), found: 547.0.

### (E)-4,5-dichloro-N-((2-(1-(naphthalen-2-yl)-1,4,5,6-tetrahydro-7H-indazol-7-ylidene)ethyl)carbamoyl)thiophene-2-sulfonamide, Compound #79

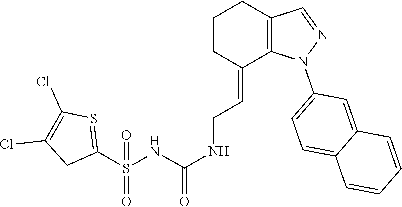

¹H NMR (400 MHz, Methanol-d₄, ppm) δ: 7.93-8.12 (m, 4H), 7.66 (s, 1H), 7.58-7.64 (m, 3H), 7.47 (d, J=8.8 Hz, 1H), 5.08 (t, J=6.8 Hz, 1H), 3.72 (d, J=6.8 Hz, 2H), 2.76 (t, J=6.4 Hz, 2H), 2.63 (t, J=5.2 Hz, 2H), 1.90-1.99 (m, 2H). Mass spectrum (ESI, m/z): Calculated for C₂₄H₂₁Cl₂N₄O₃S₂: 547.0 [M+H], found: 547.1.

### (E)-4,5-dichloro-N-((2-(1-(2,4-dichlorophenyl)-1,4,5,6-tetrahydro-7H-indazol-7-ylidene)ethyl)carbamoyl)thiophene-2-sulfonamide, Compound #84

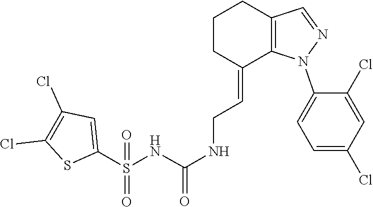 [SMILES: O=C(NC/C=C1\CCCc2cnn(-c3ccc(Cl)cc3Cl)c21)NS(=O)(=O)c1cc(Cl)c(Cl)s1]

¹H NMR (300 MHz, CD₃OD, ppm) δ: 7.58-7.61 (m, 2H), 7.37-7.48 (m, 3H), 4.83-4.94 (m, 1H), 3.69 (d, J=6.9 Hz, 2H), 2.62-2.66 (m, 2H), 2.41-2.49 (m, 2H), 1.80-1.84 (m, 2H). Mass spectrum (ESI, m/z): Calculated for C₂₀H₁₇Cl₄N₄O₃S₂: 564.9 [M+H], found: 566.9.

### (E)-4,5-dichloro-N-((2-(1-(4-(trifluoromethyl)phenyl)-1,4,5,6-tetrahydro-7H-indazol-7-ylidene)ethyl)carbamoyl)thiophene-2-sulfonamide, Compound #85

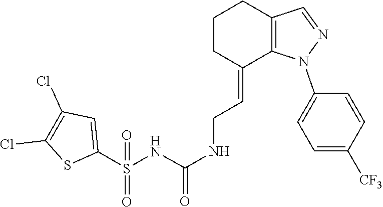 [SMILES: O=C(NC/C=C1\CCCc2cnn(-c3ccc(C(F)(F)F)cc3)c21)NS(=O)(=O)c1cc(Cl)c(Cl)s1]

¹H NMR (300 MHz, CD₃OD, ppm) δ: 7.76 (d, J=8.4 Hz, 2H), 7.62-7.68 (m, 3H), 7.49 (s, 1H), 4.98-5.02 (m, 1H), 3.72-3.77 (m, 2H), 2.64-2.68 (m, 2H), 2.54-2.57 (m, 2H), 1.81-1.90 (m, 2H). ¹⁹F NMR (300 MHz, CD₃OD, ppm) δ: −63.85. Mass spectrum (ESI, m/z): Calculated for C₂₁H₁₈Cl₂F₃N₄O₃S₂: 565.0 [M+H], found: 565.0.

### (E)-4,5-dichloro-N-((2-(1-(3-(trifluoromethyl)phenyl)-1,4,5,6-tetrahydro-7H-indazol-7-ylidene)ethyl)carbamoyl)thiophene-2-sulfonamide, Compound #92

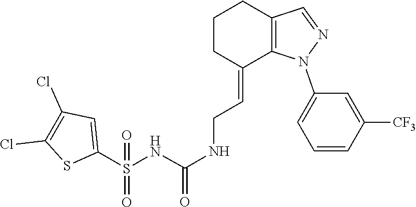 [SMILES: O=C(NC/C=C1\CCCc2cnn(-c3cccc(C(F)(F)F)c3)c21)NS(=O)(=O)c1cc(Cl)c(Cl)s1]

¹H NMR (400 MHz, CD₃OD) δ: 7.62-7.70 (m, 5H), 7.47-7.51 (m, 1H), 4.96-4.99 (m, 1H), 3.75 (d, J=6.8 Hz, 1H), 2.96-3.05 (m, 1H), 2.69-2.70 (m, 1H), 2.56-2.59 (m, 2H), 1.78-1.89 (m, 3H). ¹⁹F NMR (400 MHz, CD₃OD) δ: −64.13. Mass spectrum (ESI, m/z): Calculated for C₂₁H₁₈Cl₂F₃N₄O₃S₂: 565.0 (M+H), found: 566.9.

### (E)-4,5-dichloro-N-((2-(1-(6-methoxypyridin-3-yl)-1,4,5,6-tetrahydro-7H-indazol-7-ylidene)ethyl)carbamoyl)thiophene-2-sulfonamide, Compound #107

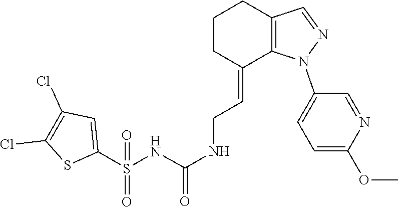 [SMILES: COc1ccc(-n2ncc3c2/C(=C/CNC(=O)NS(=O)(=O)c2cc(Cl)c(Cl)s2)CCC3)cn1]

¹H NMR (400 MHz, CHLOROFORM-d) δ: 8.17 (d, J=2.5 Hz, 1H), 7.65 (dd, J=8.8, 2.8 Hz, 1H), 7.49 (s, 1H), 7.46 (s, 1H), 6.84 (d, J=9.1 Hz, 1H), 6.17 (br s, 1H), 5.05 (br t, J=7.1 Hz, 1H), 3.95 (s, 3H), 3.86 (br t, J=6.1 Hz, 2H), 2.67 (t, J=6.3 Hz, 2H), 2.41-2.58 (m, 2H), 1.76-1.99 (m, 2H). Mass spectrum (ESI, m/z): Calculated for C₂₀H₂₀Cl₂N₅O₄S₂: 528.0 (M+H), found: 528.0.

### (E)-4,5-dichloro-N-((2-(1-(1-(4-fluorophenyl)piperidin-4-yl)-1,4,5,6-tetrahydro-7H-indazol-7-ylidene)ethyl)carbamoyl)thiophene-2-sulfonamide, Compound #123

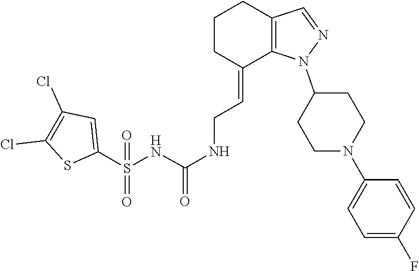 [SMILES: O=C(NC/C=C1\CCCc2cnn(C3CCN(c4ccc(F)cc4)CC3)c21)NS(=O)(=O)c1cc(Cl)c(Cl)s1]

¹H NMR (CHLOROFORM-d) δ: 7.45 (s, 1H), 7.26 (s, 1H), 6.93-7.07 (m, 4H), 6.71 (br s, 1H), 5.62 (br t, J=6.3 Hz, 1H), 4.48 (br s, 1H), 3.99-4.16 (m, 2H), 3.72 (br d, J=11.6 Hz, 2H), 2.94 (br t, J=10.6 Hz, 2H), 2.57 (br t, J=6.1 Hz, 2H), 2.30-2.50 (m, 4H), 2.12 (br d, J=12.1 Hz, 2H), 1.72-1.88 (m, 2H). Mass spectrum (ESI, m/z): Calculated for C₂₅H₂₇Cl₂FN₅O₃S₂: 598.1 (M+H), found: 598.0.

### (E)-4,5-dichloro-N-((2-(1-(4-(methylsulfonyl)phenyl)-1,4,5,6-tetrahydro-7H-indazol-7-ylidene)ethyl)carbamoyl)thiophene-2-sulfonamide, Compound #126

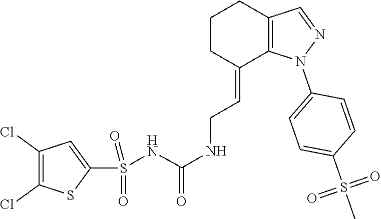 [SMILES: CS(=O)(=O)c1ccc(-n2ncc3c2/C(=C/CNC(=O)NS(=O)(=O)c2cc(Cl)c(Cl)s2)CCC3)cc1]

¹H NMR (300 MHz, DMSO) δ: 11.31 (s, 1H), 7.99 (d, J=8.7 Hz, 2H), 7.76 (s, 1H), 7.68 (d, J=8.4 Hz, 2H), 7.60 (s, 1H), 6.79 (s, 1H), 5.03 (t, J=6.0 Hz, 1H), 3.66 (t, J=6.3 Hz, 2H), 3.25 (s, 3H), 2.62 (t, J=5.7 Hz, 2H), 2.50 (t, J=1.8 Hz, 2H), 1.79-1.85 (m, 2H). Mass spectrum (ESI, m/z): Calculated for C₂₁H₂₁Cl₂N₄O₅S₃: 575.0 [M+H], found: 574.9.

### (E)-4,5-dichloro-N-((2-(1-((2-phenylpyrimidin-5-yl)methyl)-1,4,5,6-tetrahydro-7H-indazol-7-ylidene)ethyl)carbamoyl)thiophene-2-sulfonamide, Compound #138

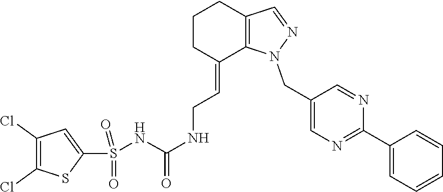 [SMILES: O=C(NC/C=C1\CCCc2cnn(Cc3cnc(-c4ccccc4)nc3)c21)NS(=O)(=O)c1cc(Cl)c(Cl)s1]

¹H NMR (400 MHz, CD₃OD) δ: 8.47 (s, 2H), 8.33-8.39 (m, 2H), 7.66 (s, 1H), 7.45-7.50 (m, 3H), 7.41 (s, 1H), 5.70 (t, J=6.8 Hz, 1H), 5.57 (s, 2H), 3.98 (d, J=6.8 Hz, 2H), 2.64 (t, J=6.4 Hz, 2H), 2.48 (t, J=5.6 Hz, 2H), 1.80-1.86 (m, 2H). Mass spectrum (ESI, m/z): Calculated for C₂₅H₂₃Cl₂N₆O₃S₂: 589.1 [M+H], found: 590.9.

### (E)-4,5-dichloro-N-((2-(1-(1-(pyridin-2-yl)piperidin-4-yl)-1,4,5,6-tetrahydro-7H-indazol-7-ylidene)ethyl)carbamoyl)thiophene-2-sulfonamide, Compound #140

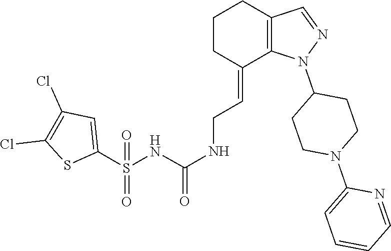 [SMILES: O=C(NC/C=C1\CCCc2cnn(C3CCN(c4ccccn4)CC3)c21)NS(=O)(=O)c1cc(Cl)c(Cl)s1]

¹H NMR (300 MHz, CD₃OD) δ: 8.06-8.12 (m, 1H), 7.98 (d, J=6.9 Hz, 1H), 7.69 (s, 1H), 7.52 (s, 1H), 7.48 (s, 1H), 7.04 (t, J=6.9 Hz, 1H), 5.90 (t, J=6.6 Hz, 1H), 4.90-5.00 (m, 1H), 4.35 (d, J=13.5 Hz, 2H), 4.01 (d, J=6.6 Hz, 2H), 3.53 (t, J=14.7 Hz, 2H), 2.64 (t, J=6.3 Hz, 2H), 2.55 (t, J=5.4 Hz, 2H), 2.21-2.31 (m, 4H), 1.88-1.92 (m, 2H). Mass spectrum (ESI, m/z): Calculated for C₂₄H₂₇Cl₂N₆O₃S₂: 581.1 [M+H], found: 581.2.

### (E)-N-((2-(1-(1-benzylpiperidin-4-yl)-1,4,5,6-tetrahydro-7H-indazol-7-ylidene)ethyl)carbamoyl)-4,5-dichlorothiophene-2-sulfonamide, Compound #148

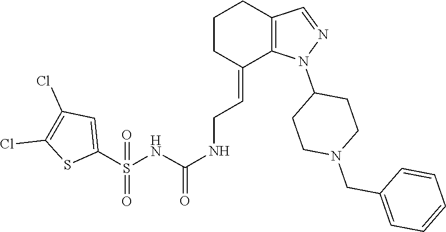 [SMILES: O=C(NC/C=C1\CCCc2cnn(C3CCN(Cc4ccccc4)CC3)c21)NS(=O)(=O)c1cc(Cl)c(Cl)s1]

¹H NMR (400 MHz, CD₃OD) δ: 7.65 (s, 1H), 7.50-7.55 (m, 5H), 7.30 (s, 1H), 5.80 (t, J=6.4 Hz, 1H), 4.74-4.79 (m, 1H), 4.37 (s, 2H), 3.94 (d, J=6.4 Hz, 2H), 3.63 (d, J=12.8 Hz, 2H), 3.29-3.31 (m, 2H), 2.59 (d, J=6.0 Hz, 2H), 2.47 (d, J=5.2 Hz, 2H), 2.32-2.39 (m, 2H), 2.22 (d, J=13.2 Hz, 2H), 1.80-1.86 (m, 2H). Mass spectrum (ESI, m/z): Calculated for C₂₆H₃₀Cl₂N₅O₃S₂: 594.1 [M+H], found: 594.1.

### (E)-4,5-dichloro-N-((2-(1-(1-methylpiperidin-4-yl)-1,4,5,6-tetrahydro-7H-indazol-7-ylidene)ethyl)carbamoyl)thiophene-2-sulfonamide, Compound #151

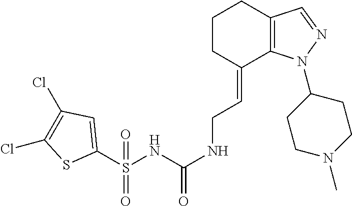 [SMILES: CN1CCC(n2ncc3c2/C(=C/CNC(=O)NS(=O)(=O)c2cc(Cl)c(Cl)s2)CCC3)CC1]

¹H NMR (300 MHz, CD₃OD) δ: 7.39 (s, 1H), 7.29 (s, 1H), 5.81 (brs, 1H), 4.78 (brs, 1H), 3.86 (d, J=6.0 Hz, 2H), 3.52-3.56 (m, 2H), 3.31-3.33 (m, 2H), 2.90 (s, 3H), 2.59 (t, J=6.3 Hz, 2H), 2.46 (t, J=5.4 Hz, 2H), 2.34-2.38 (m, 2H), 2.17-2.22 (m, 2H), 1.82-1.86 (m, 2H). Mass spectrum (ESI, m/z): Calculated for C₂₀H₂₆Cl₂N₅O₃S₂: 518.1 [M+H], found: 518.1.

### Example 6: Compound #4

### (E)-4-chloro-N-((2-(1-(2,4-dichlorobenzyl)-1,4,5,6-tetrahydro-7H-indazol-7-ylidene)ethyl)carbamoyl)benzenesulfonamide

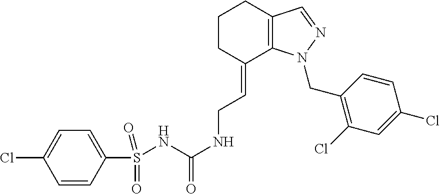 [SMILES: O=C(NC/C=C1\CCCc2cnn(Cc3ccc(Cl)cc3Cl)c21)NS(=O)(=O)c1ccc(Cl)cc1]

### Step 1. Synthesis of (E)-4-chloro-N-((2-(1-(2,4-dichlorobenzyl)-1,4,5,6-tetrahydro-7H-indazol-7-ylidene)ethyl)carbamoyl)benzenesulfonamide

To a solution of (E)-2-(1-(2,4-dichlorobenzyl)-1,4,5,6-tetrahydro-7H-indazol-7-ylidene)ethan-1-amine (66 mg, 0.205 mmol) in CH₂Cl₂ (5 mL) at 0° C. was added 4-chlorobenzenesulfonyl isocyanate (0.046 mL, 0.307 mmol). The reaction was stirred at 0° C. for 20 min before MeOH was added and the reaction was stirred at room temperature for 10 min. The reaction solution was diluted with EtOAc and washed with aq. 1N HCl and sat. aq. NaCl. The organic layer was dried over Na₂SO₄ and concentrated. Purification of the resulting residue by silica gel column with 40% EtOAc/heptane yielded (E)-4-chloro-N-((2-(1-(2,4-dichlorobenzyl)-1,4,5,6-tetrahydro-7H-indazol-7-ylidene)ethyl)carbamoyl)benzenesulfonamide.

¹H NMR (CHLOROFORM-d) δ: 7.75-7.78 (m, 1H), 7.74 (s, 1H), 7.37-7.51 (m, 4H), 7.13 (dd, J=8.1, 2.0 Hz, 1H), 6.54 (d, J=8.1 Hz, 1H), 6.42-6.50 (m, 1H), 5.49 (s, 2H), 5.20-5.26 (m, 1H), 3.90 (t, J=6.1 Hz, 2H), 2.64 (t, J=6.3 Hz, 2H), 2.33-2.46 (m, 2H), 2.05 (s, 1H), 1.77-1.95 (m, 2H). Mass spectrum (ESI, m/z): Calculated for C₂₃H₂₂Cl₃N₄O₃S, 539.0 [M+H], found 539.1.

The following representative compounds of formula (I) were similarly prepared according to the procedure described in Example 6 above, selecting and substituting suitable starting materials and reagents, as would be readily recognized by those skilled in the art.

### (E)-4-chloro-N-((2-(1-isobutyl-1,4,5,6-tetrahydro-7H-indazol-7-ylidene)ethyl)carbamoyl)benzenesulfonamide, Compound #80

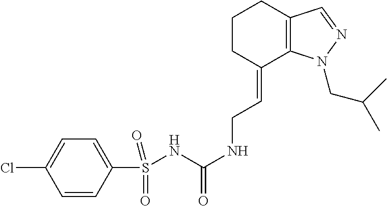 [SMILES: CC(C)Cn1ncc2c1/C(=C/CNC(=O)NS(=O)(=O)c1ccc(Cl)cc1)CCC2]

¹H NMR (CHLOROFORM-d) δ: 7.80-7.86 (m, 2H), 7.37-7.52 (m, 2H), 6.58-6.67 (m, 1H), 5.59 (t, J=6.8 Hz, 1H), 3.93-4.13 (m, 4H), 2.58 (t, J=6.1 Hz, 2H), 2.36-2.46 (m, 2H), 1.99-2.18 (m, 1H), 1.68-1.89 (m, 2H), 0.86 (d, J=6.6 Hz, 6H). Mass spectrum (ESI, m/z): Calculated for C₂₀H₂₆ClN₄O₃S: 437.1 [M+H], found: 437.1.

### (E)-N-((2-(1-(5-bromopyrimidin-2-yl)-1,4,5,6-tetrahydro-7H-indazol-7-ylidene)ethyl)carbamoyl)-4-chlorobenzenesulfonamide, Compound #94

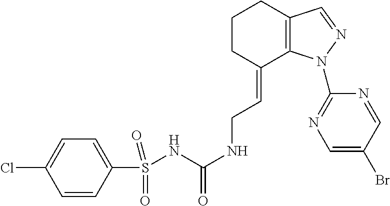 [SMILES: O=C(NC/C=C1\CCCc2cnn(-c3ncc(Br)cn3)c21)NS(=O)(=O)c1ccc(Cl)cc1]

¹H NMR (400 MHz, CHLOROFORM-d) δ: 8.80 (s, 2H), 7.79-7.87 (m, 2H), 7.60 (s, 1H), 7.41-7.50 (m, 2H), 6.61 (br s, 1H), 5.24-5.31 (m, 1H), 3.94 (dd, J=7.1, 5.6 Hz, 2H), 2.66 (t, J=6.3 Hz, 2H), 2.43-2.59 (m, 2H), 1.86 (dt, J=12.5, 6.1 Hz, 2H). Mass spectrum (ESI, m/z): Calculated for C₂₀H₁₉BrClN₆O₃S: 537.0 [M+H], found: 537.0.

### (E)-4-chloro-N-((2-(1-(6-methoxypyridin-3-yl)-1,4,5,6-tetrahydro-7H-indazol-7-ylidene)ethyl)carbamoyl)benzenesulfonamide, Compound #105

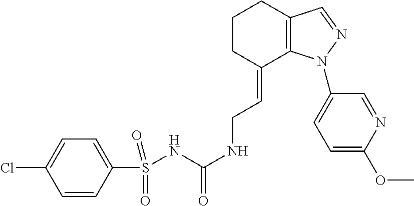 [SMILES: COc1ccc(-n2ncc3c2/C(=C/CNC(=O)NS(=O)(=O)c2ccc(Cl)cc2)CCC3)cn1]

¹H NMR (400 MHz, CHLOROFORM-d) δ: 8.17-8.20 (m, 1H), 7.75 (d, J=8.1 Hz, 2H), 7.63 (dd, J=8.8, 1.8 Hz, 1H), 7.43-7.51 (m, 3H), 6.84 (d, J=9.1 Hz, 1H), 6.25-6.34 (m, 1H), 5.03 (br t, J=6.8 Hz, 1H), 3.97 (s, 3H), 3.83 (t, J=6.1 Hz, 2H), 2.66 (t, J=6.1 Hz, 2H), 2.41-2.53 (m, 2H), 1.85 (quin, J=5.8 Hz, 3H). Mass spectrum (ESI, m/z): Calculated for C₂₂H₂₃ClN₅O₄S: 488.1 [M+H], found: 488.1.

### (E)-4-chloro-N-((2-(1-(1-(4-fluorophenyl)piperidin-4-yl)-1,4,5,6-tetrahydro-7H-indazol-7-ylidene)ethyl)carbamoyl)benzenesulfonamide, Compound #122

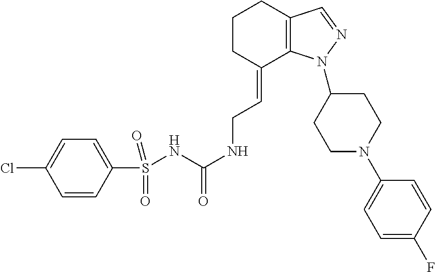 [SMILES: O=C(NC/C=C1\CCCc2cnn(C3CCN(c4ccc(F)cc4)CC3)c21)NS(=O)(=O)c1ccc(Cl)cc1]

¹H NMR (400 MHz, CHLOROFORM-d) δ: 7.80-7.78 (d, 2H), 7.39-7.37 (d, 2H), 7.32 (s, 1H), 6.89-6.99 (m, 4H), 6.69 (br s, 1H), 5.58 (t, J=6.8 Hz, 1H), 4.35 (m, 1H), 4.05 (t, J=6.1 Hz, 2H), 3.62-3.71 (m, 2H), 2.74-2.83 (m, 2H), 2.59 (t, J=6.1 Hz, 2H), 2.33-2.46 (m, 4H), 2.00-2.07 (m, 2H), 1.73-1.85 (m, 2H). Mass spectrum (ESI, m/z): Calculated for C₂₇H₃₀ClFN₅O₃S: 558.2 [M+H], found: 558.2.

### Example 7: Compound #150

### (E)-N-((2-(1-(2,4-dichlorobenzyl)-1,4,5,6-tetrahydro-7H-indazol-7-ylidene)ethyl)carbamoyl)-2,4-dimethylthiazole-5-sulfonamide

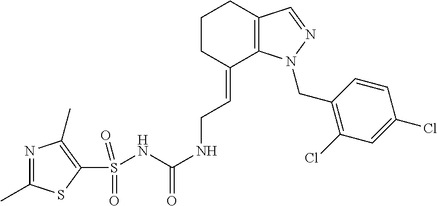 [SMILES: Cc1nc(C)c(S(=O)(=O)NC(=O)NC/C=C2\CCCc3cnn(Cc4ccc(Cl)cc4Cl)c32)s1]

### Step 1. Synthesis of phenyl (E)-(2-(1-(2,4-dichlorobenzyl)-1,4,5,6-tetrahydro-7H-indazol-7-ylidene)ethyl)carbamate

To a solution of (E)-2-(1-(2,4-dichlorobenzyl)-1,4,5,6-tetrahydro-7H-indazol-7-ylidene)ethan-1-amine (100 mg, 0.310 mmol, 1.00 equiv) in DCM (20 mL) at room temperature was added phenyl carbonochloridate (54 mg, 0.345 mmol) and TEA (38 mg, 0.376 mmol). The reaction was stirred for 4 h at room temperature. The resulting solution was concentrated under vacuum. The resulting residue was purified by silica gel column with PE:EA=1:1 to yield (E)-(2-(1-(2,4-dichlorobenzyl)-1,4,5,6-tetrahydro-7H-indazol-7-ylidene)ethyl)carbamate as white solid. Mass spectrum (ESI, m/z): Calculated for C₂₃H₂₂Cl₂N₃O₂, 442.1[M+H], found 442.1.

### Step 2. Synthesis of (E)-N-((2-(1-(2,4-dichlorobenzyl)-1,4,5,6-tetrahydro-7H-indazol-7-ylidene)ethyl)carbamoyl)-2,4-dimethylthiazole-5-sulfonamide

A solution of (E)-(2-(1-(2,4-dichlorobenzyl)-1,4,5,6-tetrahydro-7H-indazol-7-ylidene)ethyl)carbamate (120 mg, 0.271 mmol), 2,4-dimethylthiazole-5-sulfonamide (63 mg, 0.328 mmol), and DBU (50 mg, 0.328 mmol) in MeCN (20 mL) was stirred for 6 h at room temperature. The reaction was quenched with H₂O and the resulting mixture was extracted with EtOAc. The organic layers were combined and concentrated under vacuum. The resulting residue was purified by Prep-HPLC with the following conditions (1#-Waters 2767-5): Column, X Bridge C18, 5 um, 19*100 mm; mobile phase, Water with 0.05% TFA and CH₃CN (30% CH₃CN up to 50% in 10 min, up to 95% in 0.1 min, hold 95% in 0.9 min, down to 50% in 0.1 min, hold 50% in 1.4 min); Detector, UV 220 & 254 nm. The desired fraction was concentrated under vacuum. The resulting residue was dissolved in CH₃CN (5 mL) and HCl (2.5 N, 2 mL) was added. The resulting solution was concentrated under vacuum. The process was repeated again to yield (E)-N-((2-(1-(2,4-dichlorobenzyl)-1,4,5,6-tetrahydro-7H-indazol-7-ylidene)ethyl)carbamoyl)-2,4-dimethylthiazole-5-sulfonamide as a yellow solid.

¹H NMR (400 MHz, CD₃OD) δ: 7.82 (s, 1H), 7.55 (s, 1H), 7.31 (d, J=6.8 Hz, 1H), 6.68 (d, J=8.4 Hz, 1H), 5.71 (s, 2H), 5.52 (t, J=6.0 Hz, 1H), 3.85 (d, J=6.4 Hz, 2H), 2.75 (s, 3H), 2.71 (t, J=6.0 Hz, 2H), 2.62 (s, 3H), 2.53 (t, J=5.2 Hz, 2H), 1.86-1.89 (m, 2H). Mass spectrum (ESI, m/z): Calculated for C₂₂H₂₄Cl₂N₅O₃S₂, 540.1 [M+H], found 539.9.

The following representative compounds of formula (I) were similarly prepared according to the procedure described in Example 7 above, selecting and substituting suitable starting materials and reagents, as would be readily recognized by those skilled in the art.

### (E)-N-((2-(1-(2,4-dichlorobenzyl)-1,4,5,6-tetrahydro-7H-indazol-7-idene)ethyl)carbamoyl)-1-methyl-1H-pyrazole-4-sulfonamide, Compound #152

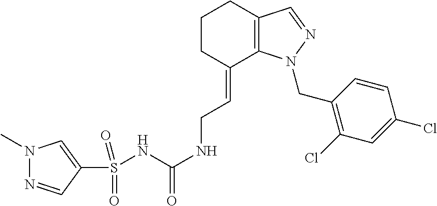 [SMILES: Cn1cc(S(=O)(=O)NC(=O)NC/C=C2\CCCc3cnn(Cc4ccc(Cl)cc4Cl)c32)cn1]

¹H NMR (400 MHz, CD₃OD) δ: 8.13 (s, 1H), 7.80 (s, 1H), 7.47 (s, 1H), 7.39 (s, 1H), 7.21 (d, J=8.4 Hz, 1H), 6.47 (d, J=8.4 Hz, 1H), 5.48 (s, 2H), 5.29 (t, J=6.0 Hz, 1H), 3.90 (d, J=6.4 Hz, 3H), 3.81 (d, J=6.8 Hz, 2H), 2.64 (t, J=6.0 Hz, 2H), 2.46 (t, J=5.2 Hz, 2H), 1.81-1.84 (m, 2H). Mass spectrum (ESI, m/z): Calculated for C₂₁H₂₃Cl₂N₆O₃S, 509.1 [M+H], found 509.0.

### (E)-N-((2-(1-(2,4-dichlorobenzyl)-1,4,5,6-tetrahydro-7H-indazol-7-ylidene)ethyl)carbamoyl)-1-methyl-1H-pyrazole-3-sulfonamide, Compound #153

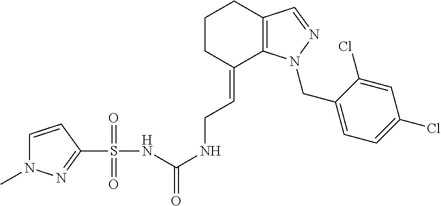 [SMILES: Cn1ccc(S(=O)(=O)NC(=O)NC/C=C2\CCCc3cnn(Cc4ccc(Cl)cc4Cl)c32)n1]

¹H NMR (400 MHz, DMSO) δ: 10.17 (s, 1H), 7.86 (s, 1H), 7.64 (s, 1H), 7.36-7.38 (m, 2H), 6.67 (s, 1H), 6.57 (d, J=8.4 Hz, 2H), 5.43 (s, 2H), 5.29-5.33 (m, 1H), 3.90 (s, 3H), 3.70-3.73 (m, 2H), 2.54-2.57 (m, 2H), 2.36-2.39 (m, 2H), 1.70-1.73 (m, 2H). Mass spectrum (ESI, m/z): Calculated for C₂₁H₂₃Cl₂N₆O₃S, 509.1[M+H], found 509.1.

### (E)-N-((2-(1-(2,4-dichlorobenzyl)-1,4,5,6-tetrahydro-7H-indazol-7-ylidene)ethyl)carbamoyl)-1-methyl-1H-pyrazole-5-sulfonamide, Compound #171

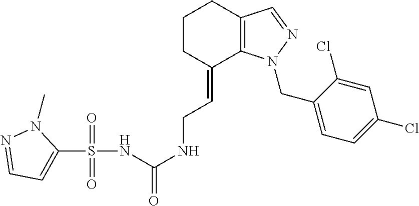 [SMILES: Cn1nccc1S(=O)(=O)NC(=O)NC/C=C1\CCCc2cnn(Cc3ccc(Cl)cc3Cl)c21]

¹H NMR (400 MHz, CD₃OD) δ: 7.52-7.53 (m, 2H), 7.49-7.51 (m, 1H), 7.25-7.27 (m, 1H), 6.87-6.88 (m, 1H), 6.52 (d, J=8.4 Hz, 1H), 5.52 (s, 2H), 5.31-5.34 (m, 1H), 4.13 (s, 3H), 3.83-3.85 (m, 2H), 2.66-2.69 (m, 2H), 2.47-2.50 (m, 2H), 1.82-1.88 (m, 2H). Mass spectrum (ESI, m/z): Calculated for C₂₁H₂₃Cl₂N₆O₃S, 509.1 [M+H], found 509.1.

### (E)-N-((2-(1-(2,4-dichlorobenzyl)-1,4,5,6-tetrahydro-7H-indazol-7-ylidene)ethyl)carbamoyl)-1,5-dimethyl-1H-pyrazole-4-sulfonamide, Compound #172

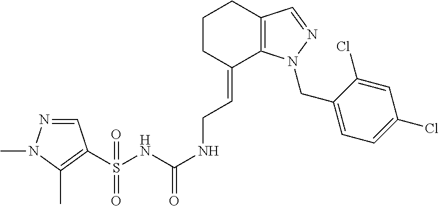 [SMILES: Cc1c(S(=O)(=O)NC(=O)NC/C=C2\CCCc3cnn(Cc4ccc(Cl)cc4Cl)c32)cnn1C]

¹H NMR (300 MHz, CD₃OD) δ: 7.76 (s, 1H), 7.48-7.49 (m, 1H), 7.41 (s, 1H), 7.21-7.25 (m, 1H), 6.49 (d, J=8.4 Hz, 1H), 5.48 (s, 2H), 5.27-5.31 (m, 1H), 3.80-3.84 (m, 5H), 2.63-2.67 (m, 2H), 2.51 (s, 3H), 2.44-2.48 (m, 2H), 1.78-1.86 (m, 2H). Mass spectrum (ESI, m/z): Calculated for C₂₂H₂₅Cl₂N₆O₃S, 523.1 [M+H], found 523.1.

### (E)-N-((2-(1-(2,4-dichlorobenzyl)-1,4,5,6-tetrahydro-7H-indazol-7-ylidene)ethyl)carbamoyl)-1-isopropyl-1H-pyrazole-4-sulfonamide, Compound #173

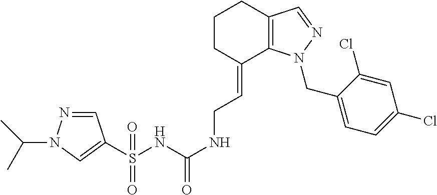 [SMILES: CC(C)n1cc(S(=O)(=O)NC(=O)NC/C=C2\CCCc3cnn(Cc4ccc(Cl)cc4Cl)c32)cn1]

¹H NMR (300 MHz, CD₃OD) δ: 8.24 (s, 1H), 7.85 (s, 1H), 7.48-7.51 (m, 2H), 7.22-7.26 (m, 1H), 6.53 (d, J=8.4 Hz, 1H), 5.59 (s, 2H), 5.33-5.37 (m, 1H), 4.53-4.62 (m, 1H), 3.84 (d, J=6.6 Hz, 2H), 2.64-2.68 (m, 2H), 2.47-2.49 (m, 2H), 1.79-1.86 (m, 2H), 1.24-1.30 (m, 6H). Mass spectrum (ESI, m/z): Calculated for C₂₃H₂₇Cl₂N₆O₃S, 537.1 [M+H], found 537.2.

### (E)-N-((2-(1-(2,4-dichlorobenzyl)-1,4,5,6-tetrahydro-7H-indazol-7-ylidene)ethyl)carbamoyl)-2-oxo-2,3-dihydrobenzo[d]oxazole-6-sulfonamide, Compound #176

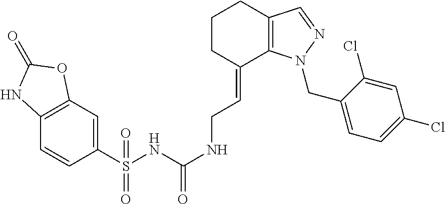 [SMILES: O=C(NC/C=C1\CCCc2cnn(Cc3ccc(Cl)cc3Cl)c21)NS(=O)(=O)c1ccc2[nH]c(=O)oc2c1]

¹H NMR (400 MHz, MeOH-d) δ: 7.77-7.85 (m, 2H), 7.36-7.43 (m, 2H), 7.19 (t, J=8.0 Hz, 2H), 6.39-6.52 (m, 1H), 5.43 (s, 2H), 5.15-5.30 (m, 1H), 3.79 (d, J=6.6 Hz, 2H), 2.63 (t, J=6.3 Hz, 2H), 2.38-2.46 (m, 2H), 1.80 (quin, J=6.1 Hz, 2H). Mass spectrum (ESI, m/z): Calculated for C₂₄H₂₂Cl₂N₅O₅S, 562.1 [M+H], found 562.0.

### (E)-N-((2-(1-(2,4-dichlorobenzyl)-1,4,5,6-tetrahydro-7H-indazol-7-ylidene)ethyl)carbamoyl)-2-methoxybenzo[d]oxazole-6-sulfonamide, Compound #177

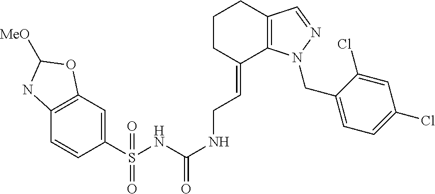 [SMILES: COC1Nc2ccc(S(=O)(=O)NC(=O)NC/C=C3\CCCc4cnn(Cc5ccc(Cl)cc5Cl)c43)cc2O1]

¹H NMR (400 MHz, CHLOROFORM-d) δ: 7.81-7.88 (m, 1H), 7.71-7.78 (m, 1H), 7.45-7.52 (m, 2H), 7.04-7.16 (m, 2H), 6.50 (d, J=8.1 Hz, 1H), 5.44 (s, 2H), 5.21 (t, J=6.8 Hz, 1H), 3.83 (d, J=7.1 Hz, 2H), 3.46 (s, 3H), 2.62 (br t, J=6.1 Hz, 2H), 2.23-2.51 (m, 2H), 1.76-1.85 (m, 2H). Mass spectrum (ESI, m/z): Calculated for C₂₅H₂₄Cl₂N₅O₅S, 576.1 [M+H], found 576.2.

### (E)-N-((2-(1-(2,4-dichlorobenzyl)-1,4,5,6-tetrahydro-7H-indazol-7-ylidene)ethyl)carbamoyl)-1,3-dimethyl-1H-pyrazole-4-sulfonamide, Compound #184

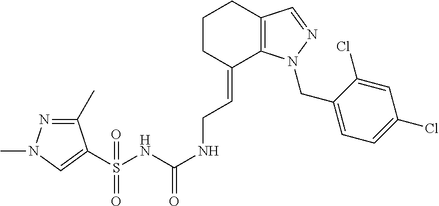 [SMILES: Cc1nn(C)cc1S(=O)(=O)NC(=O)NC/C=C1\CCCc2cnn(Cc3ccc(Cl)cc3Cl)c21]

¹H NMR (300 MHz, CD₃OD) δ: 8.07 (s, 1H), 7.47-7.48 (m, 1H), 7.42 (s, 1H), 7.22-7.25 (m, 1H), 6.49 (d, J=8.4 Hz, 1H), 5.49 (s, 2H), 5.27-5.32 (m, 1H), 3.81-3.83 (m, 5H), 2.63-2.67 (m, 2H), 2.46-2.48 (m, 2H), 2.38 (s, 3H), 1.81-1.85 (m, 2H). Mass spectrum (ESI, m/z): Calculated for C₂₂H₂₅Cl₂N₆O₃S, 523.1 [M+H], found 523.1.

### (E)-5-chloro-N-((2-(1-(2,4-dichlorobenzyl)-1,4,5,6-tetrahydro-7H-indazol-7-ylidene)ethyl)carbamoyl)-4-methylthiophene-2-sulfonamide, Compound #185

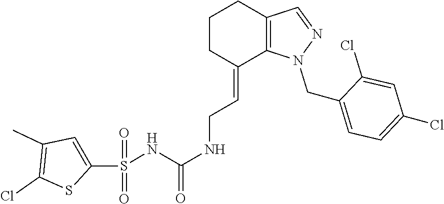 [SMILES: Cc1cc(S(=O)(=O)NC(=O)NC/C=C2\CCCc3cnn(Cc4ccc(Cl)cc4Cl)c32)sc1Cl]

¹H NMR (300 MHz, CD₃OD) δ: 7.49 (s, 1H), 7.41-7.44 (m, 2H), 7.17-7.21 (m, 1H), 6.46 (d, J=8.7 Hz, 1H), 5.45 (s, 2H), 5.26-5.28 (m, 1H), 3.81 (d, J=6.6 Hz, 2H), 2.60-2.64 (m, 2H), 2.43-2.47 (m, 2H), 2.17 (s, 3H), 1.73-1.84 (m, 2H). Mass spectrum (ESI, m/z): Calculated for C₂₂H₂₂Cl₃N₄O₃S₂, 559.0 [M+H], found 559.0.

### (E)-N-((2-(1-(2,4-dichlorobenzyl)-1,4,5,6-tetrahydro-7H-indazol-7-ylidene)ethyl)carbamoyl)-1,3-dimethyl-1H-pyrazole-5-sulfonamide, Compound #192

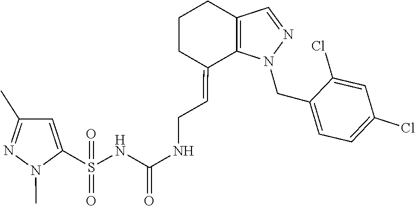 [SMILES: Cc1cc(S(=O)(=O)NC(=O)NC/C=C2\CCCc3cnn(Cc4ccc(Cl)cc4Cl)c32)n(C)n1]

¹H NMR (400 MHz, CD₃OD) δ: 7.48-7.49 (m, 1H), 7.44 (s, 1H), 7.23 (d, J=8.8 Hz, 1H), 6.64 (s, 1H), 6.48 (d, J=8.0 Hz, 1H), 5.48 (s, 2H), 5.29-5.31 (m, 1H), 4.02 (s, 3H), 3.81-3.82 (m, 2H), 2.63-2.66 (m, 2H), 2.44-2.45 (m, 2H), 2.21 (s, 3H), 1.80-1.83 (m, 2H). Mass spectrum (ESI, m/z): Calculated for C₂₂H₂₅Cl₂N₆O₃S, 523.1 [M+H], found 523.1.

### (E)-5-chloro-N-((2-(1-(2,4-dichlorophenyl)-1,4,5,6-tetrahydro-7H-indazol-7-ylidene)ethyl)carbamoyl)-4-methylthiophene-2-sulfonamide, Compound #196

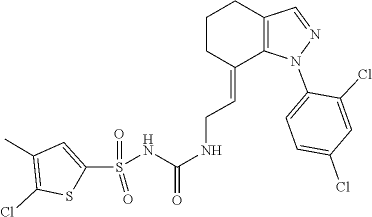 [SMILES: Cc1cc(S(=O)(=O)NC(=O)NC/C=C2\CCCc3cnn(-c4ccc(Cl)cc4Cl)c32)sc1Cl]

¹H NMR (400 MHz, CD₃OD) δ: 7.63 (s, 1H), 7.56-7.58 (m, 1H), 7.48-7.50 (m, 2H), 7.43-7.45 (m, 1H), 4.88 (s, 1H), 3.72 (d, J=6.8 Hz, 2H), 2.68 (t, J=6.0 Hz, 2H), 2.47-2.58 (m, 2H), 2.21 (s, 3H), 1.82-1.88 (m, 2H). Mass spectrum (ESI, m/z): Calculated for C₂₁H₂₀Cl₃N₄O₃S₂: 545.0 [M+H], found: 547.2.

### Example 8: Compound #34 and Compound #35

### (Z)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate and

### (E)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate

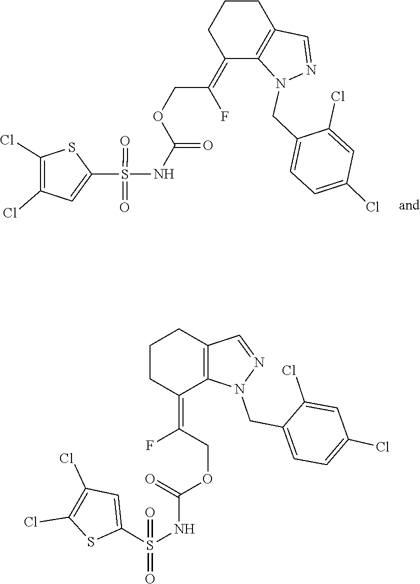 [SMILES: O=C(NS(=O)(=O)c1cc(Cl)c(Cl)s1)OC/C(F)=C1\CCCc2cnn(Cc3ccc(Cl)cc3Cl)c21 ; O=C(NS(=O)(=O)c1cc(Cl)c(Cl)s1)OC/C(F)=C1/CCCc2cnn(Cc3ccc(Cl)cc3Cl)c21]

### Step 1. Synthesis of 2-ethoxycyclohex-2-en-1-one

A solution of cyclohexane-1,2-dione (50 g, 446 mmol) and 4-methylbenzenesulfonic acid (10 g, 58.1 mmol) in EtOH (200 mL) and toluene (400 mL) was stirred at 90° C. for 2 days. The reaction was concentrated under vacuum. The resulting residue was purified by silica gel column with PE:EA=90:10 to yield 2-ethoxycyclohex-2-en-1-one. Mass spectrum (ESI, m/z): Calculated for C₈H₁₃O₂, 141.1 (M+H), found 141.2.

### Step 2. Synthesis of 3-(hydroxymethylene)cyclohexane-1,2-dione

A solution of 2-ethoxycyclohex-2-en-1-one (23 g, 0.164 mol) in N,N-dimethylformamide dimethyl acetal (200 mL) was stirred overnight at 90° C. The resulting solution was concentrated under vacuum and the resulting residue was stirred vigorously in CH₂Cl₂/aq. HCl (2 N) at room temperature for 6 hours. The resulting mixture was extracted with CH₂Cl₂. The organic layers were combined, dried over Na₂SO₄ and concentrated under vacuum to yield 3-(hydroxymethylene)cyclohexane-1,2-dione as yellow oil. Mass spectrum (ESI, m/z): Calculated for C₇H₉O₃, 141.0 (M+H), found 140.8.

### Step 3. Synthesis of 1-(2,4-dichlorobenzyl)-1,4,5,6-tetrahydro-7H-indazol-7-one

A solution of 3-(hydroxymethylene)cyclohexane-1,2-dione (1.4 g, 9.99 mmol), (2,4-dichlorobenzyl)hydrazine hydrocholride (2.50 g, 11.0 mmol), and TFA (2 mL) in 1,4-dioxane (100 mL) was heated at 60° C. for 2 hours. The reaction was concentrated under vacuum. The residue was neutralized with aq. NaHCO₃ and extracted with EtOAc. The organic layer was washed with sat. aq. NaCl and dried over Na₂SO₄, and concentrated. Purification of the resulting residue by silica gel column with 10% EtOAc/heptane yielded 1-(2,4-dichlorobenzyl)-1,4,5,6-tetrahydro-7H-indazol-7-one as a yellow solid. Mass spectrum (ESI, m/z): Calculated for C₁₄H₁₃Cl₂N₂O, 295.0 (M+H), found 249.9.

### Step 4. Synthesis of ethyl (Z)-2-(1-(2,4-dichlorobenzyl)-1,4,5,6-tetrahydro-7H-indazol-7-ylidene)-2-fluoroacetate and ethyl (E)-2-(1-(2,4-dichlorobenzyl)-1,4,5,6-tetrahydro-7H-indazol-7-ylidene)-2-fluoroacetate

To a solution of 1-(2,4-dichlorobenzyl)-1,4,5,6-tetrahydro-7H-indazol-7-one (16.0 g, 66.0 mmol) in THF (100 mL) at −78° C. under nitrogen was added n-BuLi (26.4 mL, 66.0 mmol). The reaction was stirred for 2 h at −78° C. before 1-(2,4-dichlorobenzyl)-1,4,5,6-tetrahydro-7H-indazol-7-one (16.5 g, 22.0 mmol) was added. The resulting solution was stirred for 30 min at −78° C. and was then warmed up to room temperature and stirred overnight. The reaction was quenched with EtOH and the resulting mixture was extracted with EtOAc. The organic layer was washed with sat. aq. NaCl, dried over Na₂SO₄, and concentrated under vacuum. The resulting residue was purified by silica gel column with PE:EA=70/30 to yield ethyl (Z)-2-(1-(2,4-dichlorobenzyl)-1,4,5,6-tetrahydro-7H-indazol-7-ylidene)-2-fluoroacetate and ethyl (E)-2-(1-(2,4-dichlorobenzyl)-1,4,5,6-tetrahydro-7H-indazol-7-ylidene)-2-fluoroacetate. Mass spectrum (ESI, m/z) of ethyl (Z)-2-(1-(2,4-dichlorobenzyl)-1,4,5,6-tetrahydro-7H-indazol-7-ylidene)-2-fluoroacetate: Calculated for C₁₈H₁₇Cl₂FN₂O₂, 383.1 (M+H), found 383.0. Mass spectrum (ESI, m/z) of ethyl (E)-2-(1-(2,4-dichlorobenzyl)-1,4,5,6-tetrahydro-7H-indazol-7-ylidene)-2-fluoroacetate: Calculated for C₁₈H₁₈Cl₂FN₂O₂, 383.1 (M+H), found 383.0.

### Step 5. Synthesis of (Z)-2-(1-(2,4-dichlorobenzyl)-1,4,5,6-tetrahydro-7H-indazol-7-ylidene)-2-fluoroethan-1-ol

To a solution of ethyl (Z)-2-(1-(2,4-dichlorobenzyl)-1,4,5,6-tetrahydro-7H-indazol-7-ylidene)-2-fluoroacetate (113 mg, 0.295 mmol) in THF (10 mL) at −78° C. under nitrogen was added DIBAL-H (0.79 mL, 0.79 mmol) in portions. The reaction was stirred for 30 min at −78° C. and was then stirred for 3 h at 0° C. The reaction was quenched by the addition of MeOH (1 mL) and diluted with H₂O (50 mL). The resulting mixture was extracted with DCM and the organic layers were combined and concentrated under vacuum. The resulting residue was purified by silica gel column with PE:EA=30/70 to yield (Z)-2-(1-(2,4-dichlorobenzyl)-1,4,5,6-tetrahydro-7H-indazol-7-ylidene)-2-fluoroethan-1-ol as a white solid. Mass spectrum (ESI, m/z): Calculated for C₁₆H₁₆Cl₂FN₂O, 341.1 (M+H), found 340.9.

### Step 6. Synthesis of (Z)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate

A solution of 4,5-dichlorothiophene-2-sulfonamide (95 mg, 0.409 mmol), 1,1′-carbonyldiimidazole (CDI) (67 mg, 0.414 mmol), and triethylamine (45 mg, 0.446 mmol) in THF (4 mL) was stirred for 16 hours at room temperature. (Z)-2-(1-(2,4-dichlorobenzyl)-1,4,5,6-tetrahydro-7H-indazol-7-ylidene)-2-fluoroethan-1-ol (50 mg, 0.147 mmol) was then added and the reaction was heated by microwave at 100° C. for 1 h. The reaction mixture was diluted with EtOAc and washed with sat. aq NaCl. The organic solution was concentrated under vacuum. The resulting residue was purified by Prep-HPLC with the following conditions (1#-Waters 2767-5): Column, SunFire Prep C18, 5 um, 19*100 mm; mobile phase, Water with 0.05% NH₄HCO₃ and CH₃CN (30% CH₃CN up to 70% in 10 min, up to 95% in 0.1 min, hold 95% in 0.9 min, down to 30% in 0.1 min, hold 30% in 1.4 min); Detector, UV 220 & 254 nm to yield (Z)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate as a white solid.

¹H NMR (300 MHz, Methanol-d4) δ: 7.57 (s, 1H), 7.41-7.49 (m, 2H), 7.23 (d, J=8.1 Hz, 1H), 6.72 (d, J=8.4 Hz, 1H), 5.37 (s, 2H), 4.97 (s, 1H), 4.80 (s, 1H), 2.67 (t, J=6.6 Hz, 2H), 2.44 (t, J=4.8 Hz, 2H), 1.83-1.92 (m, 2H). ¹⁹F NMR (300 MHz, Methanol-d4) δ: −107.002. Mass spectrum (ESI, m/z): Calculated for C₂₁H₁₇Cl₄FN₃O₄S₂, 597.9 (M+H), found 599.9.

### Step 7. Synthesis of (E)-2-(1-(2,4-dichlorobenzyl)-1,4,5,6-tetrahydro-7H-indazol-7-ylidene)-2-fluoroethan-1-ol

To solution of ethyl (E)-2-(1-(2,4-dichlorobenzyl)-1,4,5,6-tetrahydro-7H-indazol-7-ylidene)-2-fluoroacetate (2.30 g, 6.00 mmol) in THF (50 mL) at −78° C. under nitrogen was added DIBAL-H (18.0 mL, 18.0 mmol) in portions. The reaction was stirred for 30 min at −78° C. and was then stirred for 3 h at 0° C. The reaction was quenched by the addition of MeOH (10 mL) and diluted with H₂O (50 mL). The resulting mixture was extracted with DCM and the organic layers were combined and concentrated under vacuum. The resulting residue was purified by silica gel column with PE:EA=30/70 to yield (E)-2-(1-(2,4-dichlorobenzyl)-1,4,5,6-tetrahydro-7H-indazol-7-ylidene)-2-fluoroethan-1-ol as a yellow solid.

### Step 8. Synthesis of (E)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate

A solution of 4,5-dichlorothiophene-2-sulfonamide (95 mg, 0.409 mmol), 1,1′-carbonyldiimidazole (CDI) (67 mg, 0.414 mmol), and triethylamine (45 mg, 0.446 mmol) in THF (4 mL) was stirred for 16 hours at room temperature. (E)-2-(1-(2,4-dichlorobenzyl)-1,4,5,6-tetrahydro-7H-indazol-7-ylidene)-2-fluoroethan-1-ol (50 mg, 0.147 mmol) was then added and the reaction was heated by microwave at 100° C. for 1 h. The reaction mixture was diluted with EtOAc and washed with sat. aq NaCl. The organic solution was concentrated under vacuum. The resulting residue was purified by Prep-HPLC with the following conditions (1#-Waters 2767-5): Column, SunFire Prep C18, 5 um, 19*100 mm; mobile phase, Water with 0.05% NH₄HCO₃ and CH₃CN (30% CH₃CN up to 70% in 10 min, up to 95% in 0.1 min, hold 95% in 0.9 min, down to 30% in 0.1 min, hold 30% in 1.4 min); Detector, UV 220 & 254 nm to yield (E)-2-(1-(2,4-dichlorobenzyl)-1,4,5,6-tetrahydro-7H-indazol-7-ylidene)-2-fluoroethyl ((4,5-dichlorothiophen-2-yl)sulfonyl)carbamate as a white solid.

¹H NMR (300 MHz, Methanol-d4) δ: 7.30-7.37 (m, 3H), 7.20 (d, J=8.4 Hz, 1H), 6.78 (d, J=8.4 Hz, 1H), 5.22 (s, 2H), 4.61 (s, 1H), 4.53 (s, 1H), 2.64 (t, J=6.9 Hz, 2H), 2.45-2.50 (m, 2H), 1.84-1.92 (m, 2H). ¹⁹F NMR (300 MHz, Methanol-d4) δ: −111.97. Mass spectrum (ESI, m/z): Calculated for C₂₁H₁₇Cl₄FN₃O₄S₂, 597.9 (M+H), found 599.9.

### Example 9: Compound #93

### (Z)-3-(1-(2,4-dichlorobenzyl)-1,4,5,6-tetrahydro-7H-indazol-7-ylidene)-N-((4,5-dichlorothiophen-2-yl)sulfonyl)-3-fluoropropanamide

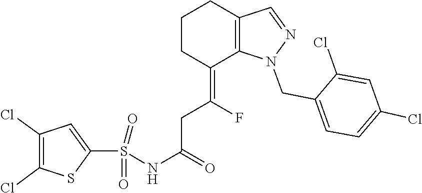 [SMILES: O=C(C/C(F)=C1\CCCc2cnn(Cc3ccc(Cl)cc3Cl)c21)NS(=O)(=O)c1cc(Cl)c(Cl)s1]

### Step 1. Synthesis of (Z)-7-(2-chloro-1-fluoroethylidene)-1-(2,4-dichlorobenzyl)-4,5,6,7-tetrahydro-1H-indazole

To a solution of (Z)-2-(1-(2,4-dichlorobenzyl)-1,4,5,6-tetrahydro-7H-indazol-7-ylidene)-2-fluoroethan-1-ol (320 mg, 0.938 mmol) in CH₂Cl₂ (50 mL) at 0° C. was added thionyl chloride (187 m g, 1.385 mmol) dropwise. The reaction was then stirred at 0° C. for 3 h. The reaction solution was concentrated under vacuum to yield crude (Z)-7-(2-chloro-1-fluoroethylidene)-1-(2,4-dichlorobenzyl)-4,5,6,7-tetrahydro-1H-indazole, which was used in the next step without further purification.

### Step 2. Synthesis of methyl (Z)-3-(1-(2,4-dichlorobenzyl)-1,4,5,6-tetrahydro-7H-indazol-7-ylidene)-3-fluoropropanoate

To a 30 mL pressure tank reactor was added (Z)-7-(2-chloro-1-fluoroethylidene)-1-(2,4-dichlorobenzyl)-4,5,6,7-tetrahydro-1H-indazole (500 mg, 1.39 mmol), Pd(PPh₃)₂Cl₂ (50 mg, 0.071 mmol), K₂CO₃ (212 mg, 1.536 mmol), MeOH (20 ml), and THF (40 ml). CO was introduced in. The reaction was stirred at 60° C. for 6 h under 20 atm. The reaction mixture was then concentrated under vacuum. The residue was suspended in H₂O and extracted with EtOAc. The organic layer was washed with sat. aq. NaCl and concentrated. The resulting residue was purified by silica gel column with PE:EA=10/90 to yield (Z)-3-(1-(2,4-dichlorobenzyl)-1,4,5,6-tetrahydro-7H-indazol-7-ylidene)-3-fluoropropanoate as a yellow solid. Mass spectrum (ESI, m/z): Calculated for C₁₈H₁₇Cl₂FN₂O₂, 383.1 (M+H), found 382.9.

### Step 3. Synthesis of (Z)-3-(1-(2,4-dichlorobenzyl)-1,4,5,6-tetrahydro-7H-indazol-7-ylidene)-3-fluoropropanoic acid

A solution of (Z)-3-(1-(2,4-dichlorobenzyl)-1,4,5,6-tetrahydro-7H-indazol-7-ylidene)-3-fluoropropanoate (365 mg, 0.952 mmol) and LiOH (220 mg, 9.17 mmol) in MeOH (20 ml), H₂O (10 ml) was stirred overnight at 25° C. The reaction was concentrated under vacuum. The residue was neutralized with aq HCl and extracted with EtOAc. The organic layer was washed with sat. aq. NaCl and concentrated to yield (Z)-3-(1-(2,4-dichlorobenzyl)-1,4,5,6-tetrahydro-7H-indazol-7-ylidene)-3-fluoropropanoic acid as a white solid. Mass spectrum (ESI, m/z): Calculated for C₁₇H₁₆Cl₂FN₂O₂, 369.0 (M+H), found 368.9.

### Step 4. Synthesis of (Z)-3-(1-(2,4-dichlorobenzyl)-1,4,5,6-tetrahydro-7H-indazol-7-ylidene)-N-((4,5-dichlorothiophen-2-yl)sulfonyl)-3-fluoropropanamide

A solution of (Z)-3-(1-(2,4-dichlorobenzyl)-1,4,5,6-tetrahydro-7H-indazol-7-ylidene)-3-fluoropropanoic acid (300 mg, 0.813 mmol), 4,5-dichlorothiophene-2-sulfonamide (245 mg, 1.06 mmol), DMAP (199 mg, 1.63 mmol), and EDCI (312 mg, 1.63 mmol) in CH₂Cl₂ (10 mL) was stirred for 16 hours at room temperature. The resulting solution was concentrated under vacuum. The resulting residue was purified by TLC with DCM:MeOH=20:1, followed by purification by Prep-HPLC with the following conditions (1#-Waters 2767-5): Column, X Bridge C18, 5 um, 19*100 mm; mobile phase, Water with 0.05% NH₄HCO₃ and CH₃CN (35% CH₃CN up to 53% in 10 min, up to 95% in 0.1 min, hold 95% in 0.9 min, down to 35% in 0.1 min, hold 35% in 1.4 min); Detector, UV 220&254 nm. The reuslting residue was purified again by Prep-HPLC with the following conditions (1#-Waters 2767-5): Column, SunFire Prep C18, 5 um, 19*100 mm; mobile phase, Water with 0.05% TFA and CH₃CN (50% CH₃CN up to 80% in 10 min, up to 95% in 0.1 min, hold 95% in 0.9 min, down to 50% in 0.1 min, hold 50% in 1.4 min); Detector, UV 220&254 nm to yield g)-3-(1-(2,4-dichlorobenzyl)-1,4,5,6-tetrahydro-7H-indazol-7-ylidene)-N-((4,5-dichlorothiophen-2-yl)sulfonyl)-3-fluoropropanamide as a white solid.

¹H NMR (300 MHz, Methanol-d4) δ: 7.68 (s, 1H), 7.35-7.39 (m, 2H), 7.19 (d, J=8.4 Hz, 1H), 6.66 (d, J=6.0 Hz, 1H), 5.57 (s, 2H), 3.59 (s, 1H), 3.50 (s, 1H), 2.63 (t, J=6.3 Hz, 2H), 2.14-2.25 (m, 2H), 1.85-1.90 (m, 2H). Mass spectrum (ESI, m/z): Calculated for C₂₁H₁₇Cl₄FN₃O₃S₂, 581.9 (M+H), found 583.8.

### Example 10: Compound #56

### (Z)-4-(1-(2,4-dichlorobenzyl)-1,4,5,6-tetrahydro-7H-indazol-7-ylidene)-N-((4,5-dichlorothiophen-2-yl)sulfonyl)-4-fluorobutanamide

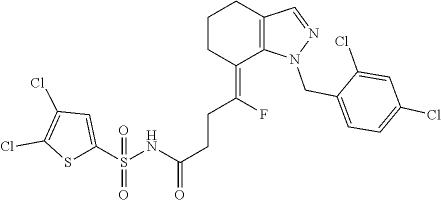 [SMILES: O=C(CC/C(F)=C1\CCCc2cnn(Cc3ccc(Cl)cc3Cl)c21)NS(=O)(=O)c1cc(Cl)c(Cl)s1]

### Step 1. Synthesis of diethyl (Z)-2-(2-(1-(2,4-dichlorobenzyl)-1,4,5,6-tetrahydro-7H-indazol-7-ylidene)-2-fluoroethyl)malonate

To a solution of diethyl malonate (326 mg, 2.04 mmol) in THF (30 mL) at 0° C. was added Cs₂CO₃ (751 mg, 2.30 mmol). The reaction was stirred for 30 min at 0° C. before (Z)-7-(2-chloro-1-fluoroethylidene)-1-(2,4-dichlorobenzyl)-4,5,6,7-tetrahydro-1H-indazole (330 mg, 0.966 mmol) was added. The reaction was stirred overnight at 50° C. The resulting solution was diluted with H₂O, and extracted with EA. The organic layer was washed with sat. aq. NaCl and concentrated under vacuum. The resulting residue was purified by silica gel column with PE:EA=80/20 to yield diethyl (Z)-2-(2-(1-(2,4-dichlorobenzyl)-1,4,5,6-tetrahydro-7H-indazol-7-ylidene)-2-fluoroethyl)malonate as yellow oil. Mass spectrum (ESI, m/z): Calculated for C₂₃H₂₆Cl₂FN₂O₄, 483.1 (M+H), found 483.1.

### Step 2. Synthesis of (Z)-4-(1-(2,4-dichlorobenzyl)-1,4,5,6-tetrahydro-7H-indazol-7-ylidene)-4-fluorobutanoic acid

To a 20 mL microwave vial was added diethyl (Z)-2-(2-(1-(2,4-dichlorobenzyl)-1,4,5,6-tetrahydro-7H-indazol-7-ylidene)-2-fluoroethyl)malonate (280 mg, 0.579 mmol), DMSO (8 mL), and a solution of LiCl (49 mg, 1.16 mmol) in H₂O (1 mL). The reaction was heated by microwave at 170° C. for 2 h. The resulting solution was diluted with H₂O and extracted with EtOAc. The organic layer was washed with sat. aq. NaCl and concentrated under vacuum. The resulting residue was dissolved in MeOH (20 mL). To the resulted solution was added a solution of NaOH (116 mg, 2.9 mmol) in H₂O (10 mL). The reaction was stirred overnight at 25° C. The resulting solution was concentrated under vacuum and then diluted with H₂O. The pH was adjusted to ˜4-5 and the resulting mixture was extracted with EtOAc. The organic layer was washed with sat. aq. NaCl and concentrated under vacuum. The resulting residue was purified by preparative TLC with DCM:MeOH=30:1 to yield (Z)-4-(1-(2,4-dichlorobenzyl)-1,4,5,6-tetrahydro-7H-indazol-7-ylidene)-4-fluorobutanoic acid as a yellow oil. Mass spectrum (ESI, m/z): Calculated for C₁₈H₁₈Cl₂FN₂O₂, 383.1 (M+H), found 383.0.

### Step 3. Synthesis of (Z)-4-(1-(2,4-dichlorobenzyl)-1,4,5,6-tetrahydro-7H-indazol-7-ylidene)-N-((4,5-dichlorothiophen-2-yl)sulfonyl)-4-fluorobutanamide

A solution of (Z)-4-(1-(2,4-dichlorobenzyl)-1,4,5,6-tetrahydro-7H-indazol-7-ylidene)-4-fluorobutanoic acid (80 mg, 0.209 mmol), 4,5-dichlorothiophene-2-sulfonamide (63 mg, 0.271 mmol), DMAP (51 mg, 0.418 mmol), and EDCl (80 mg, 0.419 mmol) in CH₂Cl₂ (3 mL) was stirred for 16 hours at room temperature. The reaction solution was diluted with EtOAc, washed with sat. aq. NaCl, and concentrated under vacuum. The resulting residue was purified by Prep-HPLC with the following conditions (1#-Waters 2767-5): Column, SunFire Prep C18, 5 um, 19*100 mm; mobile phase, Water with 0.05% TFA and CH₃CN (40% CH₃CN up to 76% in 10 min, up to 95% in 0.1 min, hold 95% in 0.9 min, down to 40% in 0.1 min, hold 40% in 1.4 min); Detector, UV 220&254 nm to yield (Z)-4-(1-(2,4-dichlorobenzyl)-1,4,5,6-tetrahydro-7H-indazol-7-ylidene)-N-((4,5-dichlorothiophen-2-yl)sulfonyl)-4-fluorobutanamide as a white solid.

¹H NMR (300 MHz, Methanol-d4) δ: 7.64 (s, 1H), 7.40 (s, 1H), 7.35 (s, 1H), 7.19 (d, J=8.4 Hz, 1H), 6.63 (d, J=8.4 Hz, 1H), 5.27 (s, 2H), 2.72 (t, J=6.6 Hz, 1H), 2.58-2.67 (m, 3H), 2.48 (t, J=6.6 Hz, 2H), 2.27 (t, J=5.4 Hz, 2H), 1.76-1.82 (m, 2H). ¹⁹F NMR (300 MHz, Methanol-d4) δ: −105.96. Mass spectrum (ESI, m/z): Calculated for C₂₂H₁₉Cl₄FN₃O₃S₂, 596.0 (M+H), found 597.9.

### Example 11, Compound #40

### (Z)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide

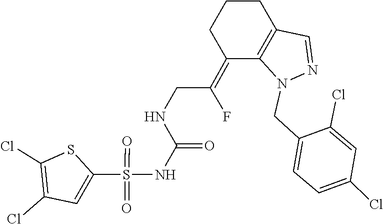 [SMILES: O=C(NC/C(F)=C1\CCCc2cnn(Cc3ccc(Cl)cc3Cl)c21)NS(=O)(=O)c1cc(Cl)c(Cl)s1]

### Step 1. Synthesis of (Z)-2-(2-(1-(2,4-dichlorobenzyl)-1,4,5,6-tetrahydro-7H-indazol-7-ylidene)-2-fluoroethyl)isoindoline-1,3-dione

(Z)-2-(2-(1-(2,4-dichlorobenzyl)-1,4,5,6-tetrahydro-7H-indazol-7-ylidene)-2-fluoroethyl)isoindoline-1,3-dione was prepared according to the procedures as described in Example 5, Step 1.

### Step 2. Synthesis of (Z)-2-(1-(2,4-dichlorobenzyl)-1,4,5,6-tetrahydro-7H-indazol-7-ylidene)-2-fluoroethan-1-amine

(Z)-2-(1-(2,4-dichlorobenzyl)-1,4,5,6-tetrahydro-7H-indazol-7-ylidene)-2-fluoroethan-1-amine was prepared according to procedures as described in Example 5, Step 2.

### Step 3. Synthesis of (Z)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide

(Z)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide was prepared according to the procedures as described in Example 5, Step 3.

¹H NMR (300 MHz, Methanol-d4) δ: 7.62 (s, 1H), 7.44-7.46 (m, 1H), 7.39 (s, 1H), 7.20 (d, J=8.4 Hz, 1H), 6.73 (d, J=8.4 Hz, 1H), 5.34 (s, 2H), 4.06 (d, J=21.3 Hz, 2H), 2.63 (t, J=6.6 Hz, 2H), 2.42 (t, J=5.4 Hz, 2H), 1.84-1.93 (m, 2H). ¹⁹F NMR (300 MHz, Methanol-d4) δ: −103.29. Mass spectrum (ESI, m/z): Calculated for C₂₁H₁₈Cl₄FN₄O₃S₂, 596.9 (M+H), found 598.9.

The following representative compounds of formula (I) were similarly prepared according to the procedure described in Example 11 (and Example 5 as referenced therein) above, selecting and substituting suitable starting materials and reagents, as would be readily recognized by those skilled in the art.

### (Z)-4-chloro-N-((2-(1-(2,4-dichlorobenzyl)-1,4,5,6-tetrahydro-7H-indazol-7-ylidene)-2-fluoroethyl)carbamoyl)benzenesulfonamide, Compound #86

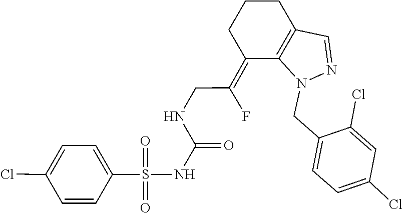 [SMILES: O=C(NC/C(F)=C1\CCCc2cnn(Cc3ccc(Cl)cc3Cl)c21)NS(=O)(=O)c1ccc(Cl)cc1]

¹H NMR (300 MHz, Methanol-d₄, ppm) δ: 7.89 (d, J=8.7 Hz, 2H), 7.48 (d, J=8.7 Hz, 2H), 7.32-7.39 (m, 2H), 7.19 (d, J=8.4 Hz, 1H), 6.68 (d, J=8.4 Hz, 1H), 5.29 (s, 2H), 4.00 (d, J=31.2 Hz, 2H), 2.59 (t, J=6.6 Hz, 2H), 2.34 (t, J=5.1 Hz, 2H), 1.73-1.77 (m, 2H). ¹⁹F NMR (300 MHz, Methanol-d₄, ppm) δ: −107.109. Mass spectrum (ESI, m/z): Calculated for C₂₃H₂₁Cl₃FN₄O₃S, 557.0 [M+H], found 558.9.

### (Z)-5-chloro-N-((2-(1-(2,4-dichlorobenzyl)-1,4,5,6-tetrahydro-7H-indazol-7-ylidene)-2-fluoroethyl)carbamoyl)-2-methoxybenzenesulfonamide, Compound #89

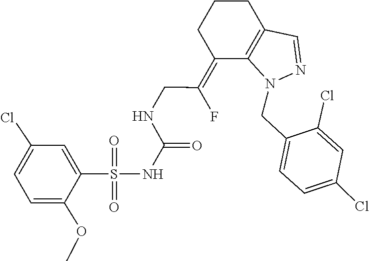 [SMILES: COc1ccc(Cl)cc1S(=O)(=O)NC(=O)NC/C(F)=C1\CCCc2cnn(Cc3ccc(Cl)cc3Cl)c21]

¹H NMR (300 MHz, DMSO-d₆, ppm) δ: 10.663 (s, 1H), 7.59-7.71 (m, 3H), 7.35-7.40 (m, 2H), 7.19 (d, J=9.0 Hz, 1H), 6.76-6.84 (m, 2H), 5.23 (s, 2H), 3.96 (d, J=21.6 Hz, 2H), 3.80 (s, 3H), 2.53 (t, J=5.7 Hz, 2H), 2.27 (s, 2H), 1.66 (s, 2H). ¹⁹F NMR (300 MHz, DMSO, ppm) δ: −106.998. Mass spectrum (ESI, m/z): Calculated for C₂₄H₂₃Cl₃FN₄O₄S, 587.0 [M+H]⁺, found 588.9.

### (Z)—N-((2-(1-(2,4-dichlorobenzyl)-1,4,5,6-tetrahydro-7H-indazol-7-ylidene)-2-fluoroethyl)carbamoyl)-5-fluoro-2-methoxybenzenesulfonamide, Compound #90

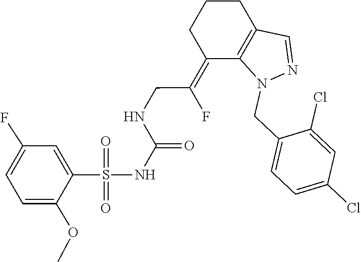 [SMILES: COc1ccc(F)cc1S(=O)(=O)NC(=O)NC/C(F)=C1\CCCc2cnn(Cc3ccc(Cl)cc3Cl)c21]

¹H NMR (300 MHz, DMSO-d₆, ppm) δ: 10.62 (s, 1H), 7.60 (s, 1H), 7.46-7.59 (m, 2H), 7.35-7.38 (m, 2H), 7.19 (d, J=9.0 Hz, 1H), 6.83 (t, J=5.7 Hz, 1H), 6.80 (d, J=8.4 Hz, 1H), 5.23 (s, 2H), 3.96 (d, J=21.9 Hz, 2H), 3.78 (s, 3H), 2.53 (t, J=6.3 Hz, 2H), 2.27 (s, 2H), 1.66-1.75 (m, 2H). ¹⁹F NMR (300 MHz, DMSO-d6, ppm) δ: −106.972, −122.864. Mass spectrum (ESI, m/z): Calculated for C₂₄H₂₁Cl₂F₂N₄O₄S, 571.1 [M+H], found 571.0.

### (Z)-3-chloro-N-((2-(1-(2,4-dichlorobenzyl)-1,4,5,6-tetrahydro-7H-indazol-7-ylidene)-2-fluoroethyl)carbamoyl)benzenesulfonamide, Compound #91

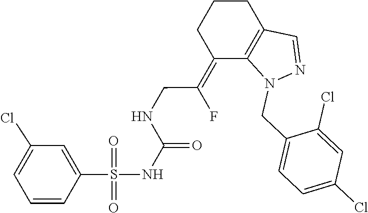 [SMILES: O=C(NC/C(F)=C1\CCCc2cnn(Cc3ccc(Cl)cc3Cl)c21)NS(=O)(=O)c1cccc(Cl)c1]

¹H NMR (300 MHz, DMSO, ppm) δ: 10.96 (s, 1H), 7.90 (s, 1H), 7.87 (d, J=11.4 Hz, 1H), 7.75 (d, J=6.0 Hz, 1H), 7.57-7.64 (m, 2H), 7.39 (s, 1H), 7.35 (d, J=8.4 Hz, 1H), 7.13 (t, J=5.1 Hz, 1H), 6.78 (d, J=8.4 Hz, 1H), 5.23 (s, 2H), 3.96 (d, J=21.9 Hz, 2H), 2.50-2.56 (m, 2H), 2.29 (t, J=6.3 Hz, 2H), 1.72 (s, 2H). ¹⁹F NMR (300 MHz, DMSO) δ: −106.38. Mass spectrum (ESI, m/z): Calculated for C₂₃H₂₁Cl₃FN₄O₃S, 557.0 [M+H], found 558.9.

### (Z)—N-((2-(1-(2,4-dichlorobenzyl)-1,4,5,6-tetrahydro-7H-indazol-7-ylidene)-2-fluoroethyl)carbamoyl)benzofuran-2-sulfonamide, Compound #111

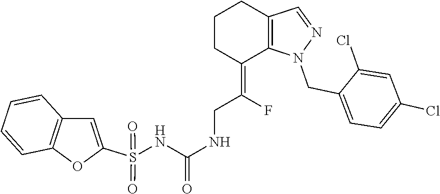 [SMILES: O=C(NC/C(F)=C1\CCCc2cnn(Cc3ccc(Cl)cc3Cl)c21)NS(=O)(=O)c1cc2ccccc2o1]

¹H NMR (400 MHz, CD₃OD) δ: 7.71 (d, J=8.0 Hz, 1H), 7.45-7.53 (m, 3H), 7.34-7.37 (m, 3H), 7.20-7.22 (m, 1H), 6.69 (d, J=8.4 Hz, 1H), 5.29 (s, 2H), 4.08 (d, J=21.6 Hz, 2H), 2.51-2.54 (m, 2H), 2.30-2.37 (m, 2H), 1.70-1.77 (m, 2H). ¹⁹F NMR (400 MHz, CD₃OD) δ: −106.75. Mass spectrum (ESI, m/z): Calculated for C₂₅H₂₂Cl₂FN₄O₄S, 563.1 [M+H], found 562.9.

### (Z)—N-((2-(1-(2,4-dichlorobenzyl)-1,4,5,6-tetrahydro-7H-indazol-7-ylidene)-2-fluoroethyl)carbamoyl)benzo[b]thiophene-2-sulfonamide, Compound #121

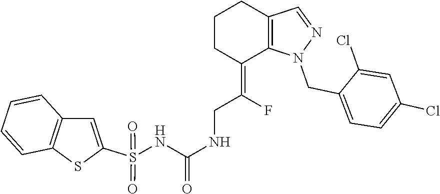 [SMILES: O=C(NC/C(F)=C1\CCCc2cnn(Cc3ccc(Cl)cc3Cl)c21)NS(=O)(=O)c1cc2ccccc2s1]

¹H NMR (400 MHz, CD₃OD) δ: 7.79-7.82 (m, 3H), 7.37-7.42 (m, 4H), 7.20 (d, J=8.0 Hz, 1H), 6.68 (d, J=8.4 Hz, 1H), 5.31 (s, 2H), 4.03 (d, J=22 Hz, 2H), 2.51-2.56 (m, 2H), 2.35-2.42 (m, 2H), 1.85-1.95 (m, 2H). ¹⁹F NMR (400 MHz, CD₃OD) δ: −105.22. Mass spectrum (ESI, m/z): Calculated for C₂₅H₂₂Cl₂FN₄O₃S₂, 579.0 [M+H], found 578.9.

### (Z)—N-((2-(1-(2,4-dichlorobenzyl)-1,4,5,6-tetrahydro-7H-indazol-7-ylidene)-2-fluoroethyl)carbamoyl)-4,5-dimethylthiophene-2-sulfonamide, Compound #142

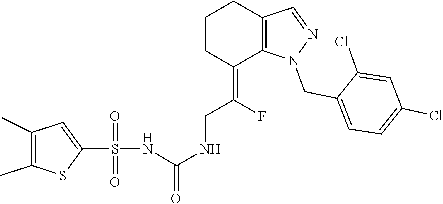 [SMILES: Cc1cc(S(=O)(=O)NC(=O)NC/C(F)=C2\CCCc3cnn(Cc4ccc(Cl)cc4Cl)c32)sc1C]

¹H NMR (300 MHz, CD₃OD) δ: 7.43-7.44 (m, 3H), 7.22-7.25 (m, 1H), 6.73 (d, J=8.4 Hz, 1H), 5.37 (s, 2H), 4.13 (s, 1H), 4.06 (s, 1H), 2.63-2.67 (m, 2H), 2.40-2.44 (m, 2H), 2.33 (s, 3H), 2.09 (s, 3H), 1.85-1.88 (m, 2H). ¹⁹F NMR (300 MHz, CD₃OD) δ: −106.48. Mass spectrum (ESI, m/z): Calculated for C₂₃H₂₄Cl₂FN₄O₃S₂, 557.1 [M+H], found 557.0.

### (Z)-3-chloro-N-((2-(1-(2,4-dichlorobenzyl)-1,4,5,6-tetrahydro-7H-indazol-7-ylidene)-2-fluoroethyl)carbamoyl)-4-methoxybenzenesulfonamide, Compound #143

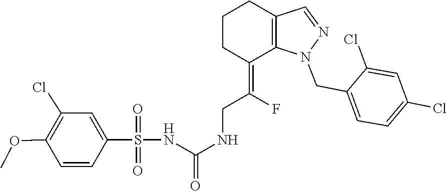 [SMILES: COc1ccc(S(=O)(=O)NC(=O)NC/C(F)=C2\CCCc3cnn(Cc4ccc(Cl)cc4Cl)c32)cc1Cl]

¹H NMR (300 MHz, CD₃OD) δ: 7.96 (s, 1H), 7.85-7.89 (m, 1H), 7.25-7.44 (m, 2H), 7.21-7.24 (m, 1H), 7.15-7.18 (m, 1H), 6.73 (d, J=8.4 Hz, 1H), 5.34 (s, 2H), 4.09 (s, 1H), 4.02 (s, 1H), 3.94 (s, 3H), 2.61-2.65 (m, 2H), 2.37-2.41 (m, 2H), 1.75-1.80 (m, 2H). ¹⁹F NMR (300 MHz, CD₃OD) δ: −106.53. Mass spectrum (ESI, m/z): Calculated for C₂₄H₂₃Cl₃FN₄O₄S, 587.0 [M+H], found 589.0.

### (Z)-5-chloro-N-((2-(1-(2,4-dichlorobenzyl)-1,4,5,6-tetrahydro-7H-indazol-7-ylidene)-2-fluoroethyl)carbamoyl)-4-methylthiophene-2-sulfonamide, Compound #149

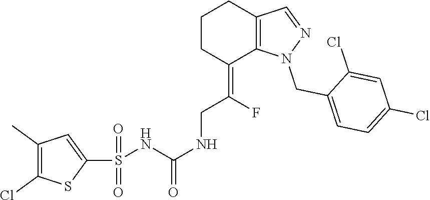 [SMILES: Cc1cc(S(=O)(=O)NC(=O)NC/C(F)=C2\CCCc3cnn(Cc4ccc(Cl)cc4Cl)c32)sc1Cl]

¹H NMR (400 MHz, DMSO) δ: 11.14 (s, 1H), 7.57-7.58 (m, 2H), 7.40 (s, 1H), 7.34-7.36 (m, 1H), 7.07 (s, 1H), 6.80 (d, J=8.4 Hz, 1H), 5.25 (s, 2H), 3.96-4.03 (m, 2H), 2.55-2.58 (m, 2H), 2.30-2.35 (m, 2H), 2.14 (s, 3H), 1.72-1.80 (m, 2H). ¹⁹F NMR (400 MHz, DMSO) δ: −106.35. Mass spectrum (ESI, m/z): Calculated for C₂₂H₂₁Cl₃FN₄O₃S₂, 577.0 [M+H], found 578.9.

### (Z)-5-chloro-N-((2-fluoro-2-(1-(4-(trifluoromethyl)benzyl)-1,4,5,6-tetrahydro-7H-indazol-7-ylidene)ethyl)carbamoyl)-2-methoxybenzenesulfonamide, Compound #131

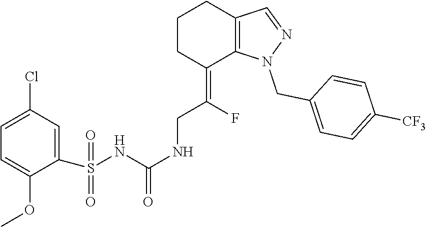 [SMILES: COc1ccc(Cl)cc1S(=O)(=O)NC(=O)NC/C(F)=C1\CCCc2cnn(Cc3ccc(C(F)(F)F)cc3)c21]

¹H NMR (400 MHz, CD₃OD) δ: 7.86 (d, J=2.8 Hz, 1H), 7.58 (d, J=8.0 Hz, 2H), 7.50-7.53 (m, 1H), 7.47 (s, 1H), 7.17 (d, J=8.0 Hz, 2H), 7.08 (d, J=8.8 Hz, 1H), 5.39 (s, 2H), 4.04 (d, J=21.2 Hz, 2H), 3.84 (s, 3H), 2.59-2.62 (m, 2H), 2.30-2.33 (m, 2H), 1.72-1.74 (m, 2H). ¹⁹F NMR (400 MHz, CD₃OD) δ: −63.98, −106.25. Mass spectrum (ESI, m/z): Calculated for C₂₅H₂₄ClF₄N₄O₄S: 587.1 [M+H], found: 587.2.

### (Z)—N-((2-fluoro-2-(1-(4-(trifluoromethyl)benzyl)-1,4,5,6-tetrahydro-7H-indazol-7-ylidene)ethyl)carbamoyl)benzofuran-2-sulfonamide, Compound #132

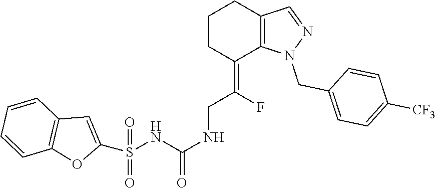 [SMILES: O=C(NC/C(F)=C1\CCCc2cnn(Cc3ccc(C(F)(F)F)cc3)c21)NS(=O)(=O)c1cc2ccccc2o1]

¹H NMR (400 MHz, CD₃OD) δ: 7.69-7.71 (m, 1H), 7.59 (s, 1H), 7.52-7.54 (m, 2H), 7.44-7.50 (m, 2H), 7.33-7.37 (m, 2H), 7.10 (d, J=7.6 Hz, 2H), 5.29 (s, 2H), 4.07 (d, J=22.4 Hz, 2H), 2.47-2.50 (m, 2H), 2.27-2.30 (m, 2H), 1.68-1.70 (m, 2H). ¹⁹F NMR (400 MHz, CD₃OD) δ: −63.98, −106.29. Mass spectrum (ESI, m/z): Calculated for C₂₆H₂₃F₄N₄O₄S: 563.1 [M+H], found: 563.2.

### (Z)—N-((2-fluoro-2-(1-(4-(trifluoromethyl)benzyl)-1,4,5,6-tetrahydro-7H-indazol-7-ylidene)ethyl)carbamoyl)benzo[b]thiophene-2-sulfonamide, Compound #133

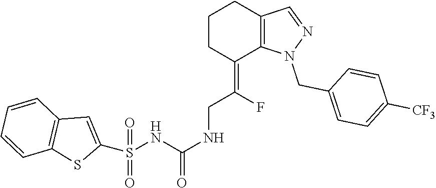 [SMILES: O=C(NC/C(F)=C1\CCCc2cnn(Cc3ccc(C(F)(F)F)cc3)c21)NS(=O)(=O)c1cc2ccccc2s1]

¹H NMR (400 MHz, CD₃OD) δ: 8.06 (s, 1H), 7.86-7.88 (m, 2H), 7.46-7.52 (m, 4H), 7.37 (s, 1H), 7.08 (d, J=8.0 Hz, 2H), 5.28 (s, 2H), 4.07 (d, J=21.2 Hz, 2H), 2.46-2.49 (m, 2H), 2.28-2.31 (m, 2H), 1.70-1.72 (m, 2H). ¹⁹F NMR (400 MHz, CD₃OD) δ: −63.99, −105.96. Mass spectrum (ESI, m/z): Calculated for C₂₆H₂₃F₄N₄O₃S₂: 579.1 [M+H], found: 579.2.

### (Z)-4,5-dichloro-N-((2-fluoro-2-(1-(4-(trifluoromethyl)benzyl)-1,4,5,6-tetrahydro-7H-indazol-7-ylidene)ethyl)carbamoyl)thiophene-2-sulfonamide, Compound #134

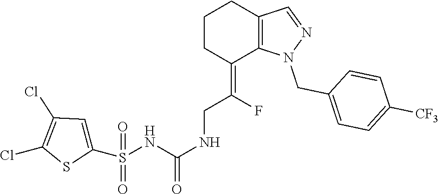 [SMILES: O=C(NC/C(F)=C1\CCCc2cnn(Cc3ccc(C(F)(F)F)cc3)c21)NS(=O)(=O)c1cc(Cl)c(Cl)s1]

¹H NMR (400 MHz, CD₃OD) δ: 7.66 (s, 1H), 7.57 (d, J=8.0 Hz, 2H), 7.46 (s, 1H), 7.18 (d, J=8.0 Hz, 2H), 5.43 (s, 2H), 4.08 (d, J=21.2 Hz, 2H), 2.63-2.67 (m, 2H), 2.38-2.41 (m, 2H), 1.86-1.88 (m, 2H). ¹⁹F NMR (400 MHz, CD₃OD) δ: −64.00, −105.75. Mass spectrum (ESI, m/z): Calculated for C₂₂H₁₉Cl₂F₄N₄O₃S₂: 597.0 [M+H], found: 597.1.

### (Z)-4,5-dichloro-N-((2-fluoro-2-(1-((6-methoxypyridin-3-yl)methyl)-1,4,5,6-tetrahydro-7H-indazol-7-ylidene)ethyl)carbamoyl)thiophene-2-sulfonamide, Compound #141

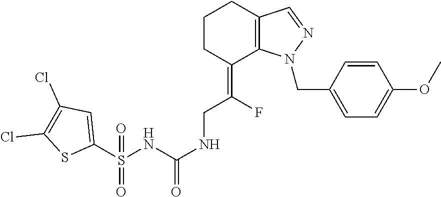 [SMILES: COc1ccc(Cn2ncc3c2/C(=C(\F)CNC(=O)NS(=O)(=O)c2cc(Cl)c(Cl)s2)CCC3)cc1]

¹H NMR (300 MHz, CD₃OD) δ: 7.86 (s, 1H), 7.46 (s, 1H), 7.33-7.39 (m, 2H), 6.70 (d, J=8.7 Hz, 1H), 5.27 (s, 2H), 4.12 (s, 1H), 4.05 (s, 1H), 3.87 (s, 3H), 2.61 (d, J=6.6 Hz, 2H), 2.37 (s, 2H), 1.81-1.92 (m, 2H). ¹⁹F NMR (300 MHz, CD₃OD) δ: −105.63. Mass spectrum (ESI, m/z): Calculated for C₂₁H₂₁Cl₂FN₅O₂S₂: 560.0 [M+H], found: 560.0.

The following representative compound of formula (I) was similarly prepared according to the procedure described in Example 11 (and Example 5 as referenced therein) above, selecting and substituting suitable starting materials and reagents (including substituting the compound prepared in Example 8, Step 7 for the compound prepared in Example 8, Step 5, as would be readily recognized by those skilled in the art.

### (E)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide, Compound #45

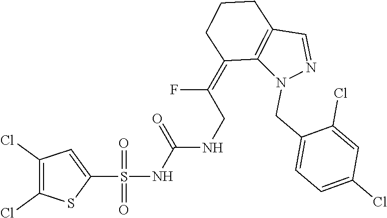 [SMILES: O=C(NC/C(F)=C1/CCCc2cnn(Cc3ccc(Cl)cc3Cl)c21)NS(=O)(=O)c1cc(Cl)c(Cl)s1]

¹H NMR (300 MHz, CD₃OD) δ: 7.61 (s, 1H), 7.36-7.44 (m, 2H), 7.20-7.24 (m, 1H), 6.80 (d, J=8.4 Hz, 1H), 5.20 (s, 2H), 3.99 (d, J=19.2 Hz, 2H), 2.60-2.64 (m, 2H), 2.39-2.45 (m, 2H), 1.84-1.91 (m, 2H). ¹⁹F NMR (300 MHz, CD₃OD) δ: −77.29, −114.11. Mass spectrum (ESI, m/z): Calculated for C₂₁H₁₈Cl₄FN₄O₃S₂, 596.9 (M+H), found 598.9.

### Example 12: Compound #156

### (Z)—N-((2-(1-(2,4-dichlorobenzyl)-1,4,5,6-tetrahydro-7H-indazol-7-ylidene)-2-fluoroethyl)carbamoyl)-3,4-difluorobenzenesulfonamide

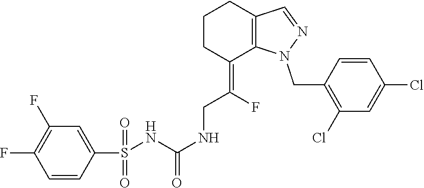 [SMILES: O=C(NC/C(F)=C1\CCCc2cnn(Cc3ccc(Cl)cc3Cl)c21)NS(=O)(=O)c1ccc(F)c(F)c1]

### Step 1. Synthesis of phenyl (Z)-(2-(1-(2,4-dichlorobenzyl)-1,4,5,6-tetrahydro-7H-indazol-7-ylidene)-2-fluoroethyl)carbamate

Phenyl (Z)-(2-(1-(2,4-dichlorobenzyl)-1,4,5,6-tetrahydro-7H-indazol-7-ylidene)-2-fluoroethyl)carbamate was prepared from (Z)-2-(1-(2,4-dichlorobenzyl)-1,4,5,6-tetrahydro-7H-indazol-7-ylidene)-2-fluoroethan-1-amine according to the procedures as described in Example 7, Step 1.

### Step 2. Synthesis of (Z)—N-((2-(1-(2,4-dichlorobenzyl)-1,4,5,6-tetrahydro-7H-indazol-7-ylidene)-2-fluoroethyl)carbamoyl)-3,4-difluorobenzenesulfonamide

(Z)—N-((2-(1-(2,4-Dichlorobenzyl)-1,4,5,6-tetrahydro-7H-indazol-7-ylidene)-2-fluoroethyl)carbamoyl)-3,4-difluorobenzenesulfonamide was prepared according to the procedures as described in Example 7, Step 2.

The following representative compound of formula (I) was similarly prepared according to the procedure described in Example 12 (and Examples referenced therein) above, selecting and substituting suitable starting materials and reagents, as would be readily recognized by those skilled in the art.

### (Z)—N-((2-(1-(2,4-Dichlorobenzyl)-1,4,5,6-tetrahydro-7H-indazol-7-ylidene)-2-fluoroethyl)carbamoyl)-2,4-dimethylthiazole-5-sulfonamide, Compound #158

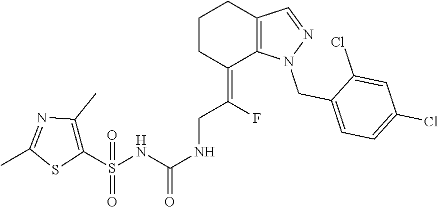 [SMILES: Cc1nc(C)c(S(=O)(=O)NC(=O)NC/C(F)=C2\CCCc3cnn(Cc4ccc(Cl)cc4Cl)c32)s1]

¹H NMR (400 MHz, DMSO) δ: 11.18 (s, 1H), 7.59 (s, 1H), 7.40 (s, 1H), 7.34-7.39 (m, 1H), 6.78 (d, J=8.4 Hz, 1H), 6.58-6.64 (m, 1H), 5.26 (s, 2H), 3.88-3.95 (m, 2H), 2.56-2.58 (m, 6H), 2.41-2.48 (m, 2H), 2.37-2.40 (m, 2H), 1.71-1.79 (m, 2H). ¹⁹F NMR (400 MHz, DMSO) δ: −105.33. Mass spectrum (ESI, m/z): Calculated for C₂₂H₂₃Cl₂FN₅O₃S₂, 558.1 [M+H], found 557.9.

(Z)—N-((2-(1-(2,4-Dichlorobenzyl)-1,4,5,6-tetrahydro-7H-indazol-7-ylidene)-2-fluoroethyl)carbamoyl)-3-fluoro-4-methoxybenzenesulfonamide, Compound #159

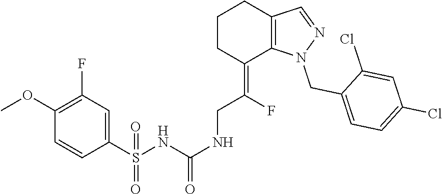 [SMILES: COc1ccc(S(=O)(=O)NC(=O)NC/C(F)=C2\CCCc3cnn(Cc4ccc(Cl)cc4Cl)c32)cc1F]

¹H NMR (400 MHz, DMSO) δ: 7.57-7.61 (m, 3H), 7.33-7.38 (m, 2H), 7.21-7.25 (m, 1H), 6.77 (d, J=8.8 Hz, 1H), 6.60-6.67 (m, 1H), 5.24 (s, 2H), 3.85-3.92 (m, 5H), 2.53-2.54 (m, 2H), 2.23-2.30 (m, 2H), 1.71-1.78 (m, 2H). ¹⁹F NMR (400 MHz, DMSO) δ: −105.33, −134.42. Mass spectrum (ESI, m/z): Calculated for C₂₄H₂₃Cl₂F₂N₄O₄S, 571.1 [M+H], found 570.9.

### (Z)—N-((2-(1-(2,4-dichlorobenzyl)-1,4,5,6-tetrahydro-7H-indazol-7-ylidene)-2-fluoroethyl)carbamoyl)-1-methyl-1H-pyrazole-5-sulfonamide, Compound #168

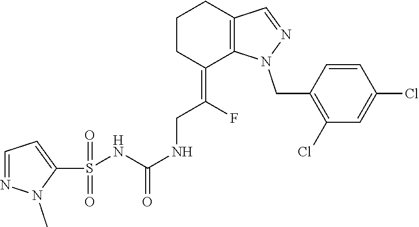 [SMILES: Cn1nccc1S(=O)(=O)NC(=O)NC/C(F)=C1\CCCc2cnn(Cc3ccc(Cl)cc3Cl)c21]

¹H NMR (300 MHz, CD₃OD) δ: 7.62 (s, 1H), 7.46 (d, J=7.2 Hz, 2H), 7.28 (d, J=7.2 Hz, 1H), 6.81-6.86 (m, 2H), 5.41 (s, 2H), 4.05-4.12 (m, 5H), 2.68 (t, J=6.3 Hz, 2H), 2.45 (s, 2H), 1.82-1.91 (m, 2H). ¹⁹F NMR (300 MHz, DMSO) δ: −106.54. Mass spectrum (ESI, m/z): Calculated for C₂₁H₂₂Cl₂FN₆O₃S, 527.1 [M+H], found: 527.1.

### (Z)—N-((2-(1-(2,4-dichlorobenzyl)-1,4,5,6-tetrahydro-7H-indazol-7-ylidene)-2-fluoroethyl)carbamoyl)-1,5-dimethyl-1H-pyrazole-4-sulfonamide, Compound #169

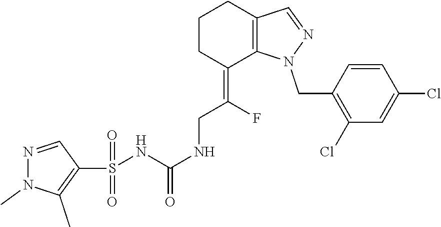 [SMILES: Cc1c(S(=O)(=O)NC(=O)NC/C(F)=C2\CCCc3cnn(Cc4ccc(Cl)cc4Cl)c32)cnn1C]

¹H NMR (300 MHz, CD₃OD) δ: 7.74 (d, J=6.9 Hz, 2H), 7.47 (d, J=2.1 Hz, 1H), 7.29 (d, J=8.4 Hz, 1H), 6.90 (d, J=8.4 Hz, 1H), 5.44 (s, 2H), 4.06 (d, J=21.0 Hz, 2H), 3.73 (s, 3H), 2.67 (t, J=6.6 Hz, 2H), 2.42-2.46 (m, 5H), 1.87-1.89 (m, 2H). ¹⁹F NMR (300 MHz, CD₃OD) δ: −102.97. Mass spectrum (ESI, m/z): Calculated for C₂₂H₂₄Cl₂FN₆O₃S, 541.1 [M+H], found: 541.1.

### (Z)—N-((2-(1-(2,4-dichlorobenzyl)-1,4,5,6-tetrahydro-7H-indazol-7-ylidene)-2-fluoroethyl)carbamoyl)-1-isopropyl-1H-pyrazole-4-sulfonamide, Compound #170

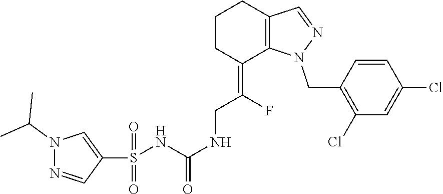 [SMILES: CC(C)n1cc(S(=O)(=O)NC(=O)NC/C(F)=C2\CCCc3cnn(Cc4ccc(Cl)cc4Cl)c32)cn1]

¹H NMR (300 MHz, CD₃OD) δ: 8.20 (s, 1H), 7.81 (s, 1H), 7.31 (s, 1H), 7.46 (d, J=2.1 Hz, 1H), 7.28 (d, J=8.4 Hz, 1H), 6.88 (d, J=8.4 Hz, 1H), 5.44 (s, 2H), 4.46-4.54 (m, 1H), 4.08 (d, J=21.0 Hz, 2H), 2.67 (t, J=6.3 Hz, 2H), 2.45 (t, J=4.5 Hz, 2H), 1.90 (brs, 2H), 1.43 (d, J=6.6 Hz, 6H). ¹⁹F NMR (300 MHz, CD₃OD) δ: −103.15. Mass spectrum (ESI, m/z): Calculated for C₂₃H₂₆Cl₂FN₆O₃S, 555.1 [M+H], found: 555.1.

### (Z)—N-((2-(1-(2,4-dichlorobenzyl)-1,4,5,6-tetrahydro-7H-indazol-7-ylidene)-2-fluoroethyl)carbamoyl)-3,4-dimethoxybenzenesulfonamide, Compound #174

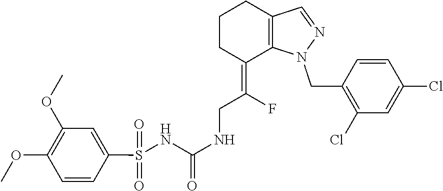 [SMILES: COc1ccc(S(=O)(=O)NC(=O)NC/C(F)=C2\CCCc3cnn(Cc4ccc(Cl)cc4Cl)c32)cc1OC]

¹H NMR (300 MHz, CD₃OD) δ: 7.48-7.55 (m, 2H), 7.41 (d, J=8.7 Hz, 2H), 7.23 (d, J=8.4 Hz, 1H), 6.97 (d, J=8.7 Hz, 1H), 6.77 (d, J=8.4 Hz, 1H), 5.32 (s, 2H), 4.03 (d, J=21.3 Hz, 2H), 3.82 (d, J=12.0 Hz, 6H), 2.60 (t, J=6.3 Hz, 2H), 2.37 (t, J=5.7 Hz, 2H), 1.78 (brs, 2H). ¹⁹F NMR (300 MHz, CD₃OD) δ: −104.95. Mass spectrum (ESI, m/z): Calculated for C₂₅H₂₆Cl₂FN₄O₅S: 583.1 [M+H], found: 583.2.

### (Z)-4-Chloro-N-((2-(1-(2,4-dichlorobenzyl)-1,4,5,6-tetrahydro-7H-indazol-7-ylidene)-2-fluoroethyl)carbamoyl)-3-methoxybenzenesulfonamide, Compound #175

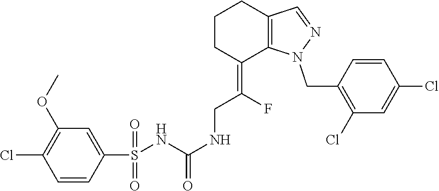 [SMILES: COc1cc(S(=O)(=O)NC(=O)NC/C(F)=C2\CCCc3cnn(Cc4ccc(Cl)cc4Cl)c32)ccc1Cl]

¹H NMR (300 MHz, CD₃OD) δ: 7.39-7.58 (m, 5H), 7.21 (d, J=8.4 Hz, 1H), 6.72 (d, J=8.4 Hz, 1H), 5.30 (s, 2H), 4.03 (d, J=21.6 Hz, 2H), 3.88 (s, 3H), 2.60 (t, J=6.3 Hz, 2H), 2.35 (t, J=5.7 Hz, 2H), 1.73-1.76 (m, 2H). ¹⁹F NMR (300 MHz, CD₃OD) δ: −106.36. Mass spectrum (ESI, m/z): Calculated for C₂₄H₂₃Cl₃FN₄O₄S: 587.0 [M+H], found: 589.0.

### (Z)—N-((2-(1-(2,4-dichlorobenzyl)-1,4,5,6-tetrahydro-7H-indazol-7-ylidene)-2-fluoroethyl)carbamoyl)-2-methoxybenzo[d]oxazole-6-sulfonamide, Compound #178

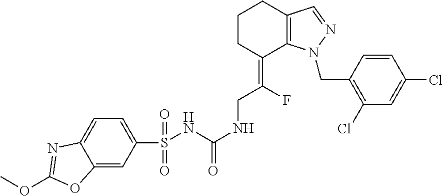 [SMILES: COc1nc2ccc(S(=O)(=O)NC(=O)NC/C(F)=C3\CCCc4cnn(Cc5ccc(Cl)cc5Cl)c43)cc2o1]

¹H NMR (400 MHz, CHLOROFORM-d) δ: 7.85 (dd, J=8.1, 1.5 Hz, 1H), 7.78 (d, J=1.5 Hz, 1H), 7.39 (s, 1H), 7.30-7.35 (m, 1H), 7.14 (dd, J=8.6, 2.0 Hz, 1H), 7.01 (d, J=8.6 Hz, 1H), 6.68 (d, J=8.6 Hz, 1H), 5.30-5.36 (m, 2H), 4.08 (s, 1H), 4.03 (s, 1H), 3.43 (s, 3H), 2.48-2.70 (m, 2H), 2.25-2.48 (m, 2H), 1.80-1.89 (m, 2H). Mass spectrum (ESI, m/z): Calculated for C₂₅H₂₃Cl₂FN₅O₅S: 594.1 [M+H], found: 594.1.

### (Z)—N-((2-(1-(2,4-dichlorobenzyl)-1,4,5,6-tetrahydro-7H-indazol-7-ylidene)-2-fluoroethyl)carbamoyl)-1,3-dimethyl-1H-pyrazole-4-sulfonamide, Compound #180

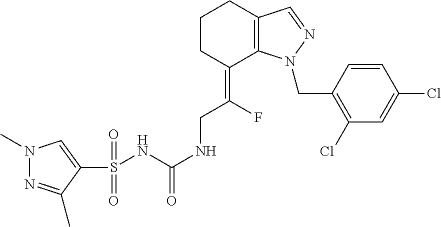 [SMILES: Cc1nn(C)cc1S(=O)(=O)NC(=O)NC/C(F)=C1\CCCc2cnn(Cc3ccc(Cl)cc3Cl)c21]

¹H NMR (300 MHz, CD₃OD) δ: 8.09 (s, 1H), 7.78-7.84 (m, 1H), 7.52 (s, 1H), 7.32-7.35 (m, 1H), 6.90-6.97 (m, 1H), 5.49 (s, 2H), 4.10 (d, J=21.0 Hz, 2H), 3.80 (s, 3H), 2.72 (t, J=6.3 Hz, 2H), 2.48 (brs, 2H), 2.35 (s, 3H), 1.92 (brs, 2H). ¹⁹F NMR (300 MHz, CD₃OD) δ: −102.46. Mass spectrum (ESI, m/z): Calculated for C₂₂H₂₄Cl₂FN₆O₃S: 541.1 [M+H], found: 541.1.

### (Z)—N-(5-(N-((2-(1-(2,4-dichlorobenzyl)-1,4,5,6-tetrahydro-7H-indazol-7-ylidene)-2-fluoroethyl)carbamoyl)sulfamoyl)-4-methylthiazol-2-yl)acetamide, Compound #182

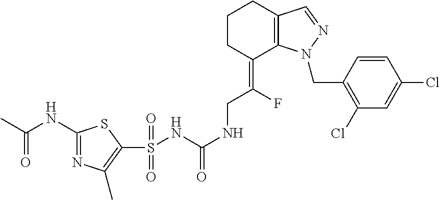 [SMILES: CC(=O)Nc1nc(C)c(S(=O)(=O)NC(=O)NC/C(F)=C2\CCCc3cnn(Cc4ccc(Cl)cc4Cl)c32)s1]

¹H NMR (400 MHz, CHLOROFORM-d) δ: 7.30-7.41 (m, 2H), 7.14 (d, J=8.1 Hz, 1H), 6.69 (d, J=8.6 Hz, 1H), 5.32 (s, 2H), 4.06-4.17 (m, 2H), 2.62 (t, J=6.3 Hz, 2H), 2.51 (s, 3H), 2.37 (m, 2H), 2.22 (s, 3H), 1.88 (m, 2H). Mass spectrum (ESI, m/z): Calculated for C₂₃H₂₄Cl₂FN₆O₄S₂: 601.1 [M+H], found: 601.0.

### (Z)—N-((2-(1-(2,4-dichlorobenzyl)-1,4,5,6-tetrahydro-7H-indazol-7-ylidene)-2-fluoroethyl)carbamoyl)-2-methylbenzo[d]oxazole-5-sulfonamide, Compound #183

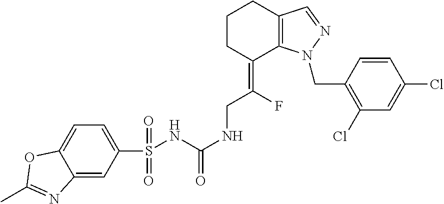 [SMILES: Cc1nc2cc(S(=O)(=O)NC(=O)NC/C(F)=C3\CCCc4cnn(Cc5ccc(Cl)cc5Cl)c43)ccc2o1]

¹H NMR (400 MJHz, CHLOROFORM-d) δ: 8.16 (d, J=2.0 Hz, 1H), 7.80 (dd, J=8.6, 2.0 Hz, 1H), 7.45 (m, 1H), 7.32 (d, J=2.0 Hz, 1H), 7.14 (dd, J=8.3, 2.3 Hz, 1H), 6.95 (t, J=5.8 Hz, 1H), 6.74 (d, J=8.6 Hz, 1H), 5.29-5.37 (m, 2H), 4.08-4.17 (m, 2H), 2.68 (s, 3H), 2.57 (t, J=6.3 Hz, 2H), 2.35 (m, 2H), 1.74-1.84 (m, 2H). Mass spectrum (ESI, m/z): Calculated for C₂₅H₂₃Cl₂FN₅O₄S: 578.1 [M+H], found: 578.1.

### (Z)—N-((2-(1-(2,4-dichlorobenzyl)-1,4,5,6-tetrahydro-7H-indazol-7-ylidene)-2-fluoroethyl)carbamoyl)-1,3-dimethyl-1H-pyrazole-5-sulfonamide, Compound #193

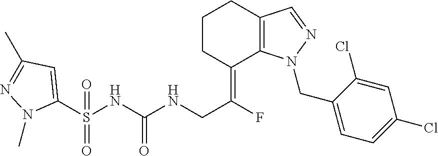 [SMILES: Cc1cc(S(=O)(=O)NC(=O)NC/C(F)=C2\CCCc3cnn(Cc4ccc(Cl)cc4Cl)c32)n(C)n1]

¹H NMR (400 MHz, CD₃OD) δ: 7.72 (s, 1H), 7.49 (s, 1H), 7.31 (d, J=8.0 Hz, 1H), 7.88 (d, J=8.4 Hz, 1H), 6.64 (s, 1H), 5.44 (s, 2H), 4.08 (d, J=21.2 Hz, 2H), 4.00 (s, 3H), 2.69 (t, J=6.4 Hz, 2H), 2.46 (t, J=5.2 Hz, 2H), 2.22 (s, 3H), 1.87 (brs, 2H). ¹⁹F NMR (400 MHz, CD₃OD) δ: −103.68. Mass spectrum (ESI, m/z): Calculated for C₂₂H₂₄Cl₂FN₆O₃S: 541.1 [M+H], found: 541.1.

### (Z)—N-((2-(1-(2,4-dichlorobenzyl)-1,4,5,6-tetrahydro-7H-indazol-7-ylidene)-2-fluoroethyl)carbamoyl)-1-methyl-1H-indazole-6-sulfonamide, Compound #194

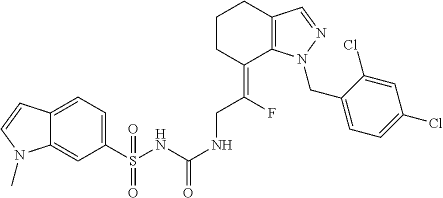 [SMILES: Cn1ccc2ccc(S(=O)(=O)NC(=O)NC/C(F)=C3\CCCc4cnn(Cc5ccc(Cl)cc5Cl)c43)cc21]

¹H NMR (400 MHz, CD₃OD) δ: 8.26 (s, 1H), 8.09 (s, 1H), 7.86 (d, J=8.4 Hz, 1H), 7.69 (s, 1H), 7.64 (d, J=8.4 Hz, 1H), 7.41 (s, 1H), 7.27 (d, J=8.4 Hz, 1H), 6.84 (d, J=8.4 Hz, 1H), 5.32 (s, 2H), 4.13 (s, 3H), 4.05 (d, J=21.2 Hz, 2H), 2.54 (t, J=6.4 Hz, 2H), 2.36 (t, J=4.8 Hz, 2H), 1.68-1.70 (m, 2H). ¹⁹F NMR (400 MHz, CD₃OD) δ: −103.32. Mass spectrum (ESI, m/z): Calculated for C₂₅H₂₄Cl₂FN₆O₃S: 577.1 [M+H], found: 577.1.

### (Z)—N-((2-(1-(2,4-dichlorobenzyl)-1,4,5,6-tetrahydro-7H-indazol-7-ylidene)-2-fluoroethyl)carbamoyl)-1-methyl-1H-indazole-5-sulfonamide, Compound #195

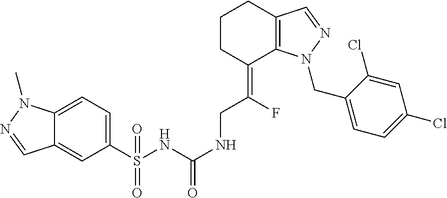 [SMILES: Cn1ncc2cc(S(=O)(=O)NC(=O)NC/C(F)=C3\CCCc4cnn(Cc5ccc(Cl)cc5Cl)c43)ccc21]

¹H NMR (400 MHz, DMSO) δ: 10.73 (s, 1H), 8.39 (s, 1H), 8.28 (s, 1H), 7.78-7.84 (m, 2H), 7.52 (s, 1H), 7.29-7.37 (m, 2H), 6.97-6.98 (m, 1H), 6.76 (d, J=8.4 Hz, 1H), 5.18 (s, 2H), 4.08 (s, 3H), 3.92 (d, J=22.0 Hz, 2H), 2.45-2.46 (m, 2H), 2.25 (brs, 2H), 1.63 (brs, 2H). ¹⁹F NMR (400 MHz, DMSO) δ: −106.27. Mass spectrum (ESI, m/z): Calculated for C₂₅H₂₄Cl₂FN₆O₃S: 577.1 [M+H], found: 577.1.

### (Z)-5-chloro-N-((2-(1-(2,4-dichlorobenzyl)-1,4,5,6-tetrahydro-7H-indazol-7-ylidene)-2-fluoroethyl)carbamoyl)-6-methylpyridine-3-sulfonamide, Compound #197

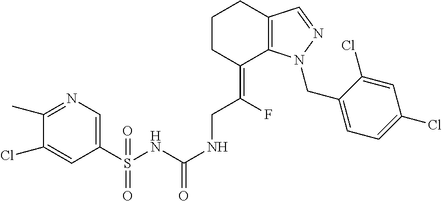 [SMILES: Cc1ncc(S(=O)(=O)NC(=O)NC/C(F)=C2\CCCc3cnn(Cc4ccc(Cl)cc4Cl)c32)cc1Cl]

¹H NMR (300 MHz, CD₃OD) δ: 8.85 (s, 1H), 8.30 (s, 1H), 7.38-7.41 (m, 2H), 7.19-7.24 (m, 1H), 6.69 (d, J=8.7 Hz, 1H), 5.31 (s, 2H), 3.99-4.08 (m, 2H), 2.55-2.68 (m, 5H), 2.31-2.42 (m, 2H), 1.70-1.90 (m, 2H). ¹⁹F NMR (300 MHz, CD₃OD) δ: −103.16. Mass spectrum (ESI, m/z): Calculated for C₂₃H₂₂Cl₃FN₅O₃S: 572.0 [M+H], found: 574.0.

### (Z)-4,5-dichloro-N-((2-fluoro-2-(1-((1-phenyl-1H-pyrazol-4-yl)methyl)-1,4,5,6-tetrahydro-7H-indazol-7-ylidene)ethyl)carbamoyl)thiophene-2-sulfonamide, Compound #191

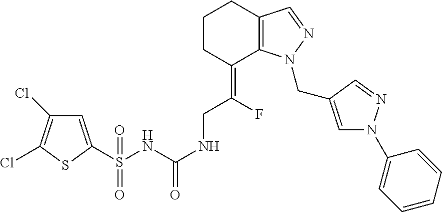 [SMILES: O=C(NC/C(F)=C1\CCCc2cnn(Cc3cnn(-c4ccccc4)c3)c21)NS(=O)(=O)c1cc(Cl)c(Cl)s1]

¹H NMR (400 MHz, Methanol-d4) δ: 8.12 (s, 1H), 7.70-7.68 (m, 2H), 7.64 (s, 1H), 7.55 (s, 1H), 7.52-7.49 (m, 1H), 7.47-7.45 (m, 2H), 7.34-7.10 (m, 1H), 5.05 (s, 2H), 4.22-4.17 (m, 2H), 2.65-2.62 (m, 2H), 2.46-2.45 (m, 2H), 1.96-1.88 (m, 2H). ¹⁹F NMR (400 MHz, Methanol-d4) δ: −105.46. Mass spectrum (ESI, m/z): Calculated for C₂₄H₂₂Cl₂FN₆O₃S₂: 595.0 [M+H], found: 595.3.

### (Z)-4,5-dichloro-N-((2-fluoro-2-(1-((2-phenylpyrimidin-5-yl)methyl)-1,4,5,6-tetrahydro-7H-indazol-7-ylidene)ethyl)carbamoyl)thiophene-2-sulfonamide, Compound #198

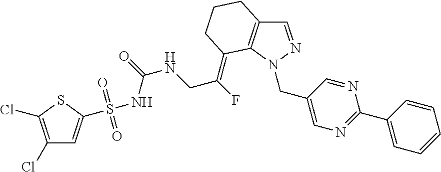 [SMILES: O=C(NC/C(F)=C1\CCCc2cnn(Cc3cnc(-c4ccccc4)nc3)c21)NS(=O)(=O)c1cc(Cl)c(Cl)s1]

¹H NMR (300 MHz, CD₃OD) δ: 8.58 (s, 2H), 8.35-8.40 (m, 2H), 7.68 (s, 1H), 7.45-7.55 (m, 4H), 5.41 (s, 2H), 4.21 (s, 1H), 4.13 (s, 1H), 2.62-2.70 (m, 2H), 2.40-2.50 (m, 2H), 1.85-1.95 (m, 2H). ¹⁹F NMR (300 MHz, CD₃OD) δ: −106.765. Mass spectrum (ESI, m/z): Calculated for C₂₅H₂₂Cl₂FN₆O₃S₂: 607.1 [M+H], found: 607.1.

### (Z)-3-chloro-N-((2-fluoro-2-(1-((2-phenylpyrimidin-5-yl)methyl)-1,4,5,6-tetrahydro-7H-indazol-7-ylidene)ethyl)carbamoyl)-4-methoxybenzenesulfonamide, Compound #199

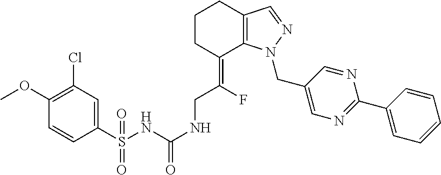 [SMILES: COc1ccc(S(=O)(=O)NC(=O)NC/C(F)=C2\CCCc3cnn(Cc4cnc(-c5ccccc5)nc4)c32)cc1Cl]

¹H NMR (300 MHz, CD₃OD) δ: 8.49 (s, 2H), 8.25-8.38 (m, 2H), 7.93 (m, 1H), 7.80-7.85 (m, 1H), 7.40-7.52 (m, 4H), 7.38 (s, 1H), 7.03-7.15 (m, 1H), 5.30 (s, 2H), 4.11 (s, 1H), 4.04 (s, 1H), 3.86 (s, 3H), 2.53-2.60 (m, 2H), 2.28-2.36 (m, 2H), 1.70-1.80 (m, 2H). ¹⁹F NMR (300 MHz, CD₃OD) δ: −77.151, −106.841. Mass spectrum (ESI, m/z): Calculated for C₂₈H₂₇ClFN₆O₄S: 597.1 [M+H], found: 597.4.

### Example 13: Compound #115 and Compound #106

### (Z)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide and

### (E)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide

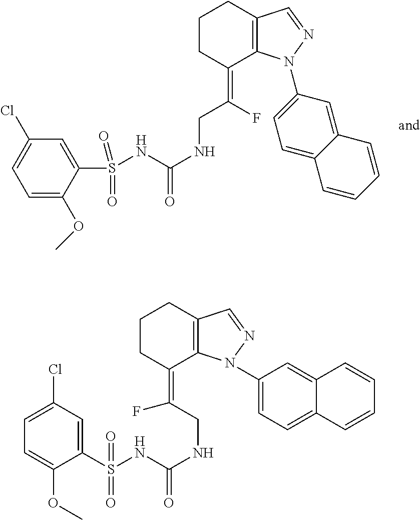 [SMILES: COc1ccc(Cl)cc1S(=O)(=O)NC(=O)NC/C(F)=C1\CCCc2cnn(-c3ccc4ccccc4c3)c21 ; COc1ccc(Cl)cc1S(=O)(=O)NC(=O)NC/C(F)=C1/CCCc2cnn(-c3ccc4ccccc4c3)c21]

### Step 1. Synthesis of ethyl 2-(2-ethoxycyclohex-2-en-1-ylidene)-2-fluoroacetate

To a solution of ethyl 2-(diethoxyphosphoryl)-2-fluoroacetate (17.1 g, 70.6 mmol) in THF (100 mL) at −78° C. under nitrogen was added n-BuLi (28.3 mL, 70.8 mmol). The reaction was stirred for 2 h at −78° C. before 2-ethoxycyclohex-2-en-1-one (3.30 g, 23.5 mmol) was added. The reaction was stirred for 30 min at −78° C. and was then warmed to room temperature and stirred overnight. The reaction was quenched with H₂O. The resulting mixture was extracted with EtOAc, and the organic layers were combined and concentrated under vacuum. The resulting residue was purified by silica gel column with PE:EA=70:30 to yield ethyl 2-(2-ethoxycyclohex-2-en-1-ylidene)-2-fluoroacetate. ¹H NMR (300 MHz, CDCl₃) δ: 5.17 (m, 1H), 4.14-4.21 (m, 2H), 3.61-3.68 (m, 2H), 2.36-2.41 (m, 2H), 2.09-2.15 (m, 2H), 1.57-1.65 (m, 2H), 1.13-1.31 (m, 6H).

### Step 2. Synthesis of ethyl 2-fluoro-2-(2-oxocyclohexylidene)acetate

A mixture of ethyl 2-(2-ethoxycyclohex-2-en-1-ylidene)-2-fluoroacetate (3.30 g, 14.5 mmol) in aq. 2N HCl (20 ml) and DCM (100 ml) was stirred at room temperature overnight. The reaction was diluted with H₂O and extracted with DCM. The organic layers were combined and concentrated under vacuum. The resulting residue was purified by silica gel column with PE:EA=70:30 to yield ethyl 2-fluoro-2-(2-oxocyclohexylidene)acetate as a yellow oil. Mass spectrum (ESI, m/z): Calculated for C₁₀H₁₄FO₃: 201.1 [M+H], found: 200.9.

### Step 3. Synthesis of ethyl 2-(3-((dimethylamino)methylene)-2-oxocyclohexylidene)-2-fluoroacetate

A solution of ethyl 2-fluoro-2-(2-oxocyclohexylidene)acetate (2.00 g, 9.99 mmol) and N,N-Dimethylformamide dimethyl acetal (4.00 g, 33.6 mmol) in DMF (50 ml) was stirred at 90° C. for 2 h. The reaction mixture was concentrated under vacuum to yield ethyl 2-(3-((dimethylamino)methylene)-2-oxocyclohexylidene)-2-fluoroacetate as a brown oil. Mass spectrum (ESI, m/z): Calculated for C₁₃H₁₉FNO₃: 256.1 [M+H], found: 255.9.

### Step 4. Synthesis of ethyl 2-fluoro-2-(1-(naphthalen-2-yl)-1,4,5,6-tetrahydro-7H-indazol-7-ylidene)acetate

A mixture of ethyl 2-(3-((dimethylamino)methylene)-2-oxocyclohexylidene)-2-fluoroacetate (2.00 g, 7.83 mmol), naphthalen-2-ylhydrazine hydrochloride (2.10 g, 10.8 mmol), and TFA (2 ml) in 1,4-dioxane (50 mL) was stirred at 60° C. for 2 h. The reaction was then quenched with H₂O. The resulting mixture was extracted with EtOAc and the organic layers were combined and concentrated under vacuum. The resulting residue was purified by preparative TLC with PE:EA=10:1 to yield ethyl 2-fluoro-2-(1-(naphthalen-2-yl)-1,4,5,6-tetrahydro-7H-indazol-7-ylidene)acetate as a mixture of the corresponding E and Z isomers. Mass spectrum (ESI, m/z): Calculated for C₂₁H₂₀FN₂O₂: 351.1 [M+H], found: 351.0.

### Step 5. Synthesis of (Z)-2-fluoro-2-(1-(naphthalen-2-yl)-1,4,5,6-tetrahydro-7H-indazol-7-ylidene)ethan-1-ol and (E)-2-fluoro-2-(1-(naphthalen-2-yl)-1,4,5,6-tetrahydro-7H-indazol-7-ylidene)ethan-1-ol

To a 250-mL 3-neck round bottle under N₂ was added ethyl 2-fluoro-2-(1-(naphthalen-2-yl)-1,4,5,6-tetrahydro-7H-indazol-7-ylidene)acetate (1.35 g, 3.85 mmol) and DCM (100 mL). The solution was cooled to −78° C. and DIBAL-H (19.2 mL, 19.2 mmol) was added in portions. The reaction was stirred for 30 min at −78° C. and then stirred for 3 h at 0° C. The reaction was quenched by the addition of MeOH (10 mL) and the resulting mixture was concentrated under vacuum. The resulting residue was purified by silica gel column with PE:EA=10:90 to yield (Z)-2-fluoro-2-(1-(naphthalen-2-yl)-1,4,5,6-tetrahydro-7H-indazol-7-ylidene)ethan-1-ol as a yellow solid and (E)-2-fluoro-2-(1-(naphthalen-2-yl)-1,4,5,6-tetrahydro-7H-indazol-7-ylidene)ethan-1-ol as a yellow solid.

(Z)-2-fluoro-2-(1-(naphthalen-2-yl)-1,4,5,6-tetrahydro-7H-indazol-7-ylidene)ethan-1-ol: Mass spectrum (ESI, m/z): Calculated for C₁₉H₁₈FN₂O, 309.1 [M+H], found 309.0. and (E)-2-fluoro-2-(1-(naphthalen-2-yl)-1,4,5,6-tetrahydro-7H-indazol-7-ylidene)ethan-1-ol: Mass spectrum (ESI, m/z): Calculated for C₁₉H₁₈FN₂O, 309.1 [M+H], found 308.9.

### Steps 6-8. Synthesis of (Z)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide

(Z)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide was prepared from the intermediate (Z)-2-fluoro-2-(1-(naphthalen-2-yl)-1,4,5,6-tetrahydro-7H-indazol-7-ylidene)ethan-1-ol according to the procedures as described in Example 5, Steps 1-3.

¹H NMR (400 MHz, CD₃OD) δ: 7.78-7.84 (m, 3H), 7.69-7.74 (m, 2H), 7.38-7.47 (m, 5H), 6.95 (d, J=8.8 Hz, 1H), 3.81 (d, J=11.6 Hz, 2H), 3.65 (s, 3H), 2.61 (t, J=6.8 Hz, 2H), 2.47 (t, J=5.6 Hz, 2H), 1.70-1.73 (m, 2H). ¹⁹F NMR (400 MHz, CD₃OD) δ: −104.77. Mass spectrum (ESI, m/z): Calculated for C₂₇H₂₅ClFN₄O₄S: 555.1 [M+H], found: 555.1.

### Steps 9-11. Synthesis of (E)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide

(E)-5-chloro-N-((2-fluoro-2-(1-(naphthalen-2-yl)-1,4,5,6-tetrahydro-7H-indazol-7-ylidene)ethyl)carbamoyl)-2-methoxybenzenesulfonamide was prepared from the intermediate (E)-2-fluoro-2-(1-(naphthalen-2-yl)-1,4,5,6-tetrahydro-7H-indazol-7-ylidene)ethan-1-ol according to the procedures as described in Example 11, Steps 1-3.

¹H NMR (400 MHz, CD₃OD) δ: 7.92 (d, J=8.8 Hz, 1H), 7.82-7.85 (m, 2H), 7.80 (s, 1H), 7.65 (s, 1H), 7.43-7.50 (m, 5H), 7.05 (d, J=8.8 Hz, 1H), 3.71 (s, 3H), 3.03 (d, J=17.6 Hz, 2H), 2.63 (t, J=6.8 Hz, 2H), 2.49-2.52 (m, 2H), 1.78-1.83 (m, 2H). ¹⁹F NMR (400 MHz, CD₃OD) δ: −116.59. Mass spectrum (ESI, m/z): Calculated for C₂₇H₂₅ClFN₄O₄S: 555.1 [M+H], found: 555.1.

The following representative compounds of formula (I) were similarly prepared according to the procedure described in Example 13 (and Example 5 as referenced therein) above, selecting and substituting suitable starting materials and reagents, as would be readily recognized by those skilled in the art.

### (Z)-5-fluoro-N-((2-fluoro-2-(1-(naphthalen-2-yl)-1,4,5,6-tetrahydro-7H-indazol-7-ylidene)ethyl)carbamoyl)-2-methoxybenzenesulfonamide, Compound #113

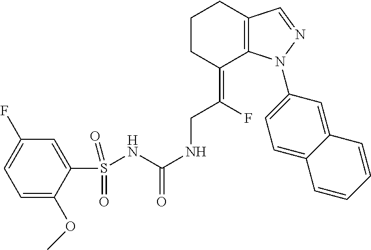 [SMILES: COc1ccc(F)cc1S(=O)(=O)NC(=O)NC/C(F)=C1\CCCc2cnn(-c3ccc4ccccc4c3)c21]

¹H NMR (400 MHz, CD₃OD) δ: 7.77-7.84 (m, 3H), 7.69 (s, 1H), 7.51 (d, J=8.0 Hz, 1H), 7.47 (s, 1H), 7.38-7.45 (m, 3H), 7.18-7.23 (m, 1H), 6.96 (d, J=9.2 Hz, 1H), 3.81 (d, J=26.8 Hz, 2H), 3.61 (s, 3H), 2.61 (t, J=6.8 Hz, 2H), 2.47 (t, J=5.6 Hz, 2H), 1.72-1.74 (m, 2H). ¹⁹F NMR (400 MHz, CD₃OD) δ: −104.56, −124.46. Mass spectrum (ESI, m/z): Calculated for C₂₇H₂₅F₂N₄O₄S: 539.1 [M+H], found: 539.1.

### (E)-5-fluoro-N-((2-fluoro-2-(1-(naphthalen-2-yl)-1,4,5,6-tetrahydro-7H-indazol-7-ylidene)ethyl)carbamoyl)-2-methoxybenzenesulfonamide, Compound #112

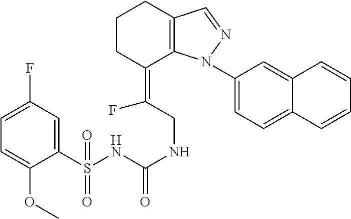 [SMILES: COc1ccc(F)cc1S(=O)(=O)NC(=O)NC/C(F)=C1/CCCc2cnn(-c3ccc4ccccc4c3)c21]

¹H NMR (400 MHz, CD₃OD) δ: 8.06 (d, J=8.8 Hz, 1H), 7.92-7.98 (m, 3H), 7.67 (s, 1H), 7.53-7.62 (m, 4H), 7.37-7.42 (m, 1H), 7.17 (d, J=9.2 Hz, 1H), 3.82 (s, 3H), 3.15 (d, J=8.4 Hz, 2H), 2.76 (t, J=6.4 Hz, 2H), 2.63 (t, J=3.6 Hz, 2H), 1.91-1.97 (m, 2H). ¹⁹F NMR (400 MHz, CD₃OD) δ: −115.80, −124.50. Mass spectrum (ESI, m/z): Calculated for C₂₇H₂₅F₂N₄O₄S: 539.1 [M+H], found: 539.1.

### (Z)—N-((2-fluoro-2-(1-(naphthalen-2-yl)-1,4,5,6-tetrahydro-7H-indazol-7-ylidene)ethyl)carbamoyl)-2,3-dihydrobenzo[b][1,4]dioxine-6-sulfonamide, Compound #114

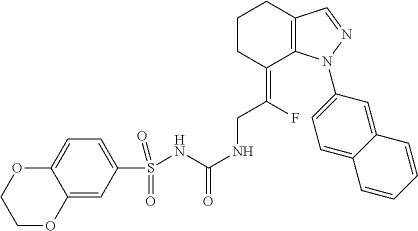 [SMILES: O=C(NC/C(F)=C1\CCCc2cnn(-c3ccc4ccccc4c3)c21)NS(=O)(=O)c1ccc2c(c1)OCCO2]

¹H NMR (400 MHz, CD₃OD) δ: 7.74-7.79 (m, 3H), 7.69 (s, 1H), 7.38-7.46 (m, 4H), 7.32 (s, 1H), 7.27 (d, J=8.4 Hz, 1H), 6.79 (d, J=8.4 Hz, 1H), 4.11-4.16 (m, 4H), 3.77 (d, J=18.0 Hz, 2H), 2.64 (t, J=6.4 Hz, 2H), 2.51 (t, J=5.6 Hz, 2H), 1.80-1.83 (m, 2H). ¹⁹F NMR (400 MHz, CD₃OD) δ: −103.81. Mass spectrum (ESI, m/z): Calculated for C₂₈H₂₆FN₄O₅S: 549.2 [M+H], found: 549.1.

### (E)-N-((2-fluoro-2-(1-(naphthalen-2-yl)-1,4,5,6-tetrahydro-7H-indazol-7-ylidene)ethyl)carbamoyl)-2,3-dihydrobenzo[b][1,4]dioxine-6-sulfonamide, Compound #110

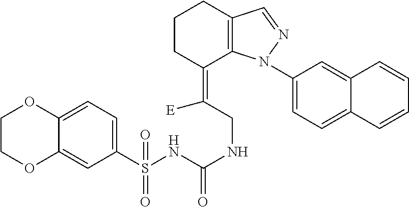

¹H NMR (400 MHz, CD₃OD) δ: 7.89-8.04 (m, 4H), 7.55-7.68 (m, 4H), 7.23-7.28 (m, 2H), 6.91 (d, J=8.4 Hz, 1H), 4.28-4.32 (m, 4H), 3.14 (d, J=18.0 Hz, 2H), 2.75 (t, J=7.2 Hz, 2H), 2.63 (t, J=4.8 Hz, 2H), 1.92-1.97 (m, 2H). ¹⁹F NMR (400 MHz, CD₃OD) δ: −114.82. Mass spectrum (ESI, m/z): Calculated for C₂₈H₂₆FN₄O₅S: 549.2 [M+H], found: 549.3.

### (Z)—N-((2-fluoro-2-(1-(4-(trifluoromethyl)phenyl)-1,4,5,6-tetrahydro-7H-indazol-7-ylidene)ethyl)carbamoyl)-2,3-dihydrobenzo[b][1,4]dioxine-6-sulfonamide, Compound #116

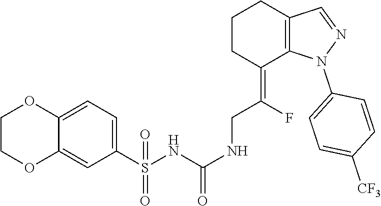 [SMILES: O=C(NC/C(F)=C1\CCCc2cnn(-c3ccc(C(F)(F)F)cc3)c21)NS(=O)(=O)c1ccc2c(c1)OCCO2]

¹H NMR (400 MHz, CD₃OD) δ: 7.58 (d, J=8.4 Hz, 2H), 7.42-7.46 (m, 3H), 7.30-7.34 (m, 2H), 6.81 (d, J=8.4 Hz, 1H), 4.12-4.19 (m, 4H), 3.82 (d, J=20.4 Hz, 2H), 2.59-2.62 (m, 2H), 2.46-2.49 (m, 2H), 1.79-1.81 (m, 2H). ¹⁹F NMR (400 MHz, CD₃OD) δ: −63.68, −103.70. Mass spectrum (ESI, m/z): Calculated for C₂₅H₂₃F₄N₄O₅S: 567.1 [M+H], found: 567.1.

### (E)-N-((2-fluoro-2-(1-(4-(trifluoromethyl)phenyl)-1,4,5,6-tetrahydro-7H-indazol-7-ylidene)ethyl)carbamoyl)-2,3-dihydrobenzo[b][1,4]dioxine-6-sulfonamide, Compound #117

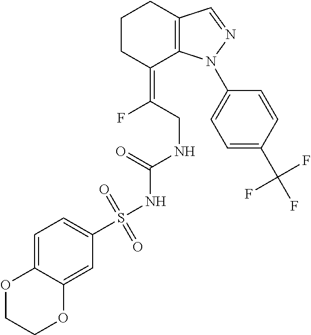 [SMILES: O=C(NC/C(F)=C1/CCCc2cnn(-c3ccc(C(F)(F)F)cc3)c21)NS(=O)(=O)c1ccc2c(c1)OCCO2]

¹H NMR (400 MHz, CD₃OD) δ: 7.81 (d, J=8.4 Hz, 2H), 7.64 (d, J=8.4 Hz, 2H), 7.58 (s, 1H), 7.32 (d, J=8.8 Hz, 2H), 6.92 (d, J=8.4 Hz, 1H), 4.28-4.31 (m, 4H), 3.22-3.30 (m, 2H), 2.60-2.70 (m, 2H), 2.52-2.59 (m, 2H), 1.89-1.96 (m, 2H). ¹⁹F NMR (400 MHz, CD₃OD) δ: −63.83, −115.70. Mass spectrum (ESI, m/z): Calculated for C₂₅H₂₃F₄N₄O₅S: 567.1 [M+H], found: 566.9.

### (Z)-5-chloro-N-((2-fluoro-2-(1-(4-(trifluoromethyl)phenyl)-1,4,5,6-tetrahydro-7H-indazol-7-ylidene)ethyl)carbamoyl)thiophene-2-sulfonamide, Compound #124

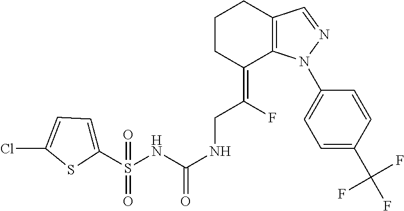 [SMILES: O=C(NC/C(F)=C1\CCCc2cnn(-c3ccc(C(F)(F)F)cc3)c21)NS(=O)(=O)c1ccc(Cl)s1]

¹H NMR (400 MHz, CD₃OD) δ: 7.68 (d, J=8.4 Hz, 2H), 7.54-7.59 (m, 4H), 7.03 (d, J=4.4 Hz, 1H), 3.96 (d, J=20.4 Hz, 2H), 2.70-2.74 (m, 2H), 2.60-2.63 (m, 2H), 1.94-2.03 (m, 2H). ¹⁹F NMR (400 MHz, CD₃OD) δ: −63.71, −104.40. Mass spectrum (ESI, m/z): Calculated for C₂₁H₁₇F₄N₄O₃S₂: 549.0 [M+H], found: 549.0.

### (Z)-4,5-dichloro-N-((2-fluoro-2-(1-(4-(trifluoromethyl)phenyl)-1,4,5,6-tetrahydro-7H-indazol-7-ylidene)ethyl)carbamoyl)thiophene-2-sulfonamide, Compound #125

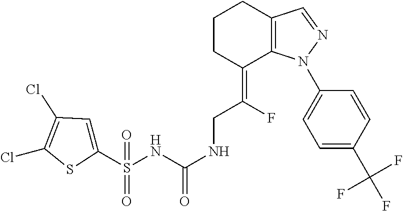 [SMILES: O=C(NC/C(F)=C1\CCCc2cnn(-c3ccc(C(F)(F)F)cc3)c21)NS(=O)(=O)c1cc(Cl)c(Cl)s1]

¹H NMR (400 MHz, CD₃OD) δ: 7.69 (d, J=8.8 Hz, 2H), 7.64 (s, 1H), 7.54-7.56 (m, 3H), 3.96 (d, J=20.4 Hz, 2H), 2.71-2.74 (m, 2H), 2.61-2.64 (m, 2H), 1.93-1.96 (m, 2H). ¹⁹F NMR (400 MHz, CD₃OD) δ: −63.73, −104.45. Mass spectrum (ESI, m/z): Calculated for C₂₁H₁₇Cl₂F₄N₄O₃S₂: 583.0 [M+H], found: 583.0.

### (Z)-4,5-dichloro-N-((2-fluoro-2-(1-(1-(4-fluorophenyl)piperidin-4-yl)-1,4,5,6-tetrahydro-7H-indazol-7-ylidene)ethyl)carbamoyl)thiophene-2-sulfonamide, Compound #154

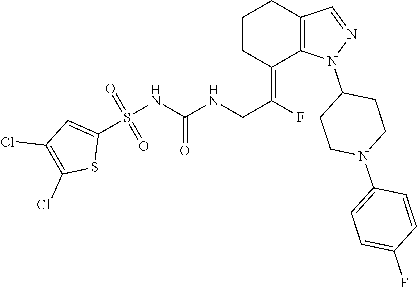 [SMILES: O=C(NC/C(F)=C1\CCCc2cnn(C3CCN(c4ccc(F)cc4)CC3)c21)NS(=O)(=O)c1cc(Cl)c(Cl)s1]

¹H NMR (400 MHz, Methanol-d4) δ: 7.53 (s, 1H), 7.31 (s, 1H), 7.01-6.93 (m, 4H), 4.25-4.23 (m, 1H), 4.19-4.10 (m, 2H), 3.67-3.47 (m, 2H), 2.79-2.73 (m, 2H), 2.62-2.59 (m, 2H), 2.45-2.43 (m, 2H), 2.31-2.23 (m, 2H), 1.91-1.88 (m, 4H). ¹⁹F NMR (400 MHz, Methanol-d4) δ: −107.50, −126.68. Mass spectrum (ESI, m/z): Calculated for C₂₅H₂₆Cl₂F₂N₅O₃S₂: 616.1 [M+H], found: 616.1.

### Example 14: Compound #52

### (E)-4-(1-([1,1′-biphenyl]-3-yl)-1,4,5,6-tetrahydro-7H-indazol-7-ylidene)-N-((4,5-dichlorothiophen-2-yl)sulfonyl)butanamide

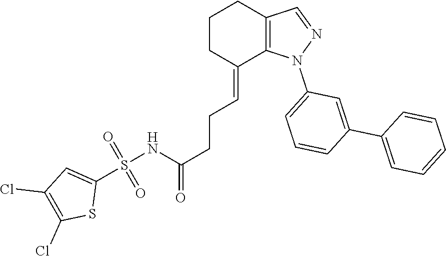 [SMILES: O=C(CC/C=C1\CCCc2cnn(-c3cccc(-c4ccccc4)c3)c21)NS(=O)(=O)c1cc(Cl)c(Cl)s1]

### Step 1. Synthesis of ethyl (E)-2-(1-(3-bromophenyl)-1,4,5,6-tetrahydro-7H-indazol-7-ylidene)acetate

Ethyl (E)-2-(1-(3-bromophenyl)-1,4,5,6-tetrahydro-7H-indazol-7-ylidene)acetate was prepared according to the procedures as described in Example 1, Step 3.

### Step 2. Synthesis of (E)-2-(1-(3-bromophenyl)-1,4,5,6-tetrahydro-7H-indazol-7-ylidene)ethan-1-ol

(E)-2-(1-(3-bromophenyl)-1,4,5,6-tetrahydro-7H-indazol-7-ylidene)ethan-1-ol was prepared according to the procedures as described in Example 1, Step 4.

### Step 3. Synthesis of (E)-1-(3-bromophenyl)-7-(2-chloroethylidene)-4,5,6,7-tetrahydro-1H-indazole

(E)-1-(3-bromophenyl)-7-(2-chloroethylidene)-4,5,6,7-tetrahydro-1H-indazole was prepared according to the procedures as described in Example 4, Step 1.

### Step 4. Synthesis of diethyl (E)-2-(2-(1-(3-bromophenyl)-1,4,5,6-tetrahydro-7H-indazol-7-ylidene)ethyl)malonate

Diethyl (E)-2-(2-(1-(3-bromophenyl)-1,4,5,6-tetrahydro-7H-indazol-7-ylidene)ethyl)malonate was prepared according to the procedures as described in Example 4, Step 2.

### Step 5. Synthesis of ethyl (E)-4-(1-(3-bromophenyl)-1,4,5,6-tetrahydro-7H-indazol-7-ylidene)butanoate

Into a 20 mL microwave vial was added diethyl (E)-2-(2-(1-(3-bromophenyl)-1,4,5,6-tetrahydro-7H-indazol-7-ylidene)ethyl)malonate (900 mg, 1.95 mmol), DMSO (10 mL), and a solution of LiCl (164 mg, 3.87 mmol) in H₂O (0.5 mL). The reaction was heated by microwave at 170° C. for 2 h. The reaction was quenched with H₂O, diluted with EtOAc, and washed with sat. aq. NaCl. The organic solution was concentrated under vacuum. The resulting residue was purified by silica gel column with PE:EA=90:10 to yield ethyl (E)-4-(1-(3-bromophenyl)-1,4,5,6-tetrahydro-7H-indazol-7-ylidene)butanoate as yellow oil. Mass spectrum (ESI, m/z): Calculated for C₁₉H₂₂BrN₂O₂: 389.1 (M+H), found: 390.7.

### Step 6. Synthesis of ethyl (E)-4-(1-([1,1′-biphenyl]-3-yl)-1,4,5,6-tetrahydro-7H-indazol-7-ylidene)butanoate

A mixture of ethyl (E)-4-(1-(3-bromophenyl)-1,4,5,6-tetrahydro-7H-indazol-7-ylidene)butanoate (100 mg, 0.257 mmol), phenylboronic acid (38 mg, 0.312 mmol), Pd(PPh₃)₄ (15 mg, 0.013 mmol), and K₃PO₄ (110 mg, 0.519 mmol) in DMF (20 mL) was stirred at 100° C. overnight. The reaction was quenched with H₂O, diluted with EtOAc, and washed with sat. aq. NaCl. The organic solution was concentrated under vacuum. The resulting residue was purified by silica gel column with PE:EA=90:10 to yield ethyl (E)-4-(1-([1,1′-biphenyl]-3-yl)-1,4,5,6-tetrahydro-7H-indazol-7-ylidene)butanoate as a yellow oil. Mass spectrum (ESI, m/z): Calculated for C₂₅H₂₇N₂O₂: 387.2 (M+H), found: 387.2.

### Step 7. Synthesis of (E)-4-(1-([1,1′-biphenyl]-3-yl)-1,4,5,6-tetrahydro-7H-indazol-7-ylidene)butanoic acid

To a solution of ethyl (E)-4-(1-([1,1′-biphenyl]-3-yl)-1,4,5,6-tetrahydro-7H-indazol-7-ylidene)butanoate (88 mg, 0.228 mmol) in THF (5 mL) was added a solution of NaOH (46 mg, 1.15 mmol) in H₂O (5 mL). The reaction was stirred overnight at room temperature. The resulting solution was concentrated under vacuum. The residue was diluted with H₂O. The pH was adjusted to ˜4-5. The resulting mixture was diluted with EtOAc, and washed with sat. aq. NaCl. The organic solution was concentrated under vacuum. The resulting residue was purified by TLC with PE:EA=75:25 to yield (E)-4-(1-([1,1′-biphenyl]-3-yl)-1,4,5,6-tetrahydro-7H-indazol-7-ylidene)butanoic acid as a white solid. Mass spectrum (ESI, m/z): Calculated for C₂₃H₂₃N₂O₂: 359.2 (M+H), found: 359.2.

### Step 8. Synthesis of (E)-4-(1-([1,1′-biphenyl]-3-yl)-1,4,5,6-tetrahydro-7H-indazol-7-ylidene)-N-((4,5-dichlorothiophen-2-yl)sulfonyl)butanamide

(E)-4-(1-([1,1′-biphenyl]-3-yl)-1,4,5,6-tetrahydro-7H-indazol-7-ylidene)-N-((4,5-dichlorothiophen-2-yl)sulfonyl)butanamide was according to the procedures as described in Example 4, Step 4, reacting (E)-4-(1-([1,1′-biphenyl]-3-yl)-1,4,5,6-tetrahydro-7H-indazol-7-ylidene)butanoic acid with 4,5-dichlorothiophene-2-sulfonamide to yield (E)-4-(1-([1,1′-biphenyl]-3-yl)-1,4,5,6-tetrahydro-7H-indazol-7-ylidene)-N-((4,5-dichlorothiophen-2-yl)sulfonyl)butanamide.

¹H NMR (300 MHz, Methanol-d4) δ: 7.57-7.72 (m, 6H), 7.47-7.50 (m, 3H), 7.38-7.45 (m, 1H), 7.27 (d, J=8.4 Hz, 1H), 5.08 (t, J=7.5 Hz, 1H), 2.68 (t, J=6.0 Hz, 2H), 2.51 (t, J=6.0 Hz, 2H), 2.31-2.34 (m, 2H), 2.16 (t, J=6.7 Hz, 2H), 1.81-1.87 (m, 2H). Mass spectrum (ESI, m/z): Calculated for C₂₇H₂₄Cl₂N₃O₃S₂: 572.1 [M+H], found: 572.1.

The following representative compound of formula (I) was similarly prepared according to the procedure described in Example 14 (and the Examples referenced therein) above, selecting and substituting suitable starting materials and reagents, as would be readily recognized by those skilled in the art.

### (E)-4-(1-(2′,4′-dichloro-[1,1′-biphenyl]-3-yl)-1,4,5,6-tetrahydro-7H-indazol-7-ylidene)-N-((4,5-dichlorothiophen-2-yl)sulfonyl)butanamide, Compound #53

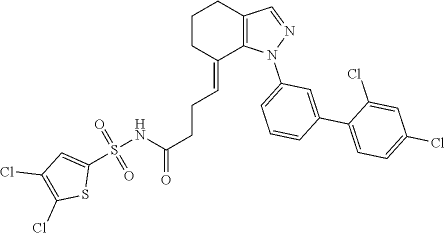 [SMILES: O=C(CC/C=C1\CCCc2cnn(-c3cccc(-c4ccc(Cl)cc4Cl)c3)c21)NS(=O)(=O)c1cc(Cl)c(Cl)s1]

¹H NMR (400 MHz, DMSO) 12.45-12.70 (brs, 1H), 7.82 (s, 1H), 7.76 (s, 1H), 7.54-7.57 (m, 1H), 7.52-7.53 (m, 1H), 7.41-7.48 (m, 3H), 7.37-7.41 (m, 2H), 5.00 (t, J=7.2 Hz, 1H), 2.59 (t, J=6.0 Hz, 2H), 2.41 (t, J=4.8 Hz, 2H), 2.19-2.24 (m, 2H), 2.14-2.17 (m, 2H), 1.72-1.75 (m, 2H). Mass spectrum (ESI, m/z): Calculated for C₂₇H₂₂Cl₄N₃O₃S₂: 640.0 [M+H], found 642.0.

### Example 15: Compound #76

### (E)-4,5-dichloro-N-((2-(1-(4′-fluoro-[1,1′-biphenyl]-3-yl)-1,4,5,6-tetrahydro-7H-indazol-7-ylidene)ethyl)carbamoyl)thiophene-2-sulfonamide

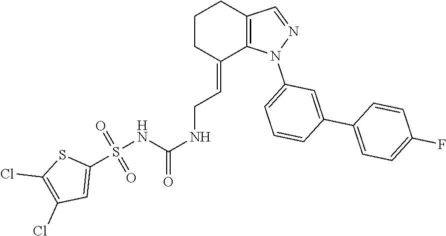 [SMILES: O=C(NC/C=C1\CCCc2cnn(-c3cccc(-c4ccc(F)cc4)c3)c21)NS(=O)(=O)c1cc(Cl)c(Cl)s1]

### Step 1. Synthesis of (E)-2-(2-(1-(3-bromophenyl)-1,4,5,6-tetrahydro-7H-indazol-7-ylidene)ethyl)isoindoline-1,3-dione

(E)-2-(2-(1-(3-bromophenyl)-1,4,5,6-tetrahydro-7H-indazol-7-ylidene)ethyl)isoindoline-1,3-dione was prepared from (E)-2-(1-(3-bromophenyl)-1,4,5,6-tetrahydro-7H-indazol-7-ylidene)ethan-1-ol was prepared according to the procedures as described in Example 5, Step 1.

### Step 2. Synthesis of (E)-2-(2-(1-(4′-fluoro-[1,1′-biphenyl]-3-yl)-1,4,5,6-tetrahydro-7H-indazol-7-ylidene)ethyl)isoindoline-1,3-dione

To a 100-mL round-bottom flask purged and maintained with an inert atmosphere of nitrogen, was added 2-(2-(1-(3-bromophenyl)-5,6-dihydro-1H-indazol-7(4H)-ylidene)ethyl)isoindoline-1,3-dione (100 mg, 0.223 mmol), 4-fluorophenylboronic acid (34 mg, 0.243 mmol), Pd(dppf)Cl₂.CH₂Cl₂ (9 mg, 0.011 mmol), Na₂CO₃ (47 mg, 0.443 mmol), DME (10 mL), and water (2 mL). The reaction was stirred overnight at 80° C. The reaction progress was monitored by LCMS. The reaction was then diluted with 20 mL of H₂O and extracted with of ethyl acetate (3×20 mL). The organic layers were combined and concentrated under vacuum. The resulting residue was purified by silica gel column with ethyl acetate/petroleum ether (2:98) to yield (E)-2-(2-(1-(4′-fluoro-[1,1′-biphenyl]-3-yl)-1,4,5,6-tetrahydro-7H-indazol-7-ylidene)ethyl)isoindoline-1,3-dione as a yellow oil. Mass spectrum (ESI, m/z): Calculated for C₂₉H₂₃FN₃O₂, 464.2 (M+H), found 463.9.

### Step 3. Synthesis of (E)-2-(1-(4′-fluoro-[1,1′-biphenyl]-3-yl)-1,4,5,6-tetrahydro-7H-indazol-7-ylidene)ethan-1-amine

(E)-2-(1-(4′-fluoro-[1,1′-biphenyl]-3-yl)-1,4,5,6-tetrahydro-7H-indazol-7-ylidene)ethan-1-amine was prepared according to the procedures as described in Example 5, Step 2.

### Step 4. Synthesis of (E)-4,5-dichloro-N-((2-(1-(4′-fluoro-[1,1′-biphenyl]-3-yl)-1,4,5,6-tetrahydro-7H-indazol-7-ylidene)ethyl)carbamoyl)thiophene-2-sulfonamide

(E)-4,5-dichloro-N-((2-(1-(4′-fluoro-[1,1′-biphenyl]-3-yl)-1,4,5,6-tetrahydro-7H-indazol-7-ylidene)ethyl)carbamoyl)thiophene-2-sulfonamide was prepared according to the procedures as described in Example 5, Step 3.

¹H NMR (300 MHz, CD₃OD) δ: 7.62-7.65 (m, 3H), 7.48-7.53 (m, 2H), 7.43 (s, 1H), 7.29-7.32 (m, 1H), 7.24 (s, 1H), 7.14-7.19 (m, 2H), 5.15-5.19 (m, 1H), 4.55-4.56 (m, 1H), 3.67-3.69 (m, 2H), 2.65 (t, J=6.0 Hz, 2H), 2.54-2.55 (m, 2H), 1.82-1.88 (m, 2H). ¹⁹F NMR (300 MHz, CD₃OD) δ: −117.16. Mass spectrum (ESI, m/z): Calculated for C₂₆H₂₂C₁₂FN₄O₃S₂, 591.0 (M+H), found 591.0.

The following representative compound of formula (I) was similarly prepared according to the procedure described in Example 15 (and Examples referenced therein) above, selecting and substituting suitable starting materials and reagents, as would be readily recognized by those skilled in the art.

### (E)-N-((2-(1-([1,1′-biphenyl]-4-yl)-1,4,5,6-tetrahydro-7H-indazol-7-ylidene)ethyl)carbamoyl)-4,5-dichlorothiophene-2-sulfonamide, Compound #99

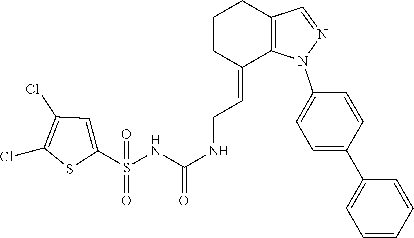 [SMILES: O=C(NC/C=C1\CCCc2cnn(-c3ccc(-c4ccccc4)cc3)c21)NS(=O)(=O)c1cc(Cl)c(Cl)s1]

¹H NMR (300 MHz, CD₃OD) δ: 7.67-7.75 (m, 4H), 7.38-7.51 (m, 7H), 5.14 (t, J=6.9 Hz, 1H), 3.76 (d, J=6.9 Hz, 2H), 2.70 (d, J=6.3 Hz, 2H), 2.58 (t, J=5.1 Hz, 2H), 1.87-1.91 (m, 2H). Mass spectrum (ESI, m/z): Calculated for C₂₆H₂₃Cl₂N₄O₃S₂: 573.1 [M+H], found 574.8.

### Example 16: Compound #88 and Compound #87

### (E)-4,5-dichloro-N-((2-(1-(naphthalen-2-ylmethyl)-1,4,5,6-tetrahydro-7H-indazol-7-ylidene)ethyl)carbamoyl)thiophene-2-sulfonamide and

### (E)-4,5-dichloro-N-((2-(2-(naphthalen-2-ylmethyl)-2,4,5,6-tetrahydro-7H-indazol-7-ylidene)ethyl)carbamoyl)thiophene-2-sulfonamide

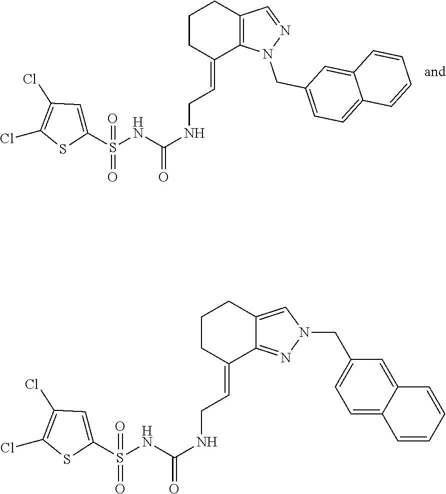 [SMILES: O=C(NC/C=C1\CCCc2cnn(Cc3ccc4ccccc4c3)c21)NS(=O)(=O)c1cc(Cl)c(Cl)s1 ; O=C(NC/C=C1\CCCc2cn(Cc3ccc4ccccc4c3)nc21)NS(=O)(=O)c1cc(Cl)c(Cl)s1]

### Step 1. Synthesis of ethyl (E)-2-(1,4,5,6-tetrahydro-7H-indazol-7-ylidene)acetate

Into a 100-mL round flask was added ethyl (E)-2-((E)-3-(hydroxymethylene)-2-oxocyclohexylidene)acetate (2 g, 9.51 mmol), hydrazine (800 mg, 16.0 mmol), 1,4-dioxane (50 mL), and TFA (2 mL). The reaction was heated at 60° C. for 2 h. The reaction mixture was neutralized with aq NaHCO₃ and extracted with EtOAc. The organic layer was washed with sat. (aq). NaCl and dried over Na₂SO₄, and concentrated. Purification of the resulting residue by silica gel column with 10% EtOAc/heptane yielded ethyl (E)-2-(1,4,5,6-tetrahydro-7H-indazol-7-ylidene)acetate as a yellow solid. Mass spectrum (ESI, m/z): Calculated for C₁₁H₁₅N₂O₂: 207.1 [M+H], found 207.0.

### Step 2. Synthesis of ethyl (E)-2-(1-(naphthalen-2-ylmethyl)-1,4,5,6-tetrahydro-7H-indazol-7-ylidene)acetate and ethyl (E)-2-(2-(naphthalen-2-ylmethyl)-2,4,5,6-tetrahydro-7H-indazol-7-ylidene)acetate

Into a 250-mL round flask was added (E)-2-(1,4,5,6-tetrahydro-7H-indazol-7-ylidene)acetate (1.6 g, 7.76 mmol), 2-(bromomethyl)naphthalene (2.6 g, 11.8 mmol), Cs₂CO₃ (3.8 g, 27.5 mmol), and DMF (100 mL). The reaction was stirred for 16 h at 80° C. The reaction was then quenched with H₂O and extracted with EtOAc. The organic layers were combined and concentrated under vacuum. The resulting residue was purified by silica gel column with PE:EA=70:30 to yield a mixture of ethyl (E)-2-(1-(naphthalen-2-ylmethyl)-1,4,5,6-tetrahydro-7H-indazol-7-ylidene)acetate and ethyl (E)-2-(2-(naphthalen-2-ylmethyl)-2,4,5,6-tetrahydro-7H-indazol-7-ylidene)acetate as a yellow solid. Mass spectrum (ESI, m/z): Calculated for C₂₂H₂₃N₂O₂ 347.2 [M+H], found: 347.2.

### Step 3. Synthesis of (E)-2-(1-(naphthalen-2-ylmethyl)-1,4,5,6-tetrahydro-7H-indazol-7-ylidene)ethan-1-ol and (E)-2-(2-(naphthalen-2-ylmethyl)-2,4,5,6-tetrahydro-7H-indazol-7-ylidene)ethan-1-ol

A mixture of (E)-2-(1-(naphthalen-2-ylmethyl)-1,4,5,6-tetrahydro-7H-indazol-7-ylidene)ethan-1-ol and (E)-2-(2-(naphthalen-2-ylmethyl)-2,4,5,6-tetrahydro-7H-indazol-7-ylidene)ethan-1-ol was prepared according to the procedures described in Example 1, Step 4.

### Step 4. Synthesis of (E)-2-(2-(1-(naphthalen-2-ylmethyl)-1,4,5,6-tetrahydro-7H-indazol-7-ylidene)ethyl)isoindoline-1,3-dione and (E)-2-(2-(2-(naphthalen-2-ylmethyl)-2,4,5,6-tetrahydro-7H-indazol-7-ylidene)ethyl)isoindoline-1,3-dione

A mixture of (E)-2-(2-(1-(naphthalen-2-ylmethyl)-1,4,5,6-tetrahydro-7H-indazol-7-ylidene)ethyl)isoindoline-1,3-dione and (E)-2-(2-(2-(naphthalen-2-ylmethyl)-2,4,5,6-tetrahydro-7H-indazol-7-ylidene)ethyl)isoindoline-1,3-dione was prepared according to the procedures described in Example 5, Step 1.

### Step 5. Synthesis of (E)-2-(1-(naphthalen-2-ylmethyl)-1,4,5,6-tetrahydro-7H-indazol-7-ylidene)ethan-1-amine and (E)-2-(2-(naphthalen-2-ylmethyl)-2,4,5,6-tetrahydro-7H-indazol-7-ylidene)ethan-1-amine

A mixture of (E)-2-(1-(naphthalen-2-ylmethyl)-1,4,5,6-tetrahydro-7H-indazol-7-ylidene)ethan-1-amine and (E)-2-(2-(naphthalen-2-ylmethyl)-2,4,5,6-tetrahydro-7H-indazol-7-ylidene)ethan-1-amine was prepared according to the procedures described in Example 5, Step 2.

### Step 6. Synthesis of (E)-4,5-dichloro-N-((2-(1-(naphthalen-2-ylmethyl)-1,4,5,6-tetrahydro-7H-indazol-7-ylidene)ethyl)carbamoyl)thiophene-2-sulfonamide and (E)-4,5-dichloro-N-((2-(2-(naphthalen-2-ylmethyl)-2,4,5,6-tetrahydro-7H-indazol-7-ylidene)ethyl)carbamoyl)thiophene-2-sulfonamide

Into a 20-mL vial was added 4,5-dichlorothiophene-2-sulfonamide (265 mg, 1.14 mmol), CDI (241 mg, 1.49 mmol), triethylamine (150 mg, 1.49 mmol), and THF (10 mL). The resulting solution was stirred for 16 h at room temperature. To the reaction was then added a mixture of (E)-2-(1-(naphthalen-2-ylmethyl)-1,4,5,6-tetrahydro-7H-indazol-7-ylidene)ethan-1-amine and (E)-2-(2-(naphthalen-2-ylmethyl)-2,4,5,6-tetrahydro-7H-indazol-7-ylidene)ethan-1-amine (150 mg, 0.471 mmol). The reaction was heated by microwave at 100° C. for 1 h. The resulting solution was concentrated under vacuum. The resulting residue was purified by TLC with DCM:MeOH=15:1 followed by Prep-HPLC with the following conditions (1#-Waters 2767-5): Column, SunFire Prep C18, 5 um, 19*100 mm; mobile phase, Water with 0.05% TFA and CH₃CN (50% CH₃CN up to 70% in 10 min, up to 100% in 0.1 min, hold 100% in 0.9 min, down to 50% in 0.1 min, hold 50% in 1.4 min); Detector, UV 220&254 nm to yield (E)-4,5-dichloro-N-((2-(1-(naphthalen-2-ylmethyl)-1,4,5,6-tetrahydro-7H-indazol-7-ylidene)ethyl)carbamoyl)thiophene-2-sulfonamide as a white solid: ¹H NMR (300 MHz, CD₃OD, ppm) δ: 7.83-7.88 (m, 3H), 7.74 (s, 1H), 7.66 (d, J=3.0 Hz, 2H), 7.48-7.54 (m, 2H), 7.36 (d, J=8.4 Hz, 1H), 6.10 (t, J=7.2 Hz, 1H), 5.52 (s, 2H), 5.93 (d, J=6.9 Hz, 2H), 2.63 (t, J=5.7 Hz, 2H), 2.54 (t, J=5.7 Hz, 2H), 1.81-1.87 (m, 2H). Mass spectrum (ESI, m/z): Calculated for C₂₅H₂₃Cl₂N₄O₃S₂: 561.1 [M+H], found 561.0.

and (E)-4,5-dichloro-N-((2-(2-(naphthalen-2-ylmethyl)-2,4,5,6-tetrahydro-7H-indazol-7-ylidene)ethyl)carbamoyl)thiophene-2-sulfonamide as a yellow solid: ¹H NMR (300 MHz, CD₃OD, ppm) δ: 7.86-7.92 (m, 3H), 7.78 (s, 2H), 7.61 (s, 1H), 7.49-7.52 (m, 2H), 7.41 (d, J=8.4 Hz, 1H), 6.02 (t, J=4.2 Hz, 1H), 5.64 (s, 2H), 3.31-3.34 (m, 2H), 2.55-2.66 (m, 4H), 2.34-2.38 (m, 2H). Mass spectrum (ESI, m/z): Calculated for C₂₅H₂₃Cl₂N₄O₃S₂: 561.1 [M+H], found: 561.0.

The following representative compound of formula (I) was similarly prepared according to the procedure described in Example 16 (and Examples referenced therein) above, selecting and substituting suitable starting materials and reagents, as would be readily recognized by those skilled in the art.

### (E)-4,5-dichloro-N-((2-(1-((5-phenylpyrimidin-2-yl)methyl)-1,4,5,6-tetrahydro-7H-indazol-7-ylidene)ethyl)carbamoyl)thiophene-2-sulfonamide, Compound #163

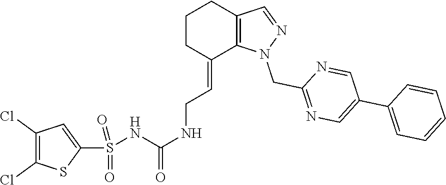 [SMILES: O=C(NC/C=C1\CCCc2cnn(Cc3ncc(-c4ccccc4)cn3)c21)NS(=O)(=O)c1cc(Cl)c(Cl)s1]

¹H NMR (400 MHz, CD₃OD) δ: 9.00 (s, 2H), 7.69 (d, J=8.8 Hz, 2H), 7.60 (s, 1H), 7.54 (s, 1H), 7.45-7.53 (m, 3H), 6.05 (t, J=7.2 Hz, 1H), 5.59 (s, 2H), 3.90 (d, J=6.8 Hz, 2H), 2.64 (t, J=6.0 Hz, 2H), 2.52 (t, J=6.0 Hz, 2H), 1.79-1.85 (m, 2H). ¹⁹F NMR (400 MHz, CD₃OD) δ: −77.71 Mass spectrum (ESI, m/z): Calculated for C₂₅H₂₃Cl₂N₆O₃S₂, 589.1 [M+H], found 589.1.

### Example 17: Compound #59

### (Z)-4,5-dichloro-N-((2-(1-(2,4-dichlorobenzyl)-1,4,5,6-tetrahydro-7H-indazol-7-ylidene)-2-fluoroethyl)(methyl)carbamoyl)thiophene-2-sulfonamide

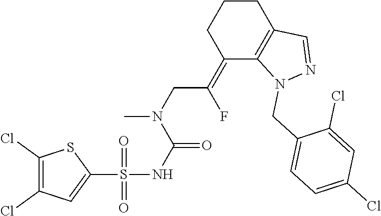 [SMILES: CN(C/C(F)=C1\CCCc2cnn(Cc3ccc(Cl)cc3Cl)c21)C(=O)NS(=O)(=O)c1cc(Cl)c(Cl)s1]

### Step 1. Synthesis of tert-butyl (Z)-(2-(1-(2,4-dichlorobenzyl)-1,4,5,6-tetrahydro-7H-indazol-7-ylidene)-2-fluoroethyl)carbamate

A solution of (Z)-2-(1-(2,4-dichlorobenzyl)-1,4,5,6-tetrahydro-7H-indazol-7-ylidene)-2-fluoroethan-1-amine (647 mg, 1.90 mmol), di-tert-butyl dicarbonate (624 mg, 2.86 mmol), and TEA (386 mg, 3.82 mmol) in DCM (20 mL) was stirred at room temperature overnight. The reaction was quenched with H₂O and extracted with EtOAc. The organic layers were combined and concentrated under vacuum. The resulting residue was purified by silica gel column with PE:EA=80:20 to yield tert-butyl (Z)-(2-(1-(2,4-dichlorobenzyl)-1,4,5,6-tetrahydro-7H-indazol-7-ylidene)-2-fluoroethyl)carbamate as a yellow solid. Mass spectrum (ESI, m/z): Calculated for C₂₁H₂₅Cl₂FN₃O₂: 440.1 [M+H], found 440.0.

### Step 2. Synthesis of tert-butyl (Z)-(2-(1-(2,4-dichlorobenzyl)-1,4,5,6-tetrahydro-7H-indazol-7-ylidene)-2-fluoroethyl)(methyl)carbamate

To tert-butyl (Z)-(2-(1-(2,4-dichlorobenzyl)-1,4,5,6-tetrahydro-7H-indazol-7-ylidene)-2-fluoroethyl)carbamate (510 mg, 1.16 mmol) in DMF (20 mL) at 0° C. was added NaH (139 mg, 3.48 mmol) in portions. The reaction was stirred at 0° C. for 30 min before iodomethane (330 mg, 2.33 mmol) was added. The reaction was warmed to room temperature and stirred for 2 h. The reaction was then quenched with H₂O and extracted with EtOAc. The organic layer was washed with sat. aq. NaCl, dried over Na₂SO₄, and concentrated. Purification of the resulting residue by silica gel column with 10% EtOAc/heptane yielded tert-butyl (Z)-(2-(1-(2,4-dichlorobenzyl)-1,4,5,6-tetrahydro-7H-indazol-7-ylidene)-2-fluoroethyl)(methyl)carbamate as a yellow solid. Mass spectrum (ESI, m/z): Calculated for C₂₂H₂₇Cl₂FN₃O₂, 454.1 (M+H), found 454.1.

### Step 3. Synthesis of (Z)-2-(1-(2,4-dichlorobenzyl)-1,4,5,6-tetrahydro-7H-indazol-7-ylidene)-2-fluoro-N-methylethan-1-amine

A solution of tert-butyl (Z)-(2-(1-(2,4-dichlorobenzyl)-1,4,5,6-tetrahydro-7H-indazol-7-ylidene)-2-fluoroethyl)(methyl)carbamate (400 mg, 0.880 mmol) and TFA (0.4 mL) in DCM (50 mL) was stirred at room temperature for 2 h. The resulting solution was concentrated under vacuum. The resulting residue was purified by TLC with PE:EA=70:30 to yield (Z)-2-(1-(2,4-dichlorobenzyl)-1,4,5,6-tetrahydro-7H-indazol-7-ylidene)-2-fluoro-N-methylethan-1-amine as a yellow solid. Mass spectrum (ESI, m/z): Calculated for C₁₇H₁₉Cl₂FN₃, 354.1 (M+H), found 354.0.

### Step 4. Synthesis of (Z)-4,5-dichloro-N-((2-(1-(2,4-dichlorobenzyl)-1,4,5,6-tetrahydro-7H-indazol-7-ylidene)-2-fluoroethyl)(methyl)carbamoyl)thiophene-2-sulfonamide

(Z)-4,5-dichloro-N-((2-(1-(2,4-dichlorobenzyl)-1,4,5,6-tetrahydro-7H-indazol-7-ylidene)-2-fluoroethyl)(methyl)carbamoyl)thiophene-2-sulfonamide was prepared according to the procedures as described in Example 5, Step 3.

¹H NMR (300 MHz, Methanol-d4) δ: 7.40-7.43 (m, 3H), 7.21 (d, J=8.4 Hz, 1H), 6.67 (d, J=8.4 Hz, 1H), 5.38 (s, 2H), 4.33 (d, J=21.0 Hz, 2H), 2.85 (s, 3H), 2.65-2.69 (m, 2H), 2.45 (t, J=6.0 Hz, 2H), 1.84-1.93 (m, 2H). ¹⁹F NMR (300 MHz, Methanol-d4) δ: −104.57. Mass spectrum (ESI, m/z): Calculated for C₂₂H₂₀Cl₄FN₄O₃S₂: 611.0 (M+H), found: 613.0.

### Example 18, Compound #33

### (E)-4-chloro-N-((2-(1-(2,4-dichlorobenzyl)-1,4,5,6-tetrahydro-7H-indazol-7-ylidene)ethyl)(methyl)carbamoyl)benzenesulfonamide

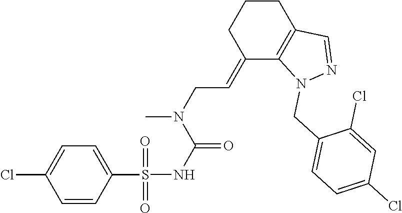 [SMILES: CN(C/C=C1\CCCc2cnn(Cc3ccc(Cl)cc3Cl)c21)C(=O)NS(=O)(=O)c1ccc(Cl)cc1]

### Steps 1-3. Synthesis of (E)-2-(1-(2,4-dichlorobenzyl)-1,4,5,6-tetrahydro-7H-indazol-7-ylidene)-N-methylethan-1-amine

(E)-2-(1-(2,4-dichlorobenzyl)-1,4,5,6-tetrahydro-7H-indazol-7-ylidene)-N-methylethan-1-amine was prepared from (E)-2-(1-(2,4-dichlorobenzyl)-1,4,5,6-tetrahydro-7H-indazol-7-ylidene)ethan-1-amine according to the procedures as described in Example 17, Steps 1-3.

### Step 4. Synthesis of (E)-4-chloro-N-((2-(1-(2,4-dichlorobenzyl)-1,4,5,6-tetrahydro-7H-indazol-7-ylidene)ethyl)(methyl)carbamoyl)benzenesulfonamide

(E)-4-chloro-N-((2-(1-(2,4-dichlorobenzyl)-1,4,5,6-tetrahydro-7H-indazol-7-ylidene)ethyl)(methyl)carbamoyl)benzenesulfonamide was prepared from (E)-2-(1-(2,4-dichlorobenzyl)-1,4,5,6-tetrahydro-7H-indazol-7-ylidene)-N-methylethan-1-amine according to the procedures as described in Example 6, Step 1.

¹H NMR (400 MHz, CHLOROFORM-d) δ: 7.99 (d, J=9.1 Hz, 2H), 7.44-7.58 (m, 2H), 7.39 (s, 1H), 7.33 (d, J=2.0 Hz, 1H), 7.11 (dd, J=8.1, 2.0 Hz, 1H), 6.52 (d, J=8.1 Hz, 1H), 5.44 (s, 2H), 5.10 (t, J=6.3 Hz, 1H), 3.94 (d, J=6.6 Hz, 2H), 2.60-2.69 (m, 5H), 2.32-2.48 (m, 2H), 1.64-1.89 (m, 2H). Mass spectrum (ESI, m/z): Calculated for C₂₄H₂₄Cl₃N₄O₃S: 553.1 (M+H), found: 553.0.

### Example 19, Compound #32

### (E)-2-(1-(2,4-dichlorobenzyl)-3-(difluoromethyl)-1,4,5,6-tetrahydro-7H-indazol-7-ylidene)ethyl ((4-chlorophenyl)sulfonyl)carbamate

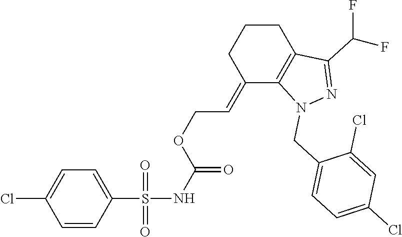 [SMILES: O=C(NS(=O)(=O)c1ccc(Cl)cc1)OC/C=C1\CCCc2c(C(F)F)nn(Cc3ccc(Cl)cc3Cl)c21]

### Step 1. Synthesis of 6-(2,2-difluoroacetyl)-2-ethoxycyclohex-2-en-1-one

To a solution of 2-ethoxycyclohex-2-en-1-one (1000 mg, 7.13 mmol) in THF (40 mL) at −78° C. was added LiHMDS (1.0 M in THF, 8.56 mL, 8.56 mmol). The reaction was stirred at −78° C. for 15 min before ethyl 2,2-difluoroacetate (0.975 mL, 9.27 mmol) was added. The reaction was kept at −78° C. for 30 min and was then warmed up to room temperature and stirred for 2 h. The reaction was quenched with aq. 1 N HCl, and the mixture was extracted with EtOAc. The organic layer was washed with sat. aq. NaCl, dried over Na₂SO₄ and concentrated to yield 6-(2,2-difluoroacetyl)-2-ethoxycyclohex-2-en-1-one.

### Step 2. Synthesis of 1-(2,4-dichlorobenzyl)-3-(difluoromethyl)-1,4,5,6-tetrahydro-7H-indazol-7-one

A mixture of 6-(2,2-difluoroacetyl)-2-ethoxycyclohex-2-en-1-one (778 mg, 3.57 mmol), (2,4-dichlorobenzyl)hydrazine hydrochloride (812 mg, 3.57 mmol), and H₂SO₄ (0.1 mL) in EtOH (10 mL) was heated at 70° C. for 30 min. The reaction was then cooled to room temperature and stirred overnight. The reaction mixture was concentrated. The resulting residue was diluted with EtOAc and washed with sat. aq. NaHCO₃ and sat. aq. NaCl. The organic solution was dried over Na₂SO₄ and concentrated. The resulting residue was purified by silica gel column with 0-20% EtOAc/heptane to yield 1-(2,4-dichlorobenzyl)-3-(difluoromethyl)-1,4,5,6-tetrahydro-7H-indazol-7-one. Mass spectrum (ESI, m/z): Calculated for C₁₅H₁₃Cl₂F₂N₂O: 345.0 (M+H), found: 345.0.

### Step 3. Synthesis of ethyl (E)-2-(1-(2,4-dichlorobenzyl)-3-(difluoromethyl)-1,4,5,6-tetrahydro-7H-indazol-7-ylidene)acetate

To a solution of ethyl 2-(diethoxyphosphoryl)acetate (568 mg, 2.90 mmol) in THF (12 mL) at 0° C. was added NaH (60% in mineral oil, 104 mg, 2.61 mmol). The reaction was warmed up to room temperature and stirred for 30 min. To the reaction was then added a solution of 1-(2,4-dichlorobenzyl)-3-(difluoromethyl)-1,4,5,6-tetrahydro-7H-indazol-7-one (250 mg, 0.724 mmol) in THF (4 mL). The reaction was heated at 50° C. overnight. The reaction was then quenched with aq. 1N HCl. The resulting mixture was extracted with EtOAc. The organic layer was washed with sat. aq. NaCl, dried over Na₂SO₄ and concentrated. The resulting residue was purified by silica gel column with 0-10% EtOAc/heptane to yield ethyl (E)-2-(1-(2,4-dichlorobenzyl)-3-(difluoromethyl)-1,4,5,6-tetrahydro-7H-indazol-7-ylidene)acetate.

### Step 4. Synthesis of (E)-2-(1-(2,4-dichlorobenzyl)-3-(difluoromethyl)-1,4,5,6-tetrahydro-7H-indazol-7-ylidene)ethan-1-ol

To a solution of ethyl (E)-2-(1-(2,4-dichlorobenzyl)-3-(difluoromethyl)-1,4,5,6-tetrahydro-7H-indazol-7-ylidene)acetate (238 mg, 0.573 mmol) in THF (8 mL) at −78° C. was added DIBAL-H (1.0 M in THF, 2.0 mL, 2.0 mmol). The reaction was kept at −78° C. for 1 h before the reaction mixture was warmed up to 0° C. and stirred for 45 min. Addition DIBAL-H (1.0 M in THF, 1.0 mL, 1.0 mmol) was added and the reaction was stirred at 0° C. for another 1.5 h. The reaction was then quenched with MeOH, followed by H₂O. The resulting mixture was diluted with EtOAc and washed with aq. 1 N HCl and sat. aq. NaCl. The organic solution was dried over Na₂SO₄ and concentrated to yield (E)-2-(1-(2,4-dichlorobenzyl)-3-(difluoromethyl)-1,4,5,6-tetrahydro-7H-indazol-7-ylidene)ethan-1-ol. Mass spectrum (ESI, m/z): Calculated for C₁₇H₁₇Cl₂F₂N₂O: 373.1 (M+H), found: 373.0.

### Step 5. Synthesis of (E)-2-(1-(2,4-dichlorobenzyl)-3-(difluoromethyl)-1,4,5,6-tetrahydro-7H-indazol-7-ylidene)ethyl ((4-chlorophenyl)sulfonyl)carbamate

(E)-2-(1-(2,4-dichlorobenzyl)-3-(difluoromethyl)-1,4,5,6-tetrahydro-7H-indazol-7-ylidene)ethyl ((4-chlorophenyl)sulfonyl)carbamate was prepared from (E)-2-(1-(2,4-dichlorobenzyl)-3-(difluoromethyl)-1,4,5,6-tetrahydro-7H-indazol-7-ylidene)ethan-1-ol according to the procedures as described in Example 2, Step 1.

¹H NMR (400 MHz, CHLOROFORM-d) δ: 7.78-8.07 (m, 2H), 7.45-7.60 (m, 2H), 7.38-7.45 (m, 1H), 7.16 (m, 1H), 6.48-6.55 (m, 1H), 5.45 (s, 2H), 5.27-5.33 (m, 1H), 4.67 (d, J=7.1 Hz, 2H), 2.78 (t, J=6.3 Hz, 2H), 2.33-2.43 (m, 2H), 1.85 (dt, J=12.3, 6.3 Hz, 2H). Mass spectrum (ESI, m/z): Calculated for C₂₄H₂₁Cl₃N₃O₄S: 590.0 (M+H), found: 590.1.

### Example 20, Compound #47

### (E)-4-chloro-N-((2-(1-(2,4-dichlorobenzyl)-3-(difluoromethyl)-1,4,5,6-tetrahydro-7H-indazol-7-ylidene)ethyl)carbamoyl)benzenesulfonamide

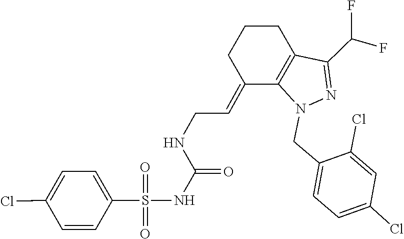 [SMILES: O=C(NC/C=C1\CCCc2c(C(F)F)nn(Cc3ccc(Cl)cc3Cl)c21)NS(=O)(=O)c1ccc(Cl)cc1]

### Steps 1-2. Synthesis of (E)-2-(1-(2,4-dichlorobenzyl)-3-(difluoromethyl)-1,4,5,6-tetrahydro-7H-indazol-7-ylidene)ethan-1-amine

(E)-2-(1-(2,4-dichlorobenzyl)-3-(difluoromethyl)-1,4,5,6-tetrahydro-7H-indazol-7-ylidene)ethan-1-amine was prepared from (E)-2-(1-(2,4-dichlorobenzyl)-3-(difluoromethyl)-1,4,5,6-tetrahydro-7H-indazol-7-ylidene)ethan-1-ol according to the procedures as described in Example 5, Steps 1-2.

### Step 3. Synthesis of (E)-4-chloro-N-((2-(1-(2,4-dichlorobenzyl)-1,4,5,6-tetrahydro-7H-indazol-7-ylidene)ethyl)carbamoyl)benzenesulfonamide

(E)-4-chloro-N-((2-(1-(2,4-dichlorobenzyl)-1,4,5,6-tetrahydro-7H-indazol-7-ylidene)ethyl)carbamoyl)benzenesulfonamide was prepared from (E)-2-(1-(2,4-dichlorobenzyl)-3-(difluoromethyl)-1,4,5,6-tetrahydro-7H-indazol-7-ylidene)ethan-1-amine according to the procedures as described in Example 6, Step 1.

¹H NMR (400 MHz, CHLOROFORM-d) δ: 7.85 (br d, J=8.1 Hz, 2H), 7.45-7.55 (m, 2H), 7.39-7.45 (m, 1H), 7.14-7.21 (m, 1H), 6.51-6.57 (m, 1H), 5.46 (s, 2H), 5.23 (br t, J=6.3 Hz, 1H), 3.78-3.91 (m, 2H), 2.71-2.80 (m, 2H), 2.40 (br s, 1H), 1.85 (br d, J=6.1 Hz, 2H). Mass spectrum (ESI, m/z): Calculated for C₂₄H₂₂Cl₃N₄O₃S: 589.0 (M+H), found: 589.0.

### Example 21, Compound #37

### (E)-2-(1-(2,4-dichlorobenzyl)-5,5-difluoro-1,4,5,6-tetrahydro-7H-indazol-7-ylidene)ethyl ((4-chlorophenyl)sulfonyl)carbamate

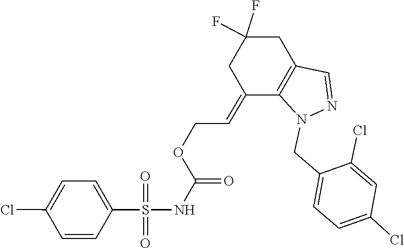 [SMILES: O=C(NS(=O)(=O)c1ccc(Cl)cc1)OC/C=C1\CC(F)(F)Cc2cnn(Cc3ccc(Cl)cc3Cl)c21]

### Step 1. Synthesis of ethyl (E)-2-(5,5-difluoro-2-oxocyclohexylidene)acetate

To a solution of 4,4-difluorocyclohexan-1-one (1060 mg, 7.90 mmol) in THF (40 mL) at −78° C. was added LiHMDS (1.0 M in THF, 9.48 mL, 9.48 mmol). The reaction was kept at −78° C. for 30 min before ethyl 2-oxoacetate (50% in toluene, 1.88 mL, 9.48 mmol) was added. The reaction was stirred at −78° C. for another 1 h. The reaction was then quenched with aq. 1N HCl, and the resulting mixture was extracted with EtOAc. The organic layer was washed with sat. aq. NaCl, dried over Na₂SO⁴, and concentrated. The resulting residue was dissolved in CH₂Cl₂ (50 mL), and the solution was cooled to 0° C. Pyridine (2.55 mL, 31.6 mmol) was added, followed by SOCl₂ (0.86 mL, 11.9 mmol). The reaction was warmed up to room temperature and stirred overnight. The reaction was concentrated, and the residue was diluted with EtOAc and washed with sat. aq. NaCl. The organic solution was dried over Na₂SO₄ and concentrated. The resulting residue was purified by silica gel column with 10% EtOAc/heptane to yield ethyl (E)-2-(5,5-difluoro-2-oxocyclohexylidene)acetate.

### Step 2. Synthesis of ethyl (E)-2-((E)-5,5-difluoro-3-(hydroxymethylene)-2-oxocyclohexylidene)acetate

Ethyl (E)-2-((E)-5,5-difluoro-3-(hydroxymethylene)-2-oxocyclohexylidene)acetate was prepared from ethyl (E)-2-(5,5-difluoro-2-oxocyclohexylidene)acetate according to the procedures as described in Example 1, Step 2.

### Step 3. Synthesis of ethyl (E)-2-(1-(2,4-dichlorobenzyl)-5,5-difluoro-1,4,5,6-tetrahydro-7H-indazol-7-ylidene)acetate

Ethyl (E)-2-(1-(2,4-dichlorobenzyl)-5,5-difluoro-1,4,5,6-tetrahydro-7H-indazol-7-ylidene)acetate was prepared from ethyl (E)-2-((E)-5,5-difluoro-3-(hydroxymethylene)-2-oxocyclohexylidene)acetate according to the procedures as described in Example 1, Step 3. Mass spectrum (ESI, m/z): Calculated for C₁₈H₁₇Cl₂F₂N₂O₂: 401.1 (M+H), found: 401.1.

### Step 4. Synthesis of (E)-2-(1-(2,4-dichlorobenzyl)-5,5-difluoro-1,4,5,6-tetrahydro-7H-indazol-7-ylidene)ethan-1-ol

To a solution of ethyl (E)-2-(1-(2,4-dichlorobenzyl)-5,5-difluoro-1,4,5,6-tetrahydro-7H-indazol-7-ylidene)acetate (66 mg, 0.164 mmol) in CH₂Cl₂ (3 mL) and toluene (3 mL) at −78° C. was added DIBAL-H (1.0 M in heptane, 0.822 mL, 0.822 mmol). The reaction was kept at −78° C. for 3 h. The reaction was then quenched with MeOH and H₂O. The resulting mixture was diluted with EtOAc and washed with aq. 1N HCl and sat. aq. NaC. The organic layer was dried over Na₂SO₄ and concentrated. The resulting residue was purified by silica gel column with 30% EtOAc/heptane to yield (E)-2-(1-(2,4-dichlorobenzyl)-5,5-difluoro-1,4,5,6-tetrahydro-7H-indazol-7-ylidene)ethan-1-ol. Mass spectrum (ESI, m/z): Calculated for C₁₆H₁₅Cl₂F₂N₂O: 359.0 (M+H), found: 359.0.

### Step 5. Synthesis of (E)-2-(1-(2,4-dichlorobenzyl)-5,5-difluoro-1,4,5,6-tetrahydro-7H-indazol-7-ylidene)ethyl ((4-chlorophenyl)sulfonyl)carbamate

(E)-2-(1-(2,4-dichlorobenzyl)-5,5-difluoro-1,4,5,6-tetrahydro-7H-indazol-7-ylidene)ethyl ((4-chlorophenyl)sulfonyl)carbamate was prepared from (E)-2-(1-(2,4-dichlorobenzyl)-5,5-difluoro-1,4,5,6-tetrahydro-7H-indazol-7-ylidene)ethan-1-ol according to the procedures as described in Example 2, Step 1.

¹H NMR (400 MHz, CHLOROFORM-d) δ: 7.85-8.00 (m, 2H), 7.43-7.57 (m, 3H), 7.41 (d, J=2.0 Hz, 1H), 7.14 (dd, J=8.3, 2.3 Hz, 1H), 6.51 (d, J=8.6 Hz, 1H), 5.45-5.54 (m, 3H), 4.66 (d, J=7.1 Hz, 2H), 3.18 (t, J=13.6 Hz, 2H), 2.90 (t, J=13.9 Hz, 2H). Mass spectrum (ESI, m/z): Calculated for C₂₃H₁₉Cl₃F₂N₃O₄S: 576.0 (M+H), found: 578.0.

### Example 22, Compound #48

### (E)-4-chloro-N-((2-(1-(2,4-dichlorobenzyl)-5,5-difluoro-1,4,5,6-tetrahydro-7H-indazol-7-ylidene)ethyl)carbamoyl)benzenesulfonamide

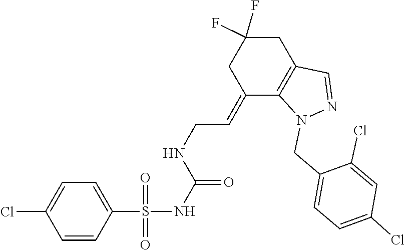 [SMILES: O=C(NC/C=C1\CC(F)(F)Cc2cnn(Cc3ccc(Cl)cc3Cl)c21)NS(=O)(=O)c1ccc(Cl)cc1]

### Steps 1-2. Synthesis of (E)-2-(1-(2,4-dichlorobenzyl)-5,5-difluoro-1,4,5,6-tetrahydro-7H-indazol-7-ylidene)ethan-1-amine

(E)-2-(1-(2,4-dichlorobenzyl)-5,5-difluoro-1,4,5,6-tetrahydro-7H-indazol-7-ylidene)ethan-1-amine was prepared from (E)-2-(1-(2,4-dichlorobenzyl)-5,5-difluoro-1,4,5,6-tetrahydro-7H-indazol-7-ylidene)ethan-1-01 according to the procedures as described in Example 5, Steps 1-2.

### Step 3. Synthesis of (E)-4-chloro-N-((2-(1-(2,4-dichlorobenzyl)-5,5-difluoro-1,4,5,6-tetrahydro-7H-indazol-7-ylidene)ethyl)carbamoyl)benzenesulfonamide

(E)-4-chloro-N-((2-(1-(2,4-dichlorobenzyl)-5,5-difluoro-1,4,5,6-tetrahydro-7H-indazol-7-ylidene)ethyl)carbamoyl)benzenesulfonamide was prepared from (E)-2-(1-(2,4-dichlorobenzyl)-5,5-difluoro-1,4,5,6-tetrahydro-7H-indazol-7-ylidene)ethan-1-amine according to the procedures as described in Example 6, Step 1.

¹H NMR (400 MHz, CHLOROFORM-d) δ: 7.82-7.90 (m, 2H), 7.39-7.53 (m, 4H), 7.15 (dd, J=8.3, 2.3 Hz, 1H), 6.53 (d, J=8.6 Hz, 1H), 5.42-5.52 (m, 3H), 3.85 (d, J=7.1 Hz, 2H), 3.17 (t, J=13.6 Hz, 2H), 2.82-3.04 (m, 2H). Mass spectrum (ESI, m/z): Calculated for C₂₃H₂₀Cl₃F₂N₄O₃S: 575.0 (M+H), found: 575.0.

The following representative compound of formula (I) was similarly prepared according to the procedure described in Example 22 (and Examples referenced therein) above, selecting and substituting suitable starting materials and reagents, as would be readily recognized by those skilled in the art.

### (E)-4-chloro-N-((2-(5,5-difluoro-1-(3-(trifluoromethyl)phenyl)-1,4,5,6-tetrahydro-7H-indazol-7-ylidene)ethyl)carbamoyl)benzenesulfonamide, Compound #70

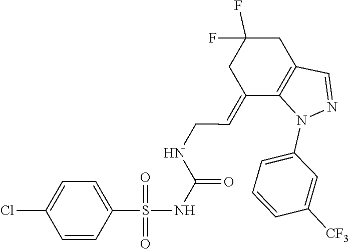 [SMILES: O=C(NC/C=C1\CC(F)(F)Cc2cnn(-c3cccc(C(F)(F)F)c3)c21)NS(=O)(=O)c1ccc(Cl)cc1]

¹H NMR (400 MHz, CHLOROFORM-d) δ: 7.57-7.73 (m, 7H), 7.37-7.46 (m, 2H), 6.40 (br t, J=5.3 Hz, 1H), 5.22 (br t, J=7.3 Hz, 1H), 3.83 (dd, J=7.1, 5.6 Hz, 2H), 3.23 (t, J=13.6 Hz, 2H), 3.04 (br t, J=13.6 Hz, 2H). Mass spectrum (ESI, m/z): Calculated for C₂₃H₁₉ClF₅N₄O₃S: 561.1 (M+H), found: 561.2.

### Example 23, Compound #50 and Compound #51

### (Z)-2-(1-(2,4-dichlorobenzyl)-6-fluoro-1,4,5,6-tetrahydro-7H-indazol-7-ylidene)ethyl ((4-chlorophenyl)sulfonyl)carbamate and

### (E)-2-(1-(2,4-dichlorobenzyl)-6-fluoro-1,4,5,6-tetrahydro-7H-indazol-7-ylidene)ethyl ((4-chlorophenyl)sulfonyl)carbamate

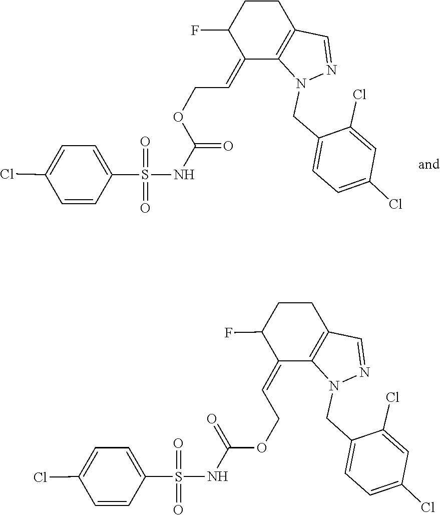 [SMILES: O=C(NS(=O)(=O)c1ccc(Cl)cc1)OC/C=C1/c2c(cnn2Cc2ccc(Cl)cc2Cl)CCC1F ; O=C(NS(=O)(=O)c1ccc(Cl)cc1)OC/C=C1\c2c(cnn2Cc2ccc(Cl)cc2Cl)CCC1F]

### Step 1. Synthesis of 1-(2,4-dichlorobenzyl)-6-fluoro-1,4,5,6-tetrahydro-7H-indazol-7-one

To a solution of 1-(2,4-dichlorobenzyl)-1,4,5,6-tetrahydro-7H-indazol-7-one (142 mg, 0.481 mmol) in THF (6 mL) at −78° C. was added LiHMDS (1.0 M in THF, 0.577 mL, 0.577 mmol). The reaction was kept at −78° C. for 30 min before a solution of N-fluoro-N-(phenylsulfonyl)benzenesulfonamide (212 mg, 0.674 mmol) in THF (2 mL) was added. The reaction was stirred at −78° C. for 1.5 h. The reaction was then quenched with aq. NaH₂PO₄ and extracted with EtOAc. The organic layer was washed with sat. aq. NaCl, dried over Na₂SO₄ and concentrated. Purification of the resulting residue by silica gel column with 20% EtOAc/heptane yielded 1-(2,4-dichlorobenzyl)-6-fluoro-1,4,5,6-tetrahydro-7H-indazol-7-one. Mass spectrum (ESI, m/z): Calculated for C₁₄H₁₂Cl₂FNO₂: 313.0 (M+H), found: 313.0.

### Step 2. Synthesis of ethyl (Z)-2-(1-(2,4-dichlorobenzyl)-6-fluoro-1,4,5,6-tetrahydro-7H-indazol-7-ylidene)acetate and ethyl (E)-2-(1-(2,4-dichlorobenzyl)-6-fluoro-1,4,5,6-tetrahydro-7H-indazol-7-ylidene)acetate

To a solution of ethyl 2-(diethoxyphosphoryl)acetate (522 mg, 2.66 mmol) in THF (6 mL) at 0° C. was added NaH (60% in mineral oil, 97 mg, 2.43 mmol). The reaction was warmed up to room temperature and stirred for 30 min. To the reaction was added a solution of 1-(2,4-dichlorobenzyl)-6-fluoro-1,4,5,6-tetrahydro-7H-indazol-7-one (238 mg, 0.76 mmol) in THF (3 mL). The reaction was stirred at room temperature for 1 h before the reaction was quenched with aq 1N HCl. The resulting mixture was extracted with EtOAc. The organic layer was washed with sat. aq. NaCl, dried over Na₂SO₄, and concentrated. Purification of the resulting residue by silica gel column with 10% EtOAc/heptane yielded ethyl (Z)-2-(1-(2,4-dichlorobenzyl)-6-fluoro-1,4,5,6-tetrahydro-7H-indazol-7-ylidene)acetate followed by ethyl (E)-2-(1-(2,4-dichlorobenzyl)-6-fluoro-1,4,5,6-tetrahydro-7H-indazol-7-ylidene)acetate.

### Step 3. Synthesis of (Z)-2-(1-(2,4-dichlorobenzyl)-6-fluoro-1,4,5,6-tetrahydro-7H-indazol-7-ylidene)ethan-1-ol

(Z)-2-(1-(2,4-dichlorobenzyl)-6-fluoro-1,4,5,6-tetrahydro-7H-indazol-7-ylidene)ethan-1-ol was prepared from ethyl (Z)-2-(1-(2,4-dichlorobenzyl)-6-fluoro-1,4,5,6-tetrahydro-7H-indazol-7-ylidene)acetate according to the procedures as described in Example 21, Step 4.

### Step 4. Synthesis of (Z)-2-(1-(2,4-dichlorobenzyl)-6-fluoro-1,4,5,6-tetrahydro-7H-indazol-7-ylidene)ethyl ((4-chlorophenyl)sulfonyl)carbamate

(Z)-2-(1-(2,4-dichlorobenzyl)-6-fluoro-1,4,5,6-tetrahydro-7H-indazol-7-ylidene)ethyl ((4-chlorophenyl)sulfonyl)carbamate was prepared from (Z)-2-(1-(2,4-dichlorobenzyl)-6-fluoro-1,4,5,6-tetrahydro-7H-indazol-7-ylidene)ethan-1-ol according to the procedures as described in Example 2, Step 1.

¹H NMR (400 MHz, CHLOROFORM-d) δ: 7.89-8.07 (m, 2H), 7.43-7.56 (m, 3H), 7.40 (d, J=2.0 Hz, 1H), 7.13 (dd, J=8.6, 2.0 Hz, 1H), 6.50 (d, J=8.6 Hz, 1H), 5.41-5.71 (m, 4H), 4.84-4.96 (m, 1H), 4.67 (ddd, J=13.3, 5.9, 3.0 Hz, 1H), 2.65-2.85 (m, 2H), 2.32-2.55 (m, 1H), 1.70-1.93 (m, 1H). Mass spectrum (ESI, m/z): Calculated for C₂₃H₂₀Cl₃FN₃O₄S: 558.0 (M+H), found: 558.0.

### Step 5. Synthesis of (E)-2-(1-(2,4-dichlorobenzyl)-6-fluoro-1,4,5,6-tetrahydro-7H-indazol-7-ylidene)ethan-1-ol

(E)-2-(1-(2,4-dichlorobenzyl)-6-fluoro-1,4,5,6-tetrahydro-7H-indazol-7-ylidene)ethan-1-ol was prepared from ethyl (E)-2-(1-(2,4-dichlorobenzyl)-6-fluoro-1,4,5,6-tetrahydro-7H-indazol-7-ylidene)acetate according to the procedures as described in Example 21, Step 4.

### Step 6. Synthesis of (E)-2-(1-(2,4-dichlorobenzyl)-6-fluoro-1,4,5,6-tetrahydro-7H-indazol-7-ylidene)ethyl ((4-chlorophenyl)sulfonyl)carbamate

(E)-2-(1-(2,4-dichlorobenzyl)-6-fluoro-1,4,5,6-tetrahydro-7H-indazol-7-ylidene)ethyl ((4-chlorophenyl)sulfonyl)carbamate was prepared from (E)-2-(1-(2,4-dichlorobenzyl)-6-fluoro-1,4,5,6-tetrahydro-7H-indazol-7-ylidene)ethan-1-ol according to the procedures as described in Example 2, Step 1.

¹H NMR (400 MHz, CHLOROFORM-d) δ: 7.55-8.28 (m, 2H), 7.45-7.55 (m, 3H), 7.23-7.30 (m, 2H), 7.10-7.19 (m, 1H), 6.71 (d, J=8.6 Hz, 1H), 5.75 (t, J=7.1 Hz, 1H), 5.08-5.25 (m, 3H), 4.61 (br d, J=7.1 Hz, 2H), 2.68-2.89 (m, 2H), 2.23-2.34 (m, 1H), 1.98-2.21 (m, 1H). Mass spectrum (ESI, m/z): Calculated for C₂₃H₂₀Cl₃FN₃O₄S: 558.0 (M+H), found: 558.0.

### Example 24, Compound #73

### (Z)-4-chloro-N-((2-(1-(2,4-dichlorobenzyl)-6-fluoro-1,4,5,6-tetrahydro-7H-indazol-7-ylidene)ethyl)carbamoyl)benzenesulfonamide

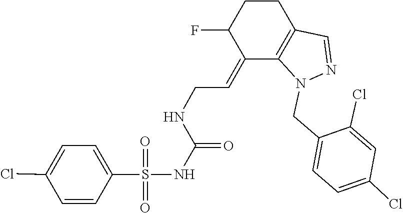 [SMILES: O=C(NC/C=C1/c2c(cnn2Cc2ccc(Cl)cc2Cl)CCC1F)NS(=O)(=O)c1ccc(Cl)cc1]

(Z)-4-chloro-N-((2-(1-(2,4-dichlorobenzyl)-6-fluoro-1,4,5,6-tetrahydro-7H-indazol-7-ylidene)ethyl)carbamoyl)benzenesulfonamide was prepared from (Z)-2-(1-(2,4-dichlorobenzyl)-6-fluoro-1,4,5,6-tetrahydro-7H-indazol-7-ylidene)ethan-1-ol according to the procedures as described in Example 22, Steps 1-3.

¹H NMR (400 MHz, CHLOROFORM-d) δ: 7.82-7.89 (m, 2H), 7.39-7.53 (m, 4H), 7.14 (dd, J=8.6, 2.0 Hz, 1H), 6.50 (d, J=8.1 Hz, 1H), 5.59-5.78 (m, 1H), 5.44-5.52 (m, 3H), 4.11 (ddd, J=15.8, 7.5, 3.5 Hz, 1H), 3.84 (ddd, J=15.7, 6.6, 3.5 Hz, 1H), 2.56-2.83 (m, 2H), 2.30-2.55 (m, 1H), 1.63-1.92 (m, 1H). Mass spectrum (ESI, m/z): Calculated for C₂₃H₂₁Cl₃FN₄O₃S: 557.0 (M+H), found: 557.0.

### Example 25: Compound #82 and Compound #81

### (Z)-2-(1-(2,4-dichlorobenzyl)-6,6-difluoro-1,4,5,6-tetrahydro-7H-indazol-7-ylidene)ethyl ((4-chlorophenyl)sulfonyl)carbamate and

### (E)-2-(1-(2,4-dichlorobenzyl)-6,6-difluoro-1,4,5,6-tetrahydro-7H-indazol-7-ylidene)ethyl ((4-chlorophenyl)sulfonyl)carbamate

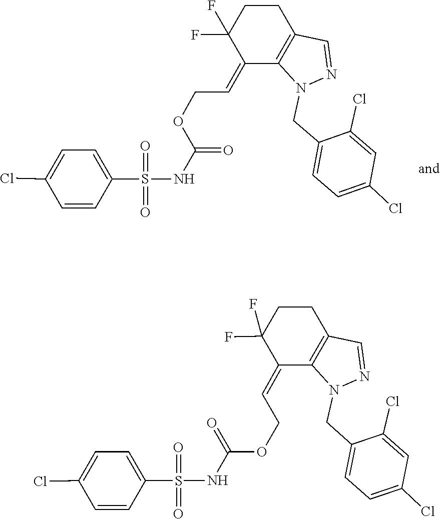 [SMILES: O=C(NS(=O)(=O)c1ccc(Cl)cc1)OC/C=C1/c2c(cnn2Cc2ccc(Cl)cc2Cl)CCC1(F)F ; O=C(NS(=O)(=O)c1ccc(Cl)cc1)OC/C=C1\c2c(cnn2Cc2ccc(Cl)cc2Cl)CCC1(F)F]

### Step 1. Synthesis of 1-(2,4-dichlorobenzyl)-6,6-difluoro-1,4,5,6-tetrahydro-7H-indazol-7-one

To a solution of 1-(2,4-dichlorobenzyl)-6-fluoro-1,4,5,6-tetrahydro-7H-indazol-7-one (50 mg, 0.16 mmol) in THF (3 mL) at −78° C. was added LiHMDS (1.0 M in THF, 0.192 mL, 0.192 mmol). The reaction was kept at −78° C. for 30 min before a solution of N-fluoro-N-(phenylsulfonyl)benzenesulfonamide (75.5 mg, 0.239 mmol) in THF (1 mL) was added. The reaction was stirred at −78° C. for 3 h. The reaction was then quenched with aq. 1N HCl and extracted with EtOAc. The organic layer was washed with sat. aq. NaCl, dried over Na₂SO₄, and concentrated. The resulting residue was purified by silica gel column with 10-20% EtOAc/heptane to yield impure 1-(2,4-dichlorobenzyl)-6,6-difluoro-1,4,5,6-tetrahydro-7H-indazol-7-one, which was suspended in heptane and filtered (repeated 3 times). The solutions were combined and concentrated to yield 1-(2,4-dichlorobenzyl)-6,6-difluoro-1,4,5,6-tetrahydro-7H-indazol-7-one. Mass spectrum (ESI, m/z): Calculated for C₁₄H₁₁Cl₂F₂N₂O: 331.0 (M+H), found: 331.0.

### Step 2. Synthesis of ethyl (Z)-2-(1-(2,4-dichlorobenzyl)-6,6-difluoro-1,4,5,6-tetrahydro-7H-indazol-7-ylidene)acetate and ethyl (E)-2-(1-(2,4-dichlorobenzyl)-6,6-difluoro-1,4,5,6-tetrahydro-7H-indazol-7-ylidene)acetate

A mixture of ethyl (Z)-2-(1-(2,4-dichlorobenzyl)-6,6-difluoro-1,4,5,6-tetrahydro-7H-indazol-7-ylidene)acetate and ethyl (E)-2-(1-(2,4-dichlorobenzyl)-6,6-difluoro-1,4,5,6-tetrahydro-7H-indazol-7-ylidene)acetate was prepared according to the procedures as described in Example 23, Step 2.

### Step 3. Synthesis of (Z)-2-(1-(2,4-dichlorobenzyl)-6,6-difluoro-1,4,5,6-tetrahydro-7H-indazol-7-ylidene)ethan-1-ol and (E)-2-(1-(2,4-dichlorobenzyl)-6,6-difluoro-1,4,5,6-tetrahydro-7H-indazol-7-ylidene)ethan-1-ol

A mixture of ethyl (Z)-2-(1-(2,4-dichlorobenzyl)-6,6-difluoro-1,4,5,6-tetrahydro-7H-indazol-7-ylidene)acetate and ethyl (E)-2-(1-(2,4-dichlorobenzyl)-6,6-difluoro-1,4,5,6-tetrahydro-7H-indazol-7-ylidene)acetate (27 mg, 0.0673 mmol) was dissolved in CH₂Cl₂ (2 mL). The resulting solution was cooled to −78° C. and DIBAL-H (1.0 M in heptane, 0.336 mL, 0.336 mmol) was added. The reaction was stirred at −78° C. for 2 h. To the reaction was added MeOH and the resulting solution was warmed up to 0° C. NaBH₄ was added and the reaction was kept at 0° C. for 20 min. The reaction mixture was concentrated and to the residue was added aq 1N HCl. The resulting mixture was extracted with CH₂Cl₂. The organic layer was washed with sat. aq. NaCl, dried over Na₂SO₄ and concentrated. Purification of the resulting residue by silica gel column with 10-40% EtOAc/heptane yielded (Z)-2-(1-(2,4-dichlorobenzyl)-6,6-difluoro-1,4,5,6-tetrahydro-7H-indazol-7-ylidene)ethan-1-ol followed by (E)-2-(1-(2,4-dichlorobenzyl)-6,6-difluoro-1,4,5,6-tetrahydro-7H-indazol-7-ylidene)ethan-1-ol.

### Step 4. (Z)-2-(1-(2,4-dichlorobenzyl)-6,6-difluoro-1,4,5,6-tetrahydro-7H-indazol-7-ylidene)ethyl ((4-chlorophenyl)sulfonyl)carbamate

(Z)-2-(1-(2,4-dichlorobenzyl)-6,6-difluoro-1,4,5,6-tetrahydro-7H-indazol-7-ylidene)ethyl ((4-chlorophenyl)sulfonyl)carbamate was prepared from (Z)-2-(1-(2,4-dichlorobenzyl)-6,6-difluoro-1,4,5,6-tetrahydro-7H-indazol-7-ylidene)ethan-1-ol according to the procedures as described in Example 2, Step 1.

¹H NMR (CHLOROFORM-d) δ: 7.93-8.00 (m, 2H), 7.39-7.57 (m, 4H), 7.15 (dd, J=8.1, 2.0 Hz, 1H), 6.52 (d, J=8.6 Hz, 1H), 5.47-5.56 (m, 1H), 5.41-5.50 (m, 2H), 4.93-5.03 (m, 2H), 2.76 (t, J=6.6 Hz, 2H), 2.17-2.37 (m, 2H). Mass spectrum (ESI, m/z): Calculated for C₂₃H₁₉Cl₃F₂N₃O₄S: 576.0 (M+H), found: 576.0.

### Step 5. Synthesis of (E)-2-(1-(2,4-dichlorobenzyl)-6,6-difluoro-1,4,5,6-tetrahydro-7H-indazol-7-ylidene)ethyl ((4-chlorophenyl)sulfonyl)carbamate

(E)-2-(1-(2,4-dichlorobenzyl)-6,6-difluoro-1,4,5,6-tetrahydro-7H-indazol-7-ylidene)ethyl ((4-chlorophenyl)sulfonyl)carbamate was prepared from (E)-2-(1-(2,4-dichlorobenzyl)-6,6-difluoro-1,4,5,6-tetrahydro-7H-indazol-7-ylidene)ethan-1-ol according to the procedures as described in Example 2, Step 1.

¹H NMR (CHLOROFORM-d) δ: 7.90-7.99 (m, 2H), 7.44-7.57 (m, 3H), 7.30 (d, J=2.0 Hz, 1H), 7.15 (dd, J=8.1, 2.0 Hz, 1H), 6.73 (d, J=8.1 Hz, 1H), 6.05 (t, J=6.8 Hz, 1H), 5.15 (s, 2H), 4.68 (br d, J=7.1 Hz, 2H), 2.84 (t, J=6.8 Hz, 2H), 2.34 (tt, J=13.6, 6.6 Hz, 2H). Mass spectrum (ESI, m/z): Calculated for C₂₃H₁₉Cl₃F₂N₃O₄S: 576.0 (M+H), found: 576.0.

### Example 26: Compound #100

### (E)-4,5-dichloro-N-((2-(1-(2,4-dichlorobenzyl)-6-fluoro-1,4,5,6-tetrahydro-7H-indazol-7-ylidene)-2-fluoroethyl)carbamoyl)thiophene-2-sulfonamide

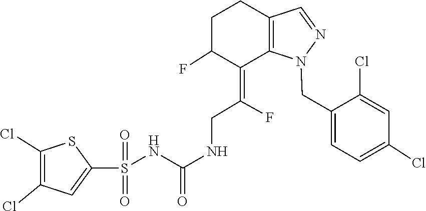 [SMILES: O=C(NC/C(F)=C1/c2c(cnn2Cc2ccc(Cl)cc2Cl)CCC1F)NS(=O)(=O)c1cc(Cl)c(Cl)s1]

### Step 1. Synthesis of ethyl (E)-2-(1-(2,4-dichlorobenzyl)-6-fluoro-1,4,5,6-tetrahydro-7H-indazol-7-ylidene)-2-fluoroacetate

To a solution of ethyl 2-(diethoxyphosphoryl)-2-fluoroacetate (815 mg, 3.37 mmol) in THF (20 ml) at −78° C. under nitrogen was added n-BuLi (1.3 ml, 3.25 mmol) dropwise with stirring. The reaction was stirred at −78° C. for 2 h before 1-(2,4-dichlorobenzyl)-6-fluoro-5,6-dihydro-1H-indazol-7(4H)-one (350 mg, 1.12 mmol) was added. The reaction was slowly warmed up to room temperature and stirred overnight. The reaction was then quenched by the addition of H₂O. The resulting mixture was extracted with ethyl acetate (3×50 mL) and the organic layers were combined and concentrated under vacuum. The residue was purified by silica gel column with ethyl acetate/petroleum ether (5/95) to yield ethyl (E)-2-(1-(2,4-dichlorobenzyl)-6-fluoro-1,4,5,6-tetrahydro-7H-indazol-7-ylidene)-2-fluoroacetate as a yellow solid. Mass spectrum (ESI, m/z): Calculated for C₁₈H₁₇Cl₂F₂N₂O₂, 401.1 (M+H), found 400.9.

### Step 2. Synthesis of (E)-2-(1-(2,4-dichlorobenzyl)-6-fluoro-1,4,5,6-tetrahydro-7H-indazol-7-ylidene)-2-fluoroacetic acid

A mixture of ethyl (E)-2-(1-(2,4-dichlorobenzyl)-6-fluoro-1,4,5,6-tetrahydro-7H-indazol-7-ylidene)-2-fluoroacetate (500 mg, 1.25 mmol) and NaOH (150 mg, 3.75 mmol) in THF (10 ml), MeOH (10 ml), and H₂O (5 ml) was stirred at room temperature for 1 h. The reaction mixture was concentrated under vacuum. The resulting residue was diluted with water and extracted with ether (3×10 mL). The aqueous layer was adjusted to pH=3 with aq. 2 N HCl. The resulting mixture was extracted with ethyl acetate (3×10 mL). The organic layers were combined, dried over anhydrous sodium sulfate, and concentrated under vacuum to yield (E)-2-(1-(2,4-dichlorobenzyl)-6-fluoro-1,4,5,6-tetrahydro-7H-indazol-7-ylidene)-2-fluoroacetic acid as a yellow oil. Mass spectrum (ESI, m/z): Calculated for C₁₆H₁₃Cl₂F₂N₂O₂, 373.0 (M+H), found 372.8.

### Step 3. Synthesis of (E)-2-(1-(2,4-dichlorobenzyl)-6-fluoro-1,4,5,6-tetrahydro-7H-indazol-7-ylidene)-2-fluoroethan-1-ol

To a solution of (E)-2-(1-(2,4-dichlorobenzyl)-6-fluoro-1,4,5,6-tetrahydro-7H-indazol-7-ylidene)-2-fluoroacetic acid (150 mg, 0.402 mmol) in THF (10 ml) at 0° C. was added 4-methylmorpholine (45 mg, 0.445 mmol, 1.1 equiv) followed by isobutyl carbonochloridate (55 mg, 0.403 mmol). The reaction was stirred at 0° C. for 15 min before a solution of NaBH₄ (61 mg, 1.61 mmol) in H₂O (0.5 ml) was added. The reaction was stirred at 0° C. for another 30 min and was then quenched with MeOH. The resulting mixture was concentrated under vacuum. The residue was purified by silica gel column with ethyl acetate/petroleum ether (30/70) to yield (E)-2-(1-(2,4-dichlorobenzyl)-6-fluoro-1,4,5,6-tetrahydro-7H-indazol-7-ylidene)-2-fluoroethan-1-ol as a yellow oil. Mass spectrum (ESI, m/z): Calculated for C₁₈H₁₅Cl₂F₂N₂O, 359.0 (M+H), found 358.9.

### Step 4. Synthesis of (E)-2-(2-(1-(2,4-dichlorobenzyl)-6-fluoro-1,4,5,6-tetrahydro-7H-indazol-7-ylidene)-2-fluoroethyl)isoindoline-1,3-dione

(E)-2-(2-(1-(2,4-dichlorobenzyl)-6-fluoro-1,4,5,6-tetrahydro-7H-indazol-7-ylidene)-2-fluoroethyl)isoindoline-1,3-dione was prepared from (E)-2-(1-(2,4-dichlorobenzyl)-6-fluoro-1,4,5,6-tetrahydro-7H-indazol-7-ylidene)-2-fluoroethan-1-ol according to the procedures as described in Example 5, Step 1.

### Step 5. Synthesis of (E)-2-(1-(2,4-dichlorobenzyl)-6-fluoro-1,4,5,6-tetrahydro-7H-indazol-7-ylidene)-2-fluoroethan-1-amine

(E)-2-(1-(2,4-dichlorobenzyl)-6-fluoro-1,4,5,6-tetrahydro-7H-indazol-7-ylidene)-2-fluoroethan-1-amine was prepared from (E)-2-(2-(1-(2,4-dichlorobenzyl)-6-fluoro-1,4,5,6-tetrahydro-7H-indazol-7-ylidene)-2-fluoroethyl)isoindoline-1,3-dione according to the procedures as described in Example 5, Step 2.

### Step 6. Synthesis of (E)-4,5-dichloro-N-((2-(1-(2,4-dichlorobenzyl)-6-fluoro-1,4,5,6-tetrahydro-7H-indazol-7-ylidene)-2-fluoroethyl)carbamoyl)thiophene-2-sulfonamide

(E)-4,5-dichloro-N-((2-(1-(2,4-dichlorobenzyl)-6-fluoro-1,4,5,6-tetrahydro-7H-indazol-7-ylidene)-2-fluoroethyl)carbamoyl)thiophene-2-sulfonamide was prepared from (E)-2-(1-(2,4-dichlorobenzyl)-6-fluoro-1,4,5,6-tetrahydro-7H-indazol-7-ylidene)-2-fluoroethan-1-amine according to the procedures as described in Example 5, Step 3.

¹H NMR (300 MHz, CD₃OD) δ: 7.65 (s, 1H), 7.50 (s, 1H), 7.43 (s, 1H), 7.20-7.23 (m, 1H), 6.68 (d, J=8.4 Hz, 1H), 5.91 (d, J=48.9 Hz, 1H), 5.33-5.47 (m, 2H), 4.31-4.46 (m, 1H), 3.92-4.03 (m, 1H), 2.74-2.79 (m, 2H), 2.39-2.45 (m, 1H), 1.74-1.96 (m, 1H). ¹⁹F NMR (300 MHz, CD₃OD) δ: −101.85, −180.79. Mass spectrum (ESI, m/z): Calculated for C₂₁H₁₇Cl₄F₂N₄O₃S₂, 614.9 (M+H), found 616.8.

The following representative compounds of formula (I) were similarly prepared according to the procedure described in Example 26 (and Examples referenced therein) above, selecting and substituting suitable starting materials and reagents, as would be readily recognized by those skilled in the art.

### (E)-4-chloro-N-((2-(1-(2,4-dichlorobenzyl)-6-fluoro-1,4,5,6-tetrahydro-7H-indazol-7-ylidene)-2-fluoroethyl)carbamoyl)benzenesulfonamide, Compound #101

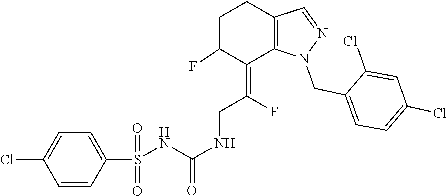 [SMILES: O=C(NC/C(F)=C1/c2c(cnn2Cc2ccc(Cl)cc2Cl)CCC1F)NS(=O)(=O)c1ccc(Cl)cc1]

¹H NMR (300 MHz, CD₃OD) δ: 7.90-7.93 (m, 2H), 7.44-7.56 (m, 4H), 7.20-7.24 (m, 1H), 6.66 (d, J=8.4 Hz, 1H), 5.83 (d, J=51 Hz, 1H), 5.31-5.39 (m, 2H), 4.26-4.41 (m, 1H), 3.87-3.96 (m, 1H), 2.70-2.75 (m, 2H), 2.31-2.36 (m, 1H), 1.59-1.97 (m, 1H). ¹⁹F NMR (300 MHz, CD₃OD) δ: −102.12, −180.84. Mass spectrum (ESI, m/z): Calculated for C₂₃H₂₀Cl₃F₂N₄O₃S, 575.0 (M+H), found 576.8.

### (E)-5-chloro-N-((2-(1-(2,4-dichlorobenzyl)-6-fluoro-1,4,5,6-tetrahydro-7H-indazol-7-ylidene)-2-fluoroethyl)carbamoyl)-2-methoxybenzenesulfonamide, Compound #103

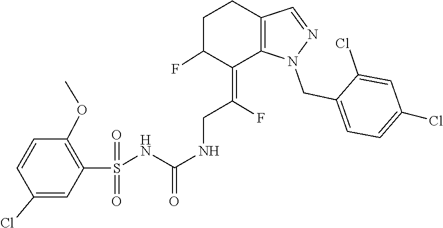 [SMILES: COc1ccc(Cl)cc1S(=O)(=O)NC(=O)NC/C(F)=C1/c2c(cnn2Cc2ccc(Cl)cc2Cl)CCC1F]

¹H NMR (400 MHz, CD₃OD) δ: 7.85 (s, 1H), 7.51-7.52 (m, 1H), 7.44-7.50 (m, 2H), 7.22 (d, J=8.8 Hz, 1H), 7.07 (d, J=8.8 Hz, 1H), 6.64 (d, J=8.0 Hz, 1H), 5.80 (d, J=48.8 Hz, 1H), 5.31-5.41 (m, 2H), 4.29-4.40 (m, 1H), 3.85-3.93 (m, 1H), 3.83 (s, 3H), 2.63-2.74 (m, 2H), 2.28-2.30 (m, 1H), 1.47-1.66 (m, 1H). ¹⁹F NMR (400 MHz, CD₃OD) δ: −102.97, −181.27. Mass spectrum (ESI, m/z): Calculated for C₂₄H₂₂Cl₃F₂N₄O₄S, 605.0 (M+H), found 606.8.

### Example 27: Compound #109

### (Z)-4,5-dichloro-N-((2-(1-(2,4-dichlorobenzyl)-3-methyl-1,4,5,6-tetrahydro-7H-indazol-7-ylidene)-2-fluoroethyl)carbamoyl)thiophene-2-sulfonamide

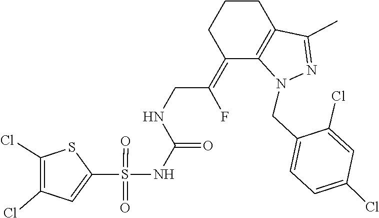 [SMILES: Cc1nn(Cc2ccc(Cl)cc2Cl)c2c1CCC/C2=C(/F)CNC(=O)NS(=O)(=O)c1cc(Cl)c(Cl)s1]

### Step 1. Synthesis of (E)-2-(4-bromobenzylidene)cyclohexan-1-one

A mixture of cyclohexanone (1 mL, 9.65 mmol) and 4-bromobenzaldehyde (1.20 g, 6.47 mmol) in aq. 1 N NaOH (6.5 mL, 6.5 mmol) and H₂O (60 mL) was heated at 65° C. for a total of 24 h. The reaction mixture was cooled to room temperature and extracted with ether. The organic layer was washed with sat. aq. NaCl, dried over Na₂SO₄, and concentrated. The residue was purified by silica gel column with 5% EtOAc/heptane to yield (E)-2-(4-bromobenzylidene)cyclohexan-1-one as a yellow solid.

### Step 2. Synthesis of ethyl (E)-2-(3-(4-bromobenzylidene)-2-oxocyclohexyl)-2-oxoacetate

To a solution of (E)-2-(4-bromobenzylidene)cyclohexan-1-one (1000 mg, 3.77 mmol) in THF (25 mL) at −78° C. was added LiHMDS (1.0 M in THF, 5.28 mL, 5.28 mmol), followed by diethyl oxalate (0.614 mL, 4.53 mmol). The reaction was stirred at −78° C. for 0.5 h before the reaction mixture was warmed up to −0° C. and kept for 2 h. The reaction was then quenched at 0° C. with aq. 1 N HCl, and extracted with ether. The organic layer was washed with sat. aq. NaCl, dried over Na₂SO₄ and concentrated to yield ethyl (E)-2-(3-(4-bromobenzylidene)-2-oxocyclohexyl)-2-oxoacetate as a yellow solid.

### Step 3. Synthesis of ethyl (E)-7-(4-bromobenzylidene)-1-(2,4-dichlorobenzyl)-4,5,6,7-tetrahydro-1H-indazole-3-carboxylate

Ethyl (E)-7-(4-bromobenzylidene)-1-(2,4-dichlorobenzyl)-4,5,6,7-tetrahydro-1H-indazole-3-carboxylate was prepared from ethyl (E)-2-(3-(4-bromobenzylidene)-2-oxocyclohexyl)-2-oxoacetate according to the procedures as described in Example 1, Step 3. Mass spectrum (ESI, m/z): Calculated for C₂₄H₂₂BrCl₂N₂O₂, 519.0 (M+H), found 519.0.

### Steps 4 and 5. Synthesis of (E)-(7-(4-bromobenzylidene)-1-(2,4-dichlorobenzyl)-4,5,6,7-tetrahydro-1H-indazol-3-yl)methanol and (E)-7-(4-bromobenzylidene)-3-(bromomethyl)-1-(2,4-dichlorobenzyl)-4,5,6,7-tetrahydro-1H-indazole

To a solution of ethyl (E)-7-(4-bromobenzylidene)-1-(2,4-dichlorobenzyl)-4,5,6,7-tetrahydro-1H-indazole-3-carboxylate (430 mg, 4.96 mmol) in THF (10 mL) at 0° C. was added DIBAL-H (1.0 M in THF, 5.0 mL, 5.0 mmol). The reaction was kept at 0° C. for 3 h before the reaction was quenched with H₂O. The resulting mixture was acidified with aq. 1 N HCl and extracted with EtOAc. The organic layer was washed with sat. aq. NaCl, dried over Na₂SO₄ and concentrated to yield (E)-(7-(4-bromobenzylidene)-1-(2,4-dichlorobenzyl)-4,5,6,7-tetrahydro-1H-indazol-3-yl)methanol.

To a solution of above prepared (E)-(7-(4-bromobenzylidene)-1-(2,4-dichlorobenzyl)-4,5,6,7-tetrahydro-1H-indazol-3-yl)methanol and PPh₃ (282 mg, 1.07 mmol) in CH₂Cl₂ (25 mL) at room temperature was added NBS (235 mg, 1.32 mmol). The reaction was stirred at room temperature for 15 min before the reaction was quenched with aq. NaHCO₃. The resulting mixture was extracted with EtOAc. The organic layer was washed with sat. aq. NaCl, dried over Na₂SO₄ and concentrated. The resulting residue was purified by silica gel column with 0-20% EtOAc/heptane to yield (E)-7-(4-bromobenzylidene)-3-(bromomethyl)-1-(2,4-dichlorobenzyl)-4,5,6,7-tetrahydro-1H-indazole. Mass spectrum (ESI, m/z): Calculated for C₂₂H₁₉Br₂Cl₂N₂, 538.9 (M+H), found 539.0.

### Step 6. Synthesis of (E)-7-(4-bromobenzylidene)-1-(2,4-dichlorobenzyl)-3-methyl-4,5,6,7-tetrahydro-1H-indazole

To (E)-7-(4-bromobenzylidene)-3-(bromomethyl)-1-(2,4-dichlorobenzyl)-4,5,6,7-tetrahydro-1H-indazole (350 mg, 0.647 mmol) in DMSO (6 mL) and THF (2 mL) at room temperature was added NaBH₄ (49 mg, 1.29 mmol). The reaction was stirred at room temperature for 5 h. The reaction was then quenched with H₂O and extracted with 50% EtOAc/heptane (3×). The organic layers were combined and washed with H₂O and sat. aq. NaCl, dried over Na₂SO₄ and concentrated to yield (E)-7-(4-bromobenzylidene)-1-(2,4-dichlorobenzyl)-3-methyl-4,5,6,7-tetrahydro-1H-indazole. Mass spectrum (ESI, m/z): Calculated for C₂₂H₂₀BrCl₂N₂, 461.0 (M+H), found 461.0.

### Step 7. Synthesis of 1-(2,4-dichlorobenzyl)-3-methyl-1,4,5,6-tetrahydro-7H-indazol-7-one

A solution of (E)-7-(4-bromobenzylidene)-1-(2,4-dichlorobenzyl)-3-methyl-4,5,6,7-tetrahydro-1H-indazole (300 mg, 0.649 mmol), OsO₄ (2.5% wt solution in t-BuOH, 0.20 mL, 0.020 mmol), 4-methylmorpholine 4-oxide (228 mg, 1.95 mmol), and 2,6-lutidine (0.15 mL, 1.30 mmol) in acetone (20 mL) and H₂O (2 mL) was stirred at room temperature for 5 days. phenyl-λ³-iodanediyl diacetate (418 mg, 1.30 mmol) was then added and the reaction was stirred at room temperature for 2 h. The reaction was then quenched with aq Na₂S₂O₃ and extracted with EtOAc. The organic layer was washed with sat. aq. NaCl, dried over Na₂SO₄ and concentrated. The resulting residue was purified with silica gel column with 20% EtOAc/heptane to yield 1-(2,4-dichlorobenzyl)-3-methyl-1,4,5,6-tetrahydro-7H-indazol-7-one. Mass spectrum (ESI, m/z): Calculated for C₁₅H₁₅Cl₂N₂O, 309.0 (M+H), found 309.0.

### Steps 8-12. Synthesis of (E)-4,5-dichloro-N-((2-(1-(2,4-dichlorobenzyl)-3-methyl-1,4,5,6-tetrahydro-7H-indazol-7-ylidene)ethyl)carbamoyl)thiophene-2-sulfonamide

(E)-4,5-dichloro-N-((2-(1-(2,4-dichlorobenzyl)-3-methyl-1,4,5,6-tetrahydro-7H-indazol-7-ylidene)ethyl)carbamoyl)thiophene-2-sulfonamide was prepared from 1-(2,4-dichlorobenzyl)-3-methyl-1,4,5,6-tetrahydro-7H-indazol-7-one according to the procedures as described in Example 8, Steps 4-5 and Example 11, Steps 1-3.

¹H NMR (400 MHz, CHLOROFORM-d) δ: 7.40-7.64 (m, 1H), 7.22-7.36 (m, 1H), 7.09 (dd, J=8.6, 2.0 Hz, 1H), 6.66 (d, J=8.1 Hz, 1H), 6.50-6.60 (m, 1H), 5.28 (d, J=14.1 Hz, 2H), 4.02-4.18 (m, 2H), 2.51 (t, J=6.6 Hz, 2H), 2.30-2.42 (m, 2H), 2.15 (s, 3H), 1.80-1.97 (m, 2H). Mass spectrum (ESI, m/z): Calculated for C₂₂H₂₀Cl₄FN₄O₃S₂, 611.0 (M+H), found 611.0.

### Example 28: Compound #157

### (Z)-4,5-dichloro-N-((2-fluoro-2-(1-(4-(trifluoromethyl)phenethyl)-1,4,5,6-tetrahydro-7H-indazol-7-ylidene)ethyl)carbamoyl)thiophene-2-sulfonamide

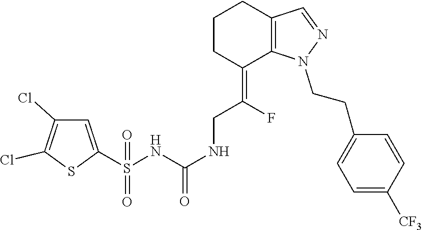 [SMILES: O=C(NC/C(F)=C1\CCCc2cnn(CCc3ccc(C(F)(F)F)cc3)c21)NS(=O)(=O)c1cc(Cl)c(Cl)s1]

### Step 1. Synthesis of ethyl (Z)-2-fluoro-2-(1,4,5,6-tetrahydro-7H-indazol-7-ylidene)acetate

To ethyl 2-(3-((dimethylamino)methylene)-2-oxocyclohexylidene)-2-fluoroacetate (600 mg, 2.35 mmol) in 1,4-dioxane (10 mL) was added hydrazine hydrate (180 mg, 3.60 mmol) and TFA (1 mL). The reaction was stirred at 60° C. for 6 h. The reaction was concentrated. The residue was purified by silica gel column with EA/PE (1:5) to yield ethyl (Z)-2-fluoro-2-(1,4,5,6-tetrahydro-7H-indazol-7-ylidene)acetate as a yellow oil. Mass spectrum (ESI, m/z): Calculated for C₁₁H₁₄FN₂O₂, 225.1 [M+H], found 224.9.

### Step 2. Synthesis of ethyl (Z)-2-fluoro-2-(1-(4-(trifluoromethyl)phenethyl)-1,4,5,6-tetrahydro-7H-indazol-7-ylidene)acetate

To a solution of ethyl (Z)-2-fluoro-2-(1,4,5,6-tetrahydro-7H-indazol-7-ylidene)acetate (120 mg, 0.535 mmol) in DMF (10 mL) at 0° C. was added NaH (27 mg, 1.12 mmol). The resulting solution was stirred for 0.5 h at 0° C. before 4-(trifluoromethyl)phenethyl methanesulfonate (300 mg, 1.12 mmol) was added. The reaction was stirred for 2 h at room temperature. The reaction was quenched with H₂O (10 mL) and the resulting mixture was extracted with EtOAc (20 mL). The organic layers were dried over Na₂SO₄ and concentrated. The residue was purified by silica gel chromatography (0-10% EtOAc/petroleum ether) to yield ethyl (Z)-2-fluoro-2-(1-(4-(trifluoromethyl)phenethyl)-1,4,5,6-tetrahydro-7H-indazol-7-ylidene)acetate. Mass spectrum (ESI, m/z): Calculated for C₂₀H₂₁F₄N₂O₂: 397.1 [M+H], found: 397.3.

### Step 3. Synthesis of (Z)-2-fluoro-2-(1-(4-(trifluoromethyl)phenethyl)-1,4,5,6-tetrahydro-7H-indazol-7-ylidene)ethan-1-ol

To a solution of ethyl (Z)-2-fluoro-2-(1-(4-(trifluoromethyl)phenethyl)-1,4,5,6-tetrahydro-7H-indazol-7-ylidene)acetate (130 mg, 0.328 mmol) in DCM (10 ml) was added DIBAL-H (0.66 mL). The reaction was stirred for 2 h at room temperature. The reaction was quenched with H₂O (10 mL) and the resulting mixture was extracted with EtOAc (10 mL). The organic layers were dried over Na₂SO₄ and concentrated. The residue obtained was purified by silica gel chromatography (0-20% EtOAc/petroleum ether) to yield (Z)-2-fluoro-2-(1-(4-(trifluoromethyl)phenethyl)-1,4,5,6-tetrahydro-7H-indazol-7-ylidene)ethan-1-ol as a yellow oil. Mass spectrum (ESI, m/z): Calculated for C₁₈H₁₉F₄N₂O: 337.1 [M+H−H₂O], found: 337.3.

### Steps 4-5. Synthesis of (Z)-2-fluoro-2-(1-(4-(trifluoromethyl)phenethyl)-1,4,5,6-tetrahydro-7H-indazol-7-ylidene)ethan-1-amine

(Z)-2-fluoro-2-(1-(4-(trifluoromethyl)phenethyl)-1,4,5,6-tetrahydro-7H-indazol-7-ylidene)ethan-1-amine was prepared from (Z)-2-fluoro-2-(1-(4-(trifluoromethyl)phenethyl)-1,4,5,6-tetrahydro-7H-indazol-7-ylidene)ethan-1-ol according to the procedures as described in Example 5, Steps 1-2.

### Steps 6-7. Synthesis of (Z)-4,5-dichloro-N-((2-fluoro-2-(1-(4-(trifluoromethyl)phenethyl)-1,4,5,6-tetrahydro-7H-indazol-7-ylidene)ethyl)carbamoyl)thiophene-2-sulfonamide

(Z)-4,5-dichloro-N-((2-fluoro-2-(1-(4-(trifluoromethyl)phenethyl)-1,4,5,6-tetrahydro-7H-indazol-7-ylidene)ethyl)carbamoyl)thiophene-2-sulfonamide was prepared from (Z)-2-fluoro-2-(1-(4-(trifluoromethyl)phenethyl)-1,4,5,6-tetrahydro-7H-indazol-7-ylidene)ethan-1-amine according to the procedures as described in Example 7, Steps 1-2.

¹H NMR (400 MHz, Methanol-d4) δ: 7.57-7.52 (m, 2H), 7.33-7.32 (m, 2H), 7.28 (s, 1H), 7.10 (s, 1H), 4.52-4.35 (m, 4H), 3.32-3.10 (m, 2H), 2.52-2.44 (m, 4H), 1.87-1.69 (m, 2H). ¹⁹F NMR (400 MHz, Methanol-d4) δ: −63.86, −111.90. Mass spectrum (ESI, m/z): Calculated for C₂₃H₂₁Cl₂F₄N₄O₃S₂, 611.0 [M+H], found 610.9.

### Example 29: Compound #6

### (E)-4-((E)-1-(2,4-dichlorobenzyl)-1,4,5,6-tetrahydro-7H-indazol-7-ylidene)-N-((4,5-dichlorothiophen-2-yl)sulfonyl)but-2-enamide

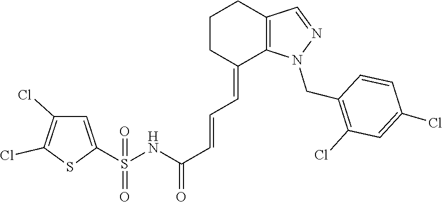 [SMILES: O=C(/C=C/C=C1\CCCc2cnn(Cc3ccc(Cl)cc3Cl)c21)NS(=O)(=O)c1cc(Cl)c(Cl)s1]

### Step 1. Synthesis of (E)-2-(1-(2,4-dichlorobenzyl)-1,4,5,6-tetrahydro-7H-indazol-7-ylidene)acetaldehyde

To a solution of (E)-2-(1-(2,4-dichlorobenzyl)-1,4,5,6-tetrahydro-7H-indazol-7-ylidene)ethan-1-ol (180 mg, 0.557 mmol) in CH₂Cl₂ (8 mL) was added MnO₂ (600 mg, 6.90 mmol). The mixture was stirred at room temperature overnight. Additional MnO₂ (500 mg, 5.75 mmol) was added. The reaction was stirred for another 6 h, then filtered. The solution was concentrated to yield (E)-2-(1-(2,4-dichlorobenzyl)-1,4,5,6-tetrahydro-7H-indazol-7-ylidene)acetaldehyde.

### Step 2. Synthesis of ethyl (E)-4-((E)-1-(2,4-dichlorobenzyl)-1,4,5,6-tetrahydro-7H-indazol-7-ylidene)but-2-enoate

A solution of the above prepared (E)-2-(1-(2,4-dichlorobenzyl)-1,4,5,6-tetrahydro-7H-indazol-7-ylidene)acetaldehyde and ethyl 2-(triphenyl-A⁵-phosphanylidene)acetate (233 mg, 0.668 mmol) in toluene (10 mL) was heated at 90° C. for 5 h. The reaction mixture was cooled to room temperature and concentrated. The residue was purified by silica gel column with 10% EtOAc/heptane to yield ethyl (E)-4-((E)-1-(2,4-dichlorobenzyl)-1,4,5,6-tetrahydro-7H-indazol-7-ylidene)but-2-enoate. Mass spectrum (ESI, m/z): Calculated for C₂₀H₂₁Cl₂N₂O₂, 391.1 [M+H], found 391.0.

### Step 3. Synthesis of (E)-4-((E)-1-(2,4-dichlorobenzyl)-1,4,5,6-tetrahydro-7H-indazol-7-ylidene)but-2-enoic acid

A mixture of ethyl (E)-4-((E)-1-(2,4-dichlorobenzyl)-1,4,5,6-tetrahydro-7H-indazol-7-ylidene)but-2-enoate (28 mg, 0.0716 mmol) and LiOH (6.9 mg, 0.286 mmol) in 1,4-dioxane (3 mL) and H₂O (1 mL) was stirred at room temperature overnight. The reaction mixture was acidified with aq, 1 N HCl and extracted with EtOAc. The organic layer was dried over Na₂SO₄ and concentrated to yield (E)-4-((E)-1-(2,4-dichlorobenzyl)-1,4,5,6-tetrahydro-7H-indazol-7-ylidene)but-2-enoic acid.

### Step 4. Synthesis of (E)-4-((E)-1-(2,4-dichlorobenzyl)-1,4,5,6-tetrahydro-7H-indazol-7-ylidene)-N-((4,5-dichlorothiophen-2-yl)sulfonyl)but-2-enamide

To a mixture of the above prepared (E)-4-((E)-1-(2,4-dichlorobenzyl)-1,4,5,6-tetrahydro-7H-indazol-7-ylidene)but-2-enoic acid, 4,5-dichlorothiophene-2-sulfonamide (25 mg, 0.107 mmol), and DMAP (17.5 mg, 0.143 mmol) in DMF (3 mL) at room temperature was added EDCI (27.4 mg, 0.143 mmol). The reaction was stirred at room temperature for 7 h. The resulting mixture was then diluted with EtOAc and washed with aq. 1N HCl and sat. aq. NaCl. The organic layer was dried over Na₂SO₄ and concentrated. The resulting residue was purified by silica gel column with 20%-50% EtOAc/heptane to yield (E)-4-((E)-1-(2,4-dichlorobenzyl)-1,4,5,6-tetrahydro-7H-indazol-7-ylidene)-N-((4,5-dichlorothiophen-2-yl)sulfonyl)but-2-enamide.

¹H NMR (400 MHz, CHLOROFORM-d) δ: 7.60-7.75 (m, 2H), 7.44 (s, 2H), 7.16 (dd, J=8.3, 1.8 Hz, 1H), 6.55 (d, J=8.6 Hz, 1H), 5.99 (br d, J=11.1 Hz, 1H), 5.75 (d, J=14.7 Hz, 1H), 5.55 (s, 2H), 2.67 (dt, J=12.0, 5.9 Hz, 4H), 1.82-1.93 (m, 2H). Mass spectrum (ESI, m/z): Calculated for C₂₂H₁₈Cl₄N₃O₃S₂, 575.9 [M+H], found 576.0.

### Example 30: Compound #36

### (E)-4-chloro-N-((2-(1-(2,4-dichlorobenzyl)-4,5-dihydro-cyclopenta[c]pyrazol-6(1H)-ylidene)ethyl)carbamoyl)benzenesulfonamide

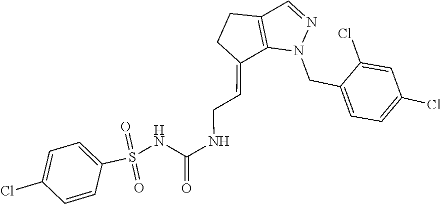 [SMILES: O=C(NC/C=C1\CCc2cnn(Cc3ccc(Cl)cc3Cl)c21)NS(=O)(=O)c1ccc(Cl)cc1]

### Step 1. Synthesis of (E)-2-(4-chlorobenzylidene)cyclopentan-1-one

(E)-2-(4-chlorobenzylidene)cyclopentan-1-one was prepared from cyclopentanone and 4-chlorobenzaldehyde according to the procedures as described in Example 27, Step 1.

### Step 2. Synthesis of 2-((E)-4-chlorobenzylidene)-6-(hydroxymethylene)cyclohexan-1-one

To a solution of (E)-2-(4-chlorobenzylidene)cyclopentan-1-one (100 mg, 0.484 mmol) and ethyl formate (0.50 mL, 6.22 mmol) in toluene (3 mL) at 0° C. was added NaOEt (21% solution in EtOH, 0.54 mL, 1.45 mmol). The reaction was kept at 0° C. for 10 min and warmed up to room temperature. The reaction was stirred at room temperature for 1 h, then quenched with aq. NaH₂PO₄. The resulting mixture was extracted with EtOAc. The organic layer was washed with sat. aq. NaCl, dried over Na₂SO₄ and concentrated to yield 2-((E)-4-chlorobenzylidene)-6-(hydroxymethylene)cyclohexan-1-one.

### Step 3. Synthesis of (E)-6-(4-chlorobenzylidene)-1-(2,4-dichlorobenzyl)-1,4,5,6-tetrahydrocyclopenta[c]pyrazole

A mixture of above prepared 2-((E)-4-chlorobenzylidene)-6-(hydroxymethylene)cyclohexan-1-one\, (2,4-dichlorobenzyl)hydrazine hydrochloride (110 mg, 0.484 mmol), and H₂SO₄ (0.1 mL) in 1,4-dioxane (5 mL) was heated at 50° C. for 1 h. The reaction mixture was cooled to room temperature and diluted with EtOAc. The resulting solution was washed with sat. aq. NaHCO₃ and sat. aq. NaCl, dried over Na₂SO₄, and concentrated. The resulting residue was purified by silica gel column with 10% EtOAc/heptane to yield (E)-6-(4-chlorobenzylidene)-1-(2,4-dichlorobenzyl)-1,4,5,6-tetrahydrocyclopenta[c]pyrazole). Mass spectrum (ESI, m/z): Calculated for C₂₀H₁₆Cl₃N₂, 389.0 [M+H], found 391.0.

### Step 4. Synthesis of 1-(2,4-dichlorobenzyl)-4,5-dihydrocyclopenta[c]pyrazol-6(1H)-one

1-(2,4-dichlorobenzyl)-4,5-dihydrocyclopenta[c]pyrazol-6(1H)-one was prepared from (E)-6-(4-chlorobenzylidene)-1-(2,4-dichlorobenzyl)-1,4,5,6-tetrahydrocyclopenta[c]pyrazole according to the procedures as described in Example 27, Step 7.

### Step 5. Synthesis of ethyl (E)-2-(1-(2,4-dichlorobenzyl)-4,5-dihydrocyclopenta[c]pyrazol-6(1H)-ylidene)acetate

Ethyl (E)-2-(1-(2,4-dichlorobenzyl)-4,5-dihydrocyclopenta[c]pyrazol-6(1H)-ylidene)acetate was prepared from 1-(2,4-dichlorobenzyl)-4,5-dihydrocyclopenta[c]pyrazol-6(1H)-one according to the procedures as described in Example 19, Step 3.

### Step 6. Synthesis of (E)-2-(1-(2,4-dichlorobenzyl)-4,5-dihydrocyclopenta[c]pyrazol-6(1H)-ylidene)ethan-1-ol

(E)-2-(1-(2,4-dichlorobenzyl)-4,5-dihydrocyclopenta[c]pyrazol-6(1H)-ylidene)ethan-1-ol was prepared from Ethyl (E)-2-(1-(2,4-dichlorobenzyl)-4,5-dihydrocyclopenta[c]pyrazol-6(1H)-ylidene)acetate according to the procedures as described in Example 19, Step 4.

### Step 7. Synthesis of (E)-2-(1-(2,4-dichlorobenzyl)-4,5-dihydro-cyclopenta[c]pyrazol-6(1H)-ylidene)ethyl ((4-chlorophenyl)sulfonyl)carbamate

(E)-2-(1-(2,4-dichlorobenzyl)-4,5-dihydrocyclopenta[c]pyrazol-6(1H)-ylidene)ethyl ((4-chlorophenyl)sulfonyl)carbamate was prepared from (E)-2-(1-(2,4-dichlorobenzyl)-4,5-dihydrocyclopenta[c]pyrazol-6(1H)-ylidene)ethan-1-ol according to the procedures as described in Example 2, Step 1.

¹H NMR (400 MHz, CHLOROFORM-d) δ: 7.91-8.00 (m, 2H), 7.45-7.55 (m, 2H), 7.37-7.43 (m, 1H), 7.32 (s, 1H), 7.14 (dd, J=8.3, 2.3 Hz, 1H), 6.60 (d, J=8.6 Hz, 1H), 5.40 (s, 2H), 5.22-5.36 (m, 1H), 4.60 (d, J=7.6 Hz, 2H), 3.09-3.18 (m, 2H), 2.70-2.79 (m, 2H). Mass spectrum (ESI, m/z): Calculated for C₂₂H₁₉Cl₃N₃O₄S, 526.0 [M+H], found 526.0.

### Example 31: Compound #136

### (Z)-4,5-dichloro-N-((2-(1-(2,4-dichlorobenzyl)-4,5-dihydro-cyclopenta[c]pyrazol-6(1H)-ylidene)-2-fluoroethyl)carbamoyl)thiophene-2-sulfonamide

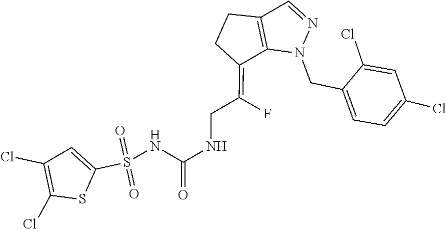 [SMILES: O=C(NC/C(F)=C1\CCc2cnn(Cc3ccc(Cl)cc3Cl)c21)NS(=O)(=O)c1cc(Cl)c(Cl)s1]

### Step 1. Synthesis of ethyl 2-(1-(2,4-dichlorobenzyl)-4,5-dihydrocyclopenta[c]pyrazol-6(1H)-ylidene)-2-fluoroacetate

To a solution of ethyl 2-(diethoxyphosphoryl)-2-fluoroacetate (227 mg, 0.939 mmol) in THF (4 mL) at 0° C. was added NaH (60% in mineral oil, 34 mg, 0.845 mmol). The reaction was stirred for 30 min at 0° C. A solution of 1-(2,4-dichlorobenzyl)-4,5-dihydrocyclopenta[c]pyrazol-6(1H)-one (88 mg, 0.313 mmol) in THF (2 mL) was then added. The reaction was stirred at 0° C. for 1 h before the reaction was quenched with aq. 1N HCl. The resulting mixture was extracted with EtOAc. The organic layer was washed with sat. aq. NaCl, dried over Na₂SO₄ and concentrated. The resulting residue was purified by silica gel column with 10% EtOAc/heptane to yield ethyl 2-(1-(2,4-dichlorobenzyl)-4,5-dihydrocyclopenta[c]pyrazol-6(1H)-ylidene)-2-fluoroacetate as a mixture of (E) and (Z) isomers.

### Step 2. Synthesis of (Z)-2-(1-(2,4-dichlorobenzyl)-4,5-dihydrocyclopenta[c]pyrazol-6(1H)-ylidene)-2-fluoroethan-1-ol

To a solution of ethyl 2-(1-(2,4-dichlorobenzyl)-4,5-dihydrocyclopenta[c]pyrazol-6(1H)-ylidene)-2-fluoroacetate (114 mg, 0.309 mmol) in CH₂Cl₂ (6 mL) at −78° C. was added DIBAL-H (1.0 M in heptane, 1.24 mL, 1.24 mmol). The reaction was kept at −78° C. for 1.5 h. The reaction was then quenched with MeOH and H₂O. The mixture was extracted with EtOAc. The organic layer was washed with sat. aq. NaCl, dried over Na₂SO₄ and concentrated. The resulting residue was purified by silica gel column with 40% EtOAc/heptane to yield (Z)-2-(1-(2,4-dichlorobenzyl)-4,5-dihydrocyclopenta[c]pyrazol-6(1H)-ylidene)-2-fluoroethan-1-ol.

### Steps 3-5. Synthesis of (Z)-4,5-dichloro-N-((2-(1-(2,4-dichlorobenzyl)-4,5-dihydrocyclopenta[c]pyrazol-6(1H)-ylidene)-2-fluoroethyl)carbamoyl)thiophene-2-sulfonamide

(Z)-4,5-dichloro-N-((2-(1-(2,4-dichlorobenzyl)-4,5-dihydrocyclopenta[c]pyrazol-6(1H)-ylidene)-2-fluoroethyl)carbamoyl)thiophene-2-sulfonamide was prepared from (Z)-2-(1-(2,4-dichlorobenzyl)-4,5-dihydrocyclopenta[c]pyrazol-6(1H)-ylidene)-2-fluoroethan-1-ol according to the procedures as described in Example 5, Steps 1-3.

¹H NMR (400 MHz, CHLOROFORM-d) δ: 7.44 (s, 1H), 7.35-7.39 (m, 1H), 7.32 (s, 1H), 7.11 (dd, J=8.1, 2.0 Hz, 1H), 6.72 (br s, 1H), 6.52 (d, J=8.6 Hz, 1H), 5.56 (s, 2H), 3.97-4.08 (m, 2H), 3.25 (br s, 2H), 2.77 (br s, 2H). Mass spectrum (ESI, m/z): Calculated for C₂₀H₁₆Cl₄FN₄O₃S₂, 582.9 [M+H], found 585.0.

The following representative compounds of formula (I) were similarly prepared according to the procedure described in Example 31 (and Examples referenced therein) above, selecting and substituting suitable starting materials and reagents, as would be readily recognized by those skilled in the art.

### (Z)-4,5-dichloro-N-((2-fluoro-2-(1-(4-(trifluoromethyl)phenyl)-4,5-dihydrocyclopenta[c]pyrazol-6(1H)-ylidene)ethyl)carbamoyl)thiophene-2-sulfonamide, Compound #130

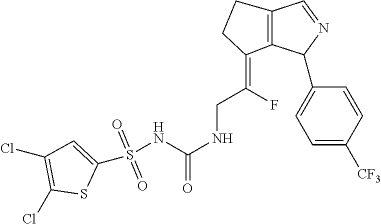 [SMILES: O=C(NC/C(F)=C1\CCC2=C1C(c1ccc(C(F)(F)F)cc1)N=C2)NS(=O)(=O)c1cc(Cl)c(Cl)s1]

¹H NMR (400 MHz, CHLOROFORM-d) δ: 7.66 (d, J=8.6 Hz, 2H), 7.43-7.58 (m, 4H), 3.95-4.07 (m, 2H), 3.30 (td, J=6.1, 2.5 Hz, 2H), 2.66-2.91 (m, 2H). Mass spectrum (ESI, m/z): Calculated for C₂₀H₁₅Cl₂F₄N₄O₃S₂, 569.0 [M+H], found 569.0.

### (E)-4,5-dichloro-N-((2-fluoro-2-(1-(4-(trifluoromethyl)phenyl)-4,5-dihydrocyclopenta[c]pyrazol-6(1H)-ylidene)ethyl)carbamoyl)thiophene-2-sulfonamide, Compound #137

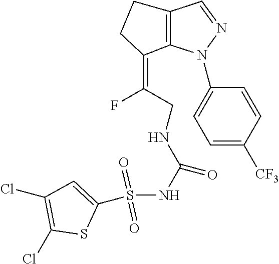 [SMILES: O=C(NC/C(F)=C1/CCc2cnn(-c3ccc(C(F)(F)F)cc3)c21)NS(=O)(=O)c1cc(Cl)c(Cl)s1]

¹H NMR (400 MHz, CHLOROFORM-d) δ: 7.75 (d, J=8.6 Hz, 2H), 7.61 (d, J=8.1 Hz, 2H), 7.51 (s, 1H), 7.47 (s, 1H), 3.40-3.57 (m, 2H), 3.27-3.40 (m, 2H), 2.70-2.85 (m, 2H). Mass spectrum (ESI, m/z): Calculated for C₂₀H₁₅Cl₂F₄N₄O₃S₂, 569.0 [M+H], found 569.0.

### (Z)-4-chloro-N-((2-(1-(2,4-dichlorobenzyl)-4,5,6,7-tetrahydrocyclohepta[c]pyrazol-8(1H)-ylidene)-2-fluoroethyl)carbamoyl)benzenesulfonamide, Compound #98

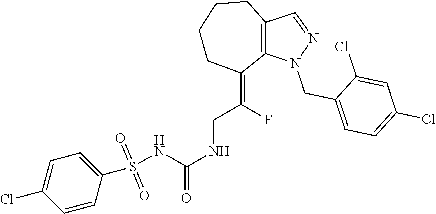 [SMILES: O=C(NC/C(F)=C1\CCCCc2cnn(Cc3ccc(Cl)cc3Cl)c21)NS(=O)(=O)c1ccc(Cl)cc1]

¹H NMR (CHLOROFORM-d) δ: 7.82-7.88 (m, 2H), 7.37-7.50 (m, 2H), 7.28-7.35 (m, 2H), 7.15 (dd, J=8.6, 2.0 Hz, 1H), 6.74 (d, J=8.1 Hz, 1H), 5.16 (s, 2H), 4.07-4.17 (m, 2H), 2.37-2.61 (m, 2H), 2.13 (br s, 2H), 1.73 (br s, 2H), 1.55-1.68 (m, 2H). Mass spectrum (ESI, m/z): Calculated for C₂₄H₂₃Cl₃FN₄O₃S, 571.0 [M+H], found 571.0.

### (E)-4-chloro-N-((2-(1-(2,4-dichlorobenzyl)-4,5,6,7-tetrahydrocyclohepta[c]pyrazol-8(1H)-ylidene)-2-fluoroethyl)carbamoyl)benzenesulfonamide, Compound #102

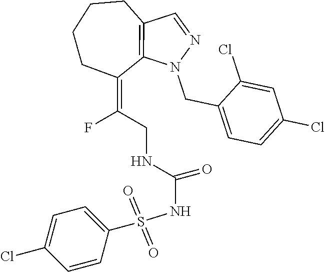 [SMILES: O=C(NC/C(F)=C1/CCCCc2cnn(Cc3ccc(Cl)cc3Cl)c21)NS(=O)(=O)c1ccc(Cl)cc1]

¹H NMR (400 MHz, CHLOROFORM-d) δ: 7.79 (d, J=8.6 Hz, 2H), 7.26-7.50 (m, 4H), 7.13 (dd, J=8.1, 2.0 Hz, 1H), 6.72 (br s, 1H), 6.62 (d, J=8.1 Hz, 1H), 5.09-5.21 (m, 2H), 3.78-4.18 (m, 2H), 2.87 (br dd, J=11.6, 4.5 Hz, 1H), 2.61 (br dd, J=14.9, 3.8 Hz, 1H), 2.37 (br t, J=13.6 Hz, 1H), 2.01-2.13 (m, 1H), 1.85-1.94 (m, 1H), 1.29-1.58 (m, 3H). Mass spectrum (ESI, m/z): Calculated for C₂₄H₂₃Cl₃FN₄O₃S, 571.0 [M+H], found 571.0.

### (Z)-4,5-dichloro-N-((2-(1-(2,4-dichlorobenzyl)-4,5,6,7-tetrahydrocyclohepta[c]pyrazol-8(1H)-ylidene)-2-fluoroethyl)carbamoyl)thiophene-2-sulfonamide, Compound #120

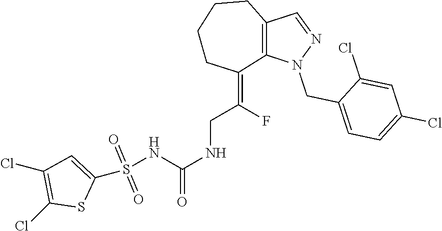 [SMILES: O=C(NC/C(F)=C1\CCCCc2cnn(Cc3ccc(Cl)cc3Cl)c21)NS(=O)(=O)c1cc(Cl)c(Cl)s1]

¹H NMR (400 MHz, CHLOROFORM-d) δ: 7.40-7.50 (m, 1H), 7.29-7.40 (m, 2H), 7.13 (dd, J=8.1, 2.0 Hz, 1H), 6.65-6.77 (m, 2H), 5.20 (s, 2H), 4.09-4.26 (m, 2H), 2.46-2.58 (m, 2H), 2.16 (br s, 2H), 1.79 (br s, 2H), 1.57-1.73 (m, 2H). Mass spectrum (ESI, m/z): Calculated for C₂₂H₂₀Cl₄FN₄O₃S₂, 611.0 [M+H], found 611.0.

### Example 32: Compound #46

### (Z)-2-(1-(2,4-dichlorobenzyl)-1,4-dihydropyrano[4,3-c]pyrazol-7(6H)-ylidene)ethyl ((4,5-dichlorothiophen-2-yl)sulfonyl)carbamate

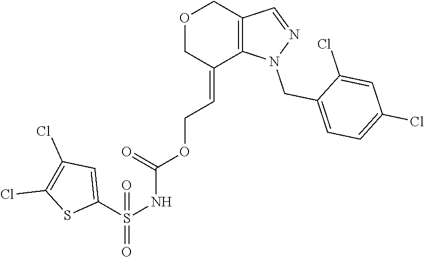 [SMILES: O=C(NS(=O)(=O)c1cc(Cl)c(Cl)s1)OC/C=C1\COCc2cnn(Cc3ccc(Cl)cc3Cl)c21]

### Step 1. Synthesis of (E)-3-benzylidenetetrahydro-4H-pyran-4-one

A mixture of tetrahydro-4H-pyran-4-one (200 mg, 2.00 mmol), benzaldehyde (233 mg, 2.20 mmol), and NaOH (20 mg, 0.500 mmol) in water (20 ml) was stirred for 2 h at room temperature. The resulting solution was then extracted with ethyl acetate (3×20 mL). The organic layers were combined and concentrated under vacuum. The resulting residue was purified by silica gel column with PE/EA (10/1) to yield ((E)-3-benzylidenetetrahydro-4H-pyran-4-one as a yellow solid.

### Step 2. Synthesis of 3-((E)-benzylidene)-5-((dimethylamino)methylene)tetrahydro-4H-pyran-4-one

A solution of ((E)-3-benzylidenetetrahydro-4H-pyran-4-one (100 mg, 0.531 mmol) in dimethoxy-N, N-dimethylmethanamine (2 mL) and toluene (10 ml) was stirred for 4 h at 100° C. The reaction mixture was concentrated under vacuum to yield 3-((E)-benzylidene)-5-((dimethylamino)methylene)tetrahydro-4H-pyran-4-one as a yellow solid. Mass spectrum (ESI, m/z): Calculated for C₁₅H₁₈NO₂, 244.1 (M+H), found 244.0.

### Step 3. Synthesis of (Z)-7-benzylidene-1-(2,4-dichlorobenzyl)-1,4,6,7-tetrahydropyrano[4,3-c]pyrazole

A solution of 3-((E)-benzylidene)-5-((dimethylamino)methylene)tetrahydro-4H-pyran-4-one (1.5 g, 6.94 mmol), (2,4-dichlorobenzyl)hydrazine hydrochloride (1.6 g, 8.37 mmol), and 2,2,2-trifluoroacetic acid (1.5 ml) in 1,4-dioxane (30 ml) was stirred for 2 h at 60° C. The reaction mixture was concentrated under vacuum. The residue purified by silica gel column with PE/EA (1/10) to yield (Z)-7-benzylidene-1-(2,4-dichlorobenzyl)-1,4,6,7-tetrahydropyrano[4,3-c]pyrazole as a yellow solid. Mass spectrum (ESI, m/z): Calculated for C₂₀H₁₇Cl₂N₂O, 371.1 (M+H), found 371.0.

### Step 4. Synthesis of 1-(2,4-dichlorobenzyl)-1,4-dihydropyrano[4,3-c]pyrazol-7(6H)-one

A solution of (Z)-7-benzylidene-1-(2,4-dichlorobenzyl)-1,4,6,7-tetrahydropyrano[4,3-c]pyrazole (1.4 g, 3.77 mmol), osmium(VIII) oxide (96 mg, 0.378 mmol), and 4-methylmorpholine 4-oxide (1.76 g, 15.0 mmol) in acetone (30 mL) and H₂O (3 ml) was stirred for 16 h at room temperature. To the reaction mixture was added phenyl-λ³-iodanediyl diacetate (2.2 g, 6.83 mmol). The reaction was stirred for 16 h at room temperature. The resulting solution was extracted with ethyl acetate (3×100 mL). The organic layers were combined and concentrated under vacuum. The residue was purified by silica gel column with PE/EA (10/1) to yield 1-(2,4-dichlorobenzyl)-1,4-dihydropyrano[4,3-c]pyrazol-7(6H)-one as a yellow solid. Mass spectrum (ESI, m/z): Calculated for C₁₃H₁₁C₁₂N₂O₂, 297.0 (M+H), found 296.9.

### Step 5. Synthesis of ethyl (Z)-2-(1-(2,4-dichlorobenzyl)-1,4-dihydropyrano[4,3-c]pyrazol-7(6H)-ylidene)acetate

To sodium hydride (363 mg, 9.08 mmol) in THF (30 mL) under nitrogen at 0° C. was added ethyl 2-(diethoxyphosphoryl)acetate (1.0 g, 4.46 mmol). The reaction was stirred for 30 min at 0° C. A solution of 1-(2,4-dichlorobenzyl)-1,4-dihydropyrano[4,3-c]pyrazol-7(6H)-one (900 mg, 3.03 mmol) in THF was then added. The reaction was warmed up to room temperature and stirred for 1 h before the reaction was quenched with NH₄Cl (aq) and extracted with EtOAc. The organic layer was concentrated under vacuum. The residue was purified by silica gel column with PE/EA (10/1) to yield ethyl (Z)-2-(1-(2,4-dichlorobenzyl)-1,4-dihydropyrano[4,3-c]pyrazol-7(6H)-ylidene)acetate as yellow solid. Mass spectrum (ESI, m/z): Calculated for C₁₇H₁₇C₁₂N₂O₃, 367.1 (M+H), found 366.9.

### Step 6. Synthesis of (Z)-2-(1-(2,4-dichlorobenzyl)-1,4-dihydropyrano[4,3-c]pyrazol-7(6H)-ylidene)ethan-1-ol

To a solution of ethyl (Z)-2-(1-(2,4-dichlorobenzyl)-1,4-dihydropyrano[4,3-c]pyrazol-7(6H)-ylidene)acetate (850 mg, 2.32 mmol) in THF (30 mL) at −78° C. under nitrogen was added DIBAL-H (1.0 M in toluene, 4.6 ml, 4.6 mmol). The reaction was warmed up to 0° C. and stirred for 2 h at 0° C. The reaction was quenched with NH₄Cl (aq) and extracted with EtOAc. The organic layer was concentrated under vacuum. The residue was purified by silica gel column with PE/EA (2/1) to yield (Z)-2-(1-(2,4-dichlorobenzyl)-1,4-dihydropyrano[4,3-c]pyrazol-7(6H)-ylidene)ethan-1-ol as a yellow oil. Mass spectrum (ESI, m/z): Calculated for C₁₅H₁₅Cl₂N₂O₂, 325.0 (M+H), found 324.9.

### Step 7. Synthesis of (Z)-2-(1-(2,4-dichlorobenzyl)-1,4-dihydropyrano[4,3-c]pyrazol-7(6H)-ylidene)ethyl ((4,5-dichlorothiophen-2-yl)sulfonyl)carbamate

To a solution of 4,5-dichlorothiophene-2-sulfonamide (100 mg, 0.431 mmol) in THF (2 mL) was added di(1H-imidazol-1-yl)methanone (67 mg, 0.413 mmol) and triethylamine (46.6 mg, 0.461 mmol). The reaction was stirred for 3 h at 30° C. before (Z)-2-(1-(2,4-dichlorobenzyl)-1,4-dihydropyrano[4,3-c]pyrazol-7(6H)-ylidene)ethan-1-ol (50 mg, 0.154 mmol) was added. The reaction mixture was heated at 100° C. by microwave for 1 h. The resulting mixture was concentrated under vacuum. The resulting residue was purified by Prep-HPLC with the following conditions (1#waters2767-5): Column, Sun Fire Prep C18,19*150 mm 5 umH PrepC-001(T)18600256819513816414 04; mobile phase, PhaseA: water with 0.05% NH₄HCO₃; PhaseB:CH₃CN (20% CH₃CN up to 60% in 10 min, up to 100% CH₃CN in 0.1 min, hold 100% in 1.9 min, down to 20% CH₃CN in 0.1 min, hold 20% in 1.9 min); Detector, UV220&254 nm to yield (Z)-2-(1-(2,4-dichlorobenzyl)-1,4-dihydropyrano[4,3-c]pyrazol-7(6H)-ylidene)ethyl ((4,5-dichlorothiophen-2-yl)sulfonyl)carbamate as a white solid.

¹H NMR (300 MHz, CD₃OD) δ: 7.47 (s, 1H), 7.40 (s, 1H), 7.32 (s, 1H), 7.17-7.21 (m, 1H), 6.40-6.48 (m, 1H), 5.59 (t, J=6.3 Hz, 1H), 5.51 (s, 2H), 4.70 (s, 2H), 4.51-4.57 (m, 2H), 4.40 (s, 2H). Mass spectrum (ESI, m/z): Calculated for C₂₀H₁₆Cl₄N₃O₅S₂, 583.9 (M+H), found 583.9.

### Example 33: Compound #49

### (Z)-4,5-dichloro-N-((2-(1-(2,4-dichlorobenzyl)-1,4-dihydropyrano[4,3-c]pyrazol-7(6H)-ylidene)ethyl)carbamoyl)thiophene-2-sulfonamide

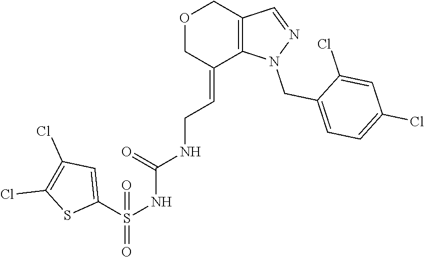 [SMILES: O=C(NC/C=C1\COCc2cnn(Cc3ccc(Cl)cc3Cl)c21)NS(=O)(=O)c1cc(Cl)c(Cl)s1]

### Step 1. Synthesis of (Z)-2-(2-(1-(2,4-dichlorobenzyl)-1,4-dihydropyrano[4,3-c]pyrazol-7(6H)-ylidene)ethyl)isoindoline-1,3-dione

(Z)-2-(2-(1-(2,4-dichlorobenzyl)-1,4-dihydropyrano[4,3-c]pyrazol-7(6H)-ylidene)ethyl)isoindoline-1,3-dione was prepared from (Z)-2-(1-(2,4-dichlorobenzyl)-1,4-dihydropyrano[4,3-c]pyrazol-7(6H)-ylidene)ethan-1-01 according to the procedures as described in Example 5, Step 1. Mass spectrum (ESI, m/z): Calculated for C₂₃H₁₈C₁₂N₃O₃, 454.1 (M+H), found 454.0.

### Step 2. Synthesis of (Z)-2-(1-(2,4-dichlorobenzyl)-1,4-dihydropyrano[4,3-c]pyrazol-7(6H)-ylidene)ethan-1-amine

(Z)-2-(1-(2,4-dichlorobenzyl)-1,4-dihydropyrano[4,3-c]pyrazol-7(6H)-ylidene)ethan-1-amine was prepared from (Z)-2-(2-(1-(2,4-dichlorobenzyl)-1,4-dihydropyrano[4,3-c]pyrazol-7(6H)-ylidene)ethyl)isoindoline-1,3-dione according to the procedures as described in Example 5, Step 2. Mass spectrum (ESI, m/z): Calculated for C₁₅H₁₅Cl₂N₃O, 325.1 (M+H), found 324.8.

### Step 3. Synthesis of (Z)-4,5-dichloro-N-((2-(1-(2,4-dichlorobenzyl)-1,4-dihydropyrano[4,3-c]pyrazol-7(6H)-ylidene)ethyl)carbamoyl)thiophene-2-sulfonamide

To a solution of 4,5-dichlorothiophene-2-sulfonamide (100 mg, 0.431 mmol) in THF (2 mL) was added di(1H-imidazol-1-yl)methanone (67 mg, 0.413 mmol) and triethylamine (46.6 mg, 0.461 mmol). The reaction was stirred for 3 h at 30° C. before (Z)-2-(1-(2,4-dichlorobenzyl)-1,4-dihydropyrano[4,3-c]pyrazol-7(6H)-ylidene)ethan-1-amine (50 mg, 0.154 mmol) was added. The reaction was heated at 100° C. by microwave for 1 h. The mixture was concentrated under vacuum. The resulting residue was purified by Prep-HPLC with the following conditions (1#waters2767-5): Column, SunFire Prep C18, 19*150 mm 5umH PrepC-001(T)18600256819513816414 04; mobile phase, PhaseA: water with 0.05% NH₄HCO₃; PhaseB:CH₃CN (20% CH₃CN up to 50% in 10 min, up to 100% CH₃CN in 0.1 min, hold 100% in 1.9 min, down to 20% CH₃CN in 0.1 min, hold 20% in 1.9 min); Detector, UV220&254 nm to yield (Z)-4,5-dichloro-N-((2-(1-(2,4-dichlorobenzyl)-1,4-dihydropyrano[4,3-c]pyrazol-7(6H)-ylidene)ethyl)carbamoyl)thiophene-2-sulfonamide as a white solid.

¹H NMR (300 MHz, CD₃OD) δ: 7.43-7.54 (m, 2H), 7.39 (s, 1H), 7.16-7.20 (m, 1H), 6.47 (d, J=8.4 Hz, 1H), 5.68 (s, 2H), 5.41-5.50 (m, 1H), 4.69 (s, 2H), 4.39 (s, 2H), 3.75 (d, J=6.6 Hz, 2H). Mass spectrum (ESI, m/z): Calculated for C₂₀H₁₇Cl₄N₄O₄S₂, 582.9 (M+H), found 582.9.

The following representative compounds of formula (I) were similarly prepared according to the procedure described in Example 33 (and Examples referenced therein) above, selecting and substituting suitable starting materials and reagents, as would be readily recognized by those skilled in the art.

### (Z)—N-((2-(1-(2,4-dichlorobenzyl)-1,4-dihydropyrano[4,3-c]pyrazol-7(6H)-ylidene)ethyl)carbamoyl)-2-methoxybenzenesulfonamide, Compound #58

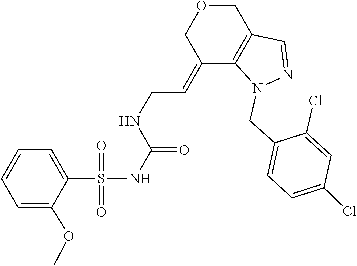 [SMILES: COc1ccccc1S(=O)(=O)NC(=O)NC/C=C1\COCc2cnn(Cc3ccc(Cl)cc3Cl)c21]

¹H NMR (300 MHz, Methanol-d4) δ: 7.92 (d, J=4.8 Hz, 1H), 7.64 (t, J=4.8 Hz, 1H), 7.51 (s, 1H), 7.44 (s, 1H), 7.18-7.26 (m, 2H), 7.07 (t, J=8.1 Hz, 1H), 6.49 (d, J=8.4 Hz, 1H), 5.51 (s, 2H), 5.42 (t, J=6.6 Hz, 1H), 4.72 (s, 2H), 4.37 (s, 2H), 3.93 (s, 3H), 3.78 (d, J=6.6 Hz, 2H). Mass spectrum (ESI, m/z): Calculated for C₂₃H₂₃Cl₂N₄O₅S, 537.1 (M+H), found 537.1.

### (Z)—N-((2-(1-(2,4-dichlorobenzyl)-1,4-dihydropyrano[4,3-c]pyrazol-7(6H)-ylidene)ethyl)carbamoyl)-4-methoxybenzenesulfonamide, Compound #62

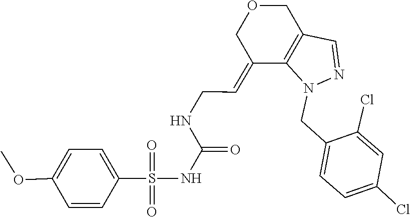 [SMILES: COc1ccc(S(=O)(=O)NC(=O)NC/C=C2\COCc3cnn(Cc4ccc(Cl)cc4Cl)c32)cc1]

¹H NMR (300 MHz, Methanol-d4) δ: 7.91 (d, J=6.9 Hz, 2H), 7.46-7.49 (m, 2H), 7.24 (d, J=8.4 Hz, 1H), 7.08 (d, J=9.0 Hz, 2H), 6.51 (d, J=8.4 Hz, 1H), 5.52 (s, 2H), 5.41 (t, J=6.6 Hz, 1H), 4.74 (s, 2H), 4.40 (s, 2H), 3.90 (s, 3H), 3.79 (d, J=6.9 Hz, 2H). Mass spectrum (ESI, m/z): Calculated for C₂₃H₂₃Cl₂N₄O₅S, 537.1 (M+H), found 537.0.

### (Z)—N-((2-(1-(2,4-dichlorobenzyl)-1,4-dihydropyrano[4,3-c]pyrazol-7(6H)-ylidene)ethyl)carbamoyl)-3-methoxybenzenesulfonamide, Compound #63

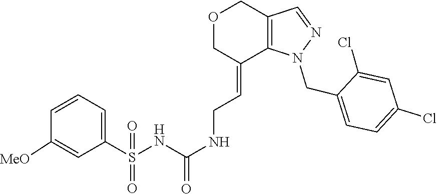 [SMILES: COc1cccc(S(=O)(=O)NC(=O)NC/C=C2\COCc3cnn(Cc4ccc(Cl)cc4Cl)c32)c1]

¹HNMR (400 Hz, DMSO) δ:10.71 (s, 1H), 7.57 (s, 1H), 7.33-7.52 (m, 5H), 7.21-7.23 (m, 1H), 6.69 (s, 1H), 6.53-6.56 (m, 1H), 5.41 (s, 2H), 5.36-5.39 (m, 1H), 4.63 (s, 2H), 4.29 (s, 2H), 3.79 (s, 3H), 3.63-3.66 (m, 2H). Mass spectrum (El, m/z): Calculated for C₂₃H₂₃C₁₂N₄O₅S, 537.1 (M+H), found 537.0.

### (Z)—N-((2-(1-(2,4-dichlorobenzyl)-1,4-dihydropyrano[4,3-c]pyrazol-7(6H)-ylidene)ethyl)carbamoyl)-5-fluoro-2-methoxybenzenesulfonamide, Compound #64

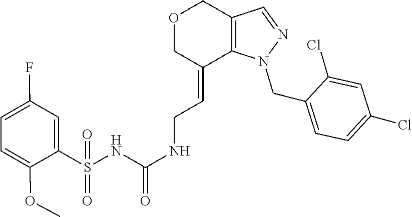 [SMILES: COc1ccc(F)cc1S(=O)(=O)NC(=O)NC/C=C1\COCc2cnn(Cc3ccc(Cl)cc3Cl)c21]

¹HNMR (300 MHz, DMSO) δ: 10.62-10.67 (m, 1H), 7.62-7.66 (m, 1H), 7.48-7.55 (m, 2H), 7.43 (s, 1H), 7.36-7.39 (m, 1H), 7.24-7.28 (m, 1H), 6.52-6.57 (m, 2H), 5.38-5.46 (m, 3H), 4.64 (s, 2H), 4.30 (s, 2H), 3.85 (s, 3H), 3.65-3.69 (m, 2H). ¹⁹F NMR (300 MHz, DMSO) δ: −122.85. Mass spectrum (ESI, m/z): Calculated for C₂₃H₂₂Cl₂FN₄O₅S, 555.1 (M+H), found 555.0.

### (Z)-2-chloro-N-((2-(1-(2,4-dichlorobenzyl)-1,4-dihydropyrano[4,3-c]pyrazol-7(6H)-ylidene)ethyl)carbamoyl)-5-methoxybenzenesulfonamide, Compound #65

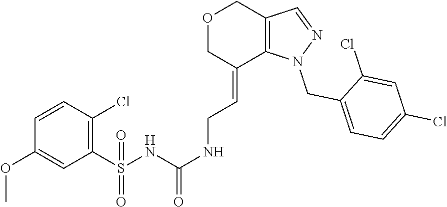 [SMILES: COc1ccc(Cl)c(S(=O)(=O)NC(=O)NC/C=C2\COCc3cnn(Cc4ccc(Cl)cc4Cl)c32)c1]

¹H NMR (300 MHz, CD₃OD) δ: 7.64-7.65 (m, 1H), 7.40-7.46 (m, 3H), 7.18-7.22 (m, 1H), 7.11-7.15 (m, 1H), 6.45 (d, J=8.4 Hz, 1H), 5.46 (s, 2H), 5.32-5.37 (m, 1H), 4.68 (s, 2H), 4.33 (s, 2H), 3.83 (s, 3H), 3.73 (d, J=6.6 Hz, 2H). Mass spectrum (ESI, m/z): Calculated for C₂₃H₂₂Cl₃N₄O₅S, 571.1 (M+H), found 572.8.

### (Z)-5-chloro-N-((2-(1-(2,4-dichlorobenzyl)-1,4-dihydropyrano[4,3-c]pyrazol-7(6H)-ylidene)ethyl)carbamoyl)-2-methoxybenzenesulfonamide, Compound #66

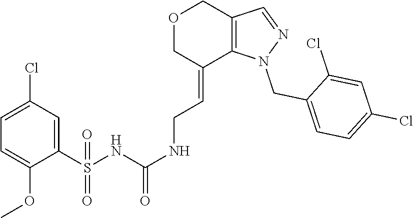 [SMILES: COc1ccc(Cl)cc1S(=O)(=O)NC(=O)NC/C=C1\COCc2cnn(Cc3ccc(Cl)cc3Cl)c21]

¹H NMR (300 MHz, DMSO) δ 10.74 (s, 1H), 7.70-7.73 (m, 2H), 7.68 (s, 1H), 7.43 (s, 1H), 7.35-7.39 (m, 1H), 7.27 (d, J=9.0 Hz, 1H), 6.56 (d, J=8.4 Hz, 2H), 5.38-5.45 (m, 3H), 4.71 (s, 2H), 4.30 (s, 2H), 3.90 (s, 3H), 3.65-3.66 (m, 2H). Mass spectrum (ESI, m/z): Calculated for C₂₃H₂₂Cl₃N₄O₅S, 572.8 (M+H), found 572.9.

### (Z)—N-((2-(1-(2,4-dichlorobenzyl)-1,4-dihydropyrano[4,3-c]pyrazol-7(6H)-ylidene)ethyl)carbamoyl)-6-methoxypyridine-3-sulfonamide, Compound #67

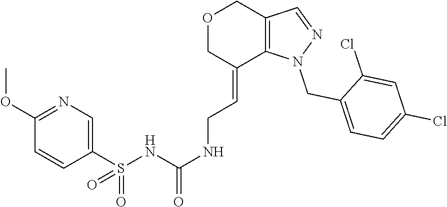 [SMILES: COc1ccc(S(=O)(=O)NC(=O)NC/C=C2\COCc3cnn(Cc4ccc(Cl)cc4Cl)c32)cn1]

¹H NMR (300 MHz, CD₃OD) δ 8.68 (s, 1H), 8.10 (d, J=8.9 Hz, 1H), 7.35-7.52 (m, 2H), 7.19 (d, J=8.3 Hz, 1H), 6.89 (d, J=8.8 Hz, 1H), 6.46 (d, J=8.5 Hz, 1H), 5.47 (s, 2H), 5.35 (t, J=6.8 Hz, 1H), 4.68 (s, 2H), 4.35 (s, 2H), 3.97 (s, 3H), 3.73 (d, J=6.8 Hz, 2H). Mass spectrum (ESI, m/z): Calculated for C₂₂H₂₂Cl₂N₅O₅S, 538.1 (M+H), found 538.1.

### (Z)—N-((2-(1-(2,4-dichlorobenzyl)-1,4-dihydropyrano[4,3-c]pyrazol-7(6H)-ylidene)ethyl)carbamoyl)-2-fluoro-5-methoxybenzenesulfonamide, Compound #68

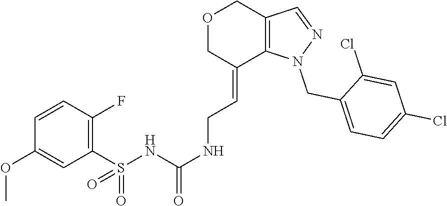 [SMILES: COc1ccc(F)c(S(=O)(=O)NC(=O)NC/C=C2\COCc3cnn(Cc4ccc(Cl)cc4Cl)c32)c1]

¹H NMR (300 MHz, DMSO) b 11.09 (s, 1H), 7.66 (s, 1H), 7.30-7.47 (m, 3H), 7.25-7.29 (m, 2H), 6.52-6.64 (m, 2H), 5.52 (s, 2H), 5.39-5.49 (m, 1H), 4.64 (s, 2H), 4.29 (s, 2H), 3.87 (s, 3H), 3.66-3.68 (m, 2H). ¹⁹F NMR (300 MHz, DMSO) δ: −73.48, −122.30. Mass spectrum (ESI, m/z): Calculated for C₂₃H₂₂Cl₂FN₄O₅S, 555.4 (M+H), found 555.1.

### Example 34: Compound #55

### (Z)-4,5-dichloro-N-((2-(1-(2,4-dichlorobenzyl)-1,4-dihydropyrano[4,3-c]pyrazol-7(6H)-ylidene)ethyl)(methyl)carbamoyl)thiophene-2-sulfonamide

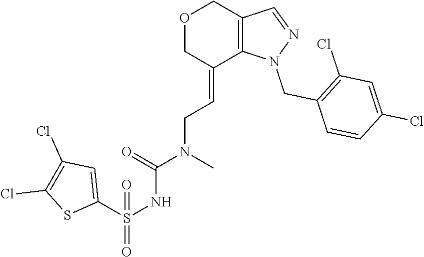 [SMILES: CN(C/C=C1\COCc2cnn(Cc3ccc(Cl)cc3Cl)c21)C(=O)NS(=O)(=O)c1cc(Cl)c(Cl)s1]

### Steps 1-4. Synthesis of (Z)-4,5-dichloro-N-((2-(1-(2,4-dichlorobenzyl)-1,4-dihydropyrano[4,3-c]pyrazol-7(6H)-ylidene)ethyl)(methyl)carbamoyl)thiophene-2-sulfonamide

(Z)-4,5-dichloro-N-((2-(1-(2,4-dichlorobenzyl)-1,4-dihydropyrano[4,3-c]pyrazol-7(6H)-ylidene)ethyl)(methyl)carbamoyl)thiophene-2-sulfonamide was prepared from (Z)-2-(1-(2,4-dichlorobenzyl)-1,4-dihydropyrano[4,3-c]pyrazol-7(6H)-ylidene)ethan-1-amine according to the procedures as described in Example 17, Steps 1-4.

¹H NMR (300 MHz, CD₃OD) δ: 7.49 (s, 1H), 7.44 (s, 1H), 7.33 (s, 1H), 7.20 (d, J=8.4 Hz, 1H), 6.45 (d, J=8.4 Hz, 1H), 5.52 (s, 2H), 5.39-5.44 (m, 1H), 4.69 (s, 2H), 4.42 (s, 2H), 3.99-4.08 (m, 2H), 2.60 (brs, 3H). Mass spectrum (ESI, m/z): Calculated for C₂₁H₁₉Cl₄N₄O₄S₂, 595.0 (M+H), found 597.0.

### Example 35: Compound #54 and Compound #60

### (E)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide and

### (Z)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide

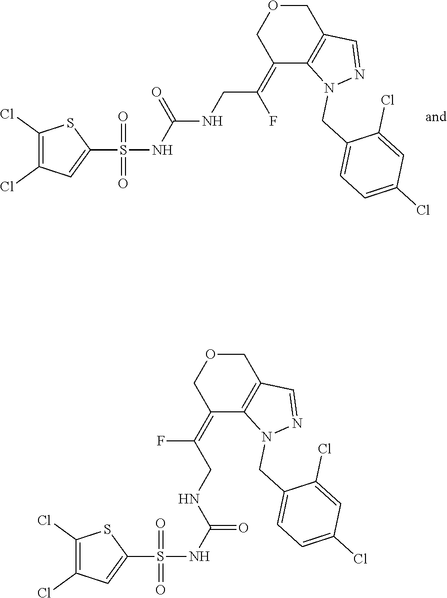 [SMILES: O=C(NC/C(F)=C1\COCc2cnn(Cc3ccc(Cl)cc3Cl)c21)NS(=O)(=O)c1cc(Cl)c(Cl)s1 ; O=C(NC/C(F)=C1/COCc2cnn(Cc3ccc(Cl)cc3Cl)c21)NS(=O)(=O)c1cc(Cl)c(Cl)s1]

### Step 1. Synthesis of ethyl 2-(1-(2,4-dichlorobenzyl)-1,4-dihydropyrano[4,3-c]pyrazol-7(6H)-ylidene)-2-fluoroacetate

To a solution of ethyl 2-(diethoxyphosphoryl)-2-fluoroacetate (5.1 g, 21.1 mmol) in THF (50 ml) at −78° C. was added n-BuLi (8.4 ml, 21.00 mmol) dropwise with stirring. The reaction was stirred for 2.0 h at −78° C. before 1-(2,4-dichlorobenzyl)-1,4-dihydropyrano[4,3-c]pyrazol-7(6H)-one (2.0 g, 6.73 mmol) was added. The reaction was stirred overnight at room temperature and monitored by LCMS. The reaction was then quenched with water and extracted with ethyl acetate (3×50 mL). The organic layers were combined and concentrated under vacuum. The residue was purified by silica gel column with ethyl acetate/petroleum ether (15/85) to yield ethyl 2-(1-(2,4-dichlorobenzyl)-1,4-dihydropyrano[4,3-c]pyrazol-7(6H)-ylidene)-2-fluoroacetate as a mixture of (E) and (Z) isomers. Mass spectrum (ESI, m/z): Calculated for C₁₇H₁₆Cl₂FN₂O₃, 385.0 (M+H), found 384.9.

### Step 2. Synthesis of (E)-2-(1-(2,4-dichlorobenzyl)-1,4-dihydropyrano[4,3-c]pyrazol-7(6H)-ylidene)-2-fluoroethan-1-01 and (Z)-2-(1-(2,4-dichlorobenzyl)-1,4-dihydropyrano[4,3-c]pyrazol-7(6H)-ylidene)-2-fluoroethan-1-ol

To a solution of ethyl 2-(1-(2,4-dichlorobenzyl)-1,4-dihydropyrano[4,3-c]pyrazol-7(6H)-ylidene)-2-fluoroacetate (100 mg, 0.260 mmol) in THF (10 ml) at −78° C. was added DIBAL-H (0.8 ml, 0.8 mmol) dropwise with stirring. The resulting solution was stirred for 3.0 h at room temperature. The reaction progress was monitored by LCMS. The reaction was then quenched with water, and the resulting mixture was adjusted to pH4 with aq. 2N HCl solution and extracted with DCM (3×10 mL). The organic layers were combined and concentrated under vacuum. The residue was purified by silica gel column with ethyl acetate/petroleum ether (30/70) to yield (Z)-2-(1-(2,4-dichlorobenzyl)-1,4-dihydropyrano[4,3-c]pyrazol-7(6H)-ylidene)-2-fluoroethan-1-ol followed by (E)-2-(1-(2,4-dichlorobenzyl)-1,4-dihydropyrano[4,3-c]pyrazol-7(6H)-ylidene)-2-fluoroethan-1-ol. Mass spectrum (ESI, m/z): Calculated for C₁₅H₁₄Cl₂FN₂O₂, 343.0 (M+H), found 343.0.

### Steps 3-5. Synthesis of (E)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide

(E)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide was prepared from (E)-2-(1-(2,4-dichlorobenzyl)-1,4-dihydropyrano[4,3-c]pyrazol-7(6H)-ylidene)-2-fluoroethan-1-ol according to the procedures as described in Example 5, Steps 1-3.

¹H NMR (300 MHz, CD₃OD) δ: 7.63 (s, 1H), 7.36-7.41 (m, 2H), 7.17-7.21 (m, 1H), 6.67 (d, J=8.4 Hz, 1H), 5.43 (s, 2H), 4.74 (s, 2H), 4.35 (d, J=2.1 Hz, 2H), 4.05 (d, J=21.6 Hz, 2H). ¹⁹F NMR (300 MHz, CD₃OD) δ: −77.31, −104.51. Mass spectrum (ESI, m/z): Calculated for C₂₀H₁₆Cl₄FN₄O₄S₂, 598.9 (M+H), found 600.8.

### Steps 6-8. Synthesis of (Z)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide

(Z)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide was prepared from (Z)-2-(1-(2,4-dichlorobenzyl)-1,4-dihydropyrano[4,3-c]pyrazol-7(6H)-ylidene)-2-fluoroethan-1-ol according to the procedures as described in Example 5, Steps 1-3.

¹HNMR (300 MHz, CD₃OD) δ: 7.62 (d, J=7.5 Hz, 1H), 7.37-7.43 (m, 2H), 7.17-7.26 (m, 1H), 6.65-6.91 (m, 1H), 5.43 (s, 1H), 5.31 (s, 1H), 4.72-4.74 (m, 2H), 4.31-4.35 (m, 2H), 4.07-4.09 (m, 1H), 4.00-4.01 (m, 1H). ¹⁹F NMR (300 MHz, CD₃OD) δ: −77.30, −104.51, −111.50. Mass spectrum (ESI, m/z): Calculated for C₂₀H₁₆Cl₄FN₄O₄S₂, 598.9 (M+H-1.76CF₃COOH), found 600.9.

The following representative compounds of formula (I) were similarly prepared according to the procedure described in Example 35 (and Examples referenced therein) above, selecting and substituting suitable starting materials and reagents, as would be readily recognized by those skilled in the art.

### (E)-4-chloro-N-((2-(1-(2,4-dichlorobenzyl)-1,4-dihydropyrano[4,3-c]pyrazol-7(6H)-ylidene)-2-fluoroethyl)carbamoyl)benzenesulfonamide, Compound #83

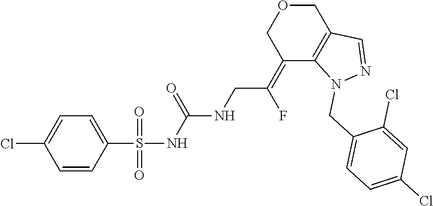 [SMILES: O=C(NC/C(F)=C1\COCc2cnn(Cc3ccc(Cl)cc3Cl)c21)NS(=O)(=O)c1ccc(Cl)cc1]

¹H NMR (300 MHz, CD₃OD) δ: 7.89-7.91 (m, 2H), 7.49-7.57 (m, 2H), 7.36-7.41 (m, 2H), 7.17-7.24 (m, 1H), 6.64 (d, J=8.4 Hz, 1H), 5.40 (s, 2H), 4.7 (s, 2H), 4.29 (s, 2H), 4.04 (s, 1H), 3.96 (s, 1H). ¹⁹F NMR (300 MHz, CD₃OD) δ: −104.52. Mass spectrum (ESI, m/z): Calculated for C₂₂H₁₉Cl₃FN₄O₄S, 559.1 (M+H), found 560.9.

### (E)-4-chloro-N-((2-fluoro-2-(1-(3-(trifluoromethyl)phenyl)-1,4-dihydropyrano[4,3-c]pyrazol-7(6H)-ylidene)ethyl)carbamoyl)benzenesulfonamide, Compound #104

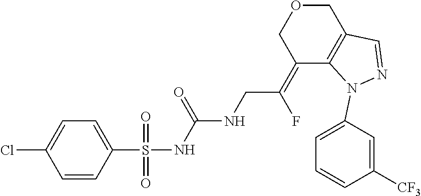 [SMILES: O=C(NC/C(F)=C1\COCc2cnn(-c3cccc(C(F)(F)F)c3)c21)NS(=O)(=O)c1ccc(Cl)cc1]

¹H NMR (CHLOROFORM-d) δ: 7.75 (d, J=8.1 Hz, 2H), 7.48-7.68 (m, 5H), 7.38 (d, J=8.6 Hz, 2H), 6.79 (br t, J=5.6 Hz, 1H), 4.77-4.89 (m, 2H), 4.49 (s, 2H), 3.88-4.05 (m, 2H). Mass spectrum (ESI, m/z): Calculated for C₂₂H₁₈ClF₄N₄O₄S, 545.1 (M+H), found 545.1.

### (E)-4-chloro-N-((2-fluoro-2-(1-(4-(trifluoromethyl)phenyl)-1,4-dihydropyrano[4,3-c]pyrazol-7(6H)-ylidene)ethyl)carbamoyl)benzenesulfonamide, Compound #118

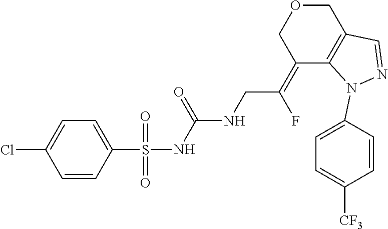 [SMILES: O=C(NC/C(F)=C1\COCc2cnn(-c3ccc(C(F)(F)F)cc3)c21)NS(=O)(=O)c1ccc(Cl)cc1]

¹H NMR (CHLOROFORM-d) δ: 7.63-7.76 (m, 4H), 7.50-7.56 (m, 3H), 7.33-7.41 (m, 2H), 6.81 (br t, J=5.6 Hz, 1H), 4.81 (s, 2H), 4.48 (d, J=2.0 Hz, 2H), 3.94-4.07 (m, 2H). Mass spectrum (ESI, m/z): Calculated for C₂₂H₁₈ClF₄N₄O₄S, 545.1 (M+H), found 545.1.

### (E)-4,5-dichloro-N-((2-fluoro-2-(1-(4-(trifluoromethyl)phenyl)-1,4-dihydropyrano[4,3-c]pyrazol-7(6H)-ylidene)ethyl)carbamoyl)thiophene-2-sulfonamide, Compound #119

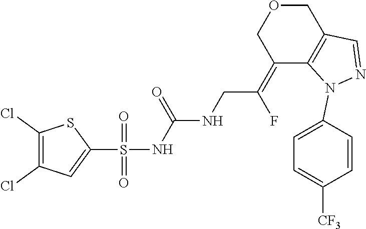 [SMILES: O=C(NC/C(F)=C1\COCc2cnn(-c3ccc(C(F)(F)F)cc3)c21)NS(=O)(=O)c1cc(Cl)c(Cl)s1]

¹H NMR (CHLOROFORM-d) δ: 7.66 (br d, J=8.6 Hz, 2H), 7.42-7.56 (m, 4H), 4.85 (s, 2H), 4.47-4.59 (m, 2H), 3.91-4.06 (m, 2H). Mass spectrum (ESI, m/z): Calculated for C₂₀H₁₅Cl₂F₄N₄O₄S, 585.0 (M+H), found 585.0.

### (E)-4,5-dichloro-N-((2-fluoro-2-(1-(1-(4-fluorophenyl)piperidin-4-yl)-1,4-dihydropyrano[4,3-c]pyrazol-7(6H)-ylidene)ethyl)carbamoyl)thiophene-2-sulfonamide, Compound #144

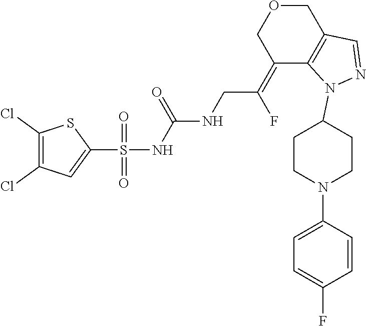 [SMILES: O=C(NC/C(F)=C1\COCc2cnn(C3CCN(c4ccc(F)cc4)CC3)c21)NS(=O)(=O)c1cc(Cl)c(Cl)s1]

¹H NMR (CHLOROFORM-d) δ: 7.47 (br s, 1H), 7.32 (s, 1H), 6.97 (m, 4H), 4.73 (br s, 2H), 4.36 (m, 3H), 4.00-4.22 (m, 2H), 3.69 (m, 2H), 2.87 (m, 2H), 2.33 (m, 2H), 1.93-2.07 (m, 2H). Mass spectrum (ESI, m/z): Calculated for C₂₄H₂₄Cl₂F₂N₅O₄S₂, 618.1 (M+H), found 618.2.

### Example 36: Compound #160

### (E)-N-((2-(1-(2,4-dichlorobenzyl)-1,4-dihydropyrano[4,3-c]pyrazol-7(6H)-ylidene)-2-fluoroethyl)carbamoyl)-2,4-dimethylthiazole-5-sulfonamide

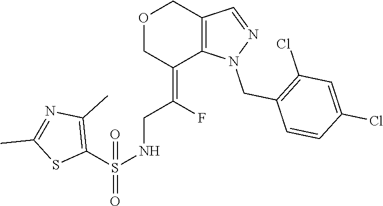 [SMILES: Cc1nc(C)c(S(=O)(=O)NC/C(F)=C2\COCc3cnn(Cc4ccc(Cl)cc4Cl)c32)s1]

### Step 1. Synthesis of phenyl (E)-(2-(1-(2,4-dichlorobenzyl)-1,4-dihydropyrano[4,3-c]pyrazol-7(6H)-ylidene)-2-fluoroethyl)carbamate

To a solution of (E)-2-(1-(2,4-dichlorobenzyl)-1,4-dihydropyrano[4,3-c]pyrazol-7(6H)-ylidene)-2-fluoroethan-1-amine (500 mg, 1.24 mmol) in DCM (20 mL) was added phenyl carbonochloridate (233 mg, 1.488 mmol) and Et₃N (150 mg, 1.485 mmol). The resulting mixture was stirred at room temperature overnight. The reaction progress was monitored by LCMS. The resulting mixture was concentrated under vacuum. The residue obtained was purified by silica gel chromatography (40-60% EtOAc/petroleum ether) to yield phenyl (E)-(2-(1-(2,4-dichlorobenzyl)-1,4-dihydropyrano[4,3-c]pyrazol-7(6H)-ylidene)-2-fluoroethyl)carbamate as a yellow solid. Mass spectrum (ESI, m/z): Calculated for C₂₂H₁₉Cl₂FN₃O₃, 462.1 [M+H], found 462.0.

### Step 2. Synthesis of (E)-N-((2-(1-(2,4-dichlorobenzyl)-1,4-dihydropyrano[4,3-c]pyrazol-7(6H)-ylidene)-2-fluoroethyl)carbamoyl)-2,4-dimethylthiazole-5-sulfonamide

A solution of phenyl (E)-(2-(1-(2,4-dichlorobenzyl)-1,4-dihydropyrano[4,3-c]pyrazol-7(6H)-ylidene)-2-fluoroethyl)carbamate (50 mg, 0.108 mmol), 2,4-dimethylthiazole-5-sulfonamide (25 mg, 0.130 mmol), and DBU (20 mg, 0.132 mmol) in CH₃CN (10 ml) was stirred for 4.0 h at room temperature. The reaction progress was monitored by LCMS. The resulting solution was concentrated under vacuum. The resulting residue was purified by Prep-HPLC with the following conditions (1#-Waters 2767-5): Column, SunFire Prep C18, 5 um, 19*100 mm; mobile phase, Water with 0.05% TFA and CH₃CN (50% CH₃CN up to 80% in 10 min, up to 100% in 0.1 min, hold 100% in 0.9 min, down to 50% in 0.1 min, hold 50% in 1.4 min); Detector, UV 220&254 nm. The desired fraction was concentrated under vacuum. The resulting residue was dissolved in CH₃CN (5 mL) and then HCl (4 N, 2 mL) was added. The resulting solution was concentrated under vacuum. This was repeated and the resulting residue lyophilized to yield (E)-N-((2-(1-(2,4-dichlorobenzyl)-1,4-dihydropyrano[4,3-c]pyrazol-7(6H)-ylidene)-2-fluoroethyl)carbamoyl)-2,4-dimethylthiazole-5-sulfonamide as a light yellow solid.

¹H NMR (400 MHz, CD₃OD) δ: 7.57 (s, 1H), 7.48-7.49 (m, 1H), 7.26-7.29 (m, 1H), 6.73 (d, J=8.4 Hz, 1H), 5.52 (s, 2H), 4.79 (s, 2H), 4.39-4.40 (m, 2H), 4.05-4.11 (m, 2H), 2.78-2.81 (m, 3H), 2.64-2.65 (m, 3H). ¹⁹F NMR (400 MHz, CD₃OD) δ: −103.28. Mass spectrum (ESI, m/z): Calculated for C₂₁H₂₁Cl₂FN₅O₄S₂, 560.0 [M+H], found 560.0.

The following representative compounds of formula (I) were similarly prepared according to the procedure described in Example 36 (and Examples referenced therein) above, selecting and substituting suitable starting materials and reagents, as would be readily recognized by those skilled in the art.

### (E)-N-((2-(1-(2,4-dichlorobenzyl)-1,4-dihydropyrano[4,3-c]pyrazol-7(6H)-ylidene)-2-fluoroethyl)carbamoyl)-3-fluoro-4-methoxybenzenesulfonamide, Compound #161

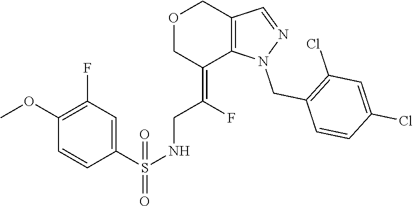 [SMILES: COc1ccc(S(=O)(=O)NC/C(F)=C2\COCc3cnn(Cc4ccc(Cl)cc4Cl)c32)cc1F]

¹H NMR (400 MHz, CD₃OD) δ: 7.78-7.79 (m, 1H), 7.69-7.76 (m, 1H), 7.43-7.46 (m, 2H), 7.20-7.26 (m, 2H), 6.70-6.72 (m, 1H), 5.45 (s, 2H), 4.77 (s, 2H), 4.34-4.35 (m, 2H), 4.01-4.08 (m, 2H), 3.93 (s, 3H). ¹⁹F NMR (400 MHz, CD₃OD) δ: −104.47, −134.40. Mass spectrum (ESI, m/z): Calculated for C₂₃H₂₁Cl₂F₂N₄O₅S, 573.0 [M+H], found 573.0.

### (E)-3-chloro-N-((2-(1-(2,4-dichlorobenzyl)-1,4-dihydropyrano[4,3-c]pyrazol-7(6H)-ylidene)-2-fluoroethyl)carbamoyl)-4-methoxybenzenesulfonamide, Compound #165

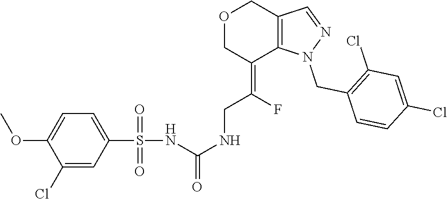 [SMILES: COc1ccc(S(=O)(=O)NC(=O)NC/C(F)=C2\COCc3cnn(Cc4ccc(Cl)cc4Cl)c32)cc1Cl]

¹H NMR (300 MHz, CD₃OD) δ: 7.97-7.98 (m, 1H), 7.89-7.92 (m, 1H), 7.42-7.45 (m, 2H), 7.18-7.23 (m, 2H), 6.72 (d, J=8.7 Hz, 1H), 5.44 (s, 2H), 4.77 (s, 2H), 4.35 (s, 2H), 4.06 (d, J=21.9 Hz, 2H), 3.95 (s, 3H). ¹⁹F NMR (300 MHz, CD₃OD) δ: −104.45. Mass spectrum (ESI, m/z): Calculated for C₂₄H₂₀Cl₃FN₄O₅S, 589.0 [M+H], found 590.9.

### (E)-5-chloro-N-((2-(1-(2,4-dichlorobenzyl)-1,4-dihydropyrano[4,3-c]pyrazol-7(6H)-ylidene)-2-fluoroethyl)carbamoyl)-4-methylthiophene-2-sulfonamide, Compound #166

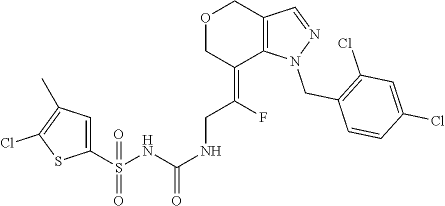 [SMILES: Cc1cc(S(=O)(=O)NC(=O)NC/C(F)=C2\COCc3cnn(Cc4ccc(Cl)cc4Cl)c32)sc1Cl]

¹H NMR (300 MHz, CD₃OD) δ: 7.55 (s, 1H), 7.45-7.48 (m, 2H), 7.23-7.26 (m, 1H), 6.72 (d, J=8.4 Hz, 1H), 5.49 (s, 2H), 4.79 (s, 2H), 4.39 (s, 2H), 4.10 (d, J=21.9 Hz, 2H), 2.18 (s, 3H). ¹⁹F NMR (300 MHz, CD₃OD) δ: −104.23. Mass spectrum (ESI, m/z): Calculated for C₂₁H₁₉Cl₃FN₄O₄S₂, 579.0 [M+H], found 580.7.

### Example 37: Compound #74

### (E)-4,5-dichloro-N-((2-(1-(2,4-dichlorobenzyl)-1,4-dihydropyrano[4,3-c]pyrazol-7(6H)-ylidene)-2-fluoroethyl)(methyl)carbamoyl)thiophene-2-sulfonamide

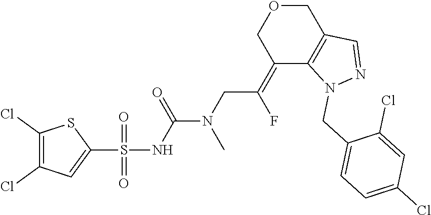 [SMILES: CN(C/C(F)=C1\COCc2cnn(Cc3ccc(Cl)cc3Cl)c21)C(=O)NS(=O)(=O)c1cc(Cl)c(Cl)s1]

### Step 1. Synthesis of tert-butyl (E)-(2-(1-(2,4-dichlorobenzyl)-1,4-dihydropyrano[4,3-c]pyrazol-7(6H)-ylidene)-2-fluoroethyl)carbamate

Tert-butyl (E)-(2-(1-(2,4-dichlorobenzyl)-1,4-dihydropyrano[4,3-c]pyrazol-7(6H)-ylidene)-2-fluoroethyl)carbamate was prepared from (E)-2-(1-(2,4-dichlorobenzyl)-1,4-dihydropyrano[4,3-c]pyrazol-7(6H)-ylidene)-2-fluoroethan-1-amine according to the procedures as described in Example 17, Step 1.

### Step 2. Synthesis of tert-butyl (E)-(2-(1-(2,4-dichlorobenzyl)-1,4-dihydropyrano[4,3-c]pyrazol-7(6H)-ylidene)-2-fluoroethyl)(methyl)carbamate

To a solution of tert-butyl (E)-(2-(1-(2,4-dichlorobenzyl)-1,4-dihydropyrano[4,3-c]pyrazol-7(6H)-ylidene)-2-fluoroethyl)carbamate (20 mg, 0.045 mmol) in THF (5 ml) at −78° C. under nitrogen was added LiHMDS (0.1 ml, 0.1 mmol) with stirring. The reaction was stirred for 30 min at −78° C. before Mel (13 mg, 0.092 mmol) was added. The reaction was then stirred for 3.0 h at room temperature and its progress was monitored by LCMS. The reaction was then quenched by the addition of MeOH. The resulting solution was concentrated under vacuum. The resulting residue was purified by TLC with PE:EA=2:1 to yield tert-butyl (E)-(2-(1-(2,4-dichlorobenzyl)-1,4-dihydropyrano[4,3-c]pyrazol-7(6H)-ylidene)-2-fluoroethyl)(methyl)carbamate as a yellow oil. Mass spectrum (ESI, m/z): Calculated for C₂₁H₂₅Cl₂FN₃O₃, 456.1(M+H), found 456.0.

### Step 3. Synthesis of (E)-2-(1-(2,4-dichlorobenzyl)-1,4-dihydropyrano[4,3-c]pyrazol-7(6H)-ylidene)-2-fluoro-N-methylethan-1-amine

(E)-2-(1-(2,4-dichlorobenzyl)-1,4-dihydropyrano[4,3-c]pyrazol-7(6H)-ylidene)-2-fluoro-N-methylethan-1-amine was prepared from tert-butyl (E)-(2-(1-(2,4-dichlorobenzyl)-1,4-dihydropyrano[4,3-c]pyrazol-7(6H)-ylidene)-2-fluoroethyl)(methyl)carbamate according to the procedures as described in Example 17, Step 3.

### Step 4. Synthesis of (E)-4,5-dichloro-N-((2-(1-(2,4-dichlorobenzyl)-1,4-dihydropyrano[4,3-c]pyrazol-7(6H)-ylidene)-2-fluoroethyl)(methyl)carbamoyl)thiophene-2-sulfonamide

(E)-4,5-dichloro-N-((2-(1-(2,4-dichlorobenzyl)-1,4-dihydropyrano[4,3-c]pyrazol-7(6H)-ylidene)-2-fluoroethyl)(methyl)carbamoyl)thiophene-2-sulfonamide was prepared from (E)-2-(1-(2,4-dichlorobenzyl)-1,4-dihydropyrano[4,3-c]pyrazol-7(6H)-ylidene)-2-fluoro-N-methylethan-1-amine according to the procedures as described in Example 17, Step 4.

¹H NMR (300 MHz, CD₃OD) δ: 7.40-7.41 (m, 2H), 7.31 (s, 1H), 7.18 (d, J=6.3 Hz, 1H), 6.62 (d, J=8.4 Hz, 1H), 5.45 (s, 2H), 4.75 (s, 2H), 4.25-4.38 (m, 4H), 2.79 (brs, 3H). ¹⁹F NMR (300 MHz, CD₃OD) δ: −101.64. Mass spectrum (ESI, m/z): Calculated for C₁₆H₁₆Cl₂FN₃O, 612.9 (M+H), found 614.9.

### Example 38: Compound #57

### (Z)-4-(1-(2,4-dichlorobenzyl)-1,4-dihydropyrano[4,3-c]pyrazol-7(6H)-ylidene)-N-((4,5-dichlorothiophen-2-yl)sulfonyl)butanamide

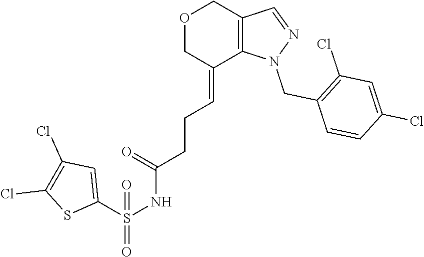 [SMILES: O=C(CC/C=C1\COCc2cnn(Cc3ccc(Cl)cc3Cl)c21)NS(=O)(=O)c1cc(Cl)c(Cl)s1]

(Z)-4-(1-(2,4-dichlorobenzyl)-1,4-dihydropyrano[4,3-c]pyrazol-7(6H)-ylidene)-N-((4,5-dichlorothiophen-2-yl)sulfonyl)butanamide was prepared from (Z)-2-(1-(2,4-dichlorobenzyl)-1,4-dihydropyrano[4,3-c]pyrazol-7(6H)-ylidene)ethan-1-ol according to the procedures as described in Example 4, Steps 1-4.

¹H NMR (300 MHz, CD₃OD) δ: 7.63 (s, 1H), 7.50-7.51 (m, 1H), 7.39 (s, 1H), 7.18-7.22 (m, 1H), 6.45 (d, J=8.7 Hz, 1H), 5.46 (s, 2H), 5.33-5.37 (m, 1H), 4.68 (s, 2H), 4.32 (s, 2H), 2.25-2.35 (m, 4H). Mass spectrum (ESI, m/z): Calculated for C₂₁H₁₈Cl₄N₃O₄S₂, 579.9 (M+H), found 581.8.

### Example 39: Compound #61

### (E)-4-(1-(2,4-dichlorobenzyl)-1,4-dihydropyrano[4,3-c]pyrazol-7(6H)-ylidene)-N-((4,5-dichlorothiophen-2-yl)sulfonyl)-4-fluorobutanamide

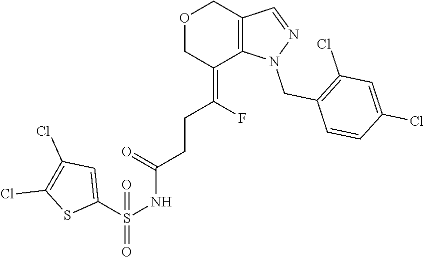 [SMILES: O=C(CC/C(F)=C1\COCc2cnn(Cc3ccc(Cl)cc3Cl)c21)NS(=O)(=O)c1cc(Cl)c(Cl)s1]

(E)-4-(1-(2,4-dichlorobenzyl)-1,4-dihydropyrano[4,3-c]pyrazol-7(6H)-ylidene)-N-((4,5-dichlorothiophen-2-yl)sulfonyl)-4-fluorobutanamide from (E)-2-(1-(2,4-dichlorobenzyl)-1,4-dihydropyrano[4,3-c]pyrazol-7(6H)-ylidene)-2-fluoroethan-1-ol according to the procedures as described in Example 4, Steps 1-4.

¹H NMR (400 MHz, CD₃OD) δ: 7.68 (d, J=8.8 Hz, 1H), 7.43-7.45 (m, 2H), 7.21-7.27 (m, 1H), 6.64 (d, J=8.4 Hz, 1H), 5.41 (s, 2H), 4.75 (s, 2H), 4.22 (d, J=8.0 Hz, 2H), 2.65-2.75 (m, 2H), 2.50-2.54 (m, 2H). ¹⁹F NMR (400 MHz, CD₃OD) δ: −100.06. Mass spectrum (ESI, m/z): Calculated for C₂₁H₁₇Cl₄FN₃O₄S₂, 597.9 (M+H), found 599.8.

### Example 40: Compound #127 and Compound #128

### (Z)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide and

### (E)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide

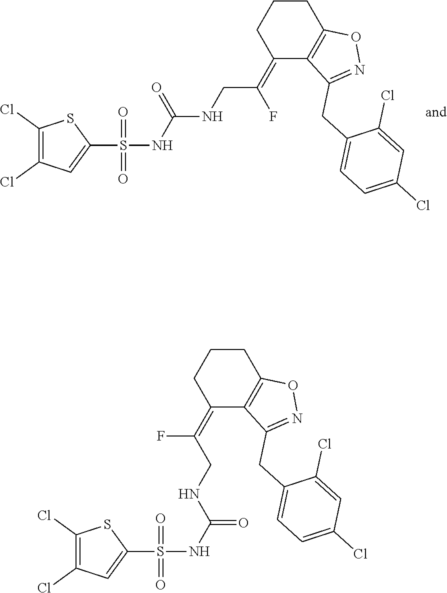 [SMILES: O=C(NC/C(F)=C1\CCCc2onc(Cc3ccc(Cl)cc3Cl)c21)NS(=O)(=O)c1cc(Cl)c(Cl)s1 ; O=C(NC/C(F)=C1/CCCc2onc(Cc3ccc(Cl)cc3Cl)c21)NS(=O)(=O)c1cc(Cl)c(Cl)s1]

### Step 1. Synthesis of 2-(2-(2,4-dichlorophenyl)acetyl)cyclohexane-1,3-dione

To 2-(2,4-dichlorophenyl)acetic acid (2.0 g, 9.75 mmol) and cyclohexane-1,3-dione (1.31 g, 11.7 mmol) in EtOAc (80 mL) at room temperature was added DCC (2.42 g, 11.7 mmol). The reaction was stirred overnight. To the reaction mixture was added heptane (150 ml). The resulting mixture was filtered and the solution was washed with H₂O, sat. aq. NaHCO₃ and sat. aq. NaCl, dried over Na₂SO₄ and concentrated to yield a resulting residue. A solution of the resulting residue, 2-hydroxy-2-methylpropanenitrile (0.134 mL, 1.46 mmol), and Et₃N (4.07 mL, 29.3 mmol) in CH₃CN (50 mL) was stirred at room temperature for 3 days. The reaction was concentrated. The residue was dissolved in EtOAc and washed with aq. 1N HCl and sat. aq. NaCl, dried over Na₂SO₄, and concentrated to yield 2-(2-(2,4-dichlorophenyl)acetyl)cyclohexane-1,3-dione.

### Step 2. Synthesis of 3-(2,4-dichlorobenzyl)-6,7-dihydrobenzo[d]isoxazol-4(5H)-one

A mixture of 2-(2-(2,4-dichlorophenyl)acetyl)cyclohexane-1,3-dione (300 mg, 1.00 mmol), hydroxylamine hydrochloride (69.7 mg, 1.00 mmol), and KOH (56.3 mg, 1.00 mmol) in EtOH (8 mL) was heated at 60° C. for 1 h. The reaction mixture was concentrated and the residue was diluted with EtOAc. The resulting solution was washed with H₂O and sat. aq. NaCl, dried over Na₂SO₄ and concentrated. The resulting residue was purified by silica gel column with 10-20% EtOAc/heptane to yield 3-(2,4-dichlorobenzyl)-6,7-dihydrobenzo[d]isoxazol-4(5H)-one. Mass spectrum (ESI, m/z): Calculated for C₁₄H₁₂Cl₂NO₂, 296.0 (M+H), found 296.0.

### Step 3. Synthesis of ethyl 2-(3-(2,4-dichlorobenzyl)-6,7-dihydrobenzo[d]isoxazol-4(5H)-ylidene)-2-fluoroacetate

To a solution of ethyl 2-(diethoxyphosphoryl)-2-fluoroacetate (263 mg, 1.08 mmol) in THF (5 mL) at 0° C. was added NaH (60% in mineral oil, 39 mg, 0.976 mmol). The reaction was stirred for 30 min at 0° C. A solution of 3-(2,4-dichlorobenzyl)-6,7-dihydrobenzo[d]isoxazol-4(5H)-one (107 mg, 0.361 mmol) in THF (2 mL) was then added. The reaction was warmed up to room temperature and stirred for overnight. LC/MS showed incomplete reaction. The reaction was cooled to 0° C. and additional ethyl 2-(diethoxyphosphoryl)-2-fluoroacetate (131 mg, 0.54 mmol) and NaH (60% in mineral oil, 20 mg, 0.50 mmol) were added. The reaction was warmed up to room temperature and stirred for 3 h. The reaction was quenched with aq. 1N HCl and the resulting mixture was extracted with EtOAc. The organic layer was washed with sat. aq. NaCl, dried over Na₂SO₄ and concentrated. The resulting residue was purified by silica gel column with 10% EtOAc/heptane to yield ethyl 2-(3-(2,4-dichlorobenzyl)-6,7-dihydrobenzo[d]isoxazol-4(5H)-ylidene)-2-fluoroacetate as a mixture of (E) and (Z) isomers.

### Step 4. Synthesis of (Z)-2-(3-(2,4-dichlorobenzyl)-6,7-dihydrobenzo[d]isoxazol-4(5H)-ylidene)-2-fluoroethan-1-ol and (E)-2-(3-(2,4-dichlorobenzyl)-6,7-dihydrobenzo[d]isoxazol-4(5H)-ylidene)-2-fluoroethan-1-ol

To a solution of ethyl 2-(3-(2,4-dichlorobenzyl)-6,7-dihydrobenzo[d]isoxazol-4(5H)-ylidene)-2-fluoroacetate (160 mg, 0.416 mmol) in CH₂Cl₂ (8 mL) at −78° C. was added DIBAL-H (1.0 M in heptane, 1.67 mL, 1.67 mmol). The reaction was stirred at −78° C. for 2 h. The reaction was then quenched with MeOH and H₂O. The resulting mixture was diluted with EtOAc and washed with aq. 1 N NaOH and sat. aq. NaCl. The organic layer was dried over Na₂SO₄ and concentrated. The resulting residue was purified by silica gel column with 10-40% EtOAc/heptane to yield (E)-2-(3-(2,4-dichlorobenzyl)-6,7-dihydrobenzo[d]isoxazol-4(5H)-ylidene)-2-fluoroethan-1-ol, then with 40% EtOAc/heptane to yield (Z)-2-(3-(2,4-dichlorobenzyl)-6,7-dihydrobenzo[d]isoxazol-4(5H)-ylidene)-2-fluoroethan-1-ol.

### Steps 5-7. Synthesis of (Z)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide

(Z)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide was prepared from (Z)-2-(3-(2,4-dichlorobenzyl)-6,7-dihydrobenzo[d]isoxazol-4(5H)-ylidene)-2-fluoroethan-1-ol according to the procedures as described in Example 5, Steps 1-3.

¹H NMR (CHLOROFORM-d) δ: 7.48-7.55 (m, 1H), 7.39 (d, J=2.5 Hz, 1H), 7.14 (dd, J=8.6, 2.0 Hz, 1H), 6.98 (d, J=8.6 Hz, 1H), 4.20 (d, J=4.0 Hz, 2H), 3.99-4.12 (m, 2H), 2.82 (t, J=6.6 Hz, 2H), 2.33-2.48 (m, 2H), 1.90-2.04 (m, 2H). Mass spectrum (ESI, m/z): Calculated for C₂₁H₁₇Cl₄FN₃O₄S₂, 597.9 (M+H), found 600.0.

### Steps 8-10. Synthesis of (E)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide

(E)-4,5-dichloro-N-((2-(3-(2,4-dichlorobenzyl)-6,7-dihydrobenzo[d]isoxazol-4(5H)-ylidene)-2-fluoroethyl)carbamoyl)thiophene-2-sulfonamide was prepared from (E)-2-(3-(2,4-dichlorobenzyl)-6,7-dihydrobenzo[d]isoxazol-4(5H)-ylidene)-2-fluoroethan-1-ol according to the procedures as described in Example 5, Steps 1-3.

¹H NMR (CHLOROFORM-d) δ: 7.47-7.55 (m, 1H), 7.37 (d, J=2.0 Hz, 1H), 7.10-7.21 (m, 2H), 3.96-4.13 (m, 4H), 2.81-2.94 (m, 2H), 2.42 (br dd, J=5.6, 3.0 Hz, 2H), 1.93-2.08 (m, 2H). Mass spectrum (ESI, m/z): Calculated for C₂₁H₁₇Cl₄FN₃O₄S₂, 597.9 (M+H), found 597.9.

The following representative compound of formula (I) was similarly prepared according to the procedure described in Example 40 (and Examples referenced therein) above, selecting and substituting suitable starting materials (including 2H-pyran-3,5(4H,6H)-dione instead of cyclohexane-1,3-dione) and reagents, as would be readily recognized by those skilled in the art.

### (E)-4,5-dichloro-N-((2-(3-(2,4-dichlorobenzyl)-5H-pyrano[4,3-d]isoxazol-4(7H)-ylidene)-2-fluoroethyl)carbamoyl)thiophene-2-sulfonamide, Compound #145

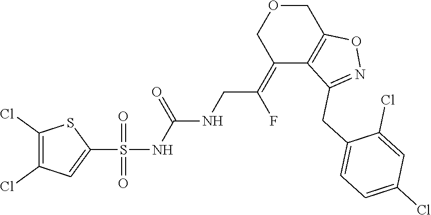 [SMILES: O=C(NC/C(F)=C1\COCc2onc(Cc3ccc(Cl)cc3Cl)c21)NS(=O)(=O)c1cc(Cl)c(Cl)s1]

¹H NMR (CHLOROFORM-d) δ: 7.53 (s, 1H), 7.41 (s, 1H), 7.16 (br d, J=8.1 Hz, 1H), 6.99 (br d, J=8.6 Hz, 1H), 4.83 (s, 2H), 4.40 (br s, 2H), 4.20-4.30 (m, 2H), 3.94-4.10 (m, 2H). Mass spectrum (ESI, m/z): Calculated for C₂₀H₁₅Cl₄FN₃O₅S₂, 599.9 (M+H), found 602.0.

### Example 41, Compound #181

### (Z)-5-chloro-N-((2-(3-(2,4-dichlorobenzyl)-6,7-dihydrobenzo[d]isoxazol-4(5H)-ylidene)-2-fluoroethyl)carbamoyl)-4-methylthiophene-2-sulfonamide

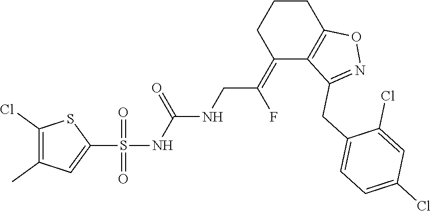 [SMILES: Cc1cc(S(=O)(=O)NC(=O)NC/C(F)=C2\CCCc3onc(Cc4ccc(Cl)cc4Cl)c32)sc1Cl]

(Z)-5-chloro-N-((2-(3-(2,4-dichlorobenzyl)-6,7-dihydrobenzo[d]isoxazol-4(5H)-ylidene)-2-fluoroethyl)carbamoyl)-4-methylthiophene-2-sulfonamide was prepared from (Z)-2-(3-(2,4-dichlorobenzyl)-6,7-dihydrobenzo[d]isoxazol-4(5H)-ylidene)-2-fluoroethan-1-amine according to the procedures as described in Example 7, Steps 1-2.

¹H NMR (CHLOROFORM-d) δ: 7.36-7.45 (m, 2H), 7.15 (dd, J=8.1, 2.0 Hz, 1H), 6.98 (d, J=8.1 Hz, 1H), 4.20 (d, J=4.0 Hz, 2H), 4.00-4.13 (m, 2H), 2.82 (t, J=6.6 Hz, 2H), 2.35-2.46 (m, 2H), 2.16 (s, 3H), 1.94-2.02 (m, 2H). Mass spectrum (ESI, m/z): Calculated for C₂₂H₂₀Cl₃FN₃O₄S₂, 578.0 (M+H), found 578.0.

### Example 42: Compound #97 and Compound #96

### (Z)-4-chloro-N-((2-(3-(4-chlorophenyl)-6,7-dihydrobenzo[d]isoxazol-4(5H)-ylidene)-2-fluoroethyl)carbamoyl)benzenesulfonamide and

### (E)-4-chloro-N-((2-(3-(4-chlorophenyl)-6,7-dihydrobenzo[d]isoxazol-4(5H)-ylidene)-2-fluoroethyl)carbamoyl)benzenesulfonamide

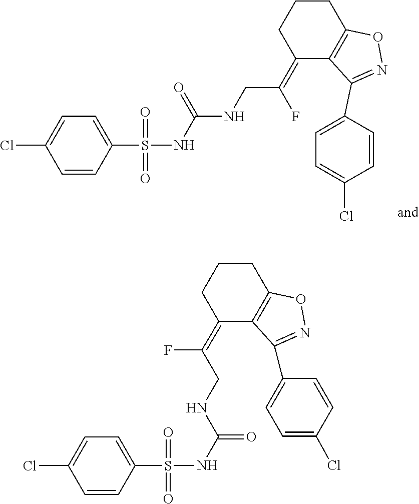 [SMILES: O=C(NC/C(F)=C1\CCCc2onc(-c3ccc(Cl)cc3)c21)NS(=O)(=O)c1ccc(Cl)cc1 ; O=C(NC/C(F)=C1/CCCc2onc(-c3ccc(Cl)cc3)c21)NS(=O)(=O)c1ccc(Cl)cc1]

### Step 1. Synthesis of 3-(4-chlorophenyl)-6,7-dihydrobenzo[d]isoxazol-4(5H)-one

To ⁱPrOH (10 mL) at 0° C. was added NaH (60% in mineral oil, 80.0 mg, 2.0 mmol), followed by cyclohexane-1,3-dione (236 mg, 2.11 mmol) and 4-chloro-N-hydroxybenzimidoyl chloride (200 mg, 1.05 mmol). The reaction was stirred at 0° C. for 1 h before the reaction was warmed up to room temperature and stirred for 1 h. The reaction was quenched with sat. aq. NaCl and extracted with EtOAc. The organic layer was dried over Na₂SO₄ and concentrated. The resulting residue was purified by silica gel column with 20% EtOAc/heptane to yield 3-(4-chlorophenyl)-6,7-dihydrobenzo[d]isoxazol-4(5H)-one as a white solid. Mass spectrum (ESI, m/z): Calculated for C₁₃H₁₁ClNO₂, 248.0 (M+H), found 247.9.

### Step 2. Synthesis of ethyl 2-(3-(2,4-dichlorobenzyl)-6,7-dihydrobenzo[d]isoxazol-4(5H)-ylidene)-2-fluoroacetate

Ethyl 2-(3-(2,4-dichlorobenzyl)-6,7-dihydrobenzo[d]isoxazol-4(5H)-ylidene)-2-fluoroacetate was prepared from 3-(4-chlorophenyl)-6,7-dihydrobenzo[d]isoxazol-4(5H)-one as a mixture of (E) and (Z) isomers according to the procedures as described in Example 40, Step 3.

### Step 3. Synthesis of (Z)-2-(3-(2,4-dichlorobenzyl)-6,7-dihydrobenzo[d]isoxazol-4(5H)-ylidene)-2-fluoroethan-1-ol and (E)-2-(3-(2,4-dichlorobenzyl)-6,7-dihydrobenzo[d]isoxazol-4(5H)-ylidene)-2-fluoroethan-1-ol

(Z)-2-(3-(2,4-dichlorobenzyl)-6,7-dihydrobenzo[d]isoxazol-4(5H)-ylidene)-2-fluoroethan-1-ol and (E)-2-(3-(2,4-dichlorobenzyl)-6,7-dihydrobenzo[d]isoxazol-4(5H)-ylidene)-2-fluoroethan-1-ol were prepared from ethyl 2-(3-(2,4-dichlorobenzyl)-6,7-dihydrobenzo[d]isoxazol-4(5H)-ylidene)-2-fluoroacetate according to the procedures as described in Example 40, Step 4.

### Step 4. Synthesis of (Z)-2-(2-(3-(4-chlorophenyl)-6,7-dihydrobenzo[d]isoxazol-4(5H)-ylidene)-2-fluoroethyl)isoindoline-1,3-dione

(Z)-2-(2-(3-(4-chlorophenyl)-6,7-dihydrobenzo[d]isoxazol-4(5H)-ylidene)-2-fluoroethyl)isoindoline-1,3-dione was prepared from (Z)-2-(3-(2,4-dichlorobenzyl)-6,7-dihydrobenzo[d]isoxazol-4(5H)-ylidene)-2-fluoroethan-1-ol according to the procedures as described in Example 5, Step 1.

### Step 5. Synthesis of (Z)-2-(3-(4-chlorophenyl)-6,7-dihydrobenzo[d]isoxazol-4(5H)-ylidene)-2-fluoroethan-1-amine

(Z)-2-(3-(4-chlorophenyl)-6,7-dihydrobenzo[d]isoxazol-4(5H)-ylidene)-2-fluoroethan-1-amine was prepared from (Z)-2-(2-(3-(4-chlorophenyl)-6,7-dihydrobenzo[d]isoxazol-4(5H)-ylidene)-2-fluoroethyl)isoindoline-1,3-dione according to the procedures as described in Example 5, Step 2.

### Step 6. Synthesis of (Z)-4-chloro-N-((2-(3-(4-chlorophenyl)-6,7-dihydrobenzo[d]isoxazol-4(5H)-ylidene)-2-fluoroethyl)carbamoyl)benzenesulfonamide

(Z)-4-chloro-N-((2-(3-(4-chlorophenyl)-6,7-dihydrobenzo[d]isoxazol-4(5H)-ylidene)-2-fluoroethyl)carbamoyl)benzenesulfonamide was prepared from (Z)-2-(3-(4-chlorophenyl)-6,7-dihydrobenzo[d]isoxazol-4(5H)-ylidene)-2-fluoroethan-1-amine according to the procedures as described in Example 6, Step 1. Mass spectrum (ESI, m/z): Calculated for C₂₂H₁₉Cl₂FN₃O₄S, 510.0 (M+H), found 510.0.

### Step 7. Synthesis of (E)-2-(2-(3-(4-chlorophenyl)-6,7-dihydrobenzo[d]isoxazol-4(5H)-ylidene)-2-fluoroethyl)isoindoline-1,3-dione

(E)-2-(2-(3-(4-chlorophenyl)-6,7-dihydrobenzo[d]isoxazol-4(5H)-ylidene)-2-fluoroethyl)isoindoline-1,3-dione was prepared from (E)-2-(3-(2,4-dichlorobenzyl)-6,7-dihydrobenzo[d]isoxazol-4(5H)-ylidene)-2-fluoroethan-1-ol according to the procedures as described in Example 5, Step 1.

### Step 8. Synthesis of (E)-2-(3-(4-chlorophenyl)-6,7-dihydrobenzo[d]isoxazol-4(5H)-ylidene)-2-fluoroethan-1-amine

(E)-2-(3-(4-chlorophenyl)-6,7-dihydrobenzo[d]isoxazol-4(5H)-ylidene)-2-fluoroethan-1-amine was prepared from (E)-2-(2-(3-(4-chlorophenyl)-6,7-dihydrobenzo[d]isoxazol-4(5H)-ylidene)-2-fluoroethyl)isoindoline-1,3-according to the procedures as described in Example 5, Step 2.

### Step 9. Synthesis of (E)-4-chloro-N-((2-(3-(4-chlorophenyl)-6,7-dihydrobenzo[d]isoxazol-4(5H)-ylidene)-2-fluoroethyl)carbamoyl)benzenesulfonamide

(E)-4-chloro-N-((2-(3-(4-chlorophenyl)-6,7-dihydrobenzo[d]isoxazol-4(5H)-ylidene)-2-fluoroethyl)carbamoyl)benzenesulfonamide was prepared from (E)-2-(3-(4-chlorophenyl)-6,7-dihydrobenzo[d]isoxazol-4(5H)-ylidene)-2-fluoroethan-1-amine according to the procedures as described in Example 6, Step 1.

¹H NMR (CHLOROFORM-d) δ: 7.70 (br d, J=8.6 Hz, 2H), 7.41-7.56 (m, 6H), 6.46 (br s, 1H), 3.46 (br dd, J=18.9, 5.3 Hz, 2H), 2.91 (br t, J=6.3 Hz, 2H), 2.54 (br s, 2H), 2.04 (br d, J=5.1 Hz, 2H). Mass spectrum (ESI, m/z): Calculated for C₂₂H₁₉Cl₂FN₃O₄S, 510.0 (M+H), found 510.0.

### Example 43: Compound #155

### (E)-4,5-dichloro-N-((2-(3-(4-(trifluoromethyl)phenyl)-6,7-dihydro-[1,2,3]triazolo[1,5-a]pyridin-4(5H)-ylidene)ethyl)carbamoyl)thiophene-2-sulfonamide

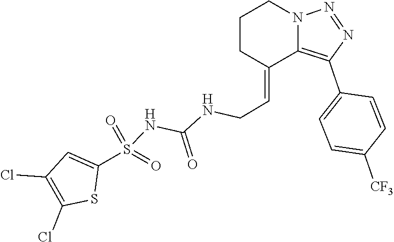 [SMILES: O=C(NC/C=C1\CCCn2nnc(-c3ccc(C(F)(F)F)cc3)c21)NS(=O)(=O)c1cc(Cl)c(Cl)s1]

### Step 1. Synthesis of 6-hydroxy-1-(4-(trifluoromethyl)phenyl)hex-1-yn-3-one

To 1-ethynyl-4-(trifluoromethyl)benzene (5.00 g, 29.4 mmol) in THF (150 mL) at −78° C. under nitrogen was added n-BuLi (11 ml, 27.5 mmol). The reaction was stirred for 30 min at −78° C. before BF₃.Et₂O (3.85 g, 27.1 mmol) was added. The reaction was stirred for another 30 min at −78° C. Dihydrofuran-2(3H)-one (2.35 g, 27.297 mmol) was then added and the reaction was warmed up to room temperature and stirred for 1 h. The reaction was quenched with aq. NH₄Cl solution and the resulting mixture was extracted with EtOAc. The organic layers were combined and concentrated under vacuum. The resulting residue was purified by silica gel column with PE:EA=70:30 to yield 6-hydroxy-1-(4-(trifluoromethyl)phenyl)hex-1-yn-3-one as a yellow solid.

### Step 2. Synthesis of 4-hydroxy-1-(4-(4-(trifluoromethyl)phenyl)-1H-1,2,3-triazol-5-yl)butan-1-one

A mixture of 6-hydroxy-1-(4-(trifluoromethyl)phenyl)hex-1-yn-3-one (6.63 g, 25.9 mmol), NaN₃ (2.02 g, 31.1 mmol), and Cul (0.50 g, 2.63 mmol) in DMSO (100 mL) was stirred at 90° C. overnight. The reaction was quenched with aq. NaClO₂ and the resulting mixture was extracted with EA. The organic layers were combined and concentrated under vacuum. The resulting residue was purified by silica gel column with PE:EA=70:30 to yield 4-hydroxy-1-(4-(4-(trifluoromethyl)phenyl)-1H-1,2,3-triazol-5-yl)butan-1-one as a yellow solid. Mass spectrum (ESI, m/z): Calculated for C₁₃H₁₃F₃N₃O₂, 298.1 [M+H], found 298.0.

### Step 3. Synthesis of 3-(4-(trifluoromethyl)phenyl)-6,7-dihydro-[1,2,3]triazolo[1,5-a]pyridin-4(5H)-one

To a solution of 4-hydroxy-1-(4-(4-(trifluoromethyl)phenyl)-1H-1,2,3-triazol-5-yl)butan-1-one (400 mg, 1.34 mmol) and PPh₃ (351 mg, 1.34 mmol) in THF (20 mL) was added DIAD (270 mg, 1.34 mmol). The reaction was stirred at room temperature overnight. The reaction was then quenched with H₂O and the resulting mixture was extracted with EtOAc. The organic layers were combined and concentrated under vacuum. The resulting residue was purified by silica gel column with PE:EA=70:30 to yield 3-(4-(trifluoromethyl)phenyl)-6,7-dihydro-[1,2,3]triazolo[1,5-a]pyridin-4(5H)-one as a yellow solid. Mass spectrum (ESI, m/z): Calculated for C₁₃H₁₁F₃N₃O, 282.1 [M+H], found 281.9.

### Step 4. Synthesis of ethyl (E)-2-(3-(4-(trifluoromethyl)phenyl)-6,7-dihydro-[1,2,3]triazolo[1,5-a]pyridin-4(5H)-ylidene)acetate

To a solution of ethyl 2-(diethoxyphosphoryl)acetate (287 mg, 1.28 mmol) in THF (30 mL) at 0° C. was added t-BuONa (144 mg, 1.29 mmol). The reaction was stirred for 1 h at 0° C. 3-(4-(Trifluoromethyl)phenyl)-6,7-dihydro-[1,2,3]triazolo[1,5-a]pyridin-4(5H)-one (300 mg, 1.07 mmol) was added and the reaction was warmed up to room temperature and stirred for 2 h. The reaction was then quenched with H₂O. The resulting solution was extracted with EA and the organic layers were combined and concentrated under vacuum. The resulting residue was purified by silica gel column with PE:EA=70:30 to yield ethyl (E)-2-(3-(4-(trifluoromethyl)phenyl)-6,7-dihydro-[1,2,3]triazolo[1,5-a]pyridin-4(5H)-ylidene)acetate as a yellow solid. Mass spectrum (ESI, m/z): Calculated for C₁₇H₁₇F₃N₃O₂, 352.1 [M+H], found 352.0.

### Step 5. Synthesis of (E)-2-(3-(4-(trifluoromethyl)phenyl)-6,7-dihydro-[1,2,3]triazolo[1,5-a]pyridin-4(5H)-ylidene)ethan-1-ol

To ethyl (E)-2-(3-(4-(trifluoromethyl)phenyl)-6,7-dihydro-[1,2,3]triazolo[1,5-a]pyridin-4(5H)-ylidene)acetate (190 mg, 0.541 mmol) in DCM (20 mL) at −78° C. was added DIBAL-H (1.6 mL, 1.6 mmol) in portions. The reaction was stirred for 30 min at −78° C. and stirred for 3 h at 0° C. The reaction was then quenched with MeOH (10 mL) and concentrated under vacuum. The resulting residue was purified by silica gel chromatography (0-10% EtOAc/petroleum ether) to yield (E)-2-(3-(4-(trifluoromethyl)phenyl)-6,7-dihydro-[1,2,3]triazolo[1,5-a]pyridin-4(5H)-ylidene)ethan-1-ol as a yellow oil. Mass spectrum (ESI, m/z): Calculated for C₁₅H₁₅F₃N₃O: 310.1 [M+H], found: 310.1.

### Step 6. Synthesis of (E)-2-(2-(3-(4-(trifluoromethyl)phenyl)-6,7-dihydro-[1,2,3]triazolo[1,5-a]pyridin-4(5H)-ylidene)ethyl)isoindoline-1,3-dione

(E)-2-(2-(3-(4-(trifluoromethyl)phenyl)-6,7-dihydro-[1,2,3]triazolo[1,5-a]pyridin-4(5H)-ylidene)ethyl)isoindoline-1,3-dione was prepared from (E)-2-(3-(4-(trifluoromethyl)phenyl)-6,7-dihydro-[1,2,3]triazolo[1,5-a]pyridin-4(5H)-ylidene)ethan-1-ol according to the procedures as described in Example 5, Step 1.

### Step 7. Synthesis of (E)-2-(3-(4-(trifluoromethyl)phenyl)-6,7-dihydro-[1,2,3]triazolo[1,5-a]pyridin-4(5H)-ylidene)ethan-1-amine

(E)-2-(3-(4-(trifluoromethyl)phenyl)-6,7-dihydro-[1,2,3]triazolo[1,5-a]pyridin-4(5H)-ylidene)ethan-1-amine was prepared from (E)-2-(2-(3-(4-(trifluoromethyl)phenyl)-6,7-dihydro-[1,2,3]triazolo[1,5-a]pyridin-4(5H)-ylidene)ethyl)isoindoline-1,3-dione according to the procedures as described in Example 5, Step 2.

### Step 8. Synthesis of phenyl (E)-(2-(3-(4-(trifluoromethyl)phenyl)-6,7-dihydro-[1,2,3]triazolo[1,5-a]pyridin-4(5H)-ylidene)ethyl)carbamate

Phenyl (E)-(2-(3-(4-(trifluoromethyl)phenyl)-6,7-dihydro-[1,2,3]triazolo[1,5-a]pyridin-4(5H)-ylidene)ethyl)carbamate was prepared from (E)-2-(3-(4-(trifluoromethyl)phenyl)-6,7-dihydro-[1,2,3]triazolo[1,5-a]pyridin-4(5H)-ylidene)ethan-1-amine according to the procedures as described in Example 7, Step 1.

### Step 9. Synthesis of (E)-4,5-dichloro-N-((2-(3-(4-(trifluoromethyl)phenyl)-6,7-dihydro-[1,2,3]triazolo[1,5-a]pyridin-4(5H)-ylidene)ethyl)carbamoyl)thiophene-2-sulfonamide

(E)-4,5-dichloro-N-((2-(3-(4-(trifluoromethyl)phenyl)-6,7-dihydro-[1,2,3]triazolo[1,5-a]pyridin-4(5H)-ylidene)ethyl)carbamoyl)thiophene-2-sulfonamide was prepared from phenyl (E)-(2-(3-(4-(trifluoromethyl)phenyl)-6,7-dihydro-[1,2,3]triazolo[1,5-a]pyridin-4(5H)-ylidene)ethyl)carbamate according to the procedures as described in Example 7, Step 2.

¹H NMR (300 MHz, CD₃OD) δ: 7.73-7.81 (m, 4H), 7.65 (s, 1H), 5.94 (t, J=6.0 Hz, 1H), 4.51 (t, J=6.0 Hz, 2H), 3.85 (d, J=6.6 Hz, 2H), 2.75 (t, J=5.1 Hz, 2H), 2.15-2.19 (m, 2H). ¹⁹F NMR (300 MHz, CD₃OD) δ: −64.11. Mass spectrum (ESI, m/z): Calculated for C₂₀H₁₇Cl₂F₃N₅O₃S₂, 566.0 [M+H], found 566.0.

### Example 44: Compound #129 and Compound #135

### (Z)—N-((2-(3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide and

### (E)-N-((2-(3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide

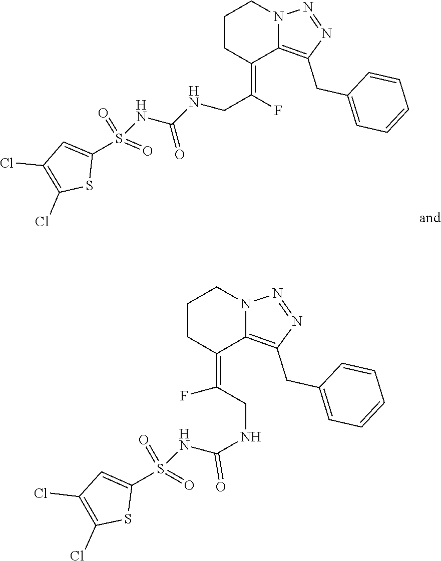 [SMILES: O=C(NC/C(F)=C1\CCCn2nnc(Cc3ccccc3)c21)NS(=O)(=O)c1cc(Cl)c(Cl)s1 ; O=C(NC/C(F)=C1/CCCn2nnc(Cc3ccccc3)c21)NS(=O)(=O)c1cc(Cl)c(Cl)s1]

### Step 1. Synthesis of 7-hydroxy-1-phenylhept-2-yn-4-one

To a solution of prop-2-yn-1-ylbenzene (0.50 mL, 4.02 mmol) in THF (15 mL) at −78° C. was added n-BuLi (2.5 M in hexane, 1.61 mL, 4.02 mmol). The reaction was stirred at −78° C. for 1 h before dihydrofuran-2(3H)-one (0.40 mL, 5.23 mmol) was added. The reaction was warmed up to 0° C. and stirred at 0° C. for 1 h. The reaction was quenched with aq. 1N HCl and extracted with EtOAc. The organic layer was washed with aq. NaCl, dried over Na₂SO₄ and concentrated to yield 7-hydroxy-1-phenylhept-2-yn-4-one.

### Step 2. Synthesis of 1-(4-benzyl-1H-1,2,3-triazol-5-yl)-4-hydroxybutan-1-one

A mixture of above prepared 7-hydroxy-1-phenylhept-2-yn-4-one and NaN₃ (261 mg, 4.02 mmol) in DMF (5 mL) was stirred at room temperature for 2 h. The reaction was quenched with aq NH₄Cl and extracted with EtOAc. The organic layer was washed with aq. NaCl, dried over Na₂SO₄ and concentrated. The resulting residue was purified by silica gel column with 60%-70% EtOAc/heptane to yield 1-(4-benzyl-1H-1,2,3-triazol-5-yl)-4-hydroxybutan-1-one. Mass spectrum (ESI, m/z): Calculated for C₁₃H₁₆N₃O₂, 246.1 [M+H], found 246.1.

### Step 3. Synthesis of 3-benzyl-6,7-dihydro-[1,2,3]triazolo[1,5-a]pyridin-4(5H)-one

To a solution of 1-(4-benzyl-1H-1,2,3-triazol-5-yl)-4-hydroxybutan-1-one (177 mg, 0.722 mmol) and PPh3 (379 mg, 1.44 mmol) in THF (15 mL) at room temperature was added DIAD (0.28 mL, 1.44 mmol). The reaction was stirred at room temperature for 2 h. The resulting mixture was then concentrated and the residue was dissolved in EtOH (4 mL) and conc. HCl (1 mL), and heated at 120° C. by microwave for 1 h. The reaction mixture was concentrated to remove most of the organic solvent and the remaining mixture was extracted with EtOAc. The organic layer was washed with H₂O and aq. NaCl, dried over Na₂SO₄, and concentrated. The resulting residue was purified by silica gel column with 30-60% EtOAc/heptane to yield 3-benzyl-6,7-dihydro-[1,2,3]triazolo[1,5-a]pyridin-4(5H)-one. Mass spectrum (ESI, m/z): Calculated for C₁₃H₁₄N₃O, 228.1 [M+H], found 228.1.

### Step 4. Synthesis of ethyl 2-(3-benzyl-6,7-dihydro-[1,2,3]triazolo[1,5-a]pyridin-4(5H)-ylidene)-2-fluoroacetate

Ethyl 2-(3-benzyl-6,7-dihydro-[1,2,3]triazolo[1,5-a]pyridin-4(5H)-ylidene)-2-fluoroacetate was prepared as a mixture of (E) and (Z) isomers from 3-benzyl-6,7-dihydro-[1,2,3]triazolo[1,5-a]pyridin-4(5H)-one according to the procedures as described in Example 40, Step 3.

### Step 5. Synthesis of (Z)-2-(3-benzyl-6,7-dihydro-[1,2,3]triazolo[1,5-a]pyridin-4(5H)-ylidene)-2-fluoroethan-1-ol and (E)-2-(3-benzyl-6,7-dihydro-[1,2,3]triazolo[1,5-a]pyridin-4(5H)-ylidene)-2-fluoroethan-1-ol

Synthesis of (Z)-2-(3-benzyl-6,7-dihydro-[1,2,3]triazolo[1,5-a]pyridin-4(5H)-ylidene)-2-fluoroethan-1-ol and (E)-2-(3-benzyl-6,7-dihydro-[1,2,3]triazolo[1,5-a]pyridin-4(5H)-ylidene)-2-fluoroethan-1-ol were prepared from ethyl 2-(3-benzyl-6,7-dihydro-[1,2,3]triazolo[1,5-a]pyridin-4(5H)-ylidene)-2-fluoroacetate according to the procedures as described in Example 40, Step 4.

### Steps 6-8. Synthesis of (Z)—N-((2-(3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide

(Z)—N-((2-(3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide was prepared from (Z)-2-(3-benzyl-6,7-dihydro-[1,2,3]triazolo[1,5-a]pyridin-4(5H)-ylidene)-2-fluoroethan-1-ol according to the procedures as described in Example 5, Steps 1-3.

¹H NMR (CHLOROFORM-d) δ: 7.48 (s, 1H), 7.03-7.22 (m, 5H), 4.23-4.40 (m, 2H), 3.92-4.19 (m, 4H), 2.52 (br s, 2H), 2.05 (s, 2H). Mass spectrum (ESI, m/z): Calculated for C₂₀H₁₉Cl₂FN₅O₃S₂, 530.0 [M+H], found 530.1.

### Steps 9-11. Synthesis of (E)-N-((2-3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide

(E)-N-((2-(3-benzyl-6,7-dihydro-[1,2,3]triazolo[1,5-a]pyridin-4(5H)-ylidene)-2-fluoroethyl)carbamoyl)-4,5-dichlorothiophene-2-sulfonamide was prepared from (E)-2-(3-benzyl-6,7-dihydro-[1,2,3]triazolo[1,5-a]pyridin-4(5H)-ylidene)-2-fluoroethan-1-ol according to the procedures as described in Example 5, Steps 1-3.

¹H NMR (CHLOROFORM-d) δ: 7.33-7.49 (m, 1H), 7.08-7.20 (m, 5H), 6.77 (br s, 1H), 4.33 (t, J=6.3 Hz, 2H), 3.87-4.08 (m, 4H), 2.55 (br s, 2H), 1.98-2.08 (m, 2H). Mass spectrum (ESI, m/z): Calculated for C₂₀H₁₉Cl₂FN₅O₃S₂, 530.0 [M+H], found 530.1.

The following representative compound of formula (I) was similarly prepared according to the procedure described in Example 44 (and Examples referenced therein) above, substituting 3-(4-(trifluoromethyl)phenyl)-6,7-dihydro-[1,2,3]triazolo[1,5-a]pyridin-4(5H)-one for 3-benzyl-6,7-dihydro-[1,2,3]triazolo[1,5-a]pyridin-4(5H)-one.

### (Z)-4,5-dichloro-N-((2-fluoro-2-(3-(4-(trifluoromethyl)phenyl)-6,7-dihydro-[1,2,3]triazolo[1,5-a]pyridin-4(5H)-ylidene)ethyl)carbamoyl)thiophene-2-sulfonamide, Compound #164

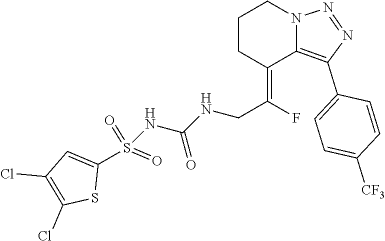 [SMILES: O=C(NC/C(F)=C1\CCCn2nnc(-c3ccc(C(F)(F)F)cc3)c21)NS(=O)(=O)c1cc(Cl)c(Cl)s1]

¹H NMR (400 MHz, CD₃OD) δ: 7.80 (d, J=12.0 Hz, 4H), 7.58 (s, 1H), 4.49 (t, J=6.0 Hz, 2H), 2.38 (d, J=16.4 Hz, 2H), 2.77 (t, J=5.2 Hz, 2H), 2.12-2.18 (m, 2H). ¹⁹F NMR (400 MHz, CD₃OD) δ: −64.15, −110.76. Mass spectrum (ESI, m/z): Calculated for C₂₀H₁₇Cl₃F₄N₅O₃S₂, 584.0 [M+H], found 584.0.

### Example 45: Compound #167

### (Z)-4,5-dichloro-N-((2-(3-(4-chloro-2-methylbenzyl)-6,7-dihydro-[1,2,3]triazolo[1,5-a]pyridin-4(5H)-ylidene)-2-fluoroethyl)carbamoyl)thiophene-2-sulfonamide

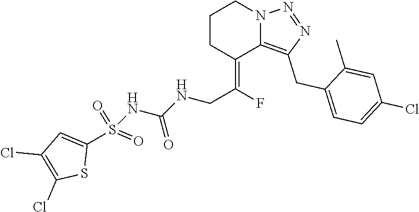 [SMILES: Cc1cc(Cl)ccc1Cc1nnn2c1/C(=C(\F)CNC(=O)NS(=O)(=O)c1cc(Cl)c(Cl)s1)CCC2]

### Step 1. Synthesis of 1-((tert-butyldimethylsilyl)oxy)-7-hydroxyhept-2-yn-4-one

1-((Tert-butyldimethylsilyl)oxy)-7-hydroxyhept-2-yn-4-one was prepared from tert-butyldimethyl(prop-2-yn-1-yloxy)silane according to the procedures as described in Example 44, Step 1.

### Step 2. Synthesis of 1-(4-(((tert-butyldimethylsilyl)oxy)methyl)-1H-1,2,3-triazol-5-yl)-4-hydroxybutan-1-one

1-(4-(((Tert-butyldimethylsilyl)oxy)methyl)-1H-1,2,3-triazol-5-yl)-4-hydroxybutan-1-one was prepared from 1-((tert-butyldimethylsilyl)oxy)-7-hydroxyhept-2-yn-4-one according to the procedures as described in Example 44, Step 2.

### Step 3. Synthesis of 3-(((tert-butyldimethylsilyl)oxy)methyl)-6,7-dihydro-[1,2,3]triazolo[1,5-a]pyridin-4(5H)-one

To a solution of 1-(4-(((Tert-butyldimethylsilyl)oxy)methyl)-1H-1,2,3-triazol-5-yl)-4-hydroxybutan-1-one (835 mg, 2.79 mmol) and PPh₃ (731 mg, 2.79 mmol) in THF (50 mL) at room temperature was added di-tert-butyl diazene-1,2-dicarboxylate (642 mg, 2.79 mmol). The reaction was stirred at room temperature for 1.5 h. The reaction solution was concentration and the resulting residue was purified by silica gel column with 50% EtOAc/heptane to yield 3-(((tert-butyldimethylsilyl)oxy)methyl)-6,7-dihydro-[1,2,3]triazolo[1,5-a]pyridin-4(5H)-one. Mass spectrum (ESI, m/z): Calculated for C₁₃H₂₄N₃O₂Si, 282.2 [M+H], found 282.1.

### Step 4. Synthesis of 3-(hydroxymethyl)-6,7-dihydro-[1,2,3]triazolo[1,5-a]pyridin-4(5H)-one

To 3-(((tert-butyldimethylsilyl)oxy)methyl)-6,7-dihydro-[1,2,3]triazolo[1,5-a]pyridin-4(5H)-one (230 mg, 0.817 mmol) in EtOH (10 mL) at room temperature was added conc. HCl (0.1 mL, 1.20 mmol). The reaction was stirred at room temperature for 1.5 h. The reaction mixture was concentrated and the residue was purified by silica gel column with 3-4% MeOH/CH₂Cl₂ to yield 3-(hydroxymethyl)-6,7-dihydro-[1,2,3]triazolo[1,5-a]pyridin-4(5H)-one. Mass spectrum (ESI, m/z): Calculated for C₇H₁₀N₃O₂, 168.1 [M+H], found 168.0.

### Step 5. Synthesis of 3-(chloromethyl)-6,7-dihydro[1,2,3]triazolo[1,5-a]pyridin-4(5H)-one

To 3-(hydroxymethyl)-6,7-dihydro-[1,2,3]triazolo[1,5-a]pyridin-4(5H)-one (124 mg, 0.742 mmol) in CH₂Cl₂ (10 mL) at 0° C. was added SOCl₂ (0.16 mL, 2.23 mmol). The reaction was stirred at 0° C. for 1 h. The resulting mixture was then concentrated and the resulting residue was dissolved in CH₂Cl₂. The solution was washed with H₂O, dried over Na₂SO₄, and concentrated. The resulting residue was purified by silica gel column with 60% EtOAc/heptane to yield 3-(chloromethyl)-6,7-dihydro-[1,2,3]triazolo[1,5-a]pyridin-4(5H)-one.

### Step 6. Synthesis of 3-(4-chloro-2-methylbenzyl)-6,7-dihydro-[1,2,3]triazolo[1,5-a]pyridin-4(5H)-one

A mixture of 3-(chloromethyl)-6,7-dihydro-[1,2,3]triazolo[1,5-a]pyridin-4(5H)-one (108 mg, 0.582 mmol), (4-chloro-2-methylphenyl)boronic acid (149 mg, 0.873 mmol), Na₂CO₃ (154 mg, 1.46 mmol), and Pd(PPh₃)₄ (33.6 mg, 0.029 mmol) in 1,4-dioxane (3 mL) and H₂O (0.6 mL) was heated at 130° C. by microwave for 30 min. The reaction mixture was diluted with CH₂Cl₂ and filtered. The solution was concentrated and the resulting residue was purified by silica gel column with 40% EtOAc/heptane to yield 3-(4-chloro-2-methylbenzyl)-6,7-dihydro-[1,2,3]triazolo[1,5-a]pyridin-4(5H)-one. Mass spectrum (ESI, m/z): Calculated for C₁₄H₁₅ClN₃O₂, 276.1 [M+H], found 276.0.

### Step 7. Synthesis of ethyl 2-(3-(4-chloro-2-methylbenzyl)-6,7-dihydro-[1,2,3]triazolo[1,5-a]pyridin-4(5H)-ylidene)-2-fluoroacetate

Ethyl 2-(3-(4-chloro-2-methylbenzyl)-6,7-dihydro-[1,2,3]triazolo[1,5-a]pyridin-4(5H)-ylidene)-2-fluoroacetate was prepared as a mixture of (E) and (Z) isomers from 3-(4-chloro-2-methylbenzyl)-6,7-dihydro-[1,2,3]triazolo[1,5-a]pyridin-4(5H)-one according to the procedures as described in Example 31, Step 1.

### Step 8. Synthesis of (Z)-2-(3-(4-chloro-2-methylbenzyl)-6,7-dihydro-[1,2,3]triazolo[1,5-a]pyridin-4(5H)-ylidene)-2-fluoroethan-1-ol

(Z)-2-(3-(4-chloro-2-methyl benzyl)-6,7-dihydro-[1,2,3]triazolo[1,5-a]pyridin-4(5H)-ylidene)-2-fluoroethan-1-ol was prepared from ethyl 2-(3-(4-chloro-2-methylbenzyl)-6,7-dihydro-[1,2,3]triazolo[1,5-a]pyridin-4(5H)-ylidene)-2-fluoroacetate according to the procedures as described in Example 31, Step 2. Mass spectrum (ESI, m/z): Calculated for C₁₆H₁₈ClFN₃O, 322.1 [M+H], found 322.0.

### Steps 9-10. Synthesis of (Z)-2-(3-(4-chloro-2-methylbenzyl)-6,7-dihydro-[1,2,3]triazolo[1,5-a]pyridin-4(5H)-ylidene)-2-fluoroethan-1-amine

(Z)-2-(3-(4-chloro-2-methyl benzyl)-6,7-dihydro-[1,2,3]triazolo[1,5-a]pyridin-4(5H)-ylidene)-2-fluoroethan-1-amine was prepared from (Z)-2-(3-(4-chloro-2-methylbenzyl)-6,7-dihydro-[1,2,3]triazolo[1,5-a]pyridin-4(5H)-ylidene)-2-fluoroethan-1-ol according to the procedures as described in Example 5, Steps 1-2.

### Steps 11-12. Synthesis of (Z)-4,5-dichloro-N-((2-(3-(4-chloro-2-methylbenzyl)-6,7-dihydro-[1,2,3]triazolo[1,5-a]pyridin-4(5H)-ylidene)-2-fluoroethyl)carbamoyl)thiophene-2-sulfonamide

(Z)-4,5-dichloro-N-((2-(3-(4-chloro-2-methylbenzyl)-6,7-dihydro-[1,2,3]triazolo[1,5-a]pyridin-4(5H)-ylidene)-2-fluoroethyl)carbamoyl)thiophene-2-sulfonamide was prepared from (Z)-2-(3-(4-chloro-2-methylbenzyl)-6,7-dihydro-[1,2,3]triazolo[1,5-a]pyridin-4(5H)-ylidene)-2-fluoroethan-1-amine according to the procedures as described in Example 7, Steps 1-2.

¹H NMR (CHLOROFORM-d) δ: 7.42-7.51 (m, 1H), 7.05 (s, 1H), 6.95 (br d, J=7.6 Hz, 1H), 6.78 (br d, J=8.1 Hz, 1H), 4.36 (br s, 2H), 3.94-4.08 (m, 4H), 2.53-2.66 (m, 2H), 2.19 (s, 3H), 2.03-2.11 (m, 2H). Mass spectrum (ESI, m/z): Calculated for C₂₁H₂₀Cl₃FN₅O₃S₂, 578.0 [M+H], found 578.0.

The following representative compound of formula (I) was similarly prepared according to the procedure described in Example 45 (and Examples referenced therein) above, selecting and substituting suitable starting materials and reagents, as would be readily recognized by those skilled in the art.

### (Z)-5-chloro-N-((2-(3-(4-chloro-2-methylbenzyl)-6,7-dihydro-[1,2,3]triazolo[1,5-a]pyridin-4(5H)-ylidene)-2-fluoroethyl)carbamoyl)-4-methylthiophene-2-sulfonamide, Compound #188

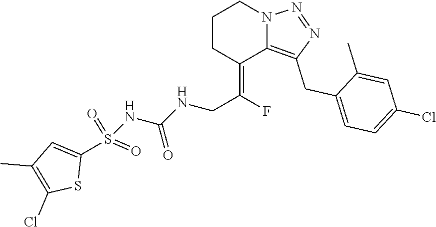 [SMILES: Cc1cc(Cl)ccc1Cc1nnn2c1/C(=C(\F)CNC(=O)NS(=O)(=O)c1cc(C)c(Cl)s1)CCC2]

¹H NMR (CHLOROFORM-d) δ: 7.35 (s, 1H), 7.06 (s, 1H), 6.90-6.98 (m, 1H), 6.80 (d, J=8.1 Hz, 1H), 4.36 (br s, 2H), 3.96-4.12 (m, 4H), 2.59 (br s, 2H), 2.20 (s, 3H), 2.00-2.15 (m, 5H). Mass spectrum (ESI, m/z): Calculated for C₂₂H₂₃Cl₂FN₅O₃S₂, 558.1 [M+H], found 558.0.

### Example 46: Compound #146 and Compound #147

### (E)-4,5-dichloro-N-((2-(3-(2,4-dichlorobenzyl)-1-(methoxymethyl)-1,7-dihydropyrano[3,4-c]pyrazol-4(5H)-ylidene)-2-fluoroethyl)carbamoyl)thiophene-2-sulfonamide and

### (E)-4,5-dichloro-N-((2-(3-(2,4-dichlorobenzyl)-1,7-dihydropyrano[3,4-c]pyrazol-4(5H)-ylidene)-2-fluoroethyl)carbamoyl)thiophene-2-sulfonamide

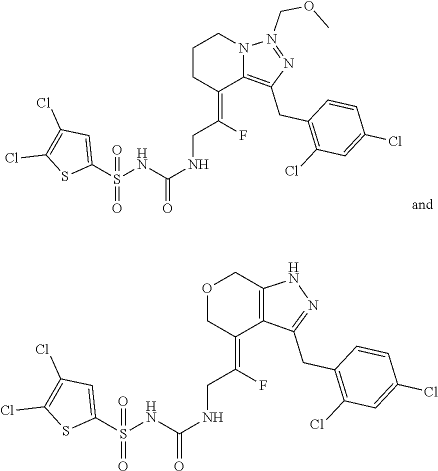

### Step 1. Synthesis of 5-oxo-5,6-dihydro-2H-pyran-3-yl 2-(2,4-dichlorophenyl)acetate

A solution of 2-(2,4-dichlorophenyl)acetic acid (1.00 g, 4.88 mmol), 2H-pyran-3,5(4H,6H)-dione (671 mg, 5.88 mmol), DCC (1.21 g, 5.88 mmol), and DMAP (897 mg, 7.35 mmol) in DCM (50 mL) was stirred at room temperature overnight. The reaction mixture was filtered and the resulting solution was concentrated under vacuum to yield crude 5-oxo-5,6-dihydro-2H-pyran-3-yl 2-(2,4-dichlorophenyl)acetate as a brown oil. Mass spectrum (ESI, m/z): Calculated for C₁₃H₁₁Cl₂O₄: 299.0 [M−H], found: 298.9.

### Step 2. Synthesis of 4-(2-(2,4-dichlorophenyl)acetyl)-2H-pyran-3,5(4H,6H)-dione

A solution of 5-oxo-5,6-dihydro-2H-pyran-3-yl 2-(2,4-dichlorophenyl)acetate (1.47 g, 4.88 mmol), 2-hydroxy-2-methylpropanenitrile (63 mg, 0.74 mmol), and TEA (1.48 g, 14.7 mmol) in MeCN (100 mL) was stirred at room temperature overnight. The resulting solution was concentrated under vacuum to yield 4-(2-(2,4-dichlorophenyl)acetyl)-2H-pyran-3,5(4H,6H)-dione as a brown oil. Mass spectrum (ESI, m/z): Calculated for C₁₃H₁₁Cl₂O₄: 299.0 [M−H], found: 298.9.

### Step 3. Synthesis of 3-(2,4-dichlorobenzyl)-1,7-dihydropyrano[3,4-c]pyrazol-4(5H)-one

A solution of 4-(2-(2,4-dichlorophenyl)acetyl)-2H-pyran-3,5(4H,6H)-dione (1.4 g, 4.65 mmol) and hydrazine hydrate (0.33 g, 6.59 mmol) in dioxane (50 mL) was stirred at room temperature for 4 h. To the reaction was added H₂O (100 mL) and the resulting mixture was extracted with EtOAc (50 mL×3). The organic layers were combined and concentrated under vacuum. The residue obtained purified by silica gel chromatography (0-50% EtOAc/petroleum ether) to yield 3-(2,4-dichlorobenzyl)-1,7-dihydropyrano[3,4-c]pyrazol-4(5H)-one as a yellow oil. Mass spectrum (ESI, m/z): Calculated for C₁₃H₁₁Cl₂N₂O₂: 295.0 [M−H], found: 294.9.

### Step 4. Synthesis of 3-(2,4-dichlorobenzyl)-1-(methoxymethyl)-1,7-dihydropyrano[3,4-c]pyrazol-4(5H)-one

A mixture of 3-(2,4-dichlorobenzyl)-1,7-dihydropyrano[3,4-c]pyrazol-4(5H)-one (50 mg, 0.168 mmol), chloro(methoxy)methane (54 mg, 0.671 mmol), and K₂CO₃ (47 mg, 0.341 mmol) in MeCN (10 mL) was stirred at 50° C. for 8 h. The reaction was then quenched with H₂O (100 mL) and the resulting mixture was extracted with EtOAc (50 mL×3). The organic layers were combined and concentrated under vacuum. The residue was purified by silica gel chromatography (0-50% EtOAc/petroleum ether) to yield 3-(2,4-dichlorobenzyl)-1-(methoxymethyl)-1,7-dihydropyrano[3,4-c]pyrazol-4(5H)-one as a yellow oil. Mass spectrum (ESI, m/z): Calculated for C₁₅H₁₅Cl₂N₂O₃: 341.0 [M+H], found: 341.0.

### Step 5. Synthesis of ethyl (E)-2-(3-(2,4-dichlorobenzyl)-1-(methoxymethyl)-1,7-dihydropyrano[3,4-c]pyrazol-4(5H)-ylidene)-2-fluoroacetate

To a solution of ethyl 2-(diethoxyphosphoryl)-2-fluoroacetate (641 mg, 2.65 mmol) in THF (30 mL) at −78° C. under nitrogen was added n-BuLi (1.06 ml, 2.65 mmol). The resulting solution was stirred for 1 h at −78° C. before 3-(2,4-dichlorobenzyl)-1-(methoxymethyl)-1,7-dihydropyrano[3,4-c]pyrazol-4(5H)-one (250 mg, 0.733 mmol) was added. The resulting solution was warmed up to room temperature and stirred for 2 h. The reaction was then quenched with H₂O and the resulting mixture was extracted with EtOAc. The organic layers were combined and concentrated under vacuum. The resulting residue was purified by silica gel column with PE:EA=70:30 to yield ethyl (E)-2-(3-(2,4-dichlorobenzyl)-1-(methoxymethyl)-1,7-dihydropyrano[3,4-c]pyrazol-4(5H)-ylidene)-2-fluoroacetate as yellow oil. Mass spectrum (ESI, m/z): Calculated for C₁₉H₂₀Cl₂FN₂O₄: 429.1 [M+H], found: 429.0.

### Step 6. Synthesis of (E)-2-(3-(2,4-dichlorobenzyl)-1-(methoxymethyl)-1,7-dihydropyrano[3,4-c]pyrazol-4(5H)-ylidene)-2-fluoroethan-1-ol

To a solution of ethyl (E)-2-(3-(2,4-dichlorobenzyl)-1-(methoxymethyl)-1,7-dihydropyrano[3,4-c]pyrazol-4(5H)-ylidene)-2-fluoroacetate (250 mg, 0.582 mmol) in CH₂Cl₂ (20 mL) at −78° C. under nitrogen was added DIBAL-H (1.8 mL, 1.80 mmol) in portions. The resulting solution was stirred for 30 min at −78° C. and stirred for 3 h at 0° C. The reaction was then quenched by the addition of MeOH (10 mL). The resulting mixture was concentrated under vacuum. The residue obtained was purified by silica gel chromatography (0-50% EtOAc/petroleum ether) to yield (E)-2-(3-(2,4-dichlorobenzyl)-1-(methoxymethyl)-1,7-dihydropyrano[3,4-c]pyrazol-4(5H)-ylidene)-2-fluoroethan-1-ol as a yellow oil. Mass spectrum (ESI, m/z): Calculated for C₁₇H₁₈Cl₂FN₂O₃: 387.1 [M+H], found: 387.0.

### Step 7. Synthesis of (E)-2-(2-(3-(2,4-dichlorobenzyl)-1-(methoxymethyl)-1,7-dihydropyrano[3,4-c]pyrazol-4(5H)-ylidene)-2-fluoroethyl)isoindoline-1,3-dione

(E)-2-(2-(3-(2,4-dichlorobenzyl)-1-(methoxymethyl)-1,7-dihydropyrano[3,4-c]pyrazol-4(5H)-ylidene)-2-fluoroethyl)isoindoline-1,3-dione was prepared from (E)-2-(3-(2,4-dichlorobenzyl)-1-(methoxymethyl)-1,7-dihydropyrano[3,4-c]pyrazol-4(5H)-ylidene)-2-fluoroethan-1-ol according to the procedures as described in Example 5, Step 1.

### Step 8. Synthesis of (E)-2-(3-(2,4-dichlorobenzyl)-1-(methoxymethyl)-1,7-dihydropyrano[3,4-c]pyrazol-4(5H)-ylidene)-2-fluoroethan-1-amine

(E)-2-(3-(2,4-dichlorobenzyl)-1-(methoxymethyl)-1,7-dihydropyrano[3,4-c]pyrazol-4(5H)-ylidene)-2-fluoroethan-1-amine was prepared from (E)-2-(2-(3-(2,4-dichlorobenzyl)-1-(methoxymethyl)-1,7-dihydropyrano[3,4-c]pyrazol-4(5H)-ylidene)-2-fluoroethyl)isoindoline-1,3-dione according to the procedures as described in Example 5, Step 2.

### Step 9. Synthesis of phenyl (E)-(2-(3-(2,4-dichlorobenzyl)-1-(methoxymethyl)-1,7-dihydropyrano[3,4-c]pyrazol-4(5H)-ylidene)-2-fluoroethyl)carbamate

Phenyl (E)-(2-(3-(2,4-dichlorobenzyl)-1-(methoxymethyl)-1,7-dihydropyrano[3,4-c]pyrazol-4(5H)-ylidene)-2-fluoroethyl)carbamate was prepared from phenyl (E)-(2-(3-(2,4-dichlorobenzyl)-1-(methoxymethyl)-1,7-dihydropyrano[3,4-c]pyrazol-4(5H)-ylidene)-2-fluoroethyl)carbamate according to the procedures as described in Example 7, Step 1.

### Step 10. Synthesis of (E)-4,5-dichloro-N-((2-(3-(2,4-dichlorobenzyl)-1-(methoxymethyl)-1,7-dihydropyrano[3,4-c]pyrazol-4(5H)-ylidene)-2-fluoroethyl)carbamoyl)thiophene-2-sulfonamide

(E)-4,5-dichloro-N-((2-(3-(2,4-dichlorobenzyl)-1-(methoxymethyl)-1,7-dihydropyrano[3,4-c]pyrazol-4(5H)-ylidene)-2-fluoroethyl)carbamoyl)thiophene-2-sulfonamide was prepared from phenyl (E)-(2-(3-(2,4-dichlorobenzyl)-1-(methoxymethyl)-1,7-dihydropyrano[3,4-c]pyrazol-4(5H)-ylidene)-2-fluoroethyl)carbamate according to the procedures as described in Example 7, Step 2.

¹H NMR (400 MHz, CHLOROFORM-d) δ: 7.29-7.37 (m, 2H), 6.99-7.08 (m, 1H), 6.86 (d, J=8.6 Hz, 1H), 5.16 (s, 2H), 4.79 (s, 2H), 4.36 (br s, 2H), 4.05-4.12 (m, 2H), 3.71-3.90 (m, 2H), 3.17-3.31 (m, 3H). Mass spectrum (ESI, m/z): Calculated for C₂₂H₂₀Cl₄FN₄O₅S₂: 643.0 [M+H]. found: 645.0.

### Step 11. Synthesis of (E)-4,5-dichloro-N-((2-(3-(2,4-dichlorobenzyl)-1,7-dihydropyrano[3,4-c]pyrazol-4(5H)-ylidene)-2-fluoroethyl)carbamoyl)thiophene-2-sulfonamide

A solution of (E)-4,5-dichloro-N-((2-(3-(2,4-dichlorobenzyl)-1-(methoxymethyl)-1,7-dihydropyrano[3,4-c]pyrazol-4(5H)-ylidene)-2-fluoroethyl)carbamoyl)thiophene-2-sulfonamide (60 mg, 0.093 mmol) and conc. HCl (0.1 ml, 1.20 mmol) in MeOH (10 mL) was stirred for 6 h at 60° C. The reaction solution was concentrated under vacuum. The resulting residue was purified by Prep-HPLC with the following conditions (1#-Waters 2767-5): Column, X Bridge C18, 5 um, 19*100 mm; mobile phase, Water with 0.05% TFA and CH₃CN (50% CH₃CN up to 70% in 10 min, up to 95% in 0.1 min, hold 95% in 0.9 min, down to 50% in 0.1 min, hold 50% in 1.4 min); Detector, UV 220&254 nm. The resulting solution was concentrated under vacuum. The resulting residue was dissolved in CH₃CN (5 mL) and then HCl (2.5 N, 2 mL) was added. The resulting solution was concentrated under vacuum. This was repeated and the resulting residue lyophilized to yield (E)-4,5-dichloro-N-((2-(3-(2,4-dichlorobenzyl)-1,7-dihydropyrano[3,4-c]pyrazol-4(5H)-ylidene)-2-fluoroethyl)carbamoyl)thiophene-2-sulfonamide as a white solid.

¹H NMR (300 MHz, DMSO) δ:7.80 (s, 1H), 7.57 (s, 1H), 7.28-7.32 (m, 1H), 7.09-7.12 (m, 1H), 6.87 (d, J=8.1 Hz, 1H), 4.70 (s, 2H), 4.34 (s, 2H), 4.12 (s, 2H), 3.90 (d, J=22.5 Hz, 2H). ¹⁹F NMR (300 MHz, DMSO) δ: −110.51. Mass spectrum (ESI, m/z): Calculated for C₂₀H₁₆Cl₄FN₄O₄S₂: 600.9 [M+H], found: 600.9.

The following representative compounds of formula (I) were similarly prepared according to the procedure described in Example 46 (and Examples referenced therein) above, selecting and substituting suitable starting materials and reagents, as would be readily recognized by those skilled in the art.

### (Z)-3-chloro-N-((2-(3-(2,4-dichlorobenzyl)-1,5,6,7-tetrahydro-4H-indazol-4-ylidene)-2-fluoroethyl)carbamoyl)-4-methoxybenzenesulfonamide, Compound #179

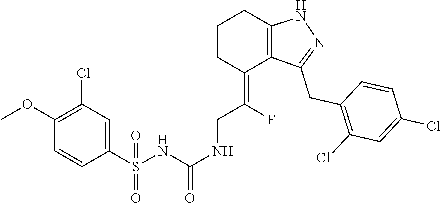 [SMILES: COc1ccc(S(=O)(=O)NC(=O)NC/C(F)=C2\CCCc3[nH]nc(Cc4ccc(Cl)cc4Cl)c32)cc1Cl]

¹H NMR (300 MHz, CD₃OD) δ:7.97 (d, J=2.4 Hz, 1H), 7.89 (d, J=8.7 Hz, 1H), 7.43 (s, 1H), 7.17-7.22 (m, 2H), 6.90 (d, J=8.1 Hz, 1H), 4.77 (s, 2H), 4.38 (d, J=2.1 Hz, 2H), 4.23 (d, J=3.0 Hz, 2H), 4.02 (s, 1H), 3.94 (s, 4H). ¹⁹F NMR (300 MHz, CD₃OD) δ: −110.79. Mass spectrum (ESI, m/z): Calculated for C₂₃H₂₁Cl₃FN₄O₅S: 589.0 [M+H]. found: 591.0.

### (E)-5-chloro-N-((2-(3-(2,4-dichlorobenzyl)-1,7-dihydropyrano[3,4-c]pyrazol-4(5H)-ylidene)-2-fluoroethyl)carbamoyl)-4-methylthiophene-2-sulfonamide, Compound #190

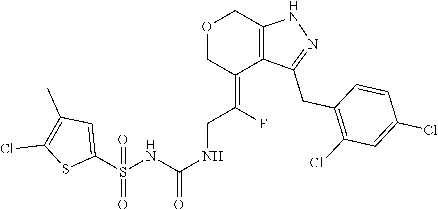 [SMILES: Cc1cc(S(=O)(=O)NC(=O)NC/C(F)=C2\COCc3[nH]nc(Cc4ccc(Cl)cc4Cl)c32)sc1Cl]

¹H NMR (300 MHz, DMSO) δ:11.10 (s, 1H), 7.57-7.58 (m, 2H), 7.30 (d, J=8.1 Hz, 1H), 7.00 (brs, 1H), 6.87 (d, J=8.4 Hz, 1H), 4.70 (s, 2H), 4.34 (s, 2H), 4.12 (s, 2H), 3.89 (d, J=23.1 Hz, 2H), 2.15 (s, 3H). ¹⁹F NMR (300 MHz, DMSO) δ: −110.50. Mass spectrum (ESI, m/z): Calculated for C₂₁H₁₉Cl₃FN₄O₄S₂: 581.0 [M+H]. found: 580.9.

### Example 47: Compound #186

### (E)-4,5-dichloro-N-((2-(3-(2,4-dichlorobenzyl)-1-methyl-1,7-dihydropyrano[3,4-c]pyrazol-4(5H)-ylidene)-2-fluoroethyl)carbamoyl)thiophene-2-sulfonamide

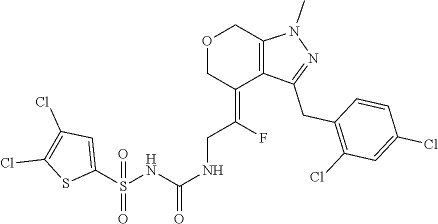 [SMILES: Cn1nc(Cc2ccc(Cl)cc2Cl)c2c1COC/C2=C(/F)CNC(=O)NS(=O)(=O)c1cc(Cl)c(Cl)s1]

### Step 1. Synthesis of 3-(2,4-dichlorobenzyl)-1-methyl-1,7-dihydropyrano[3,4-c]pyrazol-4(5H)-one and 3-(2,4-dichlorobenzyl)-2-methyl-2,7-dihydropyrano[3,4-c]pyrazol-4(5H)-one

A mixture of 3-(2,4-dichlorobenzyl)-1,7-dihydropyrano[3,4-c]pyrazol-4(5H)-one (50 mg, 0.168 mmol), iodomethane (48 mg, 0.338 mmol), and K₂CO₃ (47 mg, 0.341 mmol) in MeCN (10 mL) was stirred for 8 h at 50° C. The reaction was then quenched with H₂O (100 m L) and the resulting mixture was extracted with EtOAc (50 mL×3). The organic layers were combined and concentrated under vacuum. The residue was purified by silica gel chromatography (0-50% EtOAc/petroleum ether) to yield 3-(2,4-dichlorobenzyl)-1-methyl-1,7-dihydropyrano[3,4-c]pyrazol-4(5H)-one as a white solid and 3-(2,4-dichlorobenzyl)-2-methyl-2,7-dihydropyrano[3,4-c]pyrazol-4(5H)-one as a white solid.

### Steps 2-7. Synthesis of (E)-4,5-dichloro-N-((2-(3-(2,4-dichlorobenzyl)-1-methyl-1,7-dihydropyrano[3,4-c]pyrazol-4(5H)-ylidene)-2-fluoroethyl)carbamoyl)thiophene-2-sulfonamide hydrochloride

(E)-4,5-dichloro-N-((2-(3-(2,4-dichlorobenzyl)-1-methyl-1,7-dihydropyrano[3,4-c]pyrazol-4(5H)-ylidene)-2-fluoroethyl)carbamoyl)thiophene-2-sulfonamide hydrochloride was prepared from 3-(2,4-dichlorobenzyl)-1-methyl-1,7-dihydropyrano[3,4-c]pyrazol-4(5H)-one according to the procedures as described in Example 46, Steps 5-10.

¹H NMR (300 MHz, CD₃Cl) δ: 8.09 (s, 1H), 7.44-7.48 (m, 2H), 7.14 (s, 1H), 6.81 (brs, 2H), 4.84 (s, 2H), 4.08-4.56 (m, 6H), 3.69 (s, 3H). ¹⁹F NMR (300 MHz, CD₃Cl) δ: −109.92. Mass spectrum (ESI, m/z): Calculated for C₂₁H₁₈Cl₄FN₄O₄S₂: 614.9 [M+H], found: 614.9.

### Example 48: Compound #189

### (E)-4,5-dichloro-N-((2-(3-(2,4-dichlorobenzyl)-2-methyl-2,7-dihydropyrano[3,4-c]pyrazol-4(5H)-ylidene)-2-fluoroethyl)carbamoyl)thiophene-2-sulfonamide

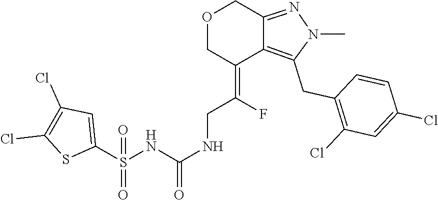 [SMILES: Cn1nc2c(c1Cc1ccc(Cl)cc1Cl)/C(=C(\F)CNC(=O)NS(=O)(=O)c1cc(Cl)c(Cl)s1)COC2]

(E)-4,5-dichloro-N-((2-(3-(2,4-dichlorobenzyl)-2-methyl-2,7-dihydropyrano[3,4-c]pyrazol-4(5H)-ylidene)-2-fluoroethyl)carbamoyl)thiophene-2-sulfonamide was prepared from 3-(2,4-dichlorobenzyl)-2-methyl-2,7-dihydropyrano[3,4-c]pyrazol-4(5H)-one according to the procedures as described in Example 46, Steps 5-10.

¹H NMR (300 MHz, DMSO) δ:7.80 (s, 1H), 7.52 (s, 1H), 7.27 (d, J=8.4 Hz, 1H), 7.12 (brs, 1H), 7.00 (d, J=8.4 Hz, 1H), 4.79 (s, 2H), 4.30 (s, 2H), 4.04 (d, J=3.3 Hz, 2H), 3.92 (d, J=22.5 Hz, 2H), 3.60 (s, 3H). ¹⁹F NMR (300 MHz, DMSO) δ: −111.18. Mass spectrum (ESI, m/z): Calculated for C₂₁H₁₈Cl₄FN₄O₄S₂: 614.9 [M+H], found: 614.8.

### Example 49: Compound #187

### (Z)-5-chloro-N-((2-(3-(2,4-dichlorobenzyl)-1,5,6,7-tetrahydro-4H-indazol-4-ylidene)-2-fluoroethyl)carbamoyl)-4-methylthiophene-2-sulfonamide

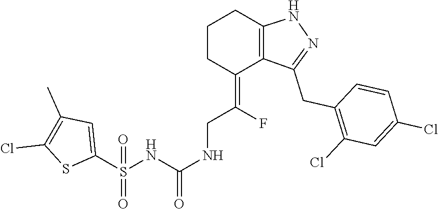 [SMILES: Cc1cc(S(=O)(=O)NC(=O)NC/C(F)=C2\CCCc3[nH]nc(Cc4ccc(Cl)cc4Cl)c32)sc1Cl]

### Step 1. Synthesis of 3-oxocyclohex-1-en-1-yl 2-acetoxyacetate

To a solution of cyclohexane-1,3-dione (1.10 g, 9.77 mmol) in CH₂Cl₂ (50 mL) at room temperature was added Et₃N (1.55 mL, 11.2 mmol) followed by 2-chloro-2-oxoethyl acetate (1.0 mL, 9.30 mmol). The reaction was stirred at room temperature overnight. The resulting mixture was then quenched with aq. NaHCO₃ and the resulting mixture was extracted with CH₂Cl₂. The organic layer was dried over Na₂SO₄ and concentrated to yield 3-oxocyclohex-1-en-1-yl 2-acetoxyacetate.

### Step 2. Synthesis of 2-(2,6-dioxocyclohexyl)-2-oxoethyl acetate

2-(2,6-Dioxocyclohexyl)-2-oxoethyl acetate was prepared from 3-oxocyclohex-1-en-1-yl 2-acetoxyacetate according to the procedures as described in Example 46, Step 2.

### Step 3. Synthesis of (4-oxo-4,5,6,7-tetrahydro-1H-indazol-3-yl)methyl acetate

To 2-(2,6-dioxocyclohexyl)-2-oxoethyl acetate (1.10 g, 5.18 mmol) in CH₃CN (20 mL) at room temperature was added hydrazine (1.63 mL, 5.18 mmol). The reaction was stirred at room temperature for 1 h. The reaction solution was concentrated. The residue was stirred in iPrOH (20 mL) and conc. HCl (1 mL) at room temperature for 1 h. The resulting solution was concentrated and neutralized with aq. NaHCO₃. The mixture was extracted with EtOAc. The organic layer was dried over Na₂SO₄ and concentrated. The resulting residue was purified by silica gel column with 4% MeOH/CH₂Cl₂ to yield (4-oxo-4,5,6,7-tetrahydro-1H-indazol-3-yl)methyl acetate\.

### Step 4. Synthesis of (1-(methoxymethyl)-4-oxo-4,5,6,7-tetrahydro-1H-indazol-3-yl)methyl acetate

A mixture of (4-oxo-4,5,6,7-tetrahydro-1H-indazol-3-yl)methyl acetate (320 mg, 1.54 mmol), chloro(methoxy)methane (0.39 mL, 4.61 mmol), and Cs₂CO₃ (751 mg, 2.31 mmol) in CH₃CN (20 mL) was heated at 50° C. for 6.5 h. The reaction mixture was concentrated. The residue was suspended in CH₂Cl₂ and filtered. The solution was concentrated and the resulting residue was purified by silica gel column with 70% EtOAc/heptane to yield (1-(methoxymethyl)-4-oxo-4,5,6,7-tetrahydro-1H-indazol-3-yl)methyl acetate.

### Step 5. Synthesis of ethyl 2-(3-(acetoxymethyl)-1-(methoxymethyl)-1,5,6,7-tetrahydro-4H-indazol-4-ylidene)-2-fluoroacetate

To a solution of (1-(methoxymethyl)-4-oxo-4,5,6,7-tetrahydro-1H-indazol-3-yl)methyl acetate (268 mg, 1.06 mmol) and ethyl 2-(diethoxyphosphoryl)-2-fluoroacetate (772 mg, 3.19 mmol) in THF (10 mL) at 0° C. was added NaH (60% in mineral oil, 115 mg, 2.87 mmol). The reaction was stirred at 0° C. for 4 h and was then warmed up to room temperature and stirred for 1 h. The reaction was quenched with aq. 1N HCl and extracted with EtOAc. The organic layer was washed with aq. NaCl, dried over Na₂SO₄ and concentrated. The resulting residue was purified by silica gel column with 30-60% EtOAc/hetpane to yield ethyl 2-(3-(acetoxymethyl)-1-(methoxymethyl)-1,5,6,7-tetrahydro-4H-indazol-4-ylidene)-2-fluoroacetate as a mixture of (E) and (Z) isomers.

### Step 6. Synthesis of methyl (Z)-2-fluoro-2-(3-(hydroxymethyl)-1-(methoxymethyl)-1,5,6,7-tetrahydro-4H-indazol-4-ylidene)acetate

A mixture of ethyl 2-(3-(acetoxymethyl)-1-(methoxymethyl)-1,5,6,7-tetrahydro-4H-indazol-4-ylidene)-2-fluoroacetate (330 mg, 0.97 mmol) and K₂CO₃ (67 mg, 0.485 mmol) in MeOH (10 mL) was stirred at room temperature for 2 h. The reaction mixture was concentrated. The residue was diluted with CH₂Cl₂ and washed with aq. 1N HCl and sat. aq. NaCl. The organic layer was dried over Na₂SO₄ and concentrated. The resulting residue was purified by silica gel column to yield methyl (Z)-2-fluoro-2-(3-(hydroxymethyl)-1-(methoxymethyl)-1,5,6,7-tetrahydro-4H-indazol-4-ylidene)acetate.

### Step 7. Synthesis of methyl (Z)-2-(3-(chloromethyl)-1-(methoxymethyl)-1,5,6,7-tetrahydro-4H-indazol-4-ylidene)-2-fluoroacetate

To a solution of methyl (Z)-2-fluoro-2-(3-(hydroxymethyl)-1-(methoxymethyl)-1,5,6,7-tetrahydro-4H-indazol-4-ylidene)acetate in CH₂Cl₂ (5 mL) at 0° C. was added SOCl2 (0.074 mL, 1.02 mmol). The reaction was stirred at 0° C. for 45 min. The reaction was quenched with H₂O and extracted with CH₂Cl₂. The organic layer was washed with sat. aq. NaCl, dried over Na₂SO₄ and concentrated to yield methyl (Z)-2-(3-(chloromethyl)-1-(methoxymethyl)-1,5,6,7-tetrahydro-4H-indazol-4-ylidene)-2-fluoroacetate.

### Step 8. Synthesis of methyl (Z)-2-(3-(2,4-dichlorobenzyl)-1-(methoxymethyl)-1,5,6,7-tetrahydro-4H-indazol-4-ylidene)-2-fluoroacetate

A mixture of methyl (Z)-2-(3-(chloromethyl)-1-(methoxymethyl)-1,5,6,7-tetrahydro-4H-indazol-4-ylidene)-2-fluoroacetate (61.6 mg, 0.204 mmol), (2,4-dichlorophenyl)boronic acid (77.9 mg, 0.408 mmol), Na₂CO₃ (54.1 mg, 0.51 mmol), and Pd(PPh₃)₄ (11.8 mg, 0.0102 mmol) in 1,4-dioxane (2 mL) and H₂O (0.4 mL) was heated at 130° C. by microwave for 1 h. The reaction mixture was diluted with CH₂Cl₂ and filtered. The solution was concentrated and the resulting residue was purified by silica gel column with 20-40% EtOAc/heptane to yield methyl (Z)-2-(3-(2,4-dichlorobenzyl)-1-(methoxymethyl)-1,5,6,7-tetrahydro-4H-indazol-4-ylidene)-2-fluoroacetate. Mass spectrum (ESI, m/z): Calculated for C₁₉H₂₀Cl₂FN₂O₃: 413.1 [M+H], found: 413.1.

### Step 9. Synthesis of (Z)-2-(3-(2,4-dichlorobenzyl)-1-(methoxymethyl)-1,5,6,7-tetrahydro-4H-indazol-4-ylidene)-2-fluoroethan-1-ol

To a solution of methyl (Z)-2-(3-(2,4-dichlorobenzyl)-1-(methoxymethyl)-1,5,6,7-tetrahydro-4H-indazol-4-ylidene)-2-fluoroacetate (64 mg, 0.155 mL) in CH₂Cl₂ (3 mL) at −78° C. was added DIBAL-H (1.0 M in heptane, 0.62 mL, 0.62 mmol). The reaction was kept at −78° C. for 1 h before the reaction was quenched with MeOH and H₂O. The resulting mixture was diluted with EtOAc and washed with aq. 1N NaOH and sat. aq. NaCl. The organic layer was dried over Na2SO4 and concentrated to yield (Z)-2-(3-(2,4-dichlorobenzyl)-1-(methoxymethyl)-1,5,6,7-tetrahydro-4H-indazol-4-ylidene)-2-fluoroethan-1-ol. Mass spectrum (ESI, m/z): Calculated for C₁₈H₂₀Cl₂FN₂O₂: 385.1 [M+H], found: 385.0.

### Steps 10-14. Synthesis of (Z)-5-chloro-N-((2-(3-(2,4-dichlorobenzyl)-1,5,6,7-tetrahydro-4H-indazol-4-ylidene)-2-fluoroethyl)carbamoyl)-4-methylthiophene-2-sulfonamide

(Z)-5-chloro-N-((2-(3-(2,4-dichlorobenzyl)-1,5,6,7-tetrahydro-4H-indazol-4-ylidene)-2-fluoroethyl)carbamoyl)-4-methylthiophene-2-sulfonamide was prepared from (Z)-2-(3-(2,4-dichlorobenzyl)-1-(methoxymethyl)-1,5,6,7-tetrahydro-4H-indazol-4-ylidene)-2-fluoroethan-1-ol according to the procedures as described in Example 46, Steps 7-11.

¹H NMR (CHLOROFORM-d) δ: 7.33-7.43 (m, 2H), 7.13 (dd, J=8.1, 2.0 Hz, 1H), 6.95 (d, J=8.1 Hz, 1H), 3.99-4.22 (m, 4H), 2.64-2.81 (m, 2H), 2.31-2.43 (m, 2H), 2.12 (s, 3H), 1.82-1.96 (m, 2H). Mass spectrum (ESI, m/z): Calculated for C₂₂H₂₁Cl₃FN₄O₃S₂: 577.0 [M+H], found: 577.0.

### BIOLOGICAL EXAMPLES

### Biological Example 1: [3H]-PGE2 Binding

EP3 competition binding was determined using the following materials and experimental conditions:
Beads=Perkin Elmer RPNQ0001
[3H]PGE2=Perkin Elmer NET428025UC
384-well plate=Perkin Elmer 6007290
EP3 membrane=Millipore HTS092M
Membrane concentration=2 μg/well
Beads concentration=0.25 mg/well
[3H]-PGE2=2 nM; DMSO—0.1%
Binding buffer=50 mM Tris, 10 mM MgCl₂, 1 mM EDTA, PH 7.4.

The reaction system was prepared by mixing 5 μl of unlabeled compound; 5 μl of diluted [3H]-PGE2; 5 μl of diluted membrane; and 15 μl of SPA beads dissolved in binding buffer (adding in the following sequence: Unlabeled compound, [3H]-PGE2, Membrane and Beads).

### Procedure:

The binding buffer was removed from refrigeration (@ 4° C.) and allowed to warm to room temperature. Test compound (@ stock concentration of 10 nM in DMSO) was serial dilutes (1:3) with 100% DMSO using Echo plate. Diluted test compound (30 nL) was dispensed to the 384-well assay plate (using Echo 550 Labcyte System). To each well was then added assay buffer (5 μl) and each well was spun. Beads were weighed out, dissolved in assay buffer to a concentration of 0.25 mg/15 μl. Original membrane was diluted with assay buffer to a concentration of 2 μg/5 μl. Original [3H]-PGE2 with diluted with assay buffer to a concentration of 12 nM. To each test well were then added diluted [3H]-PGE2 (5 μl), diluted membrane (5 μl), and dissolved beads (15 μl). The plate was equilibrated at room temperature on a plate shaker for 2 hours and then read with a TOPCOUNT scintillation plate reader. Results were analyzed using a Prism program with non-linear regression, one-site fit for Ki.

### Biological Example 2: CHO Cell cAMP Assay

This assay monitored the cAMP generation in CHO cells over-expressing the EP3 receptor in the presence or absence of antagonist stimulated with Forskolin (FSK, CAS No. 66428-89-5) and a known EP3 Agonist (sulprostone, CAS No. 60325-46-4). For detection, a homogeneous competitive immunoassay (Time Resolved Fluorescence Energy Transfer (hTR-FRET)) was used.

Cells (6K cells per well) were plated in Poly-D-Lysine coated plates the day before the assay was to be run and were incubated at 37° C. One hour before the assay the media was removed and replaced with assay buffer (500 mL HBSS (+Ca)+2.5 mL 1M HEPES+6.66 mL 7.5% BSA) (starved at 37° C.). At the start of the assay the buffer was removed from the cells and replaced with buffer containing test compound(s). Sulprostone at an EC₈₀ dose (700 nM) and FSK at an EC₇₀ dose (2 μM) were added to initiate the reaction (37° C. for 30 minutes).

FSK increases cAMP production and sulprostone decreases cAMP production produced by FSK. In this assay, EP3 antagonists will increase the cAMP to the level of cAMP generated by FSK alone.

The reaction was terminated with the addition of cAMP detection reagents (labeled cAMP and labeled cAMP antibody in lysis buffer (HTRF reagent, which utilizes a cryptate-labeled anti-cAMP and d2-labeled cAMP-d2 is an HTRF acceptor fluorophore)). Approximately one hour later the plates were read on an Envision (Perkin Elmer) in HTRF mode. Well results were calculated based on a ratio of counts at 665 nm and 615 nm (data output was a calculation of the ratio: (read @665 nm/read @ 615 nm)*1000. For test compounds dosed serially, well results were converted to nM cAMP using a cAMP standard run on each plate. nM cAMP from a set of wells dosed with test compound(s) were plotted. nM cAMP was calculated for each well from a standard curve located on each plate (P1-12 and P13-24) by first calculating the slope and deriving the intercept(b):

$$m = \frac{Δ {NS}_{cAMP\_s tan dard\_curve}}{{Δ [ s tan dard\_curve ]}_{\backslash}}$$

NS_{cAMP_s tan dard_cruve}=m(nM cAMP_s tan dard_curve)+b

NS_{P}, NS_{N}, NSₛₐₘₚₗₑ were converted to nM cAMP using the following equation:

$$nM\_cAMP = \frac{N S - b}{m}$$

IC₅₀ values were determined from a 4-point fit (Hill equation) of a single 11-point compound dosing. A best-fit curve was determined by the minimum sum of squares method plotting cAMP produced vs compound concentration.

Representative compounds of the present invention (including compounds of formula (I), compounds of formula (II) and compounds of formula (III)) were tested according to the procedures as described in Biological Example 1 and Biological Example 2 above, with results as listed in Table 8 below.

TABLE 8
Biological Assay Results

| | [3H]-PGE2 | CHO_cAMP |
|---|---|---|
| ID No. | Binding Ki (μM) | IC₅₀ (μM) |
| 2 | 0.011 | 0.092 |
| 3 | 0.003 | 0.033 |
| 4 | 0.004 | 0.012 |
| 5 | 0.001 | 0.005 |
| 6 | 0.084 | 0.110 |
| 7 | 0.5 nM | 0.011 |
| 8 | 0.1 nM | 0.002 |
| 9 | 0.006 | 0.014 |
| 10 | 0.015 | 0.028 |
| 11 | 0.001 | 0.002 |
| 12 | 0.019 | 0.048 |
| 13 | 0.010 | 0.029 |
| 14 | 2.0 | 3.7 |
| 15 | 0.001 | 0.005 |
| 16 | 0.009 | 0.027 |
| 17 | 0.019 | 0.074 |
| 18 | 0.147 | 0.662 |
| 19 | 0.014 | 0.083 |
| 20 | 0.280 | 1.0 |
| 21 | 0.058 | 0.330 |
| 22 | 0.373 | 2.9 |
| 23 | 0.256 | 0.453 |
| 24 | 0.003 | 0.006 |
| 25 | 0.051 | 0.251 |
| 26 | 0.085 | 0.699 |
| 27 | 0.005 | 0.013 |
| 28 | 0.006 | 0.027 |
| 29 | 0.001 | 0.002 |
| 30 | 0.026 | 0.035 |
| 31 | 0.007 | 0.013 |
| 32 | 0.168 | 1.3 |
| 33 | 0.006 | 0.009 |
| 34 | 0.003 | 0.011 |
| 35 | 0.542 | 2.8 |
| 36 | 0.464 | 2.9 |
| 37 | 0.108 | 0.087 |
| 38 | 0.003 | 0.007 |
| 39 | 0.062 | 0.114 |
| 40 | 0.001 | 0.001 |
| 41 | 0.006 | 0.009 |
| 42 | 0.003 | 0.006 |
| 43 | 0.003 | 0.008 |
| 44 | 0.095 | 0.177 |
| 45 | 1.3 | 6.7 |
| 46 | 0.002 | 0.008 |
| 47 | 0.065 | 0.120 |
| 48 | 0.005 | 0.014 |
| 49 | 0.4 nM | 0.001 |
| 50 | 0.029 | 0.075 |
| 51 | >10 | 15.7 |
| 52 | 0.001 | 0.003 |
| 53 | 0.004 | 0.018 |
| 54 | 0.003 | 0.024 |
| 55 | 0.003 | 0.011 |
| 56 | 0.001 | 0.004 |
| 57 | 0.001 | 0.003 |
| 58 | 0.110 | 1.3 |
| 59 | 0.005 | 0.012 |
| 60 | 0.007 | 0.004 |
| 61 | 0.007 | 0.017 |
| 62 | 0.063 | 0.245 |
| 63 | 0.044 | 0.121 |
| 64 | 0.012 | 0.081 |
| 65 | 0.027 | 0.044 |
| 66 | 0.004 | 0.021 |
| 67 | 0.026 | 0.074 |
| 68 | 0.027 | 0.032 |
| 69 | 0.179 | 0.118 |
| 70 | 1.0 | 8.2 |
| 73 | 0.003 | 0.005 |
| 74 | 0.035 | 0.031 |
| 75 | 0.001 | 0.004 |
| 76 | 0.002 | 0.009 |
| 77 | 0.012 | 0.258 |
| 78 | 0.001 | 0.010 |
| 79 | 0.4 nM | 0.002 |
| 80 | 0.920 | 4.4 |
| 81 | >10 | 14.1 |
| 82 | 0.106 | 2.6 |
| 83 | 0.097 | 0.266 |
| 84 | 0.001 | 0.005 |
| 85 | 0.4 nM | 0.004 |
| 86 | 0.028 | 0.069 |
| 87 | 0.110 | 0.435 |
| 88 | 0.104 | 0.554 |
| 89 | 0.026 | 0.160 |
| 90 | 0.037 | 0.272 |
| 91 | 0.018 | 0.034 |
| 92 | 0.017 | 0.214 |
| 93 | 0.006 | 0.017 |
| 94 | 0.750 | 11.1 |
| 95 | >10 | >20 |
| 96 | >10 | >20 |
| 97 | 4.9 | >20 |
| 98 | 0.171 | 0.120 |
| 99 | 0.001 | 0.009 |
| 100 | 0.003 | 0.007 |
| 101 | 0.053 | 0.144 |
| 102 | >10 | >20 |
| 103 | 0.054 | 0.228 |
| 104 | >10 | >20 |
| 105 | 0.238 | 0.294 |
| 106 | >10 | >20 |
| 107 | 0.002 | 0.027 |
| 108 | >10 | >20 |
| 109 | 0.007 | 0.060 |
| 110 | >10 | >20 |
| 111 | 0.059 | 0.399 |
| 112 | >10 | >20 |
| 113 | 4.1 | 6.9 |
| 114 | 14.5 | >20 |
| 115 | 1.1 | 4.2 |
| 116 | 4.1 | >20 |
| 117 | >10 | >20 |
| 118 | 2.0 | 16.0 |
| 119 | 0.043 | 0.230 |
| 120 | 0.010 | 0.012 |
| 121 | 0.017 | 0.159 |
| 122 | 0.006 | 0.045 |
| 123 | 0.001 | 0.006 |
| 124 | 0.100 | 0.798 |
| 125 | 0.003 | 0.020 |
| 126 | 0.001 | 0.030 |
| 127 | 0.001 | 0.008 |
| 128 | 0.532 | 8.4 |
| 129 | 0.029 | 0.548 |
| 130 | 0.040 | 0.182 |
| 131 | 0.094 | 0.349 |
| 132 | 0.350 | 3.9 |
| 133 | 0.289 | 1.265 |
| 134 | 0.007 | 0.052 |
| 135 | 0.472 | 4.6 |
| 136 | 0.005 | 0.046 |
| 137 | 0.220 | 0.488 |
| 138 | 0.4 nM | 0.006 |
| 139 | 0.007 | 0.009 |
| 140 | 0.008 | 0.012 |
| 141 | 0.215 | 0.750 |
| 142 | 0.008 | |
| 143 | 0.003 | 0.018 |
| 144 | 0.062 | |
| 145 | 0.006 | |
| 146 | 0.022 | |
| 147 | 0.4 nM | 0.005 |
| 148 | 0.006 | |
| 149 | 0.002 | 0.006 |
| 150 | 0.002 | 0.004 |
| 151 | 0.102 | |
| 152 | 0.009 | 0.008 |
| 153 | 0.022 | |
| 154 | 0.013 | |
| 155 | 0.002 | 0.004 |
| 156 | 0.002 | 0.004 |
| 157 | 0.467 | |
| 158 | 0.032 | |
| 159 | 0.022 | |
| 160 | 0.217 | |
| 161 | 0.057 | |
| 163 | >10 | |
| 164 | 3.2 | |
| 165 | 0.010 | |
| 166 | 0.006, 0.007 | |
| 167 | 0.007 | |
| 168 | 0.232 | |
| 169 | 0.083 | |
| 170 | 0.612 | |
| 171 | 0.018 | |
| 172 | 0.004 | |
| 173 | 0.059 | |
| 174 | 0.149 | |
| 175 | 0.016 | |
| 176 | 0.001 | |
| 177 | 0.001 | |
| 178 | 0.006 | |
| 179 | 0.4 nM | |
| 180 | 0.164 | |
| 181 | 0.006 | |
| 182 | 0.153 | |
| 183 | 0.218 | |
| 184 | 0.014 | |
| 185 | 0.001 | |
| 186 | 0.030 | |
| 187 | 0.001 | |
| 188 | 0.008 | |
| 189 | 0.001 | |
| 190 | 0.001 | |
| 191 | 0.015 | |
| 192 | 0.006 | |
| 193 | 0.075 | |
| 194 | 0.025 | |
| 195 | 0.045 | |
| 196 | 0.001 | |
| 197 | 0.008 | |

### Biological Example 3: IV Infused EP3 Antagonists on Circulating Insulin and Glucose in the Sulprostone Infusion IVGTT Model

Jugular vein and carotid artery cannulated male Sprague Dawley rats (˜250 g, available from Charles River) were housed one rat per suspended cage in a temperature-controlled room with 12-hour light/dark cycle. The rats were allowed ad libitum access to water and maintained on a regular diet. Animals were acclimated for minimum 5 days prior to the start of the experiment. Experimental procedures were carried out in accordance with institutional standards for animal care and were approved by the institute's animal care and use committee.

A blood sample for insulin and blood chemistry was collected at 20 min (t=−20) prior to the start of dosing with sulprostone and/or test compound (EP3 antagonist). Sulprostone solution was prepared in 10 U/ml heparin saline solution at a concentration of 0.3 mg/ml. Test compound solution(s) were prepared in 20% HPBCD (pH=7) 1.030 mg/ml. Final test compound solution was mixed 1:1 volume with sulprostone solution immediately prior to starting the experiment. Tested compound solution (1.053 mpk (mg/kg)) or saline bolus at 2 ml/kg was injected into the animals and a blood sample was taken for exposure analysis at 2 min after the bolus. After 18 min, another blood sample was taken for exposure analysis and also for measurement of insulin and blood chemistry (t=0). Animals were then injected (through infusion syringe) with a 50% glucose solution (1 g/kg) followed by IV infusion at 6 ml/kg/h with the vehicle (20% HPBCD, pH=7), 10 μg/kg/min sulprostone only or a mixture of 10 μg/kg/min sulprostone and the test compound at 3.09 mpk/h. Blood samples were then collected at 2, 5, 10, 15, 20, 30 min post infusion for measurement of insulin and blood chemistry. Another blood sample was collected at 31 min post infusion for exposure analysis.

Blood samples from all animals were collected into Heparin treated tubes. Background was measured via a glucometer at the 0 and 2 min time points to ensure that the animal received the glucose bolus. Plasma insulin was measured via a Meso Scale Discovery metabolic assay (available from Meso Scale). Plasma glucose, free fatty acid (FFA), triglycerides, cholesterol and ketones were measured on an OLYMPUS AU400E chemistry analyzer. Observations on animal health were recorded throughout the procedure. Samples from time points (after the bolus) 2, 20 and 50 min were submitted for measurement of exposure by plasma drug concentration.

Statistical analysis was performed using the program Prism (Graphpad, Monrovia, Calif.) with either a repeated measures 2-way ANOVA (glucose and insulin curves with Bonferroni's multiple comparison test) or a one-way ANOVA and Tukey's multiple comparison test (AUC). AUC=Integrated area under the stimulated glucose excursion and insulin curve from t=0 to t=30 minutes and from t=0 to t=10 min. Acute Insulin Response (AIR) is calculated as the mean insulin at 2, 5 and 10 min after glucose bolus—baseline.

Testing according to the procedure describe above, Compound #4, prepared for example as described in Example 6, was measured to reverse suppression of glucose stimulated insulin secretion (GSIS) by sulprostone at 2.1 mg/kg bolus followed by 3.09 mg/kg/hour infusion in SD rats.

### Formulation Example 1

### Solid, Oral Dosage Form—Prophetic Example

As a specific embodiment of an oral composition, 100 mg of, for example Compound #4, prepared as in Example 6, is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 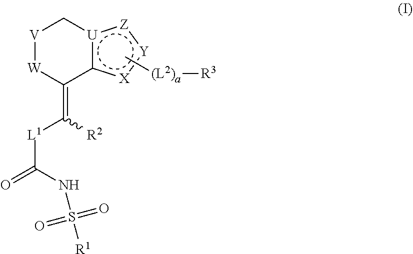
wherein 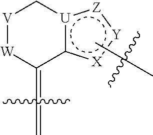
is an 8- to 10-membered, partially unsaturated ring structure selected from the group consisting of 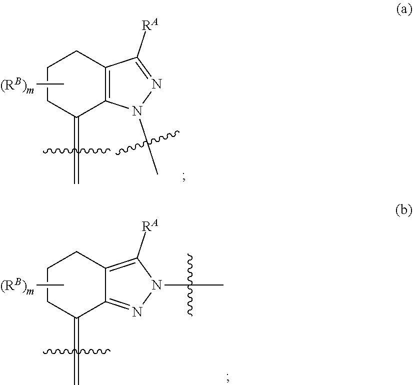
wherein R^{A} is selected from the group consisting of hydrogen, C₁₋₂alkyl and fluorinated C₁₋₂alkyl,
m is an integer from 0 to 2;
each R^{B} is selected from the group consisting of fluoro and C₁₋₂alkyl;
provided that when R^{A} is other than hydrogen, then m is 0;
provided further that each R^{B} is bound at the 5- or 6-position of the 4,5,6,7-tetrahydroindazole ring structure; and that when m is 2, then both R^{B} groups are bound to the same 5- or 6-position carbon atom; 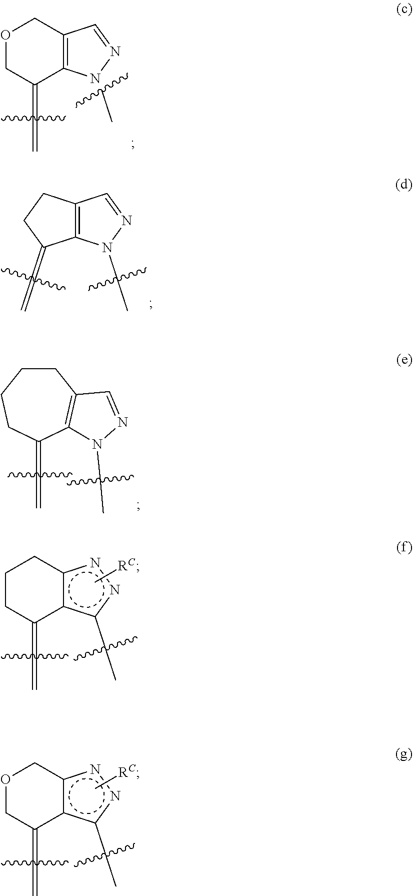
wherein R^{C} is bound to either nitrogen atom and is selected from the group consisting of hydrogen, C₁₋₂alkyl and —(C₁₋₂alkyl)-O—(C₁₋₂alkyl), 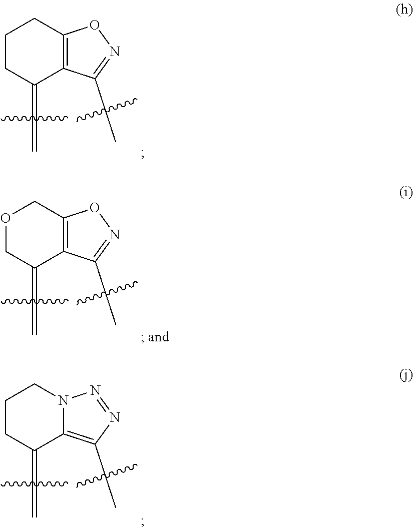 [SMILES: *c1noc2c1C(=*)CCC2 ; ";" ; *c1noc2c1C(=*)COC2 ; "; and" ; *c1nnn2c1C(=*)CCC2 ; ";"]
R¹ is selected from the group consisting of C₁₋₄alkyl, phenyl, naphthyl and heterocyclyl;
wherein the phenyl, naphthyl or heterocyclyl is optionally substituted with one or more substituents independently selected from the group consisting of halogen, oxo, cyano, C₁₋₄alkyl, fluorinated C₁₋₂alkyl, C₁₋₄alkoxy, fluorinated C₁₋₂alkoxy, nitro, —NR^{D}R^{E}, —C(O)—NR^{D}R^{E}, —S—C₁₋₂alkyl and C₃₋₅cycloalkyl;
wherein R^{D} and R^{E} are each independently selected from the group consisting of hydrogen, methyl and ethyl;
L¹ is selected from the group consisting of —CH₂—, —CH₂CH₂—, —CH═CH—, —O—CH₂—, —NH—CH₂—, —N(CH₃)—CH₂— and —N(CH₂CH₃)—CH₂—; wherein the —CH═ or —CH₂— portion of the L¹ group is bound to the double bond;
R² is selected from the group consisting of hydrogen and fluoro;
a is an integer from 0 to 1;
L² is selected from the group consisting of —CH₂— and —CH₂CH₂—;
R³ is selected from the group consisting of C₁₋₄alkyl, phenyl, naphthyl and heterocyclyl;
wherein the phenyl, naphthyl or heterocyclyl is optionally substituted with one or more substituents independently selected from the group consisting of halogen, oxo, C₁₋₄alkyl, fluorinated C₁₋₄alkyl, C₁₋₄alkoxy, fluorinated C₁₋₄alkoxy, cyano, —NR^{F}R^{G}, —C(O)—NR^{F}R^{G}, —NH—C(O)—C₁₋₄alkyl, —SO₂—C₁₋₂alkyl, phenyl, benzyl, phenylethyl, and 5- to 6-membered heteroaryl;
wherein R^{F} and R^{G} are each independently selected from the group consisting of hydrogen and C₁₋₄alkyl;
and wherein the phenyl, benzyl, phenylethyl or 5- to 6-membered heteroaryl is further optionally substituted with one to two substituents independently selected from the group consisting of halogen and C₁₋₄alkyl;
or a stereoisomer or pharmaceutically acceptable salt thereof.

2. The compound of claim 1, wherein 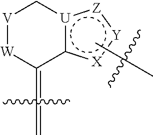
is an 8- to 10-membered, partially unsaturated ring structure selected from the group consisting of 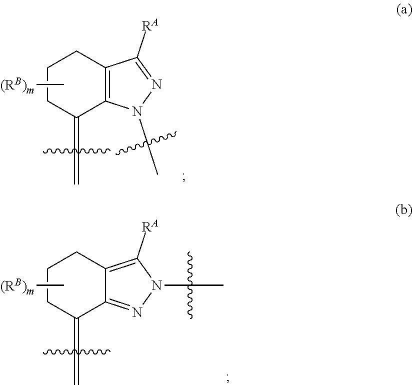
wherein R^{A} is selected from the group consisting of hydrogen, C₁₋₂alkyl and fluorinated C₁₋₂alkyl;
m is an integer from 0 to 2;
each R^{B} is fluoro;
provided that when R^{A} is other than hydrogen, then m is 0;
provided further that each R^{B} is bound at the 5- or 6-position of the 4,5,6,7-tetrahydroindazole ring structure; and that when m is 2, then both R^{B} groups are bound to the same 5- or 6-position carbon atom; 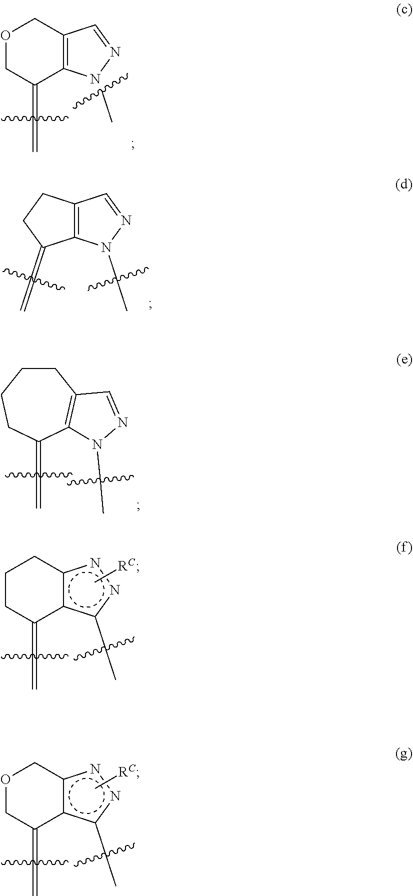
wherein R^{C} is selected from the group consisting of hydrogen, C₁₋₂alkyl and —(C₁₋₂alkylene)-O—(C₁₋₂alkyl); 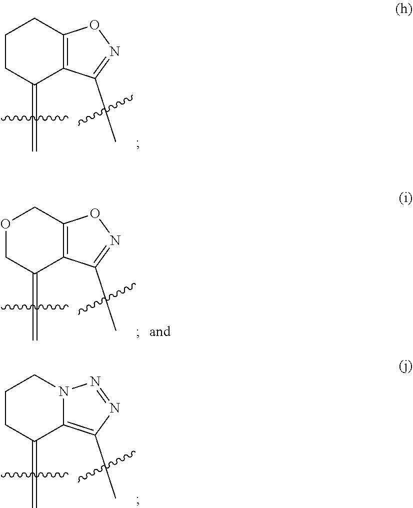 [SMILES: *c1noc2c1C(=*)CCC2 ; *c1noc2c1C(=*)COC2 ; *c1nnn2c1C(=*)CCC2]
R¹ is selected from the group consisting of C₁₋₄alkyl, phenyl, naphthyl and heterocyclyl;
wherein the phenyl, naphthyl or heterocyclyl is optionally substituted with one to two substituents independently selected from the group consisting of halogen, oxo, C₁₋₄alkyl, fluorinated C₁₋₂alkyl, C₁₋₄alkoxy, fluorinated C₁₋₂alkoxy, —NR^{D}R^{E}, —C(O)—NR^{D}R^{E} and —NH—C(O)—C₁₋₄alkyl;
wherein R^{D} and R^{E} are each independently selected from the group consisting of hydrogen and methyl;
L¹ is selected from the group consisting of —CH₂—, —CH₂CH₂—, —CH═CH—, —O—CH₂—, —NH—CH₂— and —N(CH₃)—CH₂—; wherein the —CH═ or —CH₂— portion of the L¹ group is bound to the double bond;
R² is selected from the group consisting of hydrogen and fluoro;
a is an integer from 0 to 1;
L² is selected from the group consisting of —CH₂— and —CH₂CH₂—;
R³ is selected from the group consisting of C₁₋₄alkyl, phenyl, naphthyl and heterocyclyl;
wherein the phenyl, naphthyl or heterocyclyl is optionally substituted with one to two substituents independently selected from the group consisting of halogen, C₁₋₄alkyl, fluorinated C₁₋₄alkyl, C₁₋₄alkoxy, fluorinated C₁₋₄alkoxy, —NR^{F}R^{G}, —C(O)—NR^{F}R^{G}, —NH—C(O)—C₁₋₄alkyl, —SO₂—C₁₋₂alkyl, phenyl, benzyl and 5- to 6-membered heteroaryl;
wherein R^{F} and R^{G} are each independently selected from the group consisting of hydrogen and methyl;
and wherein the phenyl, benzyl or 5- to 6-membered heteroaryl is further optionally substituted with one to two substituents independently selected from the group consisting of halogen and C₁₋₂alkyl;
or a stereoisomer or pharmaceutically acceptable salt thereof.

3. The compound of claim 2, wherein 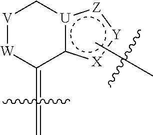
is an 8- to 10-membered, partially unsaturated ring structure selected from the group consisting of 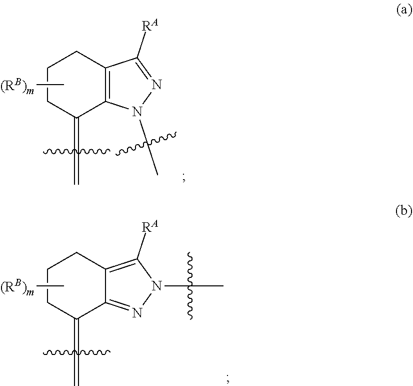
wherein R^{A} is selected from the group consisting of hydrogen, C₁₋₂alkyl and fluorinated C₁₋₂alkyl;
m is an integer from 0 to 2;
each R^{B} is fluoro;
provided that when R^{A} is other than hydrogen, then m is 0;
provided further that each R^{B} is bound at the 5- or 6-position of the 4,5,6,7-tetrahydroindazole ring structure; and that when m is 2, then both R^{B} groups are bound to the same 5- or 6-position carbon atom; 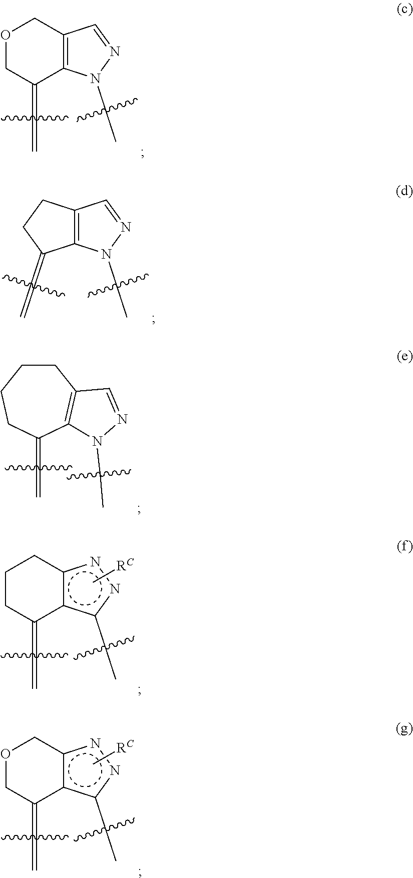
wherein R^{C} is selected from the group consisting of hydrogen, C₁₋₂alkyl and —(C₁₋₂alkylene)-O—(C₁₋₂alkyl), 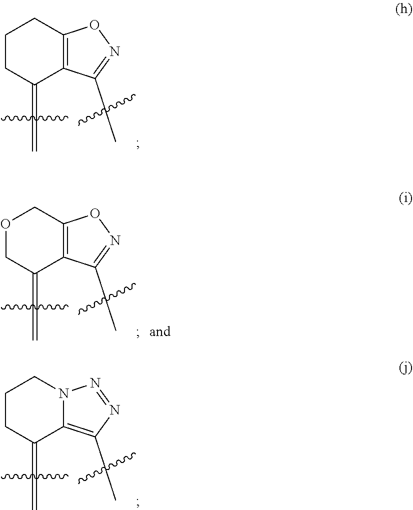 [SMILES: *c1noc2c1C(=*)CCC2 ; *c1noc2c1C(=*)COC2 ; *c1nnn2c1C(=*)CCC2]
R¹ is selected from the group consisting of C₁₋₄alkyl, phenyl, thienyl, thiazolyl, pyrazolyl, pyridiyl, indazolyl, benzofuryl, benzothienyl, benzothiazolyl, benzoxazolyl, quiunolinyl and 2,3-dihydrobenzo[b][1.4]dioxin-6-yl,
wherein the phenyl, thienyl, thiazolyl, pyrazolyl, pyridiyl, indazolyl, benzofuryl, benzothienyl, benzothiazolyl, benzoxazolyl or quinolinyl is optionally substituted with one to two substituents independently selected from the group consisting of halogen, C₁₋₃alkyl, C₁₋₂alkoxy, oxo and —NH—C(O)—(C₁₋₂alkyl);
L¹ is selected from the group consisting of —CH₂—, —CH₂CH₂—, —CH═CH—, —OCH₂—, —NH—CH₂— and —N(CH₃)—CH₂—; wherein the —CH═ or —CH₂— portion of the L¹ group is bound to the double bond;
R² is selected from the group consisting of hydrogen and fluoro;
a is an integer from 0 to 1;
L² is selected from the group consisting of —CH₂— and —CH₂CH₂—;
R³ is selected from the group consisting of C₁₋₄alkyl, phenyl, naphthyl, pyrimidinyl, pyridyl, pyrazolyl and piperidinyl;
wherein the phenyl, naphthyl, pyrimidinyl, pyrazolyl or piperidinyl is optionally substituted with one to two substituents independently selected from the group consisting of halogen, C₁₋₂alkyl, fluorinated C₁₋₂alkyl, C₁₋₂alkoxy, —SO₂—(C₁₋₂alkyl), phenyl, benzyl and pyridyl;
and wherein the phenyl, benzyl or pyridyl substituent is further optionally substituted with one to two substituents independently selected from the group consisting of halogen;
or a stereoisomer or pharmaceutically acceptable salt thereof.

4. The compound of claim 3, wherein 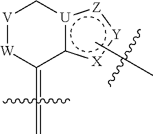
is an 8- to 10-membered, partially unsaturated ring structure selected from the group consisting of 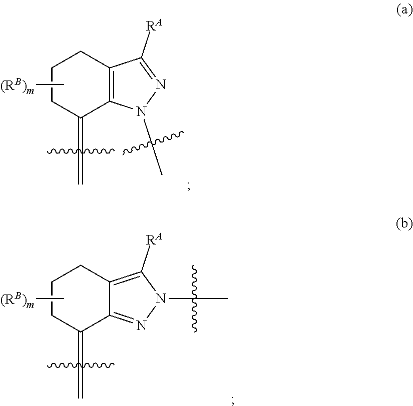
wherein R^{A} is selected from the group consisting of hydrogen, methyl and difluoro-methyl;
m is an integer from 0 to 2;
provided that when R^{A} is other than hydrogen, then m is 0;
each R^{B} is selected from the group consisting of 5-fluoro and 6-fluoro;
provided that when m is 1, R^{B} is 6-fluoro; provided further that when m is 2, both R^{B} groups are the same and are selected from the group consisting of 5-fluoro and 6-fluoro; 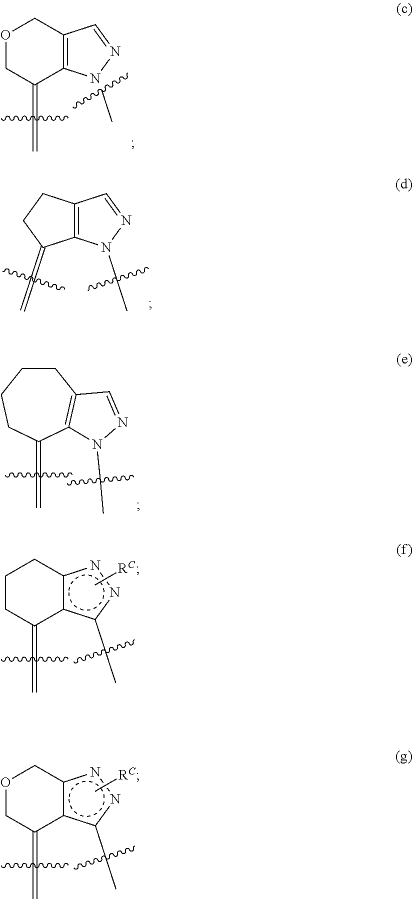
wherein R^{C} is selected from the group consisting of hydrogen, methyl and —CH₂—OCH₃, 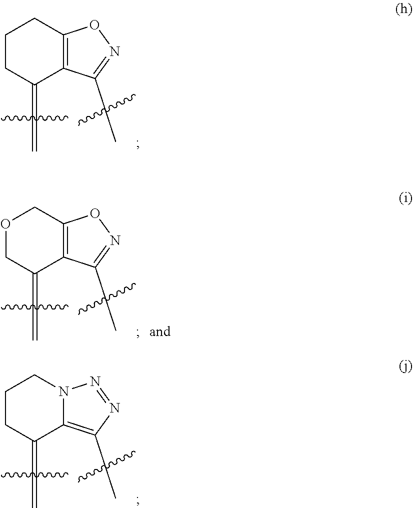 [SMILES: *c1noc2c1C(=*)CCC2 ; *c1noc2c1C(=*)COC2 ; *c1nnn2c1C(=*)CCC2]
R¹ is selected from the group consisting of isopropyl, 3-chloro-phenyl, 4-chloro-phenyl, 3,4-difluoro-phenyl, 3,5-difluoro-phenyl, 2-methoxy-phenyl, 3-methoxy-phenyl, 4-methoxy-phenyl, 3,4-dimethoxy-phenyl, 2-methoxy-4-chloro-phenyl, 2-methoxy-5-bromo-phenyl, 2-methoxy-5-chloro-phenyl, 2-methoxy-5-fluoro-phenyl, 2-chloro-5-methoxy-phenyl, 2-fluoro-5-methoxy-phenyl, 3-chloro-4-methoxy-phenyl, 3-fluoro-4-methoxy-phenyl, 3-methoxy-4-chloro-phenyl, thien-2-yl, 5-chloro-thien-2-yl, 4,5-dichloro-thien-2-yl, 4,5-dimethyl-thienyl, 4-methyl-5-chloro-thien-2-yl, 2-methyl-thiazol-5-yl, 2,4-dimethyl-thiazol-5-yl, 1-isopropyl-thiazol-4-yl, 2-(methyl-carbonyl-amino)-4-methyl-thiazol-5-yl, 1-methyl-pyrazol-3-yl, 1-methyl-pyrazol-4-yl, 1,3-dimethyl-pyrazol-4-yl, 1-isopropyl-pyrazol-4-yl, 1,5-dimethyl-pyrazol-4-yl, 1-methyl-pyrazol-5-yl, 1,3-dimethyl-pyrazol-5-yl, 5-chloro-6-methyl-pyrid-3-yl, 6-methoxy-pyrid-3-yl, 1-methyl-indazol-5-yl, 1-methyl-indazol-6-yl, benzofur-2-yl, benzothien-2-yl, benzothiazol-2-yl, 2-methyl-benzoxazol-5-yl, 2-methoxy-benzoxazol-6-yl, benzoxazol-6-yl-2-one, quinolin-3-yl and 2,3-dihydrobenzo[b][1.4]dioxin-6-yl;
L¹ is selected from the group consisting of —CH₂—, —CH₂CH₂—, —CH═CH—, —OCH₂—, —NH—CH₂— and —N(CH₃)—CH₂—; wherein the —CH═ or —CH₂— portion of the L¹ group is bound to the double bond;
R² is selected from the group consisting of hydrogen and fluoro;
a is an integer from 0 to 1;
L² is selected from the group consisting of —CH₂— and —CH₂CH₂—;
R³ is selected from the group consisting of isopropyl, phenyl, 3-bromo-phenyl, 4-bromo-phenyl, 4-chloro-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, pyridimidin-2-yl, 5-bromo-pyrimidin-2-yl, 2-phenyl-pyrimidin-5-yl, 5-phenyl-pyrimidin-2-yl, 6-methoxy-pyrid-3-yl, 1-phenyl-pyrazol-4-yl, 1-methyl-piperidin-4-yl, 1-(4-fluoro-phenyl)-piperidin-4-yl, 1-(benzyl)-piperidin-4-yl and 1-(pyrid-2-yl)-piperidin-4-yl;
or a stereoisomer or pharmaceutically acceptable salt thereof.

5. The compound of claim 4, wherein 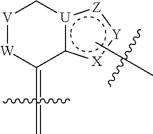
is an 8- to 10-membered, partially unsaturated ring structure selected from the group consisting of 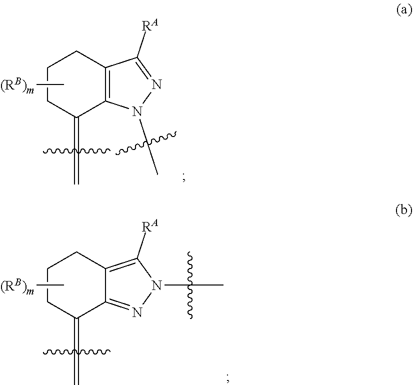
wherein R^{A} is selected from the group consisting of hydrogen, methyl and difluoro-methyl;
m is an integer from 0 to 2;
provided that when R^{A} is other than hydrogen, then m is 0;
each R^{B} is selected from the group consisting of 5-fluoro and 6-fluoro;
provided that when m is 1, R^{B} is 6-fluoro; provided further that when m is 2, both R^{B} groups are the same and are 5-fluoro; 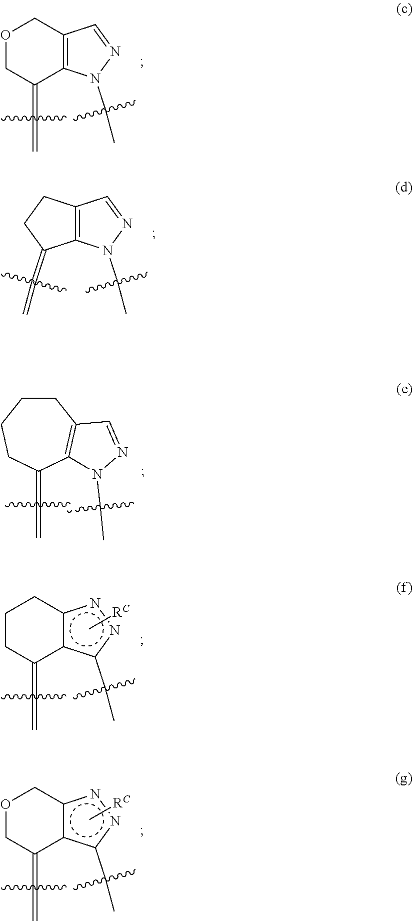
wherein R^{C} is selected from the group consisting of hydrogen, methyl and —CH₂—OCH₃, 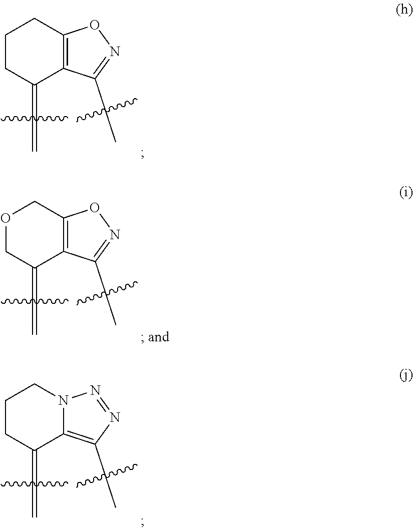 [SMILES: *c1noc2c1C(=*)CCC2 ; ";" ; *c1noc2c1C(=*)COC2 ; "; and" ; *c1nnn2c1C(=*)CCC2 ; ";"]
R¹ is selected from the group consisting of isopropyl, 3-chloro-phenyl, 4-chloro-phenyl, 3,4-difluoro-phenyl, 3,5-difluoro-phenyl, 2-methoxy-phenyl, 3-methoxy-phenyl, 4-methoxy-phenyl, 3,4-dimethoxy-phenyl, 2-methoxy-4-chloro-phenyl, 2-methoxy-5-bromo-phenyl, 2-methoxy-5-chloro-phenyl, 2-methoxy-5-fluoro-phenyl, 2-chloro-5-methoxy-phenyl, 2-fluoro-5-methoxy-phenyl, 3-chloro-4-methoxy-phenyl, 3-fluoro-4-methoxy-phenyl, 3-methoxy-4-chloro-phenyl, thien-2-yl, 5-chloro-thien-2-yl, 4,5-dichloro-thien-2-yl, 4,5-dimethyl-thienyl, 4-methyl-5-chloro-thien-2-yl, 2-methyl-thiazol-5-yl, 2,4-dimethyl-thiazol-5-yl, 1-isopropyl-thiazol-4-yl, 2-(methyl-carbonyl-amino)-4-methyl-thiazol-5-yl, 1-methyl-pyrazol-3-yl, 1-methyl-pyrazol-4-yl, 1-isopropyl-pyrazol-4-yl, 1,3-dimethyl-pyrazol-4-yl, 1,5-dimethyl-pyrazol-4-yl, 1-methyl-pyrazol-5-yl, 1,3-dimethyl-pyrazol-5-yl, 5-chloro-6-methyl-pyrid-3-yl, 6-methoxy-pyrid-3-yl, 1-methyl-indazol-5-yl, 1-methyl-indazol-6-yl, benzofur-2-yl, benzothien-2-yl, benzothiazol-2-yl, 2-methyl-benzoxazol-5-yl, 2-methoxy-benzoxazol-6-yl, benzoxazol-6-yl-2-one, quinolin-3-yl and 2,3-dihydrobenzo[b][1.4]dioxin-6-yl;
L¹ is selected from the group consisting of —CH₂—, —CH₂CH₂—, —CH═CH—, —OCH₂—, —NH—CH₂— and —N(CH₃)—CH₂—; wherein the —CH═ or —CH₂— portion of the L¹ group is bound to the double bond;
R² is selected from the group consisting of hydrogen and fluoro;
a is an integer from 0 to 1;
L² is selected from the group consisting of —CH₂— and —CH₂CH₂—;
R³ is selected from the group consisting of phenyl, 3-bromo-phenyl, 4-bromo-phenyl, 4-chloro-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl, 1-phenyl-pyrazol-4-yl, 1-methyl-piperidin-4-yl, 1-(4-fluoro-phenyl)-piperidin-4-yl, 1-(benzyl)-piperidin-4-yl and 1-(pyrid-2-yl)-piperidin-4-yl;
or a stereoisomer or pharmaceutically acceptable salt thereof.

6. The compound of claim 4, wherein 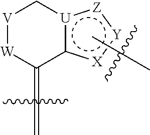
is an 8- to 10-membered, partially unsaturated ring structure selected from the group consisting of 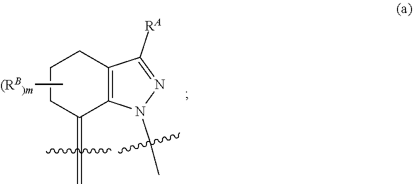
wherein R^{A} is selected from the group consisting of hydrogen and methyl;
m is an integer from 0 to 2;
provided that when R^{A} is methyl, then m is 0;
each R^{B} is selected from the group consisting of 5-fluoro and 6-fluoro;
provided that when m is 1, R^{B} is 6-fluoro; provided further that when m is 2, both R^{B} groups are the same and are 5-fluoro; 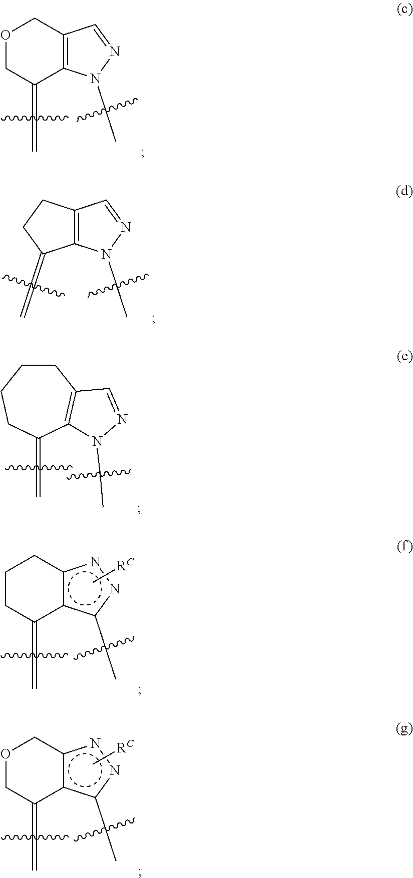
wherein R^{C} is bound to either nitrogen atom and is selected from the group consisting of hydrogen, methyl and —CH₂—OCH₃, 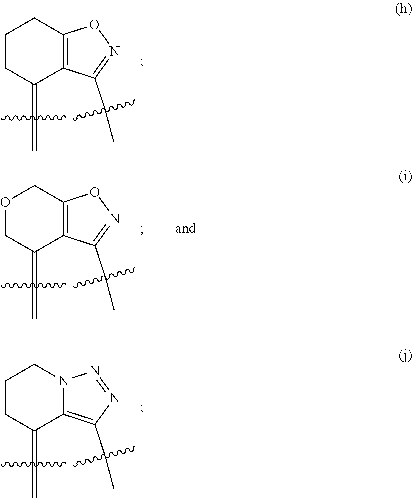 [SMILES: *c1noc2c1C(=*)CCC2 ; *c1noc2c1C(=*)COC2 ; *c1nnn2c1C(=*)CCC2]
R¹ is selected from the group consisting of 3-chloro-phenyl, 4-chloro-phenyl, 3,4-difluoro-phenyl, 3,5-difluoro-phenyl, 3-methoxy-phenyl, 4-methoxy-phenyl, 2-methoxy-4-chloro-phenyl, 2-methoxy-5-bromo-phenyl, 2-methoxy-5-chloro-phenyl, 2-methoxy-5-fluoro-phenyl, 2-chloro-5-methoxy-phenyl, 2-fluoro-5-methoxy-phenyl, 3-chloro-4-methoxy-phenyl, 3-methoxy-4-chloro-phenyl, thien-2-yl, 5-chloro-thien-2-yl, 4,5-dichloro-thien-2-yl, 4,5-dimethyl-thienyl, 4-methyl-5-chloro-thien-2-yl, 2-methyl-thiazol-5-yl, 2,4-dimethyl-thiazol-5-yl, 1-methyl-pyrazol-3-yl, 1-methyl-pyrazol-4-yl, 1,3-dimethyl-pyrazol-4-yl, 1,5-dimethyl-pyrazol-4-yl, 1-methyl-pyrazol-5-yl, 1,3-dimethyl-pyrazol-5-yl, 5-chloro-6-methyl-pyrid-3-yl, 6-methoxy-pyrid-3-yl, 1-methyl-indazol-5-yl, 1-methyl-indazol-6-yl, benzofur-2-yl, benzothien-2-yl, benzothiazol-2-yl, 2-methoxy-benzoxazol-6-yl, benzoxazol-6-yl-2-one, quinolin-3-yl and 2,3-dihydrobenzo[b][1.4]dioxin-6-yl;
L¹ is selected from the group consisting of —CH₂—, —CH₂CH₂—, —OCH₂—, —NH—CH₂— and —N(CH₃)—CH₂—; wherein the —CH₂— portion of the L¹ group is bound to the double bond;
R² is selected from the group consisting of hydrogen and fluoro;
a is an integer from 0 to 1;
L² is —CH₂—;
R³ is selected from the group consisting of phenyl, 3-bromo-phenyl, 4-bromo-phenyl, 2,4-dichloro-phenyl, 3-trifluoromethyl-phenyl, 4-trifluoromethyl-phenyl, 4-methylsulfonyl-phenyl, 2-methyl-4-chloro-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl, 1-phenyl-pyrazol-4-yl, 1-(4-fluoro-phenyl)-piperidin-4-yl, 1-(benzyl)-piperidin-4-yl and 1-(pyrid-2-yl)-piperidin-4-yl;
or a stereoisomer or pharmaceutically acceptable salt thereof.

7. The compound of claim 4, wherein 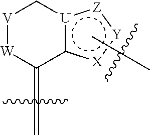
is an 8- to 10-membered, partially unsaturated ring structure selected from the group consisting of 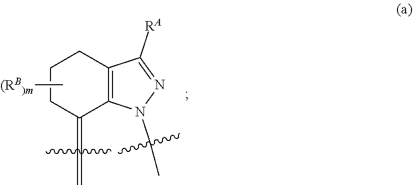
wherein R^{A} is hydrogen;
m is an integer from 0 to 2;
provided that when R^{A} is methyl, then m is 0;
each R^{B} is selected from the group consisting of 5-fluoro and 6-fluoro;
provided that when m is 1, R^{B} is 6-fluoro; provided further that when m is 2, both R^{B} groups are the same and are 5-fluoro; 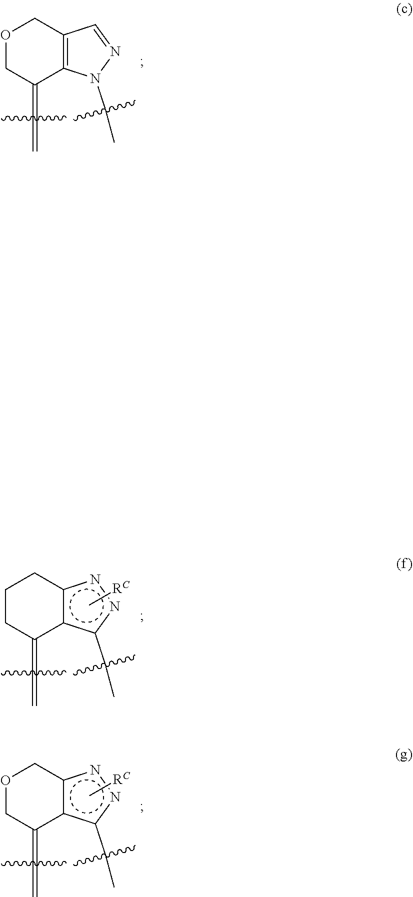
wherein R^{C} is bound to either nitrogen atom and is selected from the group consisting of hydrogen and methyl; 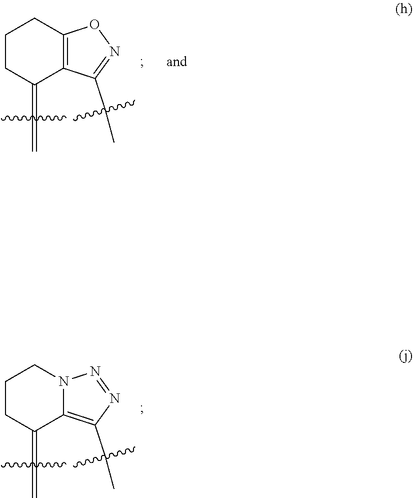 [SMILES: *c1noc2c1C(=*)CCC2 ; *c1nnn2c1C(=*)CCC2]
R¹ is selected from the group consisting of 4-chloro-phenyl, 3,4-difluoro-phenyl, 2-methoxy-4-chloro-phenyl, 2-methoxy-5-bromo-phenyl, 2-methoxy-5-chloro-phenyl, 3-chloro-4-methoxy-phenyl, 5-chloro-thien-2-yl, 4,5-dichloro-thien-2-yl, 4-methyl-5-chloro-thien-2-yl, 2,4-dimethyl-thiazol-5-yl, benzothien-2-yl, benzothiazol-2-yl, 2-methoxy-benzoxazol-6-yl and benzoxazol-6-yl-2-one;
L¹ is selected from the group consisting of —CH₂—, —CH₂CH₂—, —OCH₂—, —NH—CH₂— and —N(CH₃)—CH₂—; wherein the —CH₂— portion of the L¹ group is bound to the double bond;
R² is selected from the group consisting of hydrogen and fluor;
a is an integer from 0 to 1;
L² is —CH₂—;
R³ is selected from the group consisting of 4-bromo-phenyl, 2,4-dichloro-phenyl, 4-trifluoromethyl-phenyl, 4-methylsulfonyl-phenyl, 3-phenyl-phenyl, 3-(2,4-dichlorophenyl)-phenyl, 3-(4-fluoro-phenyl)-phenyl, 4-phenyl-phenyl, naphth-1-yl, naphth-2-yl, 2-phenyl-pyrimidin-5-yl, 6-methoxy-pyrid-3-yl and 1-(4-fluoro-phenyl)-piperidin-4-yl;
or a stereoisomer or pharmaceutically acceptable salt thereof.

8. The compound of claim 4, wherein 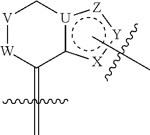
is an 8- to 10-membered, partially unsaturated ring structure selected from the group consisting of 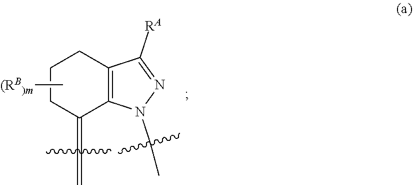
wherein R^{A} is hydrogen;
m is 0; 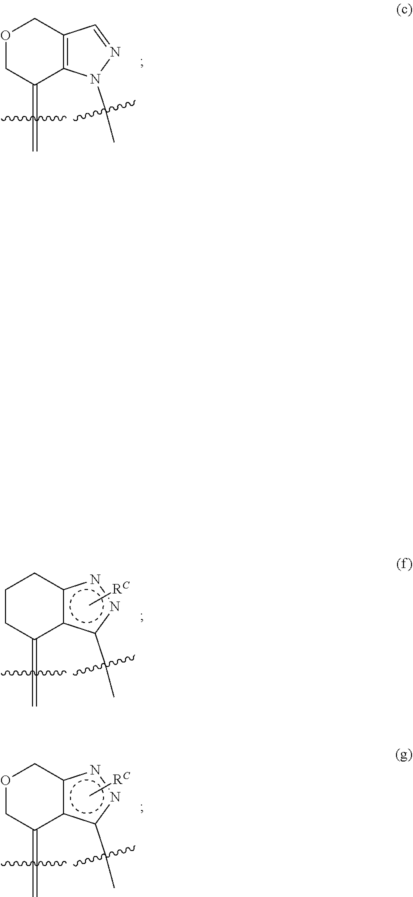
wherein R^{C} is bound to either nitrogen atom and is selected from the group consisting of hydrogen and methyl; 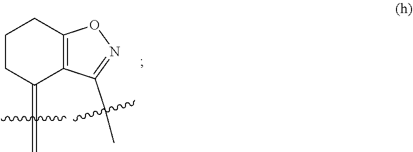 [SMILES: *c1noc2c1C(=*)CCC2]
and
R¹ is selected from the group consisting of 3-chloro-4-methoxy-phenyl, 5-chloro-thien-2-yl, 4,5-dichloro-thien-2-yl, 4-methyl-5-chloro-thien-2-yl, benzothiazol-2-yl, 2-methoxy-benzoxazol-6-yl and benzoxazol-6-yl-2-one;
L¹ is selected from the group consisting of —CH₂CH₂—, —OCH₂— and —NH—CH₂—; wherein the —CH₂— portion of the L¹ group is bound to the double bond;
R² is selected from the group consisting of hydrogen and fluoro;
a is an integer from 0 to 1;
L² is —CH₂—;
R³ is selected from the group consisting of 4-bromo-phenyl, 2,4-dichloro-phenyl, 4-trifluoromethyl-phenyl, 4-methylsulfonyl-phenyl, 3-phenyl-phenyl, 4-phenyl-phenyl, naphth-1-yl, naphth-2-yl and 1-(4-fluoro-phenyl)-piperidin-4-yl;
or a stereoisomer or pharmaceutically acceptable salt thereof.

9. The compound of claim 4, wherein 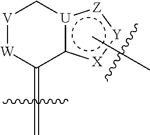
is an 8- to 10-membered, partially unsaturated ring structure selected from the group consisting of 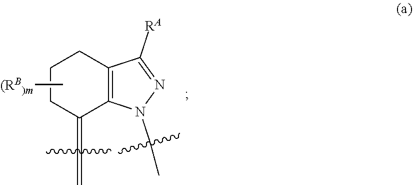
wherein R^{A} is hydrogen;
m is 0; 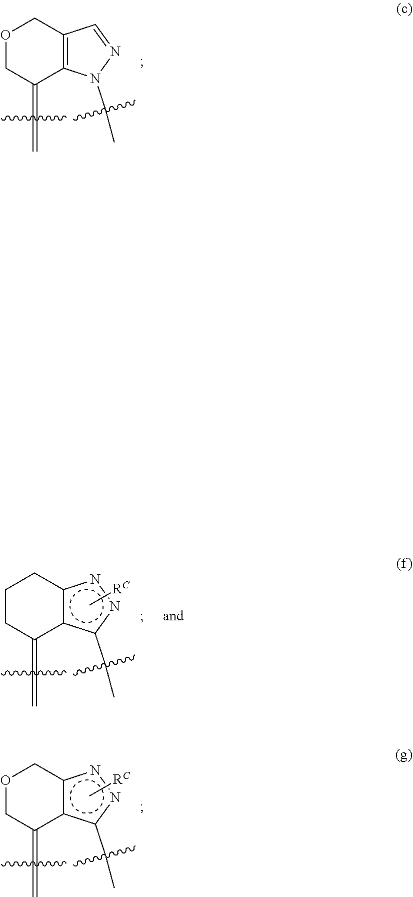
wherein R^{C} is hydrogen;
R¹ is selected from the group consisting of 4-chloro-phenyl, 3-chloro-4-methoxy-phenyl, 4,5-dichloro-thien-2-yl 4-methyl-5-chloro-thien-2-yl and 1-methyl-pyrazol-4-yl;
L¹ is —NH—CH₂—; wherein the —CH₂— portion of the L¹ group is bound to the double bond;
R² is selected from the group consisting of hydrogen and fluoro;
a is an integer from 0 to 1;
L² is —CH₂—;
R³ is selected from the group consisting of 2,4-dichloro-phenyl, 4-trifluoromethyl-phenyl and naphth-2-yl;
or a stereoisomer or pharmaceutically acceptable salt thereof.

10. The compound as in of claim 4, selected from the group consisting of
(E)-4-chloro-N-((2-(1-(2,4-dichlorobenzyl)-1,4,5,6-tetrahydro-7H-indazol-7-ylidene)ethyl)carbamoyl)benzenesulfonamide;
(Z)-4,5-dichloro-N-((2-(1-(2,4-dichlorobenzyl)-1,4,5,6-tetrahydro-7H-indazol-7-ylidene)-2-fluoroethyl)carbamoyl)thiophene-2-sulfonamide;
(E)-4,5-dichloro-N-((2-(1-(2,4-dichlorobenzyl)-1,4-dihydropyrano[4,3-c]pyrazol-7(6H)-ylidene)-2-fluoroethyl)carbamoyl)thiophene-2-sulfonamide;
(Z)-4,5-dichloro-N-((2-(1-(2,4-dichlorobenzyl)-1,4,5,6-tetrahydro-7H-indazol-7-ylidene)-2-fluoroethyl)(methyl)carbamoyl)thiophene-2-sulfonamide;
E)-4,5-dichloro-N-((2-(1-(4-(trifluoromethyl)phenyl)-1,4,5,6-tetrahydro-7H-indazol-7-ylidene)ethyl)carbamoyl)thiophene-2-sulfonamide;
Z)-3-chloro-N-((2-(1-(2,4-dichlorobenzyl)-1,4,5,6-tetrahydro-7H-indazol-7-ylidene)-2-fluoroethyl)carbamoyl)-4-methoxybenzenesulfonamide;
(Z)-5-chloro-N-((2-(1-(2,4-dichlorobenzyl)-1,4,5,6-tetrahydro-7H-indazol-7-ylidene)-2-fluoroethyl)carbamoyl)-4-methylthiophene-2-sulfonamide;
(E)-N-((2-(1-(2,4-dichlorobenzyl)-1,4,5,6-tetrahydro-7H-indazol-7-ylidene)ethyl)carbamoyl)-1-methyl-1H-pyrazole-4-sulfonamide;
E)-5-chloro-N-((2-(1-(2,4-dichlorobenzyl)-1,4-dihydropyrano[4,3-c]pyrazol-7(6H)-ylidene)-2-fluoroethyl)carbamoyl)-4-methylthiophene-2-sulfonamide;
(Z)-5-chloro-N-((2-(3-(2,4-dichlorobenzyl)-1,5,6,7-tetrahydro-4H-indazol-4-ylidene)-2-fluoroethyl)carbamoyl)-4-methylthiophene-2-sulfonamide;
E)-5-chloro-N-((2-(3-(2,4-dichlorobenzyl)-1,7-dihydropyrano[3,4-c]pyrazol-4(5H)-ylidene)-2-fluoroethyl)carbamoyl)-4-methylthiophene-2-sulfonamide;
and stereoisomers and pharmaceutically acceptable salts thereof.

11. A pharmaceutical composition comprising a pharmaceutically acceptable carrier and a compound of claim 1.

12-13. (canceled)

14. A method of treating a disorder mediated by the EP3 receptor, comprising administering to a subject in need thereof a therapeutically effective amount of the compound of claim 1.

15. The method of claim 14, wherein the disorder mediated by the EP3 receptor is selected from the group consisting of Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders, non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain and cancer.

16. A method of treating a disorder mediated by the EP3 receptor comprising administering to a subject in need thereof a therapeutically effective amount of the composition of claim 11.

17. A method of treating a condition selected from the group consisting of Type I diabetes mellitus, impaired glucose tolerance (IGT), impaired fasting glucose (IFG), gestational diabetes, Type II diabetes mellitus, Syndrome X (also known as Metabolic Syndrome), obesity, nephropathy, neuropathy, retinopathy, restenosis, thrombosis, coronary artery disease, hypertension, angina, atherosclerosis, heart disease, heart attack, ischemia, stroke, nerve damage or poor blood flow in the feet, neurodegenerative disorders, non-alcoholic steatohepatitis (NASH), non-alcoholic fatty liver disease (NAFLD), liver fibrosis, cataracts, polycystic ovarian syndrome, premature labor, irritable bowel syndrome, bladder over-activity, inflammation, pain and cancer comprising administering to a subject in need thereof, a therapeutically effective amount of the compound of claim 1.

18-25. (canceled)
